SEQUENCE LISTING (7 sequences)

SEQ ID NO: 1 - DNA, 1380 bp, Artificial Sequence
  HDNO D2
atggttagcagcaaactggcaacaccgctgagcattcagggtgaagttatttatccggatgatagcggttttgatgccattgccaatatttgggatggtcgtcatctgcagcgtccgagcctgattgcacgttgtctgagtgccggtgatgttgcaaaaagcgttcgttatgcatgtgataatggtctggaaatttcagttcgtagcggtggtcataatccgaatggttatgcaaccaatgatggtggtattgttctggatctgcgtctgatgaatagcattcatattgataccgcaggtagccgtgcacgtattggtggtggtgttattagcggtgatctggttaaagaagcagcaaaatttggtctggcagcagttaccggtatgcatccgaaagttggtttttgtggtctggccctgaatggtggtgtgggttttctgaccccgaaatatggcctggcaagcgataacattctgggtgcaaccctggttaccgcaacaggtgatgtgatttattgtagtgatgatgaacgtccggaactgttttgggcagttcgtggtgcaggtccgaattttggtgttgttaccgaagttgaagttcagctgtatgaactgcctcgtaaaatgctggcaggttttattacctgggcaccgagcgttagcgaactggcaggtctgctgaccagcctgctggatgcactgaatgaaatggcagatcatatctatccgagcgtttttgttggtgtggatgaaaatcgtgcaccgagtgttaccgtttgtgttggtcatctgggtggtctggatattgcagaacgtgatattgcacgtctgcgtggcctgggtcgtaccgttagcgatagcattgccgttcgtagctatgatgaagttgttgcgctgaatgcagaagttggtagctttgaagatggtatgagcaatctgtggattgatcgtgaaattgcaatgccgaatgcacgttttgcagaagcaattgcaggtaacctggataaatttgtgagcgaaccggcaagcggtggtagcgttaaactgttgattgatggtatgccgtttggtaatccgaaacgtacaccggcacgtcatcgtgatgcaatgggtgttctggcactggcagaatggtcaggtgcagcaccgggtagcgagaaatatcctgaactggcacgtgaactggatgcagcactgctgcgtgcgggtgttaccaccagtggttttggcctgctgaataataacagcgaagttaccgcagaaatggttgccgaagtgtataaaccggaagtttatagtcgcctggcagccgttaaacgtgaatatgatccggaaaatcgttttcgccacaactataacatcgatccggaaggtagctaa

SEQ ID NO: 2 - protein, 459 aa, Artificial Sequence
  HDNO D2
MVSSKLATPLSIQGEVIYPDDSGFDAIANIWDGRHLQRPSLIARCLSAGDVAKSVRYACDNGLEISVRSGGHNPNGYATNDGGIVLDLRLMNSIHIDTAGSRARIGGGVISGDLVKEAAKFGLAAVTGMHPKVGFCGLALNGGVGFLTPKYGLASDNILGATLVTATGDVIYCSDDERPELFWAVRGAGPNFGVVTEVEVQLYELPRKMLAGFITWAPSVSELAGLLTSLLDALNEMADHIYPSVFVGVDENRAPSVTVCVGHLGGLDIAERDIARLRGLGRTVSDSIAVRSYDEVVALNAEVGSFEDGMSNLWIDREIAMPNARFAEAIAGNLDKFVSEPASGGSVKLLIDGMPFGNPKRTPARHRDAMGVLALAEWSGAAPGSEKYPELARELDAALLRAGVTTSGFGLLNNNSEVTAEMVAEVYKPEVYSRLAAVKREYDPENRFRHNYNIDPEGS

SEQ ID NO: 3 - DNA, 1380 bp, Artificial Sequence
  HDNO D3
atggttagcagcaaactggcaacaccgctgagcattcagggtgaagttatttatccggatgatagcggttttgatgccattgccaatatttgggatggtcgtcatctgcagcgtccgagcctgattgcacgttgtctgagtgccggtgatgttgcaaaaagcgttcgttatgcatgtgataatggtctggaaatttcagttcgtagcggtggtcataatccgaatggttatgcaaccaatgatggtggtattgttctggatctgcgtctgatgaatagcattcatattgataccgcaggtagccgtgcacgtattggtggtggtgttattagcggtgatctggttaaagaagcagcaaaatttggtctggcagcagttaccggtatgcatccgaaagttggtttttgtggtctggccctgaatggtggtgtgggttttctgaccccgaaatatggcctggcaagcgataacattctgggtgcaaccctggttaccgcaacaggtgatgtgatttattgtagtgatgatgaacgtccggaactgttttgggcagttcgtggtgcaggtccgaattttggtgttgttaccgaagttgaagttcagctgtatgaactgcctcgtaaaatgctggcaggttttattacctgggcaccgagcgttagcgaactggcaggtctgctgaccagcctgctggatgcactgaatgaaatggcagatcatatctatccgagcgtttttgttggtgtggatgaaaatcgtgcaccgagtgttaccgtttgtgttggtcatctgggtggtctggatattgcagaacgtgatattgcacgtctgcgtggcctgggtcgtaccgttagcgatagcattgccgttcgtagctatgatgaagttgttgcgctgaatgcagaagttggtagctttgaagatggtatgagcaatctgtggattgatcgtgaaattgcaatgccgaatgcacgttttgcagaagcaattgcaggtaacctggataaatttgtgagcgaaccggcaagcggtggtagcgttaaactgttgattgatggtatgccgtttggtaatccgaaacgtacaccggcacgtcatcgtgatgcaatgggtgttctggcactggcagaatggtcaggtgcagcaccgggtagcgagaaatatcctgaactggcacgtgaactggatgcagcactgctgcgtgcgggtgttaccaccagtggttttggcctgctgaatcataacagcgaagttaccgcagaaatggttgccgaagtgtataaaccggaagtttatagtcgcctggcagccgttaaacgtgaatatgatccggaaaatcgttttcgccacaactataacatcgatccggaaggtagctaa

SEQ ID NO: 4 - protein, 459 aa, Artificial Sequence
  HDNO D3
MVSSKLATPLSIQGEVIYPDDSGFDAIANIWDGRHLQRPSLIARCLSAGDVAKSVRYACDNGLEISVRSGGHNPNGYATNDGGIVLDLRLMNSIHIDTAGSRARIGGGVISGDLVKEAAKFGLAAVTGMHPKVGFCGLALNGGVGFLTPKYGLASDNILGATLVTATGDVIYCSDDERPELFWAVRGAGPNFGVVTEVEVQLYELPRKMLAGFITWAPSVSELAGLLTSLLDALNEMADHIYPSVFVGVDENRAPSVTVCVGHLGGLDIAERDIARLRGLGRTVSDSIAVRSYDEVVALNAEVGSFEDGMSNLWIDREIAMPNARFAEAIAGNLDKFVSEPASGGSVKLLIDGMPFGNPKRTPARHRDAMGVLALAEWSGAAPGSEKYPELARELDAALLRAGVTTSGFGLLNHNSEVTAEMVAEVYKPEVYSRLAAVKREYDPENRFRHNYNIDPEGS

SEQ ID NO: 5 - protein, 8 aa, Artificial Sequence
  look up table sequence
GMFWKAIC

SEQ ID NO: 6 - protein, 8 aa, Artificial Sequence
  First 8 residues removed from PDB sequence
MELQQVLR

SEQ ID NO: 7 - protein, 4 aa, Artificial Sequence
  start of PDB protein sequence
MNGG

(19) United States
(12) Patent Application Publication
(10) Pub. No.: US 2024/0282401 A1
(43) Pub. Date: Aug. 22, 2024

(54) METHODS FOR ENZYME ENGINEERING

(71) Applicant: The University of Manchester, Manchester, Greater Manchester (GB)

(72) Inventors: Fabian Gilberto CANTU REINHARD, Manchester, Greater Manchester (GB); Andrew ALMOND, Manchester, Greater Manchester (GB)

(21) Appl. No.: 18/565,837

(22) PCT Filed: May 27, 2022
(86) PCT No.: PCT/GB2022/051366

§ 371 Date: Nov. 30, 2023

(30) Foreign Application Priority Data
Jun. 4, 2021 (GB) 2108011.4

(51) Int. Cl. G16B 5/00; G16B 20/50; G16B 40/20

(52) U.S. Cl. CPC G16B 5/00; G16B 20/50; G16B 40/20

(57) ABSTRACT

The present invention relates to computer-implemented methods for predicting catalytic activity for a candidate mutant enzyme comprising estimating the electrostatic component of the activation barrier for each of a plurality of conformations of each candidate mutant enzyme, for predicting catalytic activity for a candidate mutant enzyme using a machine learning model trained using data obtained using such methods, for providing a site directed mutagenesis potential map for an enzyme using the described methods, and for identifying a candidate enzyme with improved catalytic activity using the described methods. Related systems and computer-readable media are also described.

### FIELD OF THE INVENTION

The present invention relates to methods of predicting enzyme catalytic activity, methods of enzyme engineering using predictions of enzyme catalytic activity, and related methods and products.

### BACKGROUND

Enzymes are versatile catalysts that can accelerate key chemical reactions in vivo and in vitro and are found in a wide range of applications such as in the medical science for their use in diagnostic methods (e.g., PCR) or as drug targets, as well as in various industrial processes as efficient catalysts enabling sustainable chemical processes, for example by lowering energy requirements and waste production [1, 2]. Recently, more than 6300 enzyme classes had been reported for over 370000 different enzymes [117, 2]. However, key enzymes for desired chemical routes are frequently unavailable, for example, in active pharmaceutical ingredient (API) manufacture. Further, despite their natural diversity, considerable effort is often necessary to obtain new synthetic enzymes with enhanced properties such as thermal stability, catalytic activity, enantioselectivity and substrate scope. At present the de novo design of enzymes is in its infancy and the engineering process starts from an existing suboptimal enzyme that exhibits some of the required properties, and which is optimised using a process termed directed evolution.

Directed evolution (DE) is a process for developing bespoke biocatalysts, by mimicking natural selection and steering enzymes toward user-defined reaction conditions, substrate specificity and rates [83, 84]. This is performed by altering the enzyme amino acid building blocks using mutagenesis (and/or recombination), and then the generated mutants are screened for new activity (i.e., the resulting proteins are expressed, screened, and sequenced to identify variants with the desired property or activity). This process is repeated until the results are satisfactory, which normally requires several iterations [8]. Each step of DE requires the generation of a protein library by nucleic acid diversification, e.g., by using site-directed mutagenesis, error-prone polymerase chain reaction (PCR), DNA recombination, or de novo gene synthesis [13]. Methods such as error-prone PCR are intrinsically biased and consequently show a low efficiency in finding improved variants while the number of possibilities is astronomical, e.g., all mutations of amino acids at only ten positions are experimentally inaccessible as it is 20¹⁰ or ˜10¹³ Using site-directed mutagenesis experiments, specific sites of the enzyme are usually saturated using exhaustive and highly degenerate NNK codons, which produce libraries with every amino acid combination at a specific site. Large degenerate codons (such as NNK) create intrinsically non-uniform populations of amino acids [81,82]. This is exacerbated by amplification bias during PCR and the inclusion of stop codons, which results in libraries where intended variants are poorly represented, effectively meaning more screening is needed [83]. Furthermore, codons with high multiplicity allow only a few sites to be screened at each DE iteration because the libraries rapidly become unmanageable.

Notwithstanding the fact that reducing the multiplicity of the codons (even when targeting the same sites) can increase the success of DE [84], it also allows more sites to be targeted simultaneously. If sites, particularly outside the active site, and respective amino acids could be predicted in advance and correctly combined into a single library then this could significantly accelerate each DE iteration, saving resources and developing higher quality results and potentially delivering functional enzymes in fewer iterations. Moreover, focussed libraries exploring multiple beneficial sites may be able to discover synergistic epistatic effects (namely effects that are not obtained by the individual mutations but by the combination of two or more mutations due to interactions between amino acids) that can significantly improve enzyme turnover numbers. Methodologies that allow the construction and screening of multi-site combinatorial libraries with specified amino acid variability are already feasible using ligation-based, PCR-based or solid-phase full-length gene synthesis [13, 7, 85]. However, such methodologies are not fully exploited and conventional strategies in DE rely on randomly targeting enzyme residues for the generation and selection of potentially better mutant variants with varying degrees of success [120, 121].

Of the parameters that can be targeted for optimisation by DE, one of the most desirable parameters is the turnover number, but it can also be the most difficult to improve. The turnover number (also termed k_{cat}) is the number of chemical conversions of substrate that each catalytic site performs on average per second. Clearly, for any biocatalyst to be useful it needs an appreciable k_{cat} for the desired reaction. For enzymes, the catalysed chemical reaction occurs in a small region (the active site) and mutations in this area can affect both k_{cat} and substrate specificity. However, it is now understood that enzyme function involves enigmatic long-range allosteric effects and thus distal mutations also couple to the active site and are consequently also important in the DE optimisation process [9, 10]. Measuring k_{cat} and generating all possible mutations in a typical 500 amino acid enzyme is experimentally intractable. While optimisation and the design of efficient strategies of molecular biology methods, such as combinatorial active-site saturation test (CAST) [11] and iterative saturation mutagenesis (ISM) [12], have resulted in a reduction in screening effort, these methods are restricted to a handful of amino acids in the active site [13]. Newer synthetic biology approaches allow libraries of genes to be produced relatively easily, but again the number of possibilities for all mutations of amino acids at only ten positions is experimentally inaccessible as it is 20¹⁰ or ˜10¹³ [14].

Despite some progress, the DE process remains slow and expensive, with often only limited results because of the astronomical number of possibilities that exist in any enzyme sequence. Therefore, a better process is urgently needed to improve the rate at which enzymes are engineered for their incorporation into more sustainable processes.

The present invention has been devised in light of the above considerations.

### SUMMARY OF THE INVENTION

The inventors have recognised that to fully exploit the potential of methodologies that allow the construction and screening of multi-site combinatorial libraries with specified amino acid variability requires theoretical methods to direct the enzyme engineering process by allowing effective genetic variability to be introduced into the library at each step. The inventors have further recognised that theoretical approaches are vital to navigate sequence space intelligently and to effectively utilise improved and emergent experimental DE approaches [7, 15]. Although a range of rational information from protein sequence, 3D-structure, quantum mechanics (QM) and prior experimental data have been used to build efficient genetic variability into DE libraries with varying degrees of success [86-92, 19-22], the inventors recognised that production of libraries that can yield improvements in k_{cat} from multiple sites (particularly outside the active site) in a single step remains a challenge.

The inventors have further recognised that existing theoretical methods for estimating k_{cat} suffer from multiple drawbacks particularly when considered in the context of the problems to be solved in enzyme engineering. Density functional theory (DFT) cluster approaches, where only a subset of the enzyme (assumed to be most relevant to catalysis) is included in the models, allow for accurate relative-energy estimations of reaction barriers. However, these are limited to predicting the effect of mutagenesis on rate for those residues included in the cluster models (often limited to the active site) and require extensive computational resources for each mutant calculation. Alternatively, multi-scale approaches such as quantum mechanics/molecular mechanics (QM/MM) methods model different regions or phenomena at different levels of theory, allowing for larger regions to be calculated and some conformational dynamics to be considered through proper sampling. However, to calculate a representative set of structures and capture the dynamical effects of an enzyme with QM/MM would currently require vast computational resources. Molecular dynamics (MD) simulations of small proteins can access real milliseconds, but MD cannot alone fully predict catalytic phenomena. None of these approaches has been able to elucidate the contribution of enzyme dynamics and distal mutations to catalysis.

The inventors have developed a methodology that combines global enzyme dynamics and electrostatics for the prediction of k_{cat}, providing predictions that are able to sense changes in k_{cat} as the conformation and dynamics of the enzyme are altered by even distal mutations. The method uses MD to provide enzyme dynamics and then uses QM approximations to estimate catalytic energetics, thereby combining the main benefits of the two approaches. A series of QM/MM or DFT calculations followed by electrostatic calculations (based on conformations from MD simulations) are used to investigate the contribution of whole enzyme conformational and dynamical effects to the turnover rate. This is achieved by rapidly calculating the contribution to enzymatic turnover rate at every timepoint in a variable timescale, for example, a microsecond timescale MD trajectory or a 1 ns timescale, by approximating the free energy of activation from electrostatics. The inventors demonstrated (see Example 1) that the relative electrostatic component of k_{cat} can be predicted by calculating and averaging the distribution of the activation barriers from substantial numbers of dynamic conformations. The inventors further demonstrated the use of the newly developed methodology in mutants of 6-hydroxy-D-nicotine oxidase from Arthrobacter nicotinovorans, an EC1 class enzyme (oxidoreductase) and an important target for biocatalysis is the production of chiral amines, found in many active pharmaceutical ingredients (APIs).

The inventors further recognised that the newly developed methodology for the prediction of enzyme activity based on the dynamic and electrostatic effects of mutations could be used as an objective function to rank candidate mutations in a DE process, to reduce and enrich the essentially infinite mutational space of the enzyme. They demonstrated this by computationally analysing and ranking a series of mutations of 6-HDNO. Using these computational results, a functionally enhanced library is constructed employing small degenerate codons to target several sites simultaneously by using PCR-based full-length gene synthesis. Following a single screening, a variant with a significant increase in activity was found containing three amino acid substitutions outside the active site (Example 2). They further demonstrated that the approach was compatible with site directed mutagenesis and enzyme stabilization methods. Indeed, applying this combination to the 6-HDNO example, they were able to produce a fast and stable 6-HDNO derivative with a total of eight mutations from the wild type.

Further, the inventors recognised that the newly developed rapid rational computational methodology for the estimation of enzymatic activity based on molecular dynamics and electrostatics could enable the generation of large and diverse mutant datasets that could in turn be used as a basis to train a newly developed machine learning (ML) based approach for the rational design of DE libraries. Thus, in Example 3, a ML based approach to rationally drive DE experiments based on an unprecedented, larger, and more diverse dataset of over 360,000 mutants and molecular dynamics simulations generated from a series of distinct starting conformations of 6-HDNO is described. A series of ML ProSAR (protein sequence activity relationship) models were then used to model the data and produce global predictions with the aims of designing efficient DE libraries capable of discovering better and otherwise concealed enzyme variants. The efficacy of the process is experimentally validated by generating two different rationally designed and novel DE libraries exhibiting several active mutants.

In Examples 4 to 7, the inventors further validated this approach using different classes of enzymes including EC2 (transferase, Example 6), EC3 (hydrolase, Example 4), EC4 (lyase, Example 7) and EC5 (transferase, Example 5). Furthermore, in these examples some demonstrations are made to some process parameters sub-processes which may be effortlessly changed to equivalent variations, for example, in the use of equivalent software, e.g., specific computational chemistry methodologies (e.g., QM/MM and DFT cluster optimisations, DFT functionals, basis sets, or implicit solvents), length of molecular dynamic simulations (e.g., 1 ns or 50 ns), number of mutants in a database (e.g., 1000, 50000 or 360000), inclusion of solvent molecules, ML learning variations (e.g., linear regression, artificial neural networks), number of individual mutations on each mutant (e.g., 3, 6, 12, 24, 48 or more), number of seed conformations (e.g., 1, 5 or 10), number of models on each ensemble of ML models (e.g., 1, 30 or more).

Some ML methods have been described previously for the acceleration of DE processes but have suffered from major limitations. While experimental methodologies can test several thousand variants in a single screening step, a major problem stands in obtaining sufficient reliable and diverse experimental data for ML models to train on. Fitting ML models to experimental data requires a set of fully sequenced mutant variants, where each have been independently measured for the properties of interest. However, in standard DE, only the most active variants are sequenced after some initial fast and more economical screening step. Thus, obtaining sufficient experimental data to fit ML models may be very restrictive, often making random processes more attractive than ML-driven DE. Moreover, experimental ML models can only be introduced at later stages of the DE process, therefore giving even the most rigorous DE processes a slow start. Additionally, this limits the application of the ML-driven methods to a partial subset of the protein evolution landscape, as only a fraction of the protein is targeted in those later stages of DE where sufficient data is collected.

The present invention depends on a rational computational methodology to estimate catalytic activity based on protein sequences and structural data and provides an efficient alternative to experimentally led ML data generation. In particular, the invention provides a computational strategy that is fast enough to circumvent conformational sampling problems typically found in computationally based rate estimations and allows the generation of a large and diverse dataset of mutants, making it of practical use to fit ML models to automatically guide DE experiments and accelerate the discovery of new and otherwise undetected enzyme variants throughout the sequence of the enzyme. The invention finds use in protein engineering in general and applications such as the manufacture of APIs in particular.

According to a first aspect, the invention provides a method of predicting catalytic activity for a candidate mutant enzyme, wherein the candidate mutant enzyme differs from a reference enzyme by one or more amino acids, the method comprising:
providing a set of parameters from a molecular simulation of the reference enzyme, wherein a region of the enzyme (QM region) comprising at least part of the active site and a substrate of the enzyme is optimised with a quantum mechanics method;
performing a molecular dynamics simulation with the candidate mutant enzyme and a substrate of the enzyme to obtain a plurality of conformations each associated with a set of atomic coordinates;
estimating the electrostatic component of the activation barrier (ΔΔG^{‡}₂₀) for each of the plurality of conformations of the candidate mutant enzyme, using the parameters from the molecular simulation of the reference enzyme and the set of atomic coordinates associated with the respective conformation, thereby obtaining a plurality of estimates of the electrostatic component of the activation barrier (ΔΔG^{‡}_{Q20}); and determining a score (ΔΔG^{‡}_{Q20EFF}, μ_{Q20}) based on the plurality of estimates of the electrostatic component of the activation barrier, wherein the score is indicative of the effective activation barrier (ΔΔG^{‡}) of the candidate mutant enzyme.

Thus, according to the methods of the invention, the catalytic activity of any candidate mutant enzyme, including in particular candidate comprising mutations outside of the active site, can be rapidly predicted using parameters previously obtained from quantum mechanical calculations for a reference enzyme. The process can be repeated for any number of candidate mutant enzymes, re-using the same set of quantum mechanical calculations. Thus, large sets of candidate mutant enzymes comprising mutations throughout the sequence of the enzyme can be rapidly evaluated for the catalytic activity. In other words, the present methods only require quantum mechanical calculations to be made in the initial set up and not during routine prediction. During routine predictions, the molecular dynamics simulation provides enzyme dynamics information allowing to follow the transition state barrier as a function of time (combined with parameters obtained from the quantum mechanical calculations about the transition state barrier). This information can be combined into a single score that is indicative of the effective activation barrier of the candidate mutant enzyme.

The method may further comprise defining a core region that includes one or more of the atoms of the QM region, and an external region that includes the remaining atoms of the enzyme. In such embodiments, the set of parameters from the molecular simulation of the reference enzyme may comprises: the changes to the partial charges of the atoms in the core region (ΔQᵢ) that occur during the formation of the transition state for a particular conformation of the reference enzyme from the reaction complex, and partial atomic charges for atoms in the external region. A change in partial atomic charges for each atom in the core region may be obtained for each of a plurality of conformations. A representative change of partial atomic charges for each atom in the core region may be obtained as the mean value across each of the plurality of conformations. The change in charges may be calculated via a population analysis method including Mulliken population analysis, Hirshfeld population analysis, CM5 population analysis. The core region may include all of the atoms of the QM region. The core region may include a subset of atoms of the QM region. The subset of atoms of the QM region may include at least the atoms of the substrate. The subset of atoms of the QM region may include the atoms of the substrate and a subset of atoms that take part in a postulated reaction mechanism catalysed by the enzyme. These atoms may have been previously identified to participate in the chemical reaction. The subset of atoms of the QM region may include the atoms of the substrate and any atoms where a significant change in partial atomic charges has been determined to occur between the transition from the RC structure to the TS structure, based on a partial atomic charge calculation.

The set of parameters from a molecular simulation of the reference enzyme may have been obtained by optimising a reaction complex and a transition state using any electronic structure method, such as a QM/MM or DFT cluster model. The set of parameters from a molecular simulation of the reference enzyme may have been obtained by calculating the charges in the QM region (including the core region) in the reactant state (reaction complex) and in the transition state configuration. The QM/MM model may be electrostatically embedded. The difference of partial atomic charges may have been calculated using any method for the calculation of partial atomic charges.

The parameters from the molecular simulation of the reference enzyme may comprise the partial charge difference between the transition state and the reaction complex for each atom of the core region (AQᵢ) and estimating the electrostatic component of the activation barrier for a conformation of the candidate mutant enzyme may comprises calculating electrostatic Coulombic interactions between: each atom of the external region; and the partial charge difference between the transition state and the reaction complex for each atom of the core region. Estimating the electrostatic component of the activation barrier for a conformation of the candidate mutant enzyme may comprise summing the electrostatic Coulombic interactions over all pairs of external and core atoms. This may be performed using Equation (5):

$$\\ \begin{matrix}Δ Δ {G}_{Q 20}^{{}_{} ‡} = c {\sum }_{j ∈ external} {\sum }_{i ∈ core} \frac{{q}_{j} Δ {Q}_{i}}{{r}_{ji}} & ( 5 )\end{matrix}$$

where ΔΔG^{‡}_{Q20} is the estimate of the electrostatic component of the activation barrier, qⱼ is the partial charge for atom j of the external region, ΔQᵢ is the partial charge difference between the transition state and the reaction complex for atom i of the core region, external region and distances to the core atoms rⱼᵢ is the distance between atoms i and j in the set of atomic coordinates associated with the conformation, and c is a constant. The constant c may be calibrated to provide energy in kcal×mol⁻¹. For example, the constant c may be set to is 332/e² kcal×Å×mol⁻¹.

The score may be indicative of the turnover number of the candidate mutant enzyme. The turnover number may be exponentially dependent on the score for the candidate mutant enzyme. The method may further comprise obtaining a score based on the score indicative of the turnover number and one or more other properties. The one or more properties may be selected from: stability (e.g. thermal stability), pH tolerance, and substrate diffusion to the active site. Advantageously, the one or more other properties may include stability.

Determining a score (ΔΔG^{‡}_{Q20EFF}, μ_{Q20}) based on the plurality of estimates of the electrostatic component of the activation barrier may comprise calculating one or more statistical parameters of the distribution of estimates of the electrostatic component of the activation barrier (ΔΔG^{‡}_{Q20}) for the plurality of conformations of the candidate mutant enzyme. The statistical parameters may comprise the average (μ_{Q20}) and the standard deviation (σ_{Q20}) of the distribution of estimates. Determining the score (ΔΔG^{‡}_{Q20EFF}) may comprises using Equation (2):

$$\\ \begin{matrix}Δ Δ {G}_{Q 20 EFF}^{{}_{} ‡} = {μ}_{Q 20} - \frac{{σ}_{Q 20}^{{}_{} 2}}{2 RT} & ( 2 )\end{matrix}$$

wherein μ_{Q20} is the average and σ_{Q20} is the standard deviation of the distribution of estimates, and RT is the product of the gas constant and temperature. The product of the gas constant and temperature may be set to 0.593 kcal×mol⁻¹, assuming a standard temperature and pressure. The statistical parameters may comprise the average (μ_{Q20}) and the score may be the average (μ_{Q20}) or may be based on the average as the only statistical parameter of the distribution of estimates of the electrostatic component of the activation barrier.

Performing a molecular dynamics simulation with the candidate mutant enzyme and substrate may comprise performing a molecular dynamics simulation with the candidate mutant enzyme, the substrate and one or more cofactors. Performing a molecular dynamics simulation with the candidate mutant enzyme and substrate may comprise performing a molecular dynamics simulation with the candidate mutant enzyme, substrate and any cofactor in a near attack conformation. Performing a molecular dynamics simulation with the candidate mutant enzyme and substrate may comprise performing a molecular dynamics simulation using one or more harmonic constraints that maintain the enzyme, the substrate and any cofactors in a near attack conformation. A near attack conformation may be defined as a conformation that can directly convert into a transition state structure, according to an assumed reaction mechanism for the enzyme. In the near attack conformation, the substrate may be in a near attack position and the induced polarizability of the enzyme and water may act to reduce the energy required to form the active complex. Imposing a restraint on the MD simulation to hold the substrate in a near attack conformation has the effect that only the distribution of the electrostatic effects of the enzyme towards stabilizing the active complex are observed.

The candidate mutant enzyme may differ from the reference enzyme by one or more amino acids. The candidate mutant enzyme may differ from the reference enzyme by one or more amino acids outside of the active site. The candidate mutant enzyme may differs from the reference enzyme by 1, 2 or 3 amino acids, by up to 6 amino acids, by up to 12 amino acids, by up to 24 amino acids, or by 1, 2, 3, 6 or 12 amino acids. Performing a molecular dynamics simulation with the candidate mutant enzyme and substrate may comprise performing a molecular dynamics simulation for a period of at least 0.1 ns, at least 1 ns, at least 5 ns, at least 10 ns, at least 20 ns, at least 30 ns, at least 40 ns, about 1 ns or about 50 ns. The plurality of conformations may correspond to a plurality of times of the molecular dynamics simulation. Performing a molecular dynamics simulation with the candidate mutant enzyme and substrate may comprise obtaining a conformation from a molecular dynamics simulation of the reference enzyme, and substituting the one or more mutant amino acids in the conformation. Performing a molecular dynamics simulation with the candidate mutant enzyme and substrate may further comprise performing a molecular dynamics for a period of time to allow the conformation to equilibrate prior to obtaining the plurality of conformations and/or performing simulated annealing to remove steric clashes involving mutated residues and/or performing a rotamer conformation search and minimisation to remove steric clashes.

The method may further comprise providing the score or information derived therefrom, to a user through a user interface, to a database or other computer readable storage medium, or to a computing device such as e.g. for further processing, analysis or use.

According to a further aspect, there is provided a method of predicting catalytic activity for a candidate mutant enzyme, wherein the candidate mutant enzyme differs from a reference enzyme by one or more amino acids, the method comprising: providing a candidate mutant enzyme as an input to a machine learning model that has been trained to take as input a candidate enzyme sequence and produce as output a score indicative of the effective activation barrier of the candidate mutant enzyme, wherein the machine learning model has been trained using training data comprising a plurality of candidate mutant enzyme sequences and corresponding scores indicative of the effective activation barrier (ΔΔG^{‡}) of the candidate mutant enzyme obtained using the method of any embodiment of the preceding aspect.

Prior to the present invention, the ability to use machine learning for identifying mutation sites throughout a protein sequence that are likely to result in mutated enzymes with improved catalytic activity was limited at least in part by the lack of availability of suitable data (i.e., data relating protein sequence to activity) to train such machine learning algorithms. Training machine learning algorithms requires large amounts of data, which is not commonly generated experimentally for mutated enzymes (particularly for mutations outside of the active site), and no theoretical prediction of catalytic activity could be obtained at the sort of scale that is needed for a machine learning model to learn useful information about the relationship between mutations outside of the active site and catalytic activity. The use of the methods described herein to predict changes in catalytic activity throughout the sequence of an enzyme enables the rapid generation of vast data sets relating mutations to predicted changes in catalytic activity, which can be used to train machine learning models to predict the likelihood that any candidate mutation or position will be associated with an improved catalytic activity. This can be used to prioritise directed evolution efforts, for example by rationally designing experimentally manageable enriched synthetic biology libraries of genes using degenerate codons or codon mixtures. Importantly, this approach overcomes the protein engineering problem of getting trapped in otherwise inescapable local optima in directed evolution experiments, which frustrates progress toward significant turnover number (kcat) improvements. The method can study candidate mutations outside the active site, and throughout large regions (or even the whole sequence of the enzyme), avoiding getting stuck in sometimes unproductive active site focussed optimisation.

The machine learning model may comprise a plurality of individual machine learning models wherein each individual machine learning model has been trained to take as input a candidate enzyme sequence and produce as input a score indicative of the effective activation barrier of the candidate mutant enzyme. The machine learning model may comprise one or more ensembles of individual machine learning models. The scores produced for the same sequence as output by each individual machine learning model in an ensemble may be combined into a single score for each ensemble, for example a mean or median score. The machine learning process may comprise more than one individual machine learning models to model a specific set of data sourced from a particular seed conformation. The inventors have found that using ensembles of models resulted in better prediction accuracy than individual models. The inventors have further identified that the gain associated with using ensembles of models may reduce above a certain number of individual models per ensemble. The optimal number of individual models in an ensemble may depend on the enzyme, the configuration and type of the machine learning model and how the mutant enzyme data is encoded. For example, as will be explained further below, the machine learning model may comprise a plurality of ensembles each trained using data obtained with the same seed conformation, and each ensemble using data obtained using a different seed conformation. In such cases, the number of individual models per ensemble above which adding further models no longer significantly improves performance (which can be referred to as the optimal number of individual models) may depend on the number of different seed conformations used. Each ensemble may have the same number of individual machine learning models. All the individual machine learning models may have the same architecture. Each individual machine learning model may have been independently trained. In other words, the parameters of the individual machine learning models may differ (due to training), even where the general architecture of the individual machine learning models is the same. Each individual machine learning model may have been independently trained using a subset of the training data. The subsets may be partially overlapping. For example, each individual machine learning model in an ensemble may have been trained using a randomly selected subset of the training data used to train the models in the ensemble.

Each individual machine learning model may have been trained using training data comprising a plurality of candidate mutant enzyme sequences and corresponding scores indicative of the effective activation barrier of the candidate mutant enzyme obtained using the method of any embodiment of the first aspect, wherein the scores have been obtained by performing a molecular dynamics simulation with the candidate mutant enzyme and substrate using the same starting conformation from a molecular dynamics simulation of the reference enzyme. The machine learning model may comprise individual machine learning models that have been trained using training data comprising scores that have been obtained by performing a molecular dynamics simulation with the candidate mutant enzyme and substrate using a respective starting conformation from a molecular dynamics simulation of the reference enzyme, wherein the respective starting conformations used for at least two of the individual machine learning models are different from each other. Thus, the scores provided as part of the training data for each individual model may have been obtained by performing a molecular dynamics simulation for each candidate mutant enzyme in the training data using the same seed conformation from the reference enzyme as a starting point. The present inventors have identified that the individual performance of machine learning models is improved by maintaining the same seed conformation for all the training data used by the respective model, so that each model can learn differences that are due to the mutations without confounding effects due to differences between the seed conformations. The seed conformation used to obtain the training data for different individual machine learning models may be different. The present inventors have identified that individual machine learning models trained using data from one seed conformation perform better at predicting test scores obtained using the same seed conformation than other seed conformations. Further, the inventors have identified that the training data is more representative of the properties of the enzyme when combining the molecular dynamics simulations produced from a plurality of seed conformations and thus the machine learning model performed better (i.e., had a higher prediction accuracy) when combining predictions from models trained from a variety of seed conformations. However, the present inventors have also demonstrated that useful predictions could be obtained using a single seed conformation.

As described above, a molecular dynamics simulation of the reference enzyme may have been performed to identify a near attack conformation from which a quantum mechanics/molecular mechanics simulation can be performed to identify the set of parameters from a molecular simulation of the reference enzyme. The molecular dynamics simulation may be a relatively long molecular dynamics simulation, such as e.g., a 10 μs simulation. When a plurality of seed conformations is used, these may be selected to span from any point or period within a simulation, for example by selecting seed conformations at regular intervals within a time period. The seed conformation may be used by substituting the one or more mutant amino acids in the seed conformation. The modified conformation thus obtained may be used to perform a molecular dynamics simulation for a period of time to allow the conformation to equilibrate prior to obtaining the plurality of conformations for the candidate mutant enzyme (from which the score is calculated). Instead or in addition to this, the modified conformation thus obtained may be used to perform simulated annealing to remove steric clashes involving mutated residues, prior to obtaining the plurality of conformations for the candidate mutant enzyme (from which the score is calculated).

The machine learning model may comprise a plurality of ensembles of individual machine learning models, wherein each individual machine learning model has been trained using training data comprising a plurality of candidate mutant enzyme sequences and corresponding scores obtained by performing a molecular dynamics simulation with the candidate mutant enzyme and substrate using the same starting conformation from a molecular dynamics simulation of the reference enzyme. Each respective one of the plurality of ensembles of individual machine learning models may comprise individual machine learning models that have been trained using training data comprising scores obtained by performing a molecular dynamics simulation with the candidate mutant enzyme and substrate using a respective starting conformation from a molecular dynamics simulation of the reference enzyme. Thus, each ensemble of models may comprise models trained using training data comprising candidate mutated enzyme sequences and scores calculated using the same seed conformation from the reference enzyme. The model may comprise ensembles each comprising models trained using scores calculated using a seed conformation that is different from the seed conformation used for another ensemble. The machine learning model may have been trained using training data comprising a plurality of candidate mutant enzyme sequences and corresponding scores obtained by performing a molecular dynamics simulation with the candidate mutant enzyme and substrate using one of between 5 and 10 starting conformation from a molecular dynamics simulation of the reference enzyme. The inventors have identified data generated from specific conformations may not fully predict mutations toward a different conformation so there are benefits in aggregating predictions from each ensemble of conformational models to have predictions based on several sampled conformations.

The training data may have been obtained by performing molecular dynamics simulation of the plurality of candidate mutated enzymes, each molecular dynamics simulation having the same length, such as e.g. between 1 and 5 ns. The inventors have identified that using longer molecular dynamics simulation may improve the accuracy of the score that is used in the training data, which may in turn increase the accuracy of the predictions made by the machine learning model. However, within computation constraints, there is a trade-off between using more diverse seed conformations and performing longer molecular dynamics simulation. Each of these may improve the accuracy of the scores and of the predictions from the machine learning model. The choice of a number of seed conformations and a length of molecular dynamics simulation may therefore be performed for every particular system in view of the performance of the model with various combinations of these parameters that fit within available computational resources.

The scores produced by each individual machine learning model or the combined scores produced by each ensemble may be standardised. For example, the scores may be standardised using parameters defined based on scores obtained for a common set of mutant enzyme sequences. The common set of mutant enzyme sequences may comprise candidate mutant enzymes with mutations that together cover any position associated with a mutation in a candidate enzyme for which a prediction is to be obtained. Standardising the scores for an individual model or an ensemble of model may comprise identifying the mean and variance of the distribution of scores (or combined scores, in the case of an ensemble) obtained by predicting the scores for a set of mutant enzymes, and scaling and centring any score produced by the individual model or ensemble using the identified mean and variance. It is possible to use a diverse set of mutant enzymes which cover without bias the entire space where mutations may be generated. This may include a dataset where every single amino acid substitution is reflected in a single mutation for each position in the sequence, a dataset where every single amino acid substitution for each position is reflected in one of a plurality of mutations present in each mutant enzyme sequence, or a set of randomly generated multiple mutants (e.g., a list of triple mutants covering all the space with equal probability). In other words, the scores may be standardised to have an expected mean of 0 and an expected variance of 1. Standardising the scores means that the scores now represent the relative effect on catalytic activity of each candidate mutated enzyme. This enables scores to be compared across individual models or ensembles which may otherwise have different means and variances. This is particularly advantageous when individual models or ensembles are trained using data that uses different seed conformations, as the scores may then not be directly comparable across individual models/ensembles (since they reflect both an effect of the mutations and an effect of the seed conformation on the calculation of the electrostatic component of the activation barrier.

The (optionally standardised) combined scores produced for the same sequence by each ensemble of individual machine learning models may be combined into a single score for each candidate enzyme sequence, for example a mean or median score. The machine learning model may have been trained using training data comprising a plurality of candidate mutant enzyme sequences that each differ from the same reference enzyme by more than one amino acid, or by at least 1, at least 2, at least 3, between 3 and 6, between 3 and 24, between 3 and 48, between 3 and 12, 1, 2, 3, 4, 5, 6, 12, 24 or 48 amino acids. The machine learning model may have been trained using training data comprising at least 1000, at least 10,000, at least 50,000, at least 100,000, at least 200,000 or at least 300,000 candidate mutant enzyme sequences. The machine learning model may have been trained using training data comprising a plurality of candidate mutant enzyme sequences that differ from the reference enzyme by at least one amino acid, wherein the plurality of candidate mutant enzyme sequences together comprise mutations at each position of the reference enzyme apart from excluded positions. For example, excluded positions may comprise one or more of key catalytic residues, cysteine residues, N terminus residues and C terminus residues. Each candidate mutant enzyme may comprise one or more randomly selected mutations at a randomly selected position. The machine learning model or each of the individual machine learning models may be selected from: a regression model, optionally a linear regression model or derivative thereof such as a multiple linear regression model or a Lasso regularised linear regression model, a support vector regression model, and a neural network model such as a dense neural network model. Multiple mutants such as e.g. triple mutants may advantageously be used to increase the sampling density for any number of candidate mutant enzymes in the training data. For example, a training data set comprising approximately 360,000 triple mutants may effectively cover about 1 million single mutations. The number of mutants used in the training data may depend on a variety of factors including the computational resources available, the desired accuracy of the prediction, the type of machine learning model, the length of the MD simulations used, and the length of the enzyme. For example longer MD simulations may provide more accurate measurements such that similar predictions can be obtained with fewer mutants. As another example, enzymes with shorter sequences have smaller mutant spaces than longer enzymes.

The machine learning model or each individual machine learning model takes as input a candidate enzyme sequence that is encoded using an encoding dictionary where each amino acid is represented by a vector of size N. Each element of the vector may be an amino acid property from a randomly selected set of amino acid properties, optionally from the AAindex amino acid properties database. Alternatively, each element of the vector may be a random number, optionally wherein the real random number is selected between 0 and 1. Alternatively, each element of the vector may be a 0 or a 1, wherein the vector has size N equal to the number of different amino acids considered, and each vector contains a single 1 or a single 0 at a position specific for the amino acid being encoded. In other words, the enzyme sequence may be one hot encoded by a vector of size N=20 (if 20 amino acids are considered) which contains only zeros except for the amino acid being encoded, which is represented by a 1. N may be >20 for example where the number of distinct amino acid variants include additional variations to the natural amino acids, such as specific protonation states, chemical modifications and non-natural amino acids. Alternatively, each element of the vector may be a 0 or a 1, wherein the vector has size N=1, and the element is equal to 0 if the residue is not mutated and 1 otherwise, or vice-versa. Alternatively, the full enzyme sequence may be encoded by a single vector of length equal to the number of residues where each mutant is encoded to contain the same number (e.g., 0) except for the position where a mutation or mutations have been inserted and which a different number (e.g., 1) is used. This may be advantageous where relatively small amounts of mutant data are used. Relatively small amounts of mutant data may be used when computational resources and/or time are limited, for example to enable the use of longer MD simulations (e.g. 50 ns). Regardless of the encoding dictionary used, the resulting encoded sequence of numbers may be subject to a fast Fourier transform procedure for each encoded vector and the real part of the FFT result is used to encode the protein sequence data. As the skilled person understands, references to 0 and 1 encompass any pair of values that can be identified as two different states (i.e. any Boolean set).

The encoding dictionary may be defined independently for each individual machine learning model or ensemble of machine learning models. Each of these encoding strategies introduces variability into the models. The random number strategy takes into account similarity between amino acids when the amino acids are identical, but not otherwise. By contrast, strategies based on amino acid properties may preserve information regarding the similarity of amino acids even if not identical. The present inventors have found the random strategies to perform as well or better than properties-based encoding dictionaries. Further, the present inventors have found that random encoding strategies could be optimised, for example by selecting those random encoding dictionaries (complexity and/or values) that were associated with the best performing individual machine learning models.

The method of the present aspect may be repeated for a plurality of candidate mutated enzymes, thereby obtaining a score indicative of the effective activation barrier of each of the plurality of candidate mutant enzymes. The scores may together form a site directed mutagenesis potential map.

According to a third aspect, there is provided a computer-implemented method of providing a tool for predicting catalytic activity for a candidate mutant enzyme, wherein the candidate mutant enzyme differs from a reference enzyme by one or more amino acids, the method comprising: providing training data comprising a plurality of candidate mutant enzyme sequences and corresponding scores indicative of the effective activation barrier of the candidate mutant enzyme obtained using the method of any embodiment of the first aspect; and training a machine learning model to take as input a candidate enzyme sequence and produce as output a score indicative of the effective activation barrier of the candidate mutant enzyme.

The method of the present aspect may have any of the features described in relation to the previous aspect. In particular, the method may comprise defining one or more encoding dictionaries as described above, defining one or more standardisation parameters as described above, etc.

According to a fourth aspect, there is provided a method of providing a site directed mutagenesis potential map for a reference enzyme, the method comprising: providing a plurality of candidate mutated enzymes, wherein the candidate mutant enzyme differs from the reference enzyme by at least one amino acid at a plurality of positions that together form a mapped region; predicting the catalytic activity of each of the plurality of candidate mutated enzymes using the method of any embodiment of the first or second aspect thereby obtaining for each candidate mutated enzyme a score indicative of the in the effective activation barrier of the candidate mutant enzyme; and combining the scores for the plurality of candidate mutated enzymes into one or more position-specific metrics indicative of the potential for mutant-associated catalytic improvement at the position. Thus, a site directed mutagenesis potential map may comprise one or more position-specific metrics indicative derived from scores indicative of the catalytic activity of mutant enzymes comprising mutations at the respective position. The mapped region may comprise all the sequence of the reference enzyme optionally with one or more excluded positions and/or regions. In other words, the candidate mutant enzymes may differ from the reference enzyme at a single position.

Combining the scores for the plurality of candidate mutated enzymes into one or more position-specific metrics may comprise obtaining one or more position-specific metrics for each position in the mapped region based on the scores obtained for candidate mutated enzymes of the plurality of candidate mutated enzymes that comprise a mutation at the respective position. The one or more position-specific metrics may comprise a mean or median score, a maximum score and/or a minimum score for the candidate mutated enzymes of the plurality of candidate mutated enzymes that comprise a mutation at the respective position.

Thus, also described herein is a computer implemented method for obtaining the scores for a plurality of candidate mutated enzymes using the method of any preceding claim thereby obtaining for each candidate mutated enzyme a score indicative of the potential reduction in the effective activation barrier of the candidate mutant enzyme. The scores may together form a site directed potential map. Combining the scores for the plurality of candidate mutated enzymes may comprise obtaining one or more model-specific metrics based on the plurality of scores obtained for the plurality of candidate mutated enzymes, optionally wherein the model-specific metrics comprise a mean or median score, a standard deviation of scores, a variance of scores for the candidate mutated enzymes of the plurality of candidate mutated enzymes that comprise a mutation at the respective position. The model-specific mean and variance metric calculated by any ML for each plurality of candidate mutant enzymes may be stored into a computer file.

The plurality of predictions may correspond to every possible single-mutant mutation in the enzyme sequence (site directed potential mutagenesis map). The plurality of predictions may correspond to any random diverse plurality of enzyme sequences. As explained above, in any embodiment of any aspect, for each ML model, a specific mean and variance may be obtained when calculating a diverse set of mutants that target every possible site and amino acid substitution, such as that calculated for the site directed mutagenesis potential map. This mean and variance which is specific to each ML model can be used to standardise not only the site directed mutagenesis potential map, but any future prediction of the model. The model specific mean and variance may be recorded for this purpose.

As the skilled person understands, the complexity of the operations described herein (due at least to the complexity of performing the calculations as described herein, and the amount of data that is typically associated with computational chemistry calculations such as QM/MM optimisations or DFT calculations including DFT cluster models, are such that they are beyond the reach of a mental activity. Thus, unless context indicates otherwise (e.g. where sample preparation or acquisition steps are described), all steps of the methods described herein are computer implemented.

The present invention also relates to use of the methods as described herein in the engineering of an enzyme with one or more desired properties.

According to a fifth aspect, there is provided a method of providing a candidate enzyme with improved catalytic activity compared to a reference enzyme, the method comprising: providing a plurality of candidate mutated enzymes, wherein the candidate mutant enzyme differs from a reference enzyme by one or more amino acids; predicting the catalytic activity of each of the plurality of candidate mutated enzymes using the method of any embodiment of the first or second aspect, thereby obtaining for each candidate mutated enzyme a score indicative of the effective activation barrier of the candidate mutant enzyme; and ranking the plurality of candidate mutated enzymes on the basis of the scores obtained, thereby identifying candidate mutant enzymes that are likely to have improved catalytic activity. The plurality of candidate mutated enzymes may differ from the reference enzyme by at least one amino acid at a plurality of positions that together form a mapped region, and the scores may therefore form a site directed mutagenesis potential map for the reference enzyme.

Candidate mutated enzymes that are highly ranked (i.e. with more negative scores) may be more likely to have improved catalytic activity than candidate mutated enzymes that are not as highly ranked (i.e. that have less negative scores). Thus, the ranked scores can be used to enrich a library to be used in an iteration of a directed evolution process for candidate mutant enzymes that are more likely to have improved catalytic activity, by preferentially selecting candidate mutant enzymes that are more highly ranked. Identifying candidate mutant enzymes that are likely to have improved catalytic activity may comprise candidate positions that are associated with one or more mutants likely to have improved catalytic activity. The plurality of candidate mutated enzymes may comprise candidate mutated enzymes that comprise mutations in different parts of the enzyme. This may enable a more meaningful/thorough exploration of the mutation potential in the enzyme. The plurality of candidate mutated enzymes may comprise at least 50, at least 100, at least 200, at least 500, at least 1000, or several thousand candidate mutated enzymes. The plurality of candidate mutated enzymes may differ from the reference enzyme at a plurality of candidate positions that together span any region of the enzyme, optionally excluding one or more residues a priori identified to be directly involved in the mechanism of reaction and/or any cysteine residues and/or any residues in the N terminal and/or C terminal region and/or any residues known to covalently bond a cofactor and/or any residues which have been selected to impose restraints in the molecular dynamics simulation. The plurality of candidate mutated enzymes may have been selected using a site directed mutagenesis potential map generated using the method of any embodiment of the fourth aspect. The plurality of candidate mutated enzymes may target any residue including the N-ter and C-ter regions but may also optionally avoid candidate mutations in the N-ter and C-ter regions as they may be too unrestrained from the protein structure and therefore may result in weaker mutation-response signal. The plurality of candidate mutated enzymes may differ from the reference enzyme by one or more amino acids, such as e.g. 1, 2 or 3 amino acids. Analysing mutants with more than one mutation (e.g., double or triple mutants but can easily be extended to any number of mutations) may advantageously accelerate the speed of searching through candidate positions within the enzyme, as well as enable the investigation of potential synergies between mutations. The plurality of candidate mutated enzymes may each differ from the reference enzyme at one or more positions that may be randomly selected. The positions may be randomly selected from anywhere in the enzyme but may optionally be randomly selected within a predetermined set. For example, a predetermined set may exclude residues previously identified as involved in the mechanism of reaction and/or all residues within predetermined N terminal and/or C terminal regions and/or any cysteine residues and/or any residues which covalently bond a cofactor and/or any residues which have been selected to impose restraints for the molecular dynamics simulation previously described. A predetermined set may include all positions that are not specifically excluded. A predetermined set may include all positions outside of the core region that are not specifically excluded.

According to a sixth aspect, there is provided a method of providing a candidate mutant enzyme with improved catalytic activity compared to a reference enzyme, the method comprising: providing a site directed mutagenesis potential map for a reference enzyme using the method of any embodiment of the fourth aspect, and identifying one or more candidate position(s) that is/are associated with one or more candidate mutant enzymes likely to have improved catalytic activity based on the one or more position-specific metrics. The method may further comprise providing one or more candidate mutant enzymes comprising mutations at the one or more candidate position(s) and predicting their catalytic activity using the method of any embodiment of the first or second aspects. Identifying candidate positions that are associated with one or more mutants likely to have improved catalytic activity based on the one or more position-specific metrics may comprise ranking the candidate positions based on one of the one or more metrics. For example, the one or more position-specific metric may comprise an average score across mutants that comprise a mutation at the respective position, and the candidate positions may be ranked by order of the most negative average score. For example, candidate positions that have more negative average scores may be more likely to have improved catalytic activity than candidate positions that have less negative average scores. A set of candidate mutant enzymes may together be referred to as a library. The method may further comprise repeating the step of predicting catalytic activity with another library (or one or more further libraries), and comparing the predictions for the respective libraries. Comparing the predictions for the respective libraries may comprise determining a summary statistic for the respective libraries, such as e.g. the mean or median score across candidate mutant enzymes in the library. The method may further comprise selecting a library based on the comparing step, such as e.g., the library that is associated with the highest mean or median score. The method of the second aspect may advantageously be used to predict catalytic activity for candidate mutants/libraries of candidate mutants comprising more than one individual mutations.

Once a set of sites are selected, further optimisation to choose a specific set of codons (to be used to generate diversity at the selected positions) may be performed. Each possible library may include mutants with more than one individual mutation. The mutants may be individually predicted based on each ML model. The predictions of each machine learning model may be corrected for standardisation based on the specific means and variances previously calculated during the full-enzyme saturation potential map prediction. Each library may then be scored based on the median score of all the included mutants composing the library based on a specific selection of codons.

Thus, also described herein is a method of providing a candidate mutant enzyme with improved catalytic activity compared to a reference enzyme, the method comprising: providing a plurality of machine learning models as described in relation to the second aspect, providing a model-specific mean and variance metric for each machine learning model based on predictions for a common set of candidate mutated enzymes, predicting a score for a specific candidate mutant enzyme sequence, and adjusting the score based on the previously stored model-specific mean and model-specific standard deviation by subtracting the model-specific mean metric and dividing the result over the model-specific standard deviation which may be obtained from the model-specific variance metric previously stored. The method may further comprise aggregating the results of all the models as a mean calculation of all the metric-adjusted final scores.

The method of any aspect may further comprise identifying key catalytic residues by any recombinant technique such as site directed mutagenesis, wherein the reference enzyme comprises the key catalytic residues. The method of the present aspect may comprise selecting one or more candidate positions in the enzyme for experimental validation. The selection may be based on a combination of criteria including: the ranked scores associated with the candidate mutant enzymes or the one or more position-specific metrics; and one or more of: the location of the positions in the enzyme, and one or more criteria associated with a specific gene synthesis methodology. The one or more criteria associated with a specific gene synthesis methodology comprise one or more of: avoidance of oligonucleotide overlap regions, availability of a degenerate codon that includes both the reference amino acid and the mutated amino acid, and efficiency by which the degeneracy can be substituted into the sequence by using minimal new oligonucleotide synthesis.

The method may further comprise designing and/or providing a library for PCR-based gene synthesis and/or solid phase gene synthesis and/or full de novo gene synthesis and/or site directed mutagenesis that comprises degenerate codons for the selected candidate positions. A plurality of candidate positions may be selected based in part on the location of the positions in the enzyme. For example, the plurality of candidate positions may be selected to be distributed throughout the enzyme sequence.

The degenerate codons may have a predetermined multiplicity, for example a multiplicity of 12 or lower. The degenerate codons may contain no stop codons. The degenerate codons may code only once for any amino acid. Limiting the codon multiplicity may advantageously enable to explore more sites per screening iteration. Higher multiplicity codons containing all 20 amino acids (e.g., NNK) may easily be used instead and are a common choice in directed evolution experiments, when the intention is to test every possible amino acid in a selected position. The method may comprise designing and/or providing a synthetic gene library that includes one or more of the identified candidate mutant enzymes and/or mutations at one or more candidate positions in the enzyme. Strategies like PCR-based gene synthesis which explore degenerate codons for specific sites may be particularly useful in the context of the invention as the rational predictions may be good enough to increase the chances of a successful hit, but not strictly accurate on an individual basis, for example because other factors than the activation barrier may influence enzyme kinetics (e.g., stability). Alternatively, fully synthetic genes or full de novo protein design may be used. The process of designing a library may be automated. Thus, all of the steps involved in identifying candidate mutant enzymes and designing a library accordingly may be computer-implemented.

The method may further comprise obtaining one or more of the identified candidate mutant enzymes, optionally by expressing a gene library designed based on the one or more identified candidate mutants. The method may further comprise testing one or more of the identified candidate mutant enzymes for one or more properties including catalytic activity. The method may further comprise testing one or more of the identified candidate mutant enzymes for one or more properties for a property other than catalytic activity. The one or more properties may comprise stability in a predetermined condition or sets of conditions, efficiency of expression, and/or catalytic activity towards one or more substrates of interest. The steps of obtaining and/or testing candidate mutant enzymes may be automated. For example, the steps of obtaining and/or testing candidate mutant enzymes may be performed by a computing device controlling one or more automated laboratory equipment such as e.g., one or more liquid handling robots, plate readers, etc. Thus, the method may comprise outputting information identifying one or more candidate mutant enzymes, a gene library, instructions to obtain and/or test one or more candidate mutated enzymes. The steps of obtaining and/or testing one or more of the identified candidate mutant enzymes may be repeated using a different set of the identified candidate mutant enzymes. For example, mutated positions that did not result in an improved catalytic activity may be excluded from a next round of investigation. The method may further comprise subjecting an identified candidate enzyme to further optimisation and/or a stabilisation process. The stabilisation process may be selected from random mutagenesis, stabilisation of flexible regions, generation of salt bridges, introduction of disulphide bonds, and enzyme supercharging, preferably wherein the stabilisation process is enzyme supercharging. The method may further comprise selecting an identified candidate mutant enzyme or a further optimised version thereof and repeating the method of the present aspect using the selected enzyme as a reference enzyme.

According to a seventh aspect, there is provided a system comprising: a processor; and a computer readable medium comprising instructions that, when executed by the processor, cause the processor to perform the steps of the method of any embodiment of any preceding aspect. The system may further comprise one or more automated laboratory equipment, for example to perform the steps of obtaining and/or testing candidate enzymes.

According to an eighth aspect, there is provided one or more computer readable media comprising instructions that, when executed by one or more processors, cause the one or more processors to perform the steps of the method of any embodiment of any of the first to sixth aspects.

According to a further aspect, there is provided a computer program comprising code which, when the code is executed on a computer, causes the computer to perform the steps of any method described herein.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying Figures in which:

FIG. 1 A and B show schematic flow charts showing in general terms methods of predicting enzyme catalytic activity as described herein.

FIG. 2 is a schematic flow chart showing in general terms a method of engineering an enzyme as described herein.

FIG. 3 shows an embodiment of a system for implementing the methods described herein.

FIG. 4 shows the structure of an enzyme and substrate optimised in Example 1. (a) 6-hydoxy-D-nicotine oxidase (6-HDNO) protein with covalently bound flavin adenine dinucleotide (FAD) cofactor in orange and docked (R)-2-phenyl pyrrolidine (PPY) substrate in blue. (b) Close-up of the active-site region of with closest residues labelled in cyan, the substrate in blue and FAD in orange. (c) Chemical schematic of the PPY substrate in a hydride-transfer orientation with key atoms labelled in red (H1 and C1 of PPY and N5 and N10 of FAD).

FIG. 5 shows the results of MD simulations for a mutant (E350L/E352D) of the enzyme of FIG. 4, referred to as D2 enzyme. All-atom root-mean-square deviation (RMSD) of MD simulations from the crystal structure: (a) is the free 6-HDNO D2 enzyme, of length 1 μs started from the crystal structure, (b) is the enzyme with free PPY substrate within the active site, of length 10 μs and started from the coordinates taken from end of the simulation shown in (a), (c) is enzyme with PPY restrained in a near attack conformation (NAC) within the active site, of length 5 μs and started from 1212 ns of the simulation in (b).

FIG. 6 shows (a) PPY-H1 to FAD-N5 distance during the unrestrained 10 μs MD simulation of the D2-PPY complex showing near attack conformations (labelled N) and two stable orientations that are in not in the correct geometry for direct hydride transfer (labelled X & Y). (b) 2D histogram of distance (PPY-H1 to FAD-N5) and angle (FAD-N1-N5 to PPY-H1) during the unconstrained 10 μs MD simulation. (c) Near attack conformation taken from the unrestrained MD simulation at 1212 ns (grey). The two stable orientations (X) and (Y), in light blue (d) and ochre (e), respectively. The H1 positions are shown in red and are found pointing away from the FAD isoalloxazine ring in configurations (X) and (Y) but in (N) H1 is close and pointing towards FAD, and hence (N) is predisposed for hydride transfer.

FIG. 7 shows (a) schematic of the quantum mechanical barrier for formation of the transition state (TS) from the reaction complex (RC), showing typical configurations used in density functional theory (DFT) model calculations of the free energy of activation for hydride transfer (AG^{‡}), including the atoms used in the truncated FAD moiety. (b) A schematic of the chemical mechanism of hydride transfer to produce the first intermediate of the oxidised PPY proposed for 6-HDNO and used here in the theoretical modelling of enzyme activation energy. (c) The attack angle (a) as defined by three atoms in the FAD (N5 and N10) and PPY (H1). (d) A graph of AG^{‡} (the height of the TS barrier above the RC) as a function of attack angle (a) calculated using density functional theory (DFT). The optimal hydride transfer angle α was estimated to be 120°.

FIG. 8 shows the correlation of activation energy barriers as calculated by the QM/MM methodology (ΔE^{‡}_{QMMM}) versus the Q20 electrostatic approximation (ΔΔG^{‡}_{Q20}). A good correlation is observed with a coefficient of determination of 0.82, meaning electrostatics mostly explain the variability of the activation energies throughout the conformational dynamical fluctuations of the system.

FIG. 9 shows (a) instantaneous ΔΔG‡_{Q20} values for a 5 μs MD simulation of D2 with a restrained substrate (light grey trace), moving average μ_{Q20} over 100 ΔΔG^{‡}_{Q20} values (red trace), and running average of ΔΔG^{‡}_{Q20EFF} using all preceding data (orange trace). (b, c and d) Histograms of ΔΔG^{‡}_{Q20} based on 5 μs of MD simulation with a restrained substrate (light grey) versus a theoretical normal distribution (red), for three enzymes D2 (b), D113N (c) and A270G (d). The population of electrostatically calculated barrier contributions are normally distributed, and the effective barrier contribution (ΔΔG^{‡}_{Q20EFF}), from Equation (2), is approximately 10 kcal×mol⁻¹ lower than the calculated mean barrier μ_{Q20}, placing it close to the lower limits of the distribution (−16.0 kcal×mol⁻¹ for D2).

FIG. 10 shows electrostatic contributions per residue over 5 μs of substrate-restrained MD as calculated by the Q20 methodology. Residue 460 corresponds to FAD. Residues 461 to 583 correspond to distant and ineffectual counter-ions. Residues starting at 584 correspond to solvent molecules. Residue D352 produces very strong barrier-reducing contributions of −8.5 kcal×mol⁻¹, with opposing effects from, e.g., the external region of the FAD moiety, but also from other residues such as K348 and R367, which have an unfavourable effect of +2.4 kcal×mol⁻¹ and +3.2 kcal×mol⁻¹, respectively, while residue D316 has a favourable effect of −3.0 kcal×mol⁻¹. In this case, solvent water molecules are detrimental to catalysis and raise the barrier by +5.1 kcal×mol⁻¹.

FIG. 11 shows (a) temperature-factors of C_{α}-atoms calculated on a per residue basis for: the 6-HDNO D2 enzyme (orange), a D113N mutant (blue), and a A270G mutant (black), showing that even mutations close to the surface cause global changes in dynamics. (b) Generalised correlation matrix based on C_{α}-atoms of the D2 enzyme, from a 5 μs restrained MD simulation, showing the complex interactions between residues, by which dynamics may propagate through the enzyme. Overlaid on this are examples of the network of communication from distal residues to the active site (depicted with blue arrows); example network 1: R34→W31→F306; example network 2: R34→P74→S416. (c) Example 3D-visualization of the network of communication (some residues hidden for improved visibility) from distal residues to the active site (shown with purple arrows).

FIG. 12 shows a general schematic of the process of estimating catalytic barriers based on electrostatic effects and dynamics with the current technology.

FIG. 13 shows (A) QM region of the QM/MM models as optimised by ChemShell/Turbomole/DL_Poly with the B3LYP functional and the def2-SVP basis set for the QM region and a CHARMM forcefield for the MM region. The QM region was defined including the flavin moiety, the substrate, and amino acid residues H72, M129, H130, W314 and N414. (B) Core region of Q20, including only the substrate, the flavin moiety and the covalently bonded H72 residue.

FIG. 14 shows the enzyme 6-HDNO D2 with a covalently bound FAD cofactor (light blue) and substrate PPY (blue) docked into the active site. All mutations of this first protein optimisation round were performed near the active site. Residues highlighted in red were included in the site directed mutagenesis (SDM) process which included a variety of small degenerate codons. A new variant now termed 6-HDNO D3 was found by the mutation of N414H which resulted in an enantioselective rate increase of 1.7-fold towards PPY.

FIG. 15 shows: (A, B) enzyme variants 6-HDNO D2 and D3 (N414H) in the biocatalytic oxidation of 2-phenylpyrrolidine showing that the improved activity gained on 6-HDNO-D3 did not affect enantioselectivity. Reaction conditions: 10 mM substrate, 0.2 mg/ml enzyme, 30° C. in pH 8 100 mM buffer. Conversions determined by GC-FID. Enantiomeric excess (ee) determined by HPLC. (C) Shows turnover frequencies (TOF) for HDNO D2 (dark grey) and D3 (grey) in the oxidation of different 2-substituted pyrrolidines. Reaction conditions: 10 mM substrate, 0.2 mg ml⁻¹ enzyme, 30° C. in pH 8 100 mM buffer. Conversions determined by GC-FID. TOF calculated using conversions after 10 min.

FIG. 16 shows a heatmap of enzyme 6-HDNO D2 showing the coverage of residues and the estimated best activity at each site as obtained by the calculation of changes in barrier by molecular dynamics (MD) and the insertion of conservative mutations. Sites untested are shown as low activity sites for better visualisation. The tested space is reduced to 149 sites only containing any of the conservative amino acids (Ala, Cys, lie, Lys, Met, Phe, Ser, Thr, Tyr and Val). Even with the reduction the space of possible mutant variants is practically infinite (>10²⁰⁰).

FIG. 17 shows a scatter plot depicting the estimated changes in activity by mutation at each residue versus mean distance to the active site on the 6-HDNO D2 enzyme. The enzyme activity is estimated based on changes in dynamics and electrostatics effected through conservative mutations. No significant correlation is observed by this methodology. Distances were measured by calculating the centre of geometry of each residue as a reference to the hydride receptor N atom in FAD. This was done as an average over 10000 frames sampled with a 1 ns difference across 10 μs of MD.

FIG. 18 shows amino acid sequence of the D3 protein mapping the construction of the PCR-based synthesis oligonucleotide library for DE. Highlighted in orange are overlap areas where no codon degeneracy is allowed for the current design accounting roughly for ⅓ of the sequence. The 7 amino acids targeted in the design of the degenerate library are marked in blue, where a diverse set of 7 degenerate codons were employed in the construction distributed over 5 degenerate oligos.

FIG. 19 shows the enzyme 6-HDNO D2 highlighting sites targeted during a round of rational directed evolution with a PCR based gene synthesis library. These sites were selected rationally from a ranked list of residues obtained by dynamic barrier change estimations and within the experimental limitations of the full-length genetic construct. Only small degenerate codons were selected. A new active variant was found with mutations A43S, A238T and V431A, which are marked in red. No hit was found on mutations performed on residues Y242, A329, V347 and A437 marked in orange.

FIG. 20 shows the turnover frequencies (min⁻¹) and activity increase over HDNO D2 for the HDNO D3 and HDNO D6 variants across a series of secondary amines. Reaction conditions: 10 mM substrate, 0.2 mg ml⁻¹ enzyme (D2) or 0.05 mg ml⁻¹ (D6), 30° C. in pH 8 100 mM buffer. Turnover frequencies (TOF, min-1) calculated using conversions after 10 min.

FIG. 21 shows the turnover frequencies (min⁻¹) and activity increase over HDNO D2 of HDNO D6 across a series of secondary and primary amines. Reaction conditions: 10 mM substrate, 0.2 mg ml⁻¹ enzyme (D2) or 0.05 mg ml-1 (D6), 30° C. in pH 8 100 mM buffer. TOF calculated using conversions after 10 min. n.d.: not determined. n.a.: no activity or too low to be accurately determined.

FIG. 22 shows the thermal stability changes by surface supercharging of HDNO by insertion of a K208E mutation into the HDNO D3 variant. Thermal stability for K208E D3 versus D3 variants is measured by residual activity and conversion as quantified by GC every 15 minutes following incubation at 50° C.

FIG. 23 shows the thermal stability changes by surface supercharging of HDNO by insertion of a K208E mutation into the HDNO D3 variant. Thermal stability for K208E D3 versus D3 variants measured is measured by residual activity and conversion as quantified by GC every 15 minutes following incubation at 45° C.

FIG. 24 shows the thermal stability changes by surface supercharging of HDNO by insertion of D308E, R207E, R282E, and K428E mutations into the HDNO-D6 variant. Thermal stability is measured by residual activity and conversion as quantified by GC every 15 minutes following incubation at 45° C. as compared to D6.

FIG. 25 shows the thermal stability changes by surface supercharging of HDNO by insertion of a double mutant K208E/R282E into HDNO D6 variant, now termed HDNO D8. The thermal stability is measured by residual activity and conversion as quantified by GC every 15 minutes following incubation at 45° C. and compared to HDNO D6.

FIG. 26 illustrates the HRP-ABTS assay used in Example 2.

FIG. 27 shows histograms of the individual mutant variant ΔΔG^{≠} scores (kcal mol⁻¹) as obtained for the full set of data (over 360000 mutants) from the different starting conformations in Example 3. All datasets followed the same random generation method. Clearly a strong dependence in the scores is observed with respect to each starting conformation evident in the mean and variance of each subset. Datasets were smoothed to a normal distribution.

FIG. 28 (A) Shows a meta-correlation between individual correlation coefficients for the unseen test and validation subsets on randomly encoded LR-FFT models on the dataset of the conformation from 570 ns. A clear correlation shows that the variability in the encoding vectors significantly changes the model fitness capacity of individual ML models rather than just creating random fluctuations in testing performance. (B) Shows that random data splitting into train/test/validation subsets is not a true source of variation in model fitness, and instead only generates random variability in the individual correlation coefficients with no meta-correlation observed when using an unchanged set of encoding vectors for each amino acid in the protein sequence.

FIG. 29 shows the convergence of the ensemble correlation coefficient towards the unseen validation set of an increasing number of LR Models included in the aggregation prediction. The slope of the plot shows that further additions of models to the consensus will result in diminished returns towards performance. For each x-axis coordinate an average of 20 (sampled out of 740) ensemble models were calculated.

FIG. 30 shows the self-(diagonal) and cross-correlation (non-diagonal elements) matrix displaying the cross and self-correlation coefficients of a series of ML models as calculated on data generated from different seed conformations. A total of 250 regularised Lasso models were used with random FFT encoding for each set of consensus calculations encoded by a randomly generated dictionary with 28 entries per amino acid.

FIG. 31 shows the two-level ensemble modelling process (multi conformation and multi-model for each seed conformation) used for the prediction of the best target residues for DE. In Example 3, this process was based on the current data of over 360000 1 ns MD simulations scored for ΔΔG^{≠} improvement and sourced from 10 different seed conformations from the last microsecond of a 10 μs MD simulation on the parent variant 6-HDNO D3. A set of 25 ML models were employed for each conformation (resulting in a total of 250 models). The score for every possible mutation at every single site was first predicted and this set of scores was standardised to a mean of zero and standard deviation of one using a suitable offset and scaling for each model. These offset and scaling values (specific to each model) were used in all further model calculations to produce standardised model scores. In Examples 4 to 6 this process was based on five conformations, while Example 5 used only one ML model for each seed conformation. The process was then used to produce standardised scores for the individual mutants in each codon library (for the final codon optimisation) once a series of high-ranking sites had been identified in Examples 4 to 6.

FIG. 32 shows the one hot encoding table used for ML models in Example 3 (A) and an exemplification of the one hot encoding of the sequence of amino acids “GMFWKAIC” (SEQ ID NO:5) (B).

FIG. 33 shows a full in silico site directed mutagenesis (SDM) potential map based on the ensemble ML models of the 6-HDNO data based on ΔΔG^{≠} calculations of over 360000 MD simulations (A), and a heatmap of consensus ΔΔG^{≠} values as measured in mean standard deviations obtained for all mutations possible at each site based on the consensus output (B). A total of 250 neural network models are part of the ensemble. Sites 69, 70, 113, 138, 242, 244, 348, 367 and 413 are highlighted as examples of potential beneficial sites. B. Red indicates a mean beneficial ΔΔG^{≠} estimation while blue indicates a detrimental mean ΔΔG^{≠} effect.

FIG. 34 Several active variants containing a diversity of mutations were produced using the rationally guided methodology. (A) Shows a microtitre plate containing rationally guided de novo full gene synthesis library ‘1’ based on an HDNO D3 oligonucleotide construct including degenerate codons 242 (KWC), 348 (VAS) and 353 (RDC) with a maximum diversity of 144 variants including the wild type (WT). Coloured wells demonstrate enzyme activity. (B) Shows a histogram of optical density readings for the activity of 7 enzyme variants towards PPY (plate well reference shown on x-axis). A dotted line indicates the average reading for blank wells. (C) Shows an example microtitre plate containing rationally guided de novo full gene synthesis library ‘2’ based on an HDNO D3 oligonucleotide construct including degenerate codons 109 (RWG), 112 (RBC) and 113 (RDC) with a maximum diversity of 144 variants including the WT. Coloured wells demonstrate enzyme activity. (D) Shows a histogram of optical density readings of 18 enzyme variants towards PPY (plate well reference shown on x-axis). A dotted line indicates the average reading for blank wells.

FIG. 35 shows 2D histograms depicting encoding complexity versus model performance. (A) Shows the correlation coefficients from LR-NonFFT models on test sets with varying size of random encoding dictionaries based on dataset for the conformation at 570 ns. The best performance is observed between 12 and 17 encoding vectors and over-training occurs on larger encoding complexity leading to models with no predictive capacity. (B) Shows the LR-FFT encoded correlation coefficients on test sets with varying size of random encoding dictionaries based on dataset of the conformation at 570 ns. The FFT encoded models are more resilient to over-training, with decreasing gains on more than 28 encoding vectors.

FIG. 36 shows the performance of a series of ML Lasso models with different a hyperparameter values (where α=10^{-x} and x is the horizontal axis value). All models were based on randomly split data from the 570 ns seed conformation, with 80% of the data set used for model fitting and 20% for performance evaluation by correlation coefficient calculation. (A) Shows the results based on a random FFT encoding methodology with encoding complexity of N=28 (best performance of α when x=4.0). (B) Shows the results based on a one hot encoding methodology (best performance of α when x=2.75).

FIG. 37 shows the site directed potential mutagenesis map representing the best targets for DE. A total of 25 models were trained on each data subset (a total of 250 artificial neural network models). Predictions were standardised for each conformation before a final normalised average was obtained.

FIG. 38 shows a dense neural network architecture consisting of a series of LeakyRelu activation functions and the Adam optimiser (learning rate=0.01, decay=e⁻⁴) with a mean square loss function.

FIG. 39 shows the RMSD of 1 μs MD for the wild-type amylase system in Example 4. A low RMSD of under 2.2 Å demonstrates a stable and equilibrated system was obtained.

FIG. 40 shows histograms depicting the distributions of the rate contributions of distinct populations of mutants generated from different parent conformations of the full dataset. Data containing over 45000 distinct mutant 1 ns MD simulations were scored with the Q20 methodology.

FIG. 41 shows the self-(diagonal) and cross-correlation (non-diagonal elements) matrix displaying the cross and self-correlation coefficients of a series of ML models as calculated on data generated from different seed conformations.

FIG. 42 shows: (A) full visualisation of an in-silico site directed mutagenesis potential map based on over 45000 MD simulations, scored by the Q20 methodology and aggregated for conformational representation by an ML approach, and (B) a heatmap of ranked target sites according to normalised aggregated potential for enzymatic rate improvement.

FIG. 43 shows the DFT-optimised cluster model of the transition state for the amylase system in Example 4. Optimisations were performed with the BP86 functional and the 3-21G basis set. An imaginary frequency confirmation shows a simultaneous proton transfer towards the glycosidic oxygen and nucleophilic attack towards the adjacent carbon towards a carbonyl oxygen of Asp197.

FIG. 44 shows: (A) the optimised reactant complex (RC), and (B) the optimised transition state (TS) complex for the proton transfer activation step for the isomerisation of 5-androstene-3,7-dione by ketosteroid isomerase; optimisations were performed with the BP86 functional and the 3-21G basis set.

FIG. 45 shows histograms depicting the distributions of the rate contributions of distinct populations of mutants generated from different parent conformations of the full dataset.

FIG. 46 shows Q20 scores for every 0.1 ns for the 1 μs simulation of the wild-type enzyme based on either Hirshfeld-based or Mulliken-based parameterisation. A correlation between the methods indicates that an alternative method for the calculation of partial atomic charges is possible with a minor impact on the results and predictions.

FIG. 47 shows a grid search of encoding complexity (the number of random encoding vectors) for Lasso models versus model performance on data of the conformation form 900 ns. An increasing performance of the models with higher encoding complexity indicates the benefit of a fully random encoding process that has an unlimited diversity of encoding vectors.

FIG. 48 shows a regularised Lasso performance grid search for the α hyperparameter for a fully randomly encoded FFT transformed set of data (using the conformation form 900 ns) based on an encoding complexity of N=750, compared to the regularised Lasso performance grid search for the AAindex encoded variation based on 553 amino acid properties. The plotted data is an average based on 300 data points, each binned into 30 equal size bins for the average calculation.

FIG. 49 shows the self-(diagonal) and cross-correlation (non-diagonal elements) matrix displaying the cross and self-correlation coefficients of a series of ML models as calculated on data generated from different seed conformations. This analysis was based on one regularised Lasso model (FFT random ProSAR) for each conformation set (N=750 random vectors).

FIG. 50 shows a full visualisation of an in silico SDM potential map, using 50000 MD simulations and generated from the ML aggregation process, based on regularised Lasso models (random FFT, N=750 random vectors).

FIG. 51 shows: (A) the optimised reactant complex (RC), and (B) and the optimised transition state (TS) complex for the methyl transfer activation step in xanthosine transferase. Optimisations were performed by a DFT methodology with the BP86 functional and the 3-21G basis set.

FIG. 52 shows histograms depicting the distributions of the rate contributions of distinct populations of mutants generated from different parent conformations of the full dataset.

FIG. 53 shows the self-(diagonal) and cross-correlation (non-diagonal elements) matrix displaying the cross and self-correlation coefficients of a series of ML models as calculated on data generated from different seed conformations. These data were based on 30 regularised Lasso ensemble FFT Random ProSAR models for each conformation set (with an encoding complexity of N=40 random vectors).

FIG. 54 shows: (A) the water box size after 1 ns NPT MD runs for a set of 4167 triple mutants corresponding to seed conformation 1000 ns. (B) Shows the μ_{Q20,Protein} scores for tripled mutants from the NPT dataset correlated with the scores for the same triple mutants from the NVT (standard method) generated from the conformation at 1000 ns. A correlation coefficient of 0.78 indicates that these methods could be used interchangeably.

FIG. 55 shows a full visualisation of the in silico SDM potential based on an ensemble of ML models (30 regularised Lasso models per conformation), resulting in a total of 150 models (random FFT, N=40 random vectors, and α=0.01), repeated for each dataset that was scored. (A) Based on μ_{Q20,Protein} scores. (B) Based μ_{Q20} scores dataset. (C) Based on the ΔΔG^{‡}_{Q20EFF,Protein} scores dataset. (D) Based on the ΔΔG^{‡}_{Q20EFF} scores dataset.

FIG. 56 shows optimised DFT cluster models for: (A) the reactant complex (RC), and (B) the transition state (TS) structures corresponding to the coordinates at 3653 ns from a 10 μs MD simulation.

FIG. 57 shows a grid search 2D-histogram plot for the optimisation of the α regularisation hyperparameter on a series of 10000 regularised Lasso models measured by correlation coefficients on random data test sets. Data was split randomly for training based on 92% of data and the remaining 8% was used for testing (a was chosen randomly within the range of 10⁰ to 10⁻⁶ and the best α-value was 10-2.85)

FIG. 58 shows the standardised results for visualisation of the in silico SDM potential maps obtained from data modelling on the scored values of 1000 MD simulations of length 50 ns. (A) Based on a random FFT encoding and an ensemble of 30 Lasso models. (B) Based on a one hot encoded per-site method and an ensemble of 2000 regularised Lasso models (α=10^{-2.85})

### DETAILED DESCRIPTION OF THE INVENTION

### Definitions

As used herein, the terms “computer system” includes the hardware, software and data storage devices for embodying a system or carrying out a method according to the above-described embodiments. For example, a computer system may comprise one or more processing units (such as a central processing unit (CPU), graphical processing unit (GPU), etc.), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

As used herein, the term “molecular dynamics simulation” refers to a computer simulation method for analysing the movement of atoms and molecules. The trajectories of atoms within the system may be determined by numerically solving Newton's equation of motion for a system of interacting particles. The forces between the particles and their potential energies may be calculated using interatomic potentials or molecular mechanics force fields. Molecules in a solvent may be simulated using explicit or implicit solvent. Using an explicit solvent model (such as e.g., the TIP3P, SPC/E and SPC-f water models), explicit solvent particles are calculated by the force field. In implicit solvent models, a mean-field approach is used to calculate the contribution of the solvent. In embodiments, the molecular dynamics simulations used herein use an explicit solvent model such as TIP3P. Molecular systems may be simulated in conditions of constant amount of moles (N), volume (V) and energy (E), also referred to a s “microcanonical ensemble” or “NVE”. Molecular systems may be simulated in conditions of constant amount of moles (N), volume (V) and temperature (T), also referred to a s “canonical ensemble” or “NVT”. Molecular systems may be simulated in conditions of constant amount of moles (N), pressure (P) and temperature (T), also referred to as “isothermal-isobaric ensemble” or “NPT”. In embodiments, the molecular dynamics simulations herein use NVT and NPT conditions.

As used herein, the term “enzyme” refers to a biological molecule (usually a protein or protein derivative) that acts as a catalyst. A catalyst is a compound that accelerates chemical reactions. The molecules that enzymes act upon are referred to as “substrates” and the enzyme converts the substrates into different molecules referred to as “products.” Some enzymes contain a second chemical compound or metallic ion that is required for catalysis, which is referred to herein as a “cofactor”. The International Union of Biochemistry and Molecular Biology have developed a nomenclature for enzymes using EC numbers (for “Enzyme Commission”), which is used herein. Each enzyme can be classified by “EC” followed by a sequence of numbers. The first number classifies the enzyme based on its mechanism: EC-1 for “oxidoreductases” that catalyse oxidation/reduction reactions, EC-2 for “transferases” that transfer a functional group (e.g., a methyl or phosphate group), EC-3 for “hydrolases” that catalyse the hydrolysis of bonds, EC-4 for “lyases” that cleave bonds by means other than hydrolysis and oxidation, EC-5 for “isomerases” that catalyse isomerisation within a single molecule and EC-6 for “ligases” that join two molecules using covalent bonds. Most proteins comprise 20 amino acids that include: Alanine, Arginine, Asparagine, Aspartic Acid, Cysteine, Glutamic acid, Glutamine, Glycine, Histidine, Isoleucine, Leucine, Lysine, Methionine, Phenylalanine, Proline, Serine, Threonine, Tryptophan, Tyrosine and Valine. However, enzymes may contain non-proteinogenic amino acids, which are those not naturally encoded or found in the genetic code of any organism. Over 140 amino acids occur naturally in proteins and thousands more may occur in nature or be synthesized in the laboratory, and all can be used in the methodologies described herein. Furthermore, proteins may be post-translationally modified, which refers to the covalent and generally enzymatic modification of proteins following protein biosynthesis. Post-translational modifications can extend the chemical repertoire of the 20 standard amino acids by modifying an existing functional group or introducing a new one such as phosphate. Eukaryotic and prokaryotic proteins can also be glycosylated or lipidated (by attachment of carbohydrate or lipid molecules, respectively). Post-translationally modified amino acids can be used in the methodologies described herein. Thus, within the context of the present disclosure, the term “amino acid” encompasses any of the 20 standard amino acids, any non-standard amino acid, any non-standard amino acids, and any modified version thereof such as post-translationally modified versions thereof (e.g. phosphorylated, methylated, glycosylated or lapidated amino acids). Amino acids may be referred to herein using the IUPAC one letter code, or three letter code, as provided in Table 1.

TABLE 1
Abbreviations of amino acids based on the
IUPAC one letter code or three letter code.

| IUPAC One letter code | Three letter Code | Amino acid |
|---|---|---|
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Nucleotide sequences may be described herein using the IUPAC nucleotide code (including possible degeneracy), as provided in Table 2. As used herein, “a codon” is a trinucleotide sequence of DNA (or RNA) bases. Codons may correspond to a specific amino acid according to a genetic code. A genetic code describes the relationship between a sequence of bases (A, C, G, and T or the degenerate versions R, Y, S, W, K, M, B, O, H, V, and N) in a gene and the corresponding protein sequence(s) that it encodes.

TABLE 2
Abbreviations of nucleic acid bases based on the IUPAC
nucleotide code (including possible degeneracy).

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or - | gap |

As used herein, the term “quantum mechanics/molecular mechanics simulation” (QM/MM simulation) refers to a calculation using both a quantum mechanics method and a molecular mechanics method to model respective parts of a molecule. A QM/MM may use mechanical embedding, electrostatic embedding or polarised embedding to model the electrostatic interactions between the QM and the MM region. The part that is modelled using a quantum dynamics method may be referred to as the “QM region” or “reactive centre”. The part that is modelled using a molecular mechanics method may be referred to as the “MM region”. The reactive centre may comprise a few atoms include at least one atom involved in a reaction and one or more atoms close to the reactive atom, a substrate and optionally one or more atoms of a cofactor. The reactive centre may comprise a substrate, one or more amino acid residues that are key to the reaction mechanism. The reactive centre may comprise one or more water residues. A quantum mechanics method uses principles of quantum mechanics to model a system. For example, a QM method may use density functional theory (DFT). DFT models the property of the system as functionals of the spatially dependent electron density of the atoms in the system. A molecular mechanics method uses principles of classical mechanics to model a molecular system. For example, the potential energy of all systems may be calculated as a function of the nuclear coordinates of the atoms in the system using force fields. A DFT cluster method is a DFT model that is used to model chemical reactions in enzymes by incorporating a fraction of the residues that are identified as the most relevant towards the model (such as e.g. the active site, optionally including substrates, cofactors, solvent and ions). These may be referred to as “QM region” herein by analogy with the region that is modelled with a full QM method in a QM/MM model. Optionally, a series of constraints are imposed on the border atoms (atoms that are linked to atoms included in the DFT model), delimiting the model to preserve the original geometry (e.g., if it came from a molecular dynamics simulation). In other words, the “effect” of residues other than those explicitly included in the model can be exerted by imposing restraints to some atoms. For example, any atoms belonging to a backbone in the enzyme may be fixed using the coordinates from a particular frame (e.g. from an MD conformation). When the model includes the active site, the active site conformation is therefore kept in place by these constraints, and a particular transition state (TS) can be calculated using the DFT model. Large-enough DFT cluster models are believed to be very accurate even to model mutagenesis directly (given that the residues mutated are in the model itself). As described further below, QM/MM or DFT cluster model optimised coordinates can be used to estimate the electrostatic component of the activation barrier based on a purely electrostatic methodology (referred to as Q20 here). This parameterisation step is very robust, and in particular does not need a QM/MM optimisation that models the entire enzyme. In particular, the use of a DFT Cluster model focussed on the active site (including the substrate and any cofactor) was found by the inventors to perform adequately for this step, particularly in combination with constraints associated with coordinates for backbone atoms from a molecular dynamics conformation. Briefly, in the Q20 method the Coulombic interaction of a (static) external region and the changes to the partial charges of the reactive region that occur during the formation of the transition state from the reactant complex are used to provide an estimate for the electrostatic component of the activation barrier (ΔΔG^{‡}_{Q20}). For this process, the enzyme-substrate-cofactor system (or enzyme-substrate, if no cofactor is present) can be split into a “core region” and an “external region”. The core region may include the main atoms involved in a change in partial atomic charges during the activation step. The external region may include the rest of the system. Note that the core region need not be the same as the QM region (or the region comprising the atoms explicitly included in the DFT model). For example, the core region may include a subset of the atoms in the QM region. The Q20 methodology described herein may be used to assess the impact on activation barrier of any mutation outside of the core region. In embodiments, the core region may be limited to the substrate and cofactor. In other embodiments, the core region may include one or more atoms/residues of the enzyme, such as e.g. one or more residues that form a direct bond with a cofactor.

As used herein, the terms “N terminus” (Nter) and “C terminus” (Cter) refer to the regions of a protein that are located at the amino terminal and carboxy terminal ends, respectively, of an amino acid chain of the protein. These regions may comprise a fixed number of amino acids. For example, the Nter and Cter regions may refer to the first/last 10 amino acids of an amino acid chain. The Nter and/or Cter regions may correspond to regions that have a higher dynamic variability than the other regions of the protein. The Nter and Cter regions of a protein may have different lengths. The lengths of the Nter and Cter regions may be determined by performing a molecular dynamics simulation of the protein and quantifying the dynamic variability associated with different positions (i.e., different residues, together forming regions) of the protein.

As used herein, the term “key catalytic residue” refers to a residue (i.e., a particular amino acid at a particular position within an amino acid chain) of a protein that is particularly important to the catalytic activity of the protein. A residue may be considered to be particularly important to the catalytic activity of the protein if it binds to a cofactor, has been identified as essential for the chemical reactions, has been demonstrated to be associated with an increased catalytic activity compared to a corresponding reference (e.g., wild type) residue, and/or its mutation has been demonstrated to result in a decreased catalytic activity.

As used herein, the term “near attack conformation” (NAC) refers to a structure which is not necessarily a ground state but corresponds to a low energy conformation that lies on the transition path of a chemical reaction (reaction coordinate), as opposed to the “transition state” (TS) corresponding to a configuration that has the highest potential energy along the reaction coordinate. The “reactant complex” (RC) corresponds to a ground state conformation, which is a local energy minimum in the reaction coordinate.

### Prediction of Enzyme Catalytic Activity

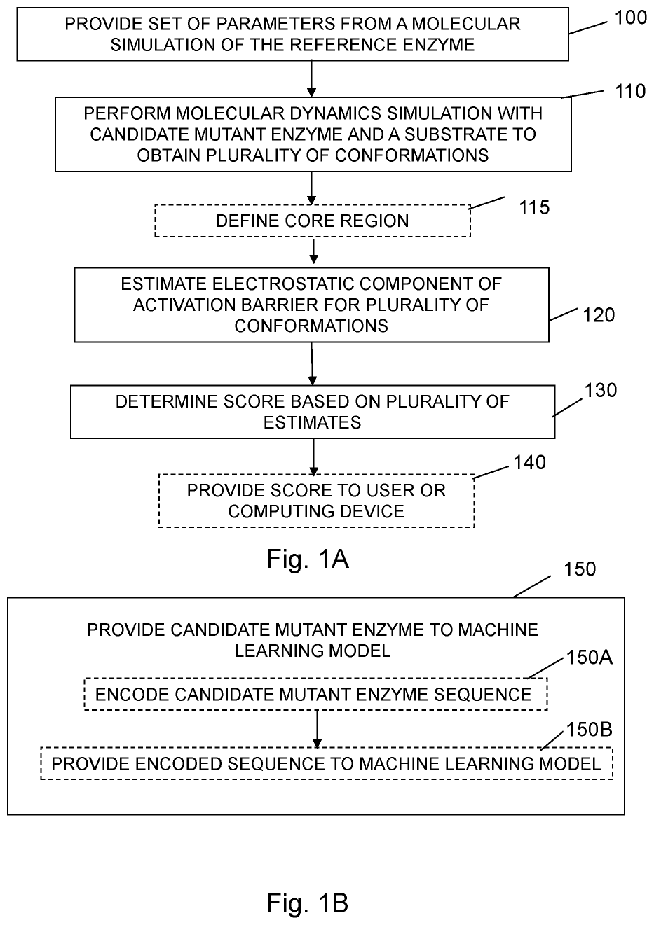

FIGS. 1A and 1B illustrate schematically methods of predicting enzyme catalytic activity for a candidate mutant enzyme as described herein. The method of FIG. 1A comprises providing, at step 100, a set of parameters from a molecular simulation (also referred to herein as “optimisation”) of a reference enzyme wherein the candidate mutant enzyme differs from the reference enzyme by one or more amino acids, wherein a region of the enzyme (QM region) comprising at least part of the active site and a substrate of the enzyme is optimised with a quantum mechanics method. At step 110, a molecular dynamics simulation is performed with the candidate mutant enzyme and a substrate of the enzyme to obtain a plurality of conformations each associated with a set of atomic coordinates. At step 120, the electrostatic component of the activation barrier (ΔΔG^{‡}_{Q20}) is estimated for each of the plurality of conformations of the candidate mutant enzyme, using the parameters from the molecular simulation of the reference enzyme and the set of atomic coordinates associated with the respective conformation, thereby obtaining a plurality of estimates of the electrostatic component of the activation barrier (ΔΔG^{‡}_{Q20}). At step 130, a score (ΔΔG^{‡}_{Q20EFF}, μ_{Q20}) is determined based on the plurality of estimates of the electrostatic component of the activation barrier, wherein the score is indicative of the effective activation barrier (ΔΔG^{‡}) of the candidate mutant enzyme.

The set of parameters from a molecular simulation of the reference enzyme may have been previously determined. Thus, the step of providing the set of parameters may comprise retrieving the set of parameters from a memory, receiving the set of parameters from a computing device, receiving the set of parameters from a user interface, etc. Alternatively, the set of parameters from a molecular simulation of the reference enzyme may be determined by performing a molecular simulation of the reference enzyme, wherein a core region of the enzyme comprising at least part of the active site is optimised with a quantum mechanics method, and a remaining external region is optimised with a molecular mechanics method (or coordinates from a molecular dynamics simulation are included in a DFT model by use of constraints) and determining the set of parameters from the molecular simulation. Performing a molecular simulation of the reference enzyme may comprise: providing a crystal structure of the reference enzyme and of the substrate (for example by obtaining a crystal structure from a database, from a user interface, etc.), performing a molecular dynamics simulation of the reference enzyme and substrate (optionally using one or more constraints to maintain the substrate and enzyme in a near attack conformation), preferably for a period of time between 1 and 10 μs, selecting a conformation from the molecular dynamics simulation, and using the conformation to obtain a quantum mechanics model of the transition state structure. This may be performed using a DFT model.

As used herein, references to predicting catalytic activity for a candidate mutant enzyme may refer to predicting an effective change in activation barrier (ΔΔG^{‡}), due to the presence of the enzyme (typically a reduction in the effective activation barrier). This may also be referred to herein simply as “activation barrier” or “effective activation barrier”. Thus, the terms “activation barrier”, “effective change in activation barrier”, “change in activation barrier” and “effective activation barrier” are used interchangeably herein unless context indicates otherwise. The activation barrier (also referred to as free energy activation barrier height or Gibbs energy ΔG^{‡}) of a reaction is the Gibbs energy of activation to achieve the transition state. The effective change in activation barrier may be considered to be indicative of the activation energy in the Arrhenius equation and as such may be used in such an equation. In contrast to the activation energy ΔE^{‡}_{QMMM}, ΔΔG^{‡} is generally negative because it encompasses the favourable effect of the enzyme.

The candidate mutant enzyme differs from a reference enzyme by one or more amino acids, wherein the one or more amino acids may be independently selected from: a proteinogenic amino acid, a non-proteinogenic amino acid, a chemical derivative of a proteinogenic amino acid linked to another moiety via a peptide bond, an amino acid modified by glycosylation, a phosphorylated amino acid, an amino acid that is or has the potential to be cross-linked via a disulphide linkage, and an amino acid that is cross-linked via a chemical crosslinker other than a disulphide linkage. The enzyme may be of any class. The enzyme may be selected from: an enzyme that belongs to an oxidoreductase class, an enzyme that belongs to a transferase class, an enzyme that belongs to a hydrolase class, an enzyme that belongs to a lyase class, and an enzyme that belongs to an isomerase class. References to enzyme classes may refer to enzyme classes as defined by the Enzyme Commission (i.e. “EC” enzyme classes). The reference enzyme may be any enzyme for which structural coordinates (i.e. molecular structure data) may be obtained and for which a postulated reaction mechanism is available. Structural coordinates may be obtained by experiment (e.g. using X-ray crystallography or NMR), may have been previously obtained or may be calculated from previously obtained structural coordinates. A postulated reaction mechanism may be obtained from e.g. the literature, or may be obtained based on e.g., other similar substrates or enzyme or optionally based on purely theoretical considerations (e.g., by DFT calculations).

The region of the enzyme that is optimised with a quantum mechanics method (referred to herein as “QM region”) may further comprise at least a fragment of a cofactor, or all of the atoms of a cofactor. The QM region may comprise one or more water residues. For example, one or more water residues may be included in the QM region in embodiments where the one or more residues are involved in the postulated reaction mechanism of the enzyme.

The step of providing a set of parameters from a molecular simulation of the reference enzyme may comprise performing a molecular simulation of the reference enzyme in order to obtain the set of parameters. Alternatively, the set of parameters may have been obtained from a previously performed molecular simulation. The molecular simulation may be a QM/MM molecular simulation, wherein a region of the enzyme (QM region) comprising at least part of the active site and a substrate of the enzyme is optimised with a quantum mechanics method, and the remaining of the enzyme (or enzyme-substrate, or enzyme-substrate-cofactor system) is optimised with a molecular mechanics method (“MM region”). Alternatively, the molecular simulation may be a purely QM molecular simulation, such as a simulation using a DFT cluster method. The molecular simulation of the reference enzyme may be based on molecular structure data of the enzyme that has been previously obtained, such as e.g. crystal structure data (such as e.g. obtained by X-ray crystallography), NMR structure data, or a combination or derivative thereof, such as e.g. molecular structure data that has been obtained by homology modelling using previously obtained molecular structure data for a similar enzyme. Preferably, the molecular simulation of the reference enzyme may be based on a crystal structure or a homology model derived from a crystal structure (such as a crystal structure of a related enzyme). Molecular structure data for an enzyme may have been obtained from one or more databases (such as e.g. PDB), or may be obtained as part of the present method or prior to the present method, for example by homology modelling.

In the illustrated embodiment, the method further comprises step 115 of defining a core region that includes one or more of the atoms of the QM region, and an external region that includes the remaining atoms of the enzyme. In this embodiment, the set of parameters from the molecular simulation of the reference enzyme comprises: the changes to the partial charges of the atoms in the core region (ΔQᵢ) that occur during the formation of the transition state for a particular conformation of the reference enzyme from the reaction complex, and partial atomic charges for atoms in the external region. The step of estimating the electrostatic component of the activation barrier may in such embodiment comprise determining a change in partial atomic charges for each atom in the core region for each of a plurality of conformations, each optimised by electronic structure methods, such as a DFT cluster methodology or a QM/MM methodology. A representative change of partial atomic charges for each atom in the core region may be obtained as the mean value across each of the plurality of conformations. The change in charges may be calculated via a population analysis method including Mulliken population analysis, Hirshfeld population analysis, CM5 population analysis or other equivalent methods. Providing a set of parameters from a molecular simulation of the reference enzyme may comprise optimising a reaction complex and a transition state using any electronic structure method, such as a QM/MM or DFT cluster model. The set of parameters from a molecular simulation of the reference enzyme may be obtained by calculating the charges in the QM region (including the core region) in the reactant state (reaction complex) and in the transition state configuration. The difference of partial atomic charges may be calculated or may have been calculated using any method for the calculation of partial atomic charges. For example, the difference of partial atomic charges may be calculated using a Hirshfeld population analysis (as exemplified in Examples 4 to 7 below), a CM5 population analysis (as exemplified in Examples 1 to 3 below), or a Mulliken population analysis as exemplified in Example 5 below, on the core region atoms as optimised by electronic structure methods, such as DFT cluster methodology (Examples 4 to 7) or a QM/MM methodology (Examples 1 to 3). The difference of partial atomic charges may be calculated from a plurality of conformations as demonstrated in Example 6 below, where three distinct conformations were used and a DFT cluster model was obtained for each to determine the transition state and reactant complexes, and the mean of the change of atomic charges was obtained for each atom in the core region and used to parameterise the electrostatic mutant scoring (Q20) calculations. The set of parameters from a molecular simulation of the reference enzyme may be calculated or may have been calculated using a quantum mechanics model that does not include counter-ions and solvent. The set of parameters from a molecular simulation of the reference enzyme may be calculated or may have been calculated using a quantum mechanics model that only includes a fraction of the enzyme including at least the core region. This may enable more efficient calculations which may be advantageous when computational resources are limited. Faster computers may advantageously allow the use of larger models. Thus, the set of parameters from a molecular simulation of the reference enzyme may have been calculated using a quantum mechanics model that includes atoms of the core region, and any other atoms of the enzyme. Instead or in addition to this, the set of parameters from a molecular simulation of the reference enzyme may have been calculated using a quantum mechanics model that includes water molecules.

The candidate mutant enzyme may differ from the reference enzyme by one or more amino acids that may be located anywhere in the enzyme. Advantageously, the candidate mutant enzyme may differ from the reference enzyme by one or more amino acids may differ from the reference enzyme by one or more amino acids outside of the active site, and/or outside of the N terminus and/or C terminus region(s). The candidate mutant enzyme may differ from the reference enzyme by one or more amino acids that are not key catalytic residues. Key catalytic residues may be residues that have been a priori identified to be involved in a postulated reaction mechanism for the enzyme. The candidate mutant enzyme may differ from the reference enzyme by one or more amino acids excluding one or more residues specifically selected to restrain the substrate and/or cofactor during molecular dynamics simulations. The candidate mutant enzyme may differ from the reference enzyme by any number of amino acids. For example, in Examples 3 to 7 below, three mutations were inserted for each mutant sequence. In Example 6 below a series of additional data analysis were performed on additional datasets for one seed conformation, by either inserting 6 single mutations per mutant (namely set XMT6) or 12 single mutants (namely set XMT12). A total of 4250 mutants were generated for each and the same ML procedure as that used for the triple mutant data sets was followed. No significant change in performance was found in the XMT6 and XMT12 data sets versus the triple mutant dataset from the 1000 ns conformation, suggesting that any number of mutants can be interchangeably used which in turn presents no major technical challenge.

Performing a molecular dynamics simulation with the candidate mutant enzyme and substrate may comprise performing a molecular dynamics simulation for a period of at least 0.1 ns, at least 1 ns, at least 5 ns, at least 10 ns, at least 20 ns, at least 30 ns, at least 40 ns, about 1 ns or about 50 ns. The plurality of conformations may correspond to a plurality of times of the molecular dynamics simulation. The plurality of conformations corresponds to a plurality of times sample from the molecular dynamics simulation. The plurality of conformations may be sampled at regular intervals during a period of the molecular dynamics simulation. The plurality of conformations may comprise at least 10, at least 20, at least 30, at least 40, or at least 50 conformations. Molecular dynamic simulations may be performed for 0.1 ns or more. Without wishing to be bound by theory, the inventors believe that longer the molecular dynamics (MD) simulations may be associated with better signal to noise ratio than shorter ones. Further, the inventors believe that there is no upper limit to the length of molecular dynamics simulation that would be suitable, which is primarily limited by the available computational resources. The data of the molecular dynamics simulation may be saved and/or scored every 0.1 ns if considered practical for the computational resources. Depending on the length of the MD simulations, it may be more practical to save and score the data less frequently, such as e.g. every 1.0 ns. The molecular dynamics simulation may be performed in NVT, or at NPT conditions. An example of this is provided in Example 6 below.

As described above, a molecular dynamics simulation of the reference enzyme may have been performed or may be performed as part of this method to identify a near attack conformation from which an electronic structure method optimisation, such as DFT cluster or a quantum mechanics/molecular mechanics optimisation, can be performed to identify the set of parameters from a molecular simulation of the reference enzyme. The molecular dynamics simulation may be a relatively long molecular dynamics simulation, such as e.g. a 10 μs simulation, although this is not necessary. The conformation used as a starting point to perform the molecular dynamics simulation with the candidate mutant enzyme may be selected from a period at the end of this simulation, for example from the last 1 μs of the reference enzyme molecular dynamics simulation. The molecular dynamics simulation of the reference enzyme may be constrained to hold the enzyme, substrate, and any cofactor in a near attack conformation.

The method may further comprise step 140 of providing the score or information derived therefrom, to a user through a user interface, to a database or other computer readable storage medium, or to a computing device such as e.g. for further processing, analysis or use. For example, the score may be provided to a computing device to train a machine learning model to take as input a candidate enzyme sequence and produce as output a score indicative of the effective activation barrier of the candidate mutant enzyme, as will be described in relation to FIG. 1B. The method of FIG. 1B comprises step 150 of providing a candidate mutant enzyme as an input to a machine learning model that has been trained to take as input a candidate enzyme sequence and produce as output a score indicative of the effective activation barrier of the candidate mutant enzyme, wherein the machine learning model has been trained using training data comprising a plurality of candidate mutant enzyme sequences and corresponding scores indicative of the effective activation barrier (ΔΔG^{‡}) of the candidate mutant enzyme obtained using a method as described in relation to FIG. 1A. Providing a candidate mutant enzyme as an input to a machine learning model may comprise providing a candidate mutant enzyme sequence to the machine learning model. The candidate mutant enzyme sequence may be provided to the machine learning model at step 150B as a sequence encoded at step 150A using an encoding scheme.

The machine learning model may comprise one or more ensembles of individual machine learning models. Each ensemble of individual machine learning models may comprise at least 2 individual machine learning models, at least 5 individual machine learning models, at least 10 individual machine learning models, at least 15 individual machine learning models, at least 20 individual machine learning models, at least 25 individual machine learning models, between 5 and 30 individual machine learning models, between 10 and 30 individual machine learning models, or between 20 and 30 individual machine learning models. Any number of ensembles and any number of models per ensemble may be used. The machine learning model may comprise at least 100, between 100 and 300, or between 150 and 250 individual machine learning models, which may be grouped in ensembles. Without wishing to be bound by theory, the inventors believe that increasing the number of models (in total or per ensemble) may be associated with diminishing returns at least above a number of models that is problem-dependent. Further, while the number of models (in total or per ensemble) is not limited in theory, it may in practice be limited with computational time and memory limitations. The optimal number of individual models in an ensemble may depend on the enzyme, the configuration and type of the machine learning model and how the mutant enzyme data is encoded. For example, a machine learning model comprising models trained using 5 different seed conformations may have 30 models per seed conformation as an optimal number of individual models (i.e., 150 individual models in total), whereas a machine learning model comprising models trained using 10 different seed conformations may have 20 models per seed conformation as an optimal number of individual models (i.e., 200 individual models in total). In another example (see Example 5) for machine learning models trained on 5 seed conformations, a single machine learning model may be used for each seed conformation, totaling to 5 individual machine learning models. Conversely, an ensemble of models trained using a single seed conformation may be used (as demonstrated in Example 7). Note that the overall performance of these models may not be the same and the choice of the number of seed conformations and individual models to use may depend on factors such as the desired level of accuracy, computation limitations (e.g., available computing power/time), etc. Any type of machine learning model may be used, including Bayesian models, random forest, k-nearest neighbour models, deep learning models, regression models, support vector machines, neural network models, etc. For example, the machine learning model may comprise a plurality of Lasso regularised linear regression models, or a plurality of dense neural network models.

At step 150A, the candidate mutant enzyme sequence may be encoded using an encoding dictionary where each amino acid is represented by a vector of size N. For example, each element of the vector may be an amino acid property from a randomly selected set of amino acid properties, optionally from the AAindex amino acid properties database (this may be referred to as “randomly selected AAindex” encoding scheme). Alternatively, each element of the vector may be a random number, optionally wherein the real random number is selected between 0 and 1 (this may be referred to as “random” encoding scheme). Alternatively, each element of the vector may be a 0 or a 1, wherein the vector has size N equal to the number of different amino acids considered, and each vector contains a single 1 or a single 0 at a position specific for the amino acid being encoded (this may be referred to as one hot encoded”). Alternatively, each element of the vector may be a 0 or a 1, wherein the vector has size N=1, and the element is equal to 0 if the residue is not mutated and 1 otherwise, or vice-versa. Alternatively, the full enzyme sequence may be encoded by a single vector of length equal to the number of residues where each mutant is encoded to contain the same number (e.g., 0) except for the position where a mutation or mutations have been inserted and which a different number (e.g., 1) is used. Regardless of the encoding dictionary used, the resulting encoded sequence of numbers may be subject to a fast Fourier transform procedure for each encoded vector and the real part of the FFT result is used to encode the protein sequence data. For example, any of the following encoding methods (and associated dictionaries) may be used: random FFT (i.e. each element of the vector being a random number, in combination with a subsequent FFT step), random NonFFT (i.e. each element of the vector being a random number, without a subsequent FFT step), randomly selected AAindex FFT (i.e. each element of the vector being an AAindex property from a randomly selected set, in combination with a subsequent FFT step) and one hot encoded. Each of this are exemplified in the examples below. In the random encoding methodology a lookup table of variable size N×M may be used, where M is the number of types of amino acids (e.g., 20 for only all the natural amino acids), And N is the encoding complexity, which can be 1 (or any larger integer number). Hence M encoding vectors of size N are generated. Each resulting matrix may then be filled up with numerical values such that for each amino acid and for each random encoding vector, a series of real numbers (each between 0 and 1) are generated randomly to construct a look up table. When an FFT is additionally performed, the FFT transform may be performed on each encoded vector independently. The first datapoint of each transform may be ignored. Only a subset of datapoints of each transform may be included up to a specific number of datapoints, based on the following rules: IF an even number of residues are encoded THEN the number of datapoints included is the total number of residues divided by 2 (and ignoring the first datapoint of the FFT), OR IF an odd number of residues are encoded THEN the number of included datapoints is the total encoded minus 1 and then divided by 2 (and ignoring the first datapoint of the FFT). The AAindex encoding methodology may comprise defining an encoding complexity N, and randomly selecting a series of N properties from the AAindex database (although other databases can be substituted). A lookup table can then generated based on these vectors, resulting in a similar table to that used in the random encoding approach. The remaining steps may be identical to the fully random encoding (as in that case an optional FFT step may be performed). The encoding complexity may be selected using a grid search, as demonstrated in Example 5. N=The encoding complexity N means that for each amino acid type in the residue, a distinct vector of size N is defined that is then used to encode the enzyme, selecting for each residue in the enzyme the corresponding vector and joining them all together into a final array for each mutant.

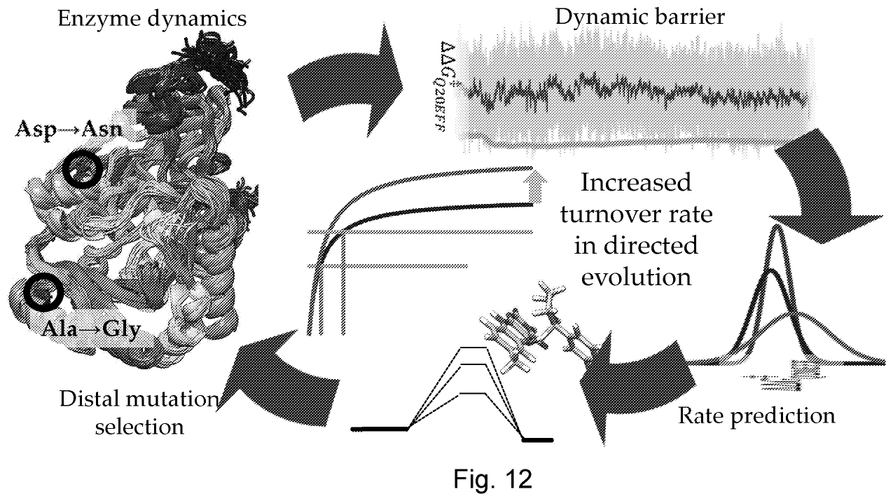

FIG. 12 shows a general schematic of the process of estimating catalytic barriers based on electrostatic effects and dynamics with the technology described herein. The method illustrates the general principle of introducing mutations in the structure of a reference enzyme, simulating enzyme dynamics of the mutated enzyme, estimating the dynamic barrier for a plurality of conformations along the dynamics simulation, using this information to predict the catalytic rate, and selecting mutations (including mutations that are distal to the catalytic site) based on these predictions to obtain a candidate enzyme with increased turnover rate.

The methods of predicting enzyme catalytic activity for a candidate mutant enzyme described above and in relation to FIG. 1 may be used to provide scores for a plurality of candidate mutated enzymes. These may comprise mutations at a plurality of positions in the reference enzyme, thereby providing information about the mutagenesis potential at these positions. Thus, the methods of predicting enzyme catalytic activity for a candidate mutant enzyme described above and in relation to FIG. 1 may be used to provide a site directed mutagenesis potential map for a reference enzyme, by predicting the catalytic activity of each of a plurality of candidate mutated enzymes that differ from the reference enzyme by at least one amino acid at a plurality of positions that together form a mapped region. In other words, the methods described above and in relation to FIG. 1 can be used to predict the effect of mutations at each position in a mapped region (which can cover the entire sequence of the enzyme). This may be used in the context of enzyme engineering as will be described further below.

### Enzyme Engineering

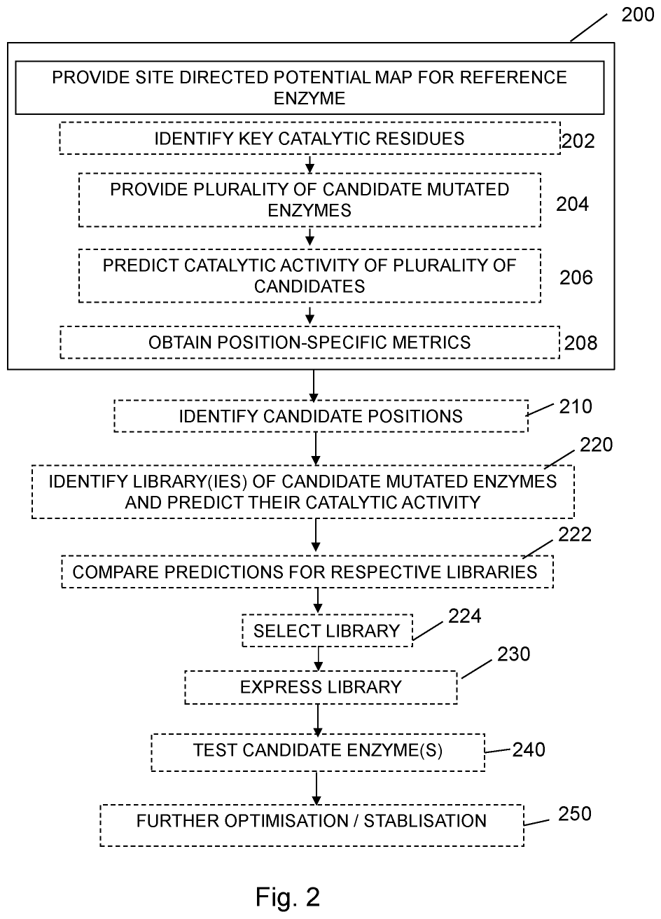

The method of predicting enzyme catalytic activity and/or the results thereof find use in any context where characterisation of the catalytic activity of an enzyme may be desirable, such as for example in the context of enzyme engineering. FIG. 2 is a schematic flow chart showing in general terms a method of engineering an enzyme (i.e. a method of providing a candidate mutant enzyme with improved catalytic activity compared to a reference enzyme) as described herein.

The method comprises step 200 of providing a site directed mutagenesis potential map for a reference enzyme. This may comprise optional step 202 of identifying key catalytic residues (e.g. particular amino acids at particular positions that are important for catalytic activity) by any recombinant technique such as site directed mutagenesis, and including these key catalytic residues in the reference enzyme. Step 200 may further comprise step 204 of providing a plurality of candidate mutated enzymes, wherein the candidate mutant enzyme differs from the reference enzyme by at least one amino acid at a plurality of positions that together form a mapped region; step 206 of predicting the catalytic activity of each of the plurality of candidate mutated enzymes using the methods of predicting catalytic activity described herein and by reference to FIG. 1, thereby obtaining for each candidate mutated enzyme a score indicative of the in the effective activation barrier of the candidate mutant enzyme; and step 208 of combining the scores for the plurality of candidate mutated enzymes into one or more position-specific metrics indicative of the potential for mutant-associated catalytic improvement at the position.

At step 210, one or more candidate position(s) that is/are associated with one or more candidate mutant enzymes likely to have improved catalytic activity are identified based on the one or more position-specific metrics. For example, all positions in the mapped region may be ranked based on one of the one or more position-specific metrics. For example, the one or more position-specific metric may comprise an average score across mutants that comprise a mutation at the respective position, and the candidate positions may be ranked by order of the most negative average score. For example, candidate positions that have more negative average scores may be more likely to have improved catalytic activity than candidate positions that have less negative average scores. A plurality of candidate positions may be selected based in part on the location of the positions in the enzyme. For example, the plurality of candidate positions may be selected to be distributed throughout the enzyme sequence or to be located in different parts of the enzyme. This may enable a more meaningful/thorough exploration of the mutation potential in the enzyme. Additional practical criteria may be considered when selecting candidate positions, such as e.g. criteria related to the feasibility of obtaining a library that targets these positions (e.g. one or more criteria associated with a specific gene synthesis methodology).

At optional step 220, one or more candidate mutant enzymes comprising mutations at the one or more candidate position(s) are identified and their catalytic activity is predicted using the methods of predicting catalytic activity described herein and by reference to FIG. 1. The one or more candidate mutant enzymes may together form a library. The method may further comprise repeating step 220 with another library (or one or more further libraries), and comparing the predictions for the respective libraries at step 222. Comparing the predictions for the respective libraries may comprise determining a summary statistic for the respective libraries, such as e.g. the mean or median score across candidate mutant enzymes in the library. The method may further comprise selecting a library at step 224 based on the comparing step, such as e.g, the library that is associated with the highest mean or median score.

At optional step 230, one or more of the identified candidate mutant enzymes are obtained, for example by expressing a gene library designed based on the one or more identified candidate mutants. At optional step 240, the candidate mutant enzymes obtained are tested for one or more properties including catalytic activity and/or for one or more properties for a property other than catalytic activity. At optional step 250, an identified candidate enzyme may be subjected to a further optimisation and/or a stabilisation process. For example, the identified candidate enzyme may be used as a new reference enzyme and the method of FIG. 2 may be repeated.

### Systems

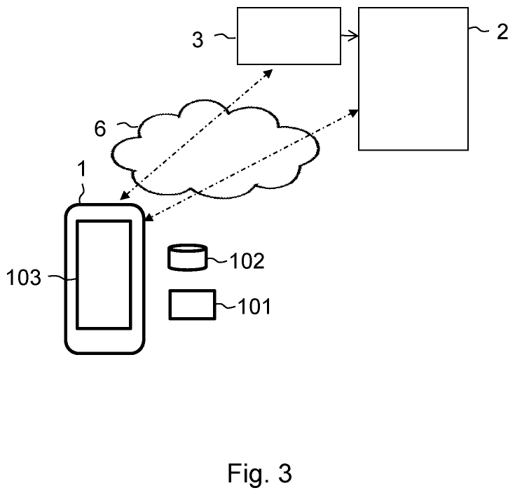

FIG. 3 shows an embodiment of a system for predicting the catalytic activity of an enzyme, and/or for enzyme engineering based at least in part on the prediction of enzyme catalytic activity, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g., through audible or visual signals. The computing device 1 may be communicably connected, such as e.g., through a network 6, to automated laboratory equipment 3, such as one or more robotic liquid handlers and/or analytical equipment, and/or to one or more databases 2 storing analytical, sequence and/or prediction data. The one or more databases may additionally store other types of information that may be used by the computing device 1, such as e.g., reference sequences, parameters, etc. The computing device may be a smartphone, tablet, personal computer or other computing device. The computing device is configured to implement a method for predicting enzyme catalytic activity and/or a method for enzyme engineering, as described herein. In alternative embodiments, the computing device 1 is configured to communicate with a remote computing device (not shown), which is itself configured to implement a method for predicting enzyme catalytic activity and/or a method for enzyme engineering, as described herein. In such cases, the remote computing device may also be configured to send the result of the method to the computing device. Communication between the computing device 1 and the remote computing device may be through a wired or wireless connection, and may occur over a local or public network such as e.g., over the public internet or over WiFi.

The laboratory equipment 3 means may be in wired connection with the computing device 1, or may be able to communicate through a wireless connection, such as e.g., through a network 6, as illustrated. The connection between the computing device 1 and the automated laboratory equipment 3 may be direct or indirect (such as e.g., through a remote computer). The automated laboratory equipment 3 may be configured to produce and/or test an enzyme. Any sample preparation process that is suitable for use in producing an enzyme having a particular sequence and/or testing one or more properties of an enzyme may be used within the context of the present invention. The automated laboratory equipment 3 may be in direct or indirect connection with one or more databases 2, on which analytical data (raw or partially processed) may be stored.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1: Using Dynamics to Predict Enzyme Catalytic Turnover Number and Application to the Prioritization of Directed Evolution Distal Amino Acid Mutations in an Oxidoreductase (EC-1)

### Introduction

Theoretical methods have been extensively used to estimate the turnover rate of enzymes to different degrees of accuracy, relying in general on a mixture of electronic structure methods and molecular dynamics (MD) simulations that employ transition state theory to calculate the rate of reaction as a function of the free energy barrier between the reactant complex state and the activated complex, or transition state [16-18]. Density functional theory (DFT) cluster approaches, where only a subset of the enzyme is included in the models, allow for accurate relative-energy estimations of reaction barriers. While sufficiently large DFT-cluster models can predict the effect of active-site mutagenesis on rate (or of any residues included in the cluster models), extensive modelling is required to find the lowest possible reaction energy for each potential mechanistic pathway [19]. Alternatively, multi-scale approaches such as quantum mechanics/molecular mechanics (QM/MM) methods benefit from breaking up the system and modelling different regions or phenomena at different levels of theory, allowing for larger regions to be calculated and some conformational dynamics to be considered through proper sampling [20-22]. On the one hand, to calculate a representative set of structures and capture the dynamical effects of an enzyme with QM/MM would currently require vast computational resources [23, 24]. On the other hand, MD simulations of small proteins can access real milliseconds [25], but MD cannot alone fully predict catalytic phenomena. Further, the contribution of enzyme dynamics and distal mutations to catalysis remains an open question [26]. While some theoretical consideration of conformational dynamics is essential to predict the free energy of activation [27-29], some studies propose that molecular dynamics are the key to understanding the effects of distal mutations [10, 30-35].

The aim of the work in this example is to develop a new methodology to estimate k_{cat} (also known as the enzyme turnover number). The methodology combines global enzyme dynamics and electrostatics for the prediction of k_{cat} and can sense changes in k_{cat} as the conformation and dynamics of the enzyme are altered by even distal mutations. The strategy is to use MD to provide enzyme dynamics and then to estimate catalytic energetics using QM approximations. Such an approach melds the main benefits of the two approaches. A series of QM/MM (equivalent results to QM/MM models could be obtained by DFT calculations instead) and electrostatic calculations (based on conformations from MD simulations) are used to investigate the contribution of whole enzyme conformational and dynamical effects to the turnover rate. This is achieved by rapidly calculating the contribution to enzymatic turnover rate at sampled timepoints from an MD trajectory by approximating the free energy of activation from electrostatics. In this example, the inventors demonstrate that the relative electrostatic component of k_{cat} can be predicted by calculating and averaging the distribution of the activation barriers from substantial numbers of dynamic conformations. The work described in this example further demonstrates the use of the newly developed methodology in mutants of the EC-1 oxidoreductase: 6-hydroxy-D-nicotine oxidase from Arthrobacter nicotinovorans.

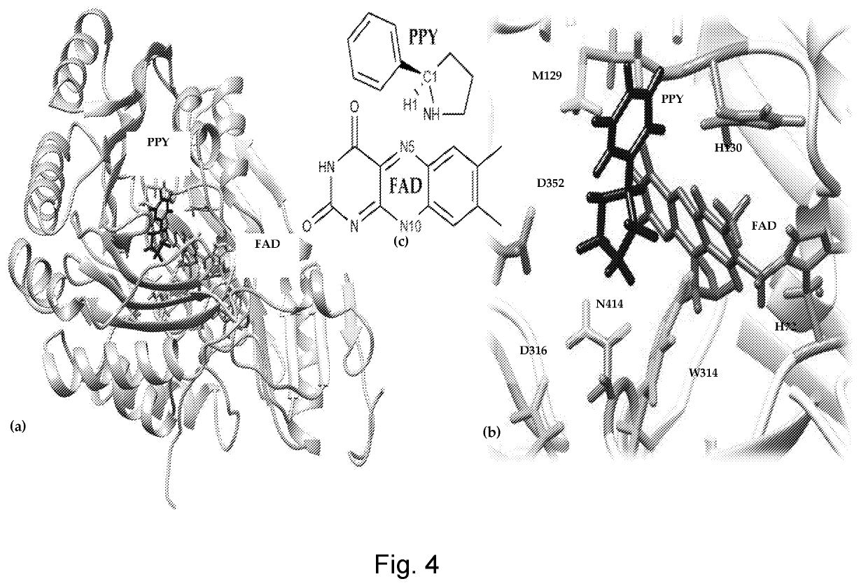

An important target for biocatalysis is the production of chiral amines, found in many active pharmaceutical ingredients (APIs). While enzymes have been shown to readily produce some of these enantiomerically-pure intermediates [3], simultaneously tackling the selective synthesis of a broad range of amines and their (R)- and (S)-enantiomers is a real challenge. Flavin-containing monoamine oxidase variants have been developed that are highly (S)-selective and have a broad substrate specificity that encompasses a wide range of amines [4, 5], but few (R)-selective amine oxidases have been reported. One such enzyme is 6-hydroxy-D-nicotine oxidase from Arthrobacter nicotinovorans, which is a highly (R)-selective amine oxidase (FIG. 4). While this enzyme has recently had its substrate-scope broadened (by site directed mutagenesis) to include molecules such as (R)-2-phenylpyrrolidine (FIG. 4c) [6], no optimisation of the catalytic turnover rate has been performed.

### Results

Establishing a Near Attack Conformation and Consistency with the Hydride Transfer Reaction

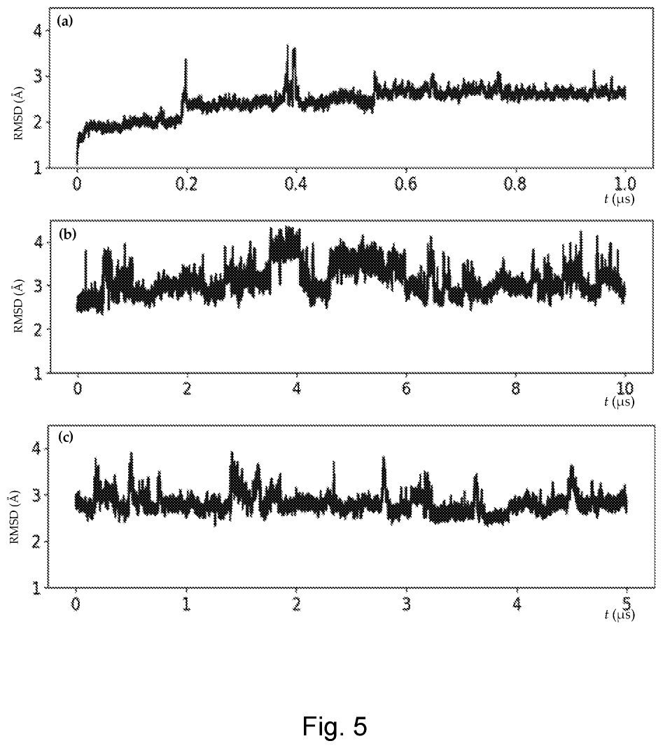

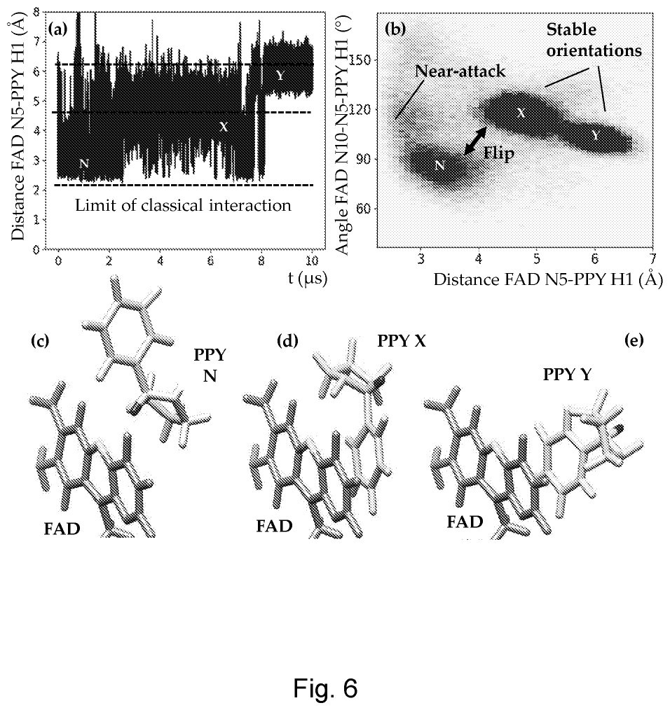

A 1 μs molecular dynamics (MD) simulation was performed on a crystal structure of 6-hydroxy-D-nicotine oxidase to solvate the sidechains and protein loops. Two mutations were inserted into the wildtype enzyme from Arthrobacter nicotinovorans (E350L/E352D) to produce a mutant with broadened substrate-scope (hereinafter referred to as D2) [6]. The D2 protein equilibrated at 3 Å from the crystal structure (FIG. 5) as assessed by the all-atom root-mean-square deviation (RMSD); there was no evidence of any significant unfolding of the protein core. Following this, a single (R)-2-phenylpyrrolidine substrate (referred to as PPY, see FIG. 4) was placed into the active site and a further 10 μs of unrestrained MD simulation was performed. Again, the RMSD was stable in the range 3-3.5 Å throughout this long simulation (FIG. 5). Several substrate orientations were observed, with distinct distances (PPY H1 to FAD N5) and angles (PPY H1, FAD N5 and N10) to the flavin cofactor (FIG. 6), with most of the time spent with H1-N5 in 4-6 Å range (see FIG. 4c for atom name definitions). Two stable positions were observed for the substrate within the enzyme pocket that would not be amenable to hydride transfer. However, the substrate also repeatedly reached close hydride-transfer near-attack conformations with respect to FAD, where the H1-N5 distances were in the limit of their classical interaction (around 2 Å). In these orientations, the attack angle was localized to a tight region around ˜120°. The substrate could move rapidly between a stable pocket orientation and near-attack conformations simply by rotationally flipping into the right orientation. This ability, which is dictated by the shape and size of the pocket, could be a key determiner of substrate specificity and enantiomeric selectivity. A set of suitable near attack conformations were extracted from the D2·PPY complex simulation, including one at 1212 ns (FIG. 6c), for further modelling with MD and QM/MM methods.

Measuring the Near Attack Angle Dependency with QM

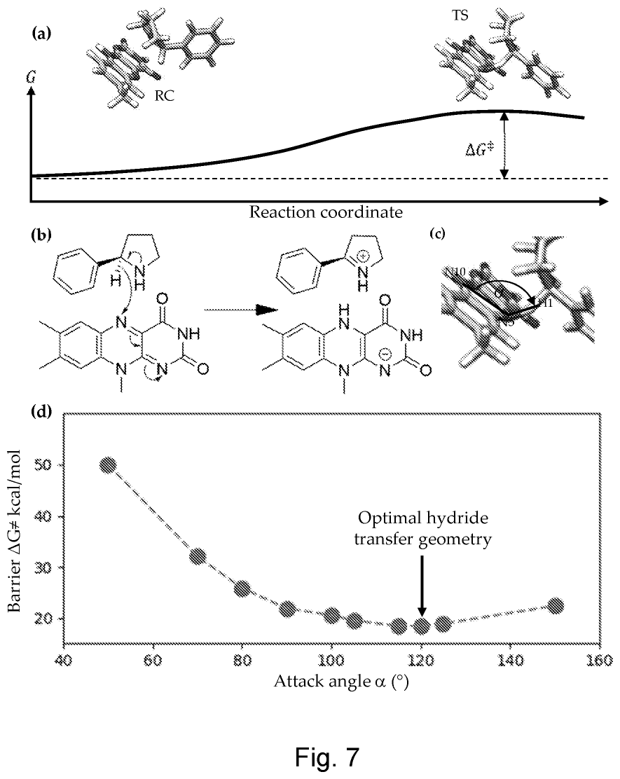

A series of minimal models were constructed for a range of attack angles (defined by the atoms PPY H1, FAD N5 and N10, see FIG. 7c) and optimised using density functional theory (DFT), in the absence of enzyme. A clear dependence of the free energy activation barrier height (ΔG^{‡}) for the hydride transfer mechanism (FIG. 7a & 7b) on this attack angle (α, see FIG. 7c) was observed. In particular, ΔG^{‡} was found to display a minimum for a in the 90-1500 range (FIG. 7d). A ΔG^{‡}-value as low as +18.4 kcal×mol⁻¹ was calculated for α=120°. The activation barrier increased steeply for attack angles below 90°. For example, ΔG^{‡} was +32.1 kcal×mol⁻¹ at α=70°, which would result in a k_{cat} reduction of over ˜10³ at room temperature. Further decreasing a to 500 resulted in a free energy of activation for hydride transfer of +50.1 kcal×mol⁻¹, which is nonreactive. All the near-attack conformations observed in the unrestrained MD simulation of PPY (labelled N in FIG. 6) were found to have attack angles that corresponded to low free energies of activation, confirming that the substrate-enzyme complex was already pre-organized towards a low energy reaction pathway for the hydride transfer mechanism.

### The Energetics of the Enzyme Hydride Transfer Activation Barrier

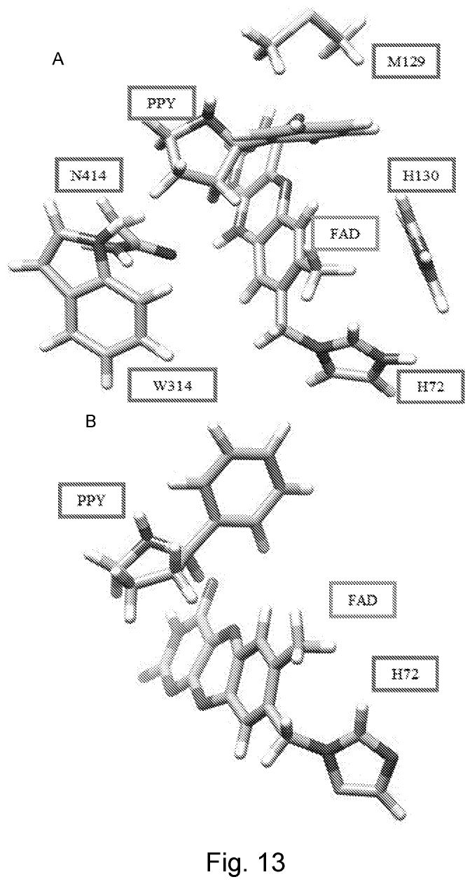

A set of QM/MM models were prepared from near attack coordinates (sampled from the unrestrained MD simulation, see FIG. 13) to model the activation barrier for the hydride transfer mechanism in the presence of the full enzyme and solvent. Alternatively, DFT models could be used for this purpose, as demonstrated in Examples 4 to 7. The optimised reaction complexes (RC) and transition states (TS) exhibited N5-H1 distances in the ranges 2.0-2.6 Å and 1.2-1.4 Å. The localisation of the TS in the N5-H1 reaction coordinate had a standard deviation of 0.046 Å, and a mean QM/MM activation barrier (ΔE^{‡}_{QMMM}) of +11.7 kcal×mol⁻¹ was calculated. Furthermore, as the sampled coordinates in all models were sourced from the 1.1-2.9 μs period of the unrestrained D2·PPY complex MD simulation, a clear spread of conformations and a consequent variance of activation energies were observed in the range of −5.4 to +29.1 kcal×mol⁻¹ (see Table 3). Therefore, calculations based on this methodology reflect the dependence of the hydride transfer barrier on overall enzyme conformational dynamics.

TABLE 3
Relative QM/MM energies as optimised by Chemshell/
Turbomole/DL_Poly at the B3LYP/def2-SVP level of theory with a
CHARMM forcefield for the MM region and Q20 electrostatic results
at each frame, based on QM/MM optimised or non-optimised coordinates.

| | QM/MM_{QM} | QM/MM | ΔΔG_{Q20}^{‡} − | ΔΔG_{Q20}^{‡} − |
|---|---|---|---|---|
| | Energy | Energy | Optimised | Nonoptimised |
| Frame | (kcal/mol) | (kcal/mol) | Coordinates | Coordinates |
| 11178 | 7.96 | 9.64 | −10.2 | −13.9 |
| 11597 | 5.89 | 3.85 | −10.4 | −11.1 |
| 12101 | 8.22 | 8.91 | −10.8 | −14.7 |
| 12109 | 3.15 | −5.43 | −11.4 | −10.4 |
| 12111 | 4.81 | 6.83 | −11.5 | −15.0 |
| 12140 | 6.85 | 9.51 | −10.0 | −13.4 |
| 12141 | 17.33 | 15.44 | −5.3 | −6.5 |
| 25807 | 23.85 | 27.53 | −3.0 | −3.7 |
| 25842 | 26.24 | 29.16 | 1.7 | −6.3 |

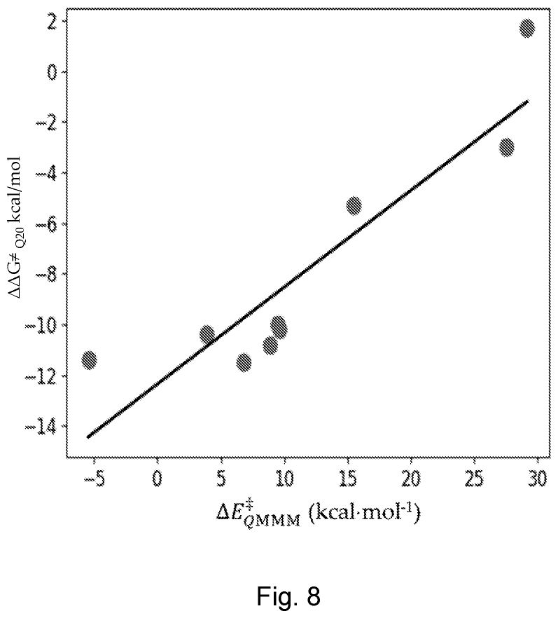

The QM/MM optimised coordinates were used to estimate the electrostatic component of the activation barrier based on a purely electrostatic methodology (referred to as Q20 here). Briefly, in the Q20 method the Coulombic interaction of the (static) MM region and the changes to the partial charges (of the reactive region) that occur during the formation of the TS from the RC are used to provide an estimate for the electrostatic component of the activation barrier (ΔΔG^{‡}_{Q20}). This process is distinct to other processes reported elsewhere at least in that a larger region (optionally including several fragments other than the substrate) are included in the core region, which encompasses all the main atoms involved in a change in partial atomic charges during the activation step [28, 36, 37]. Further differentiation is found in the application of the process in the wider context detailed in this example, including the addition of restraints, scoring of zero-charged mutants (by effect of MD simulation), scoring of fully equilibrated mutants during molecular dynamics including mutants outside of the active site and the lognormal correction to the measured effects, as well as in the application to evaluate a plurality of mutants. Based on the scoring, a good correlation (FIG. 8) was obtained between the ΔΔG₂ and ΔE^{‡}_{QMMM} results (a coefficient of determination of 0.82). In contrast to ΔE^{‡}Q_{MMM}, ΔΔG^{‡}_{Q20} is generally negative because it encompasses the favourable effect of the enzyme. Taken together, these findings support the hypothesis that the variability of ΔE^{‡}_{QMMM} (due to dynamics) is mostly explained by the electrostatic contributions from the D2 enzyme, which justifies the use of a purely electrostatic calculation to approximate the activation barrier. While these results are somewhat specific to D2 (6-hydroxy-D-nicotine oxidase), it is noted that a similar conclusion has been reached for other systems [38, 39], including monoamine oxidase [40]. This supports the primary role of electrostatics in a variety of enzymes [41].

The Q20 methodology allows the estimation of the activation barrier in a significantly less computationally intensive manner (and hence faster, given the same available computational resources) than using full QM/MM calculations. The approach allows the dynamics of the barrier height to be calculated over significant timescales, hence allowing for the incorporation of dynamic effects into the rate of reaction calculation. It is important to note that in any approach comparable to Q20, where there is no energy minimization, the estimation only has meaning in a reactive configuration; that is, when the substrate is in a near attack conformation and the induced polarizability of the enzyme and water act to reduce the energy required to form the active complex. This limitation may be practically overcome by imposing a restraint on the MD simulation to hold the substrate in a near attack conformation, such that only the distribution of the electrostatic effects of the enzyme towards stabilizing the active complex are observed.

Enzyme k_{cat} Prediction Using Restrained Molecular Dynamics

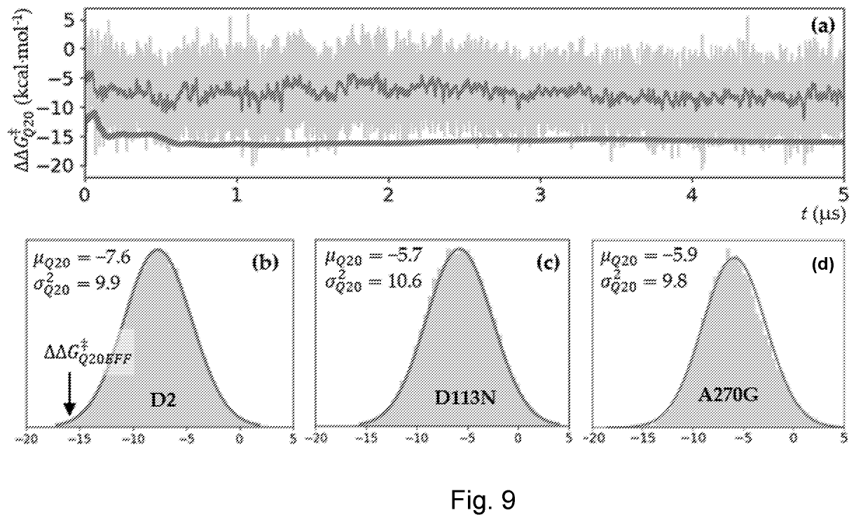

Therefore, to increase the population of near attack conformations, a harmonic restraint of 2.2 Å was added between PPY H1 and FAD N5 (using the same near attack coordinates taken from the unrestrained simulation at 1212 ns) and a further 5 μs of MD simulation performed with the enzyme set free to move. The restrained MD simulation accomplished the intended aim (resulting in a dramatic increase in near attack conformations), All geometries from the restrained MD fall into the low activation energy barrier region of the attack angle calculated from the minimal DFT model approach. This MD trajectory was used to produce coordinates (sampled every 0.1 ns, but could have been sampled at higher frequencies, for example any frequency up to the time between each integration step in the MD, 4×10⁻⁶ ns in this case, or at lower frequencies such as e.g. 0.5 ns) for the estimation of activation barriers during enzyme dynamics using the Q20 electrostatic methodology (grey trace labelled 900 in FIG. 9a for instantaneous barrier calculations, red trace labelled 910 in FIG. 9a for moving average of 100 frames, orange trace labelled 920 for accumulated ΔΔG^{‡}_{Q20EFF} based on the lognormal correction of Equation (2) in FIG. 9a).

In FIG. 9a (grey trace, 900), the fluctuation of ΔΔG^{‡}_{Q20} becomes evident, with values ranging from +5 kcal×mol⁻¹ to −20 kcal×mol⁻¹ (recalling that a lower effective barrier translates into faster rates, ΔΔG^{‡}_{Q20} is generally negative in this system, as Q20 does not comprise other generally positive and mostly constant contributions to ΔG^{‡}). The resulting distribution of ΔΔG^{‡}_{Q20} yields an arithmetic mean electrostatic contribution to the activation barrier (μ_{Q20}) of −7.6 kcal×mol⁻¹ with a variance (σ_{Q20}²) of 9.9 kcal²×mol⁻². It is therefore concluded that ΔΔG^{‡}_{Q20} forms a statistical distribution of values, and therefore the effective change in energy of activation (ΔΔG^{‡}_{Q20EFF}) affecting the rate equation must be calculated by a suitable measure of this statistical distribution. A normal distribution generated with the same arithmetic mean and variance results in a good fit (FIG. 9b). If the distribution is a normal distribution, for example, then the correct average measure to describe the effective barrier height is the log-normal mean because rate is exponentially dependent on activation barrier energy. Other statistical measures of centrality may be used for example if the distribution deviates from a normal distribution (e.g. if it exhibits significant skew or kurtosis). The effective energy to be used in an Arrhenius-type rate Equation (1), is therefore given by (2), dependent on both μ_{Q20} and σ_{Q20}², where RT is the product of the gas constant and temperature (which is 0.593 kcal×mol⁻¹ at standard temperature and pressure). From Equation (2), the effective electrostatic contribution to the barrier energy ΔΔG^{‡}_{Q20EFF} can be estimated to be −16.0 kcal×mol⁻¹. The orange trace in FIG. 9a shows that the running average of this effective energy is closer to the lower edge of the dynamic distribution of the normally distributed dynamic ΔΔG^{‡}_{Q20} values, rather than the mean (red trace), which is intuitive based on the exponential dependence of rate on activation barrier energy. Equation (2) can then be substituted into the Arrhenius rate Equation (1), to provide an estimate of k_{cat} subject to a pre-exponential factor. An important consequence of Equation (3) is that not only is the rate dependent on the average of the instantaneous barrier height energy, but it is also determined by the spread of the barrier energies, which is in turn dictated by enzyme global dynamics. However, in some cases an equivalent to Equation (3) without the second term (Equation (3a) below) may be used, and similarly for Equation (2), the term μ_{Q20} alone may be used. For example, this may be appropriate in cases where dynamics are not significant (as then the term in Equation (3) involving spread will be small compared to the term involving the mean) or when there is insufficient data to define the statistical distribution (and hence the spread of said distribution) accurately. For example, when using the log-normal mean and short molecular dynamics simulations, the variance may be noisier than the mean as it is takes more data to converge to its true value.

$$\\ \begin{matrix}{k}_{cat} ∝ exp ( - \frac{Δ Δ {G}_{Q 20 EFF}^{{}_{} ‡}}{RT} ) & ( 1 )\end{matrix}$$

$$\\ \begin{matrix}Δ Δ {G}_{Q 20 EFF}^{{}_{} ‡} = {μ}_{Q 20} - \frac{{σ}_{Q 20}^{{}_{} 2}}{2 RT} & ( 2 )\end{matrix}$$

$$\\ \begin{matrix}{k}_{cat} ∝ exp ( - \frac{{μ}_{Q 20}}{RT} + \frac{{σ}_{Q 20}^{{}_{} 2}}{2 {( RT )}^{2}} ) & ( 3 )\end{matrix}$$

$$\\ \begin{matrix}{k}_{cat} ∝ exp ( - \frac{{μ}_{Q 20}}{RT} ) & ( 3 a )\end{matrix}$$

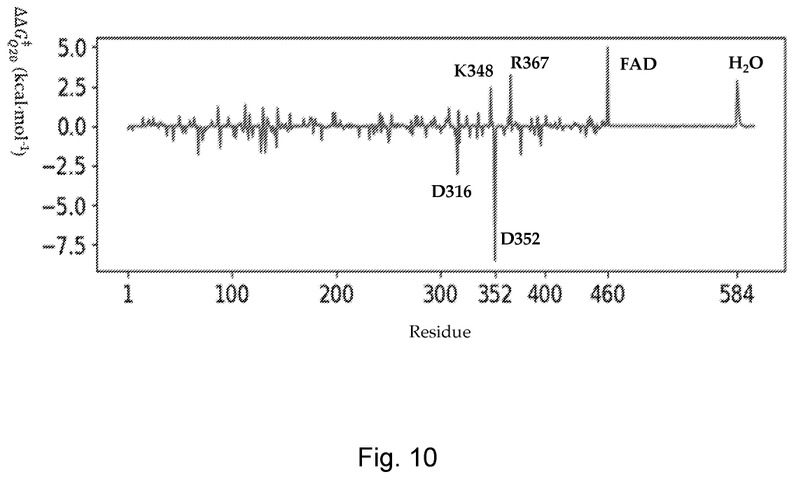

Another feature of the electrostatic approach is that the contributions of individual components (for example, individual D2-enzyme amino acids) can be assessed in isolation. FIG. 10 provides an intricate picture of the influence of each amino acid residue on the electrostatic barrier. Residue D352 is the most effective at lowering the overall reaction activation barrier (−8.5 kcal×mol⁻¹), while solvent serves to partially reverse this effect by enhancing the barrier height (+5.1 kcal×mol⁻¹). The flavin cofactor (FAD, residue 460) also has the tendency to increase the barrier, as might be expected due to its proximity to the substrate. FIG. 10 also shows other residues that directly contribute to the activation barrier energetics: residues K348 and R367 have an unfavourable effect of +2.4 kcal×mol⁻¹ and +3.2 kcal×mol⁻¹, respectively, while residue D316 has a favourable effect of −3.0 kcal×mol⁻¹. Moreover, the contribution of the solvent and counter ion molecules can also be selectively introduced into the scoring function (see Example 6 for a comparison between different scoring examples).

Towards Delineating the Effect of Mutations on Conformation, Dynamics and k_{cat}

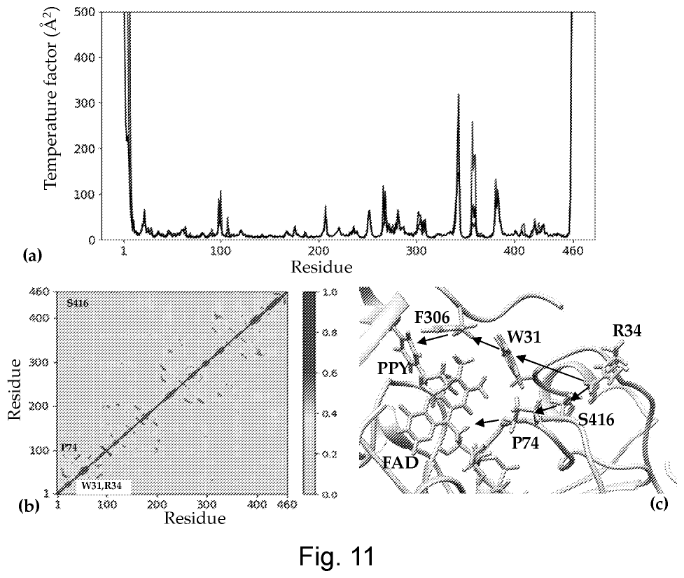

To exemplify how the Q20 method might work in practice, and to show basic proof-of-concept, two arbitrary mutations were chosen: D113N and A270G. While neither of these has been tested experimentally to our knowledge, they could be envisaged as possible trial mutations in a DE experiment, but they would not be chosen by standard methodology as they are not close to the active site; in fact, they reside close to the enzyme surface. Two additional 5 μs restrained simulations were performed starting from identical conformations but with these distal mutations inserted. First, the temperature (B−) factors were calculated for D2, D113N and A270G (FIG. 11a). Comparing the traces, the distal mutations cause changes in the temperature factors all over the enzyme, including within the active site. One key residue (D352) had a temperature factor of 6.2 Å² in D2 and this reduced to 5.4 Å² in D113N and 4.9 Å² in A270G. Both mutants also showed decreased temperature factors for a range of active-site residues (e.g., M129, H130, and N414), in contrast to increased enzyme flexibility introduced by the glycine mutation where a temperature factor of 25.9 Å² was observed for G270; the equivalent residue A270 in D2 had a lower temperature factor of 18.5 Å². One possible explanation for this effect in A270G is entropy-enthalpy compensation (a hypothesised consequence of basic thermodynamic laws) [42, 43], where increased flexibility in the distal α-helix containing G270 is counterbalanced by tighter structure in the active site. These distinct changes show how distal mutations cause changes in global dynamics (including that of the active site), with knock-on impacts on ΔΔG^{‡}_{Q20EFF}. Global Q20 calculations for each mutant reveal different effective electrostatic contributions to the barrier compared with D2 (−16.0 kcal×mol⁻¹) and are commensurate with the differences in overall dynamics. For D113N μ_{Q20} was calculated at −6.6 kcal×mol¹ and σ_{Q20}² at 9.7 kcal²×mol⁻² (FIG. 9c), producing a ΔΔG^{‡}_{Q20EFF}=−14.8 kcal×mol⁻¹. For A270G μ_{Q20} was calculated to be −5.9 kcal×mol⁻¹ with a σ_{Q20}² of 9.8 kcal²×mol⁻² (FIG. 9d), producing a ΔΔG^{‡}_{Q20EFF}=−14.0 kcal×mol⁻¹. In this case both mutations were predicted to have a deleterious influence on the effective lowering of the electrostatic component of the activation barrier, by up to 2 kcal×mol⁻¹.

FIG. 11b shows the generalised correlation coefficient matrix for C_{α} atoms throughout the D2 enzyme, showing a network of dynamical correlations. Overall, the patterns are characteristic of the myriad of complex interactions within and between α-helices, β-sheets and loops. These could be both the basis of the long-range cooperative motions within the protein (that result in global changes in temperature factor) and other phenomena such as allosteric effects. For example, there is a network of connections between residues of the external loop around residue R34, which strongly correlate to residue W31 of the same loop. In turn, residue W31 has an inward orientation and strongly correlates to active-site residue F306; thereby connecting an outer loop to the active site. Other less tractable connections exist such as W31↔S416↔P74↔FAD, which could also potentially affect the active site dynamic environment. This matrix shows, in principle, how a network of interactions between residues can allow dynamics to be propagated from distal residues, for example on the surface, to the active site. This underpins the view that a methodology combining MD and electrostatics can effectively detect and quantify potential changes in the enzyme turnover in response to distal mutations affecting global protein conformation and dynamics.

### Materials and Methods

### Protein 3D Models

A protein 3D model was built from the A-chain of the crystal structure (protein data bank accession code 2BVF) of 6-hydoxy-D-nicotine oxidase (6-HDNO) from Arthrobacter nicotinovorans [44]. Two mutations were inserted (E350L/E352D), in order to agree with the amino acid sequence of the functional enzyme considered here (referred to as D2) [6], and amino acids missing from the crystal structure (within loops) were inserted using MODELLER [45] but any suitable homology modelling software could be used. Crystal water molecules were maintained where possible and the protonation state of titratable amino acids were calculated using PROPKA at pH 7.0 [46], but other software is also available for performing this calculation and could be used, such as H++[163]. The 6-HDNO enzyme is a flavoprotein, containing a flavin adenine dinucleotide (FAD) cofactor, which is covalently attached to H72 via an 8a-(N3-histidyl)-riboflavin linkage [47]. A model of the complete histidine-FAD molecule was parameterized and optimised using the general AMBER force field (GAFF [48]), with partial charges calculated using Gaussian 09 and RESP [49, 50]. H72 was replaced by this molecular patch and the FAD coordinates fixed to the crystal structure; the remainderthe protein was parameterised using the FF14SB force-field [51]. To create a preliminary aqueous configuration suitable for molecular modelling (MM), water molecules were added (parameterised by the TIP3P model [52]) to place the protein in a cubic box of edge ˜10 nm. Ions (Na⁺ and Cl⁻) were added to neutralize any residual protein charge and to achieve a roughly physiological ionic concentration of ˜100 mM.

### Molecular Dynamics Simulations

Molecular dynamics (MD) simulations were performed using OpenMM software [53]. It is noted that OpenMM is not a specific requirement and any molecular dynamics software (such as CHARMm, AMBER, Tinker, Gromacs etc.) or energy-based ensemble generating algorithm (such as Monte Carlo and enhanced sampling techniques) could be used if suitable parameters and protein and water models can be constructed. The molecular modelling configuration was subjected to energy minimisation followed by 50 ns of MD simulation with constant temperature (298 K) and pressure (1 atm) in the isothermic-isobaric (NPT) ensemble. Following this, the cube edge was recorded to be 9.8 nm. This was fixed and 50 ns of equilibration was performed at constant volume and temperature in the NVT ensemble (NPT could also be used at this stage but NVT is used in these calculations because it is generally faster and yields a similar result, see Example 6). At this stage 1 μs of production dynamics was performed. Electrostatics were modelled by the particle mesh Ewald (PME) method with a 0.9 nm cut-off, switched at 0.75 nm, and error tolerance 5×10⁻⁴. Hydrogen atoms were fixed with SHAKE and water molecules kept rigid (constraint tolerance 1×10⁻⁵). The hydrogen mass was increased to 4 amu using the hydrogen mass repartitioning method [54], allowing a time-step of 4 fs with the Langevin integrator. Temperature was kept constant using a collision rate of 0.1 μs⁻¹ and coordinates were saved at 0.1 ns intervals.

A molecular mechanics model of (R)-2-phenylpyrrolidine (PPY) was parameterized and geometry optimised using the general AMBER force field GAFF [48] and partial charges were calculated using Gaussian 09 and RESP [49, 50], for both the substrate and the FAD cofactor. However, any reasonable structure can also be used which need not be optimised by DFT methods. Additionally, any means to produce partial atomic charges parameters may be utilised instead, such as by calculation de novo by DFT methodologies (e.g., by a CM5 population analysis). This was docked into the protein configuration calculated after 1 μs of MD, removing overlapping water molecules. Any method of docking, including manual docking, may be used to find an approximate near attack conformation based on atomic distances of the residues involved in the transition state formation. For example, a plurality of non-clashing conformations may be automatically provided and a suitable conformation may be manually selected from these, for example one that has a key atoms (e.g. atoms involved in the reaction mechanism) sufficiently close to each other while avoiding a van der Waal radius overlap. This complex was minimised, and a 10 μs production MD simulation was performed. During this simulation, the substrate remained within the region of the active site. The simulation was scanned for near-attack reaction configurations, where the H1 of PPY made a close contact with N5 of the isoalloxazine ring, with a ˜2 Å distance. The most suitable near attack coordinates based on distance and angle criteria were deemed to be those taken from the unrestrained simulation at 1212 ns. To this configuration a harmonic restraint potential (with an equilibrium distance of 2.2 Å and a force constant 1×10⁵ kJ×nm⁻¹) was applied and a further 5 μs of partially restrained MD for the D2 enzyme was performed for further analysis.

The partially restrained simulations for D113N and A270G were performed in an identical manner, albeit with longer equilibration periods such that normal distributions were obtained for each (although as described above it is not necessary for equilibrium to be reached and/or for a normal distribution of outputs to be used as a criterion for assessing equilibrium). Mutations were inserted into the protein structure based on the full coordinates from the unrestrained simulation of D2 at 1212 ns (including water molecules) and subsequently energy minimised with PME electrostatics active (in the case of D113N, a single Na-ion was removed to ensure charge neutrality). The harmonic restraint potential between PPY and FAD was then reapplied prior to 1 μs of partially restrained equilibration (discarded to allow for conformational rearrangements) followed by 5 μs of partially restrained MD (where coordinates were saved at 0.1 ns intervals for later analysis) in each case.

### Quantum Mechanics and QM/MM Calculations

A hydride transfer mechanism from a nitrogen bonded carbon atom on the substrate towards the flavin cofactor has been proposed as the dominant activation path of amine oxidation in flavoproteins (polar nucleophilic and single electron transfer reactions have also been proposed but are not presently favoured mechanisms) [55-59]. All theoretical activation barriers calculated were therefore based on a hydride transfer mechanism (FIG. 7b).

Density functional theory (DFT) models consisting of the substrate and a truncated FAD moiety (FIG. 7) were prepared to calculate the angle dependency of the hydride transfer mechanism at the B3LYP/def2-SVP+D3BJ level of theory using ORCA [60-65]. An angle constraint was applied to the starting coordinates obtained from the optimised reactant complex at different angular values, and the reaction coordinate was scanned to locate the saddle point for each model. All the obtained transition states were optimised to a single imaginary frequency, as corroborated by frequency calculations. The absolute and relative energies for the DFT models are shown in Table 4.

To obtain reactant complex and transition state structures, QM/MM optimisations were performed with an electrostatically embedded method using ChemShell [66, 67], where the QM component was calculated with TURBOMOLE [68], at the B3LYP/def2-SVP level of theory, where alternative DFT functionals and basis sets can also be employed with similar results. The MM region was calculated by DL_POLY [69, 70], using the CHARMM forcefield with CGenFF and C36-protein parameters [71-73]. There are several equivalent software alternatives for QM/MM calculations (e.g., NWChem, Q-Chem or ONIOM), which optionally will use alternative force fields for the MM region, such as AMBER instead of CHARMM. Alternatively, a DFT Cluster approach can be employed instead of a QM/MM methodology (including a variety of software options such as ORCA, Gaussian, Q-Chem, Turbomole, and SwissParam etc.) was employed to parameterise the substrate and FAD moiety [74]. The QM region was defined as those atoms shown in FIG. 13, including a truncated FAD, the substrate, and amino acid residues H72, M129, H130, W314 and N414. Alternatively, a larger QM region can also be defined (subject to more expensive computational calculations), or a smaller QM region may be defined. The absolute and relative activation barrier energies of the QM/MM models are shown in Table 5.

TABLE 4
Absolute and relative energies for the DFT models as optimised
at the B3LYP/def2-SVP + D3BJ level of theory by
ORCA 4.0. Models including the substrate, the FAD moiety
and covalently bound H72 (see FIG. 7). A minimal barrier
is found for an approach angle of circa 120°.

| | | Relative | | Relative |
|---|---|---|---|---|
| | Absolute | Electronic | Absolute | Free |
| | Electronic | Energy | Free | Energy |
| | Energy | (kcal/ | Energy | (kcal/ |
| Model | (a.u.) | mol) | (a.u.) | mol) |
| RC | −1235.62617784 | N/A | −1235.27885146 | N/A |
| TS 50 deg | −1235.54265569 | 52.4 | −1235.19904428 | 50.08 |
| TS 70 deg | −1235.57160597 | 34.2 | −1235.22776565 | 32.06 |
| TS 80 deg | −1235.5813443 | 28.1 | −1235.23759077 | 25.89 |
| TS 90 deg | −1235.58865863 | 23.5 | −1235.24401987 | 21.86 |
| TS 100 deg | −1235.58936822 | 23.1 | −1235.2461127 | 20.54 |
| TS 105 deg | −1235.59110955 | 22.0 | −1235.24777494 | 19.50 |
| TS 115 deg | −1235.59278473 | 21.0 | −1235.24943181 | 18.46 |
| TS 120 deg | −1235.59269357 | 21.0 | −1235.24947582 | 18.43 |
| TS 125 deg | −1235.59190973 | 21.5 | −1235.24877577 | 18.87 |
| TS 150 deg | −1235.58588272 | 25.3 | −1235.24318322 | 22.38 |

TABLE 5
Absolute QM/MM energies at the B3LYP/def2-SVP level
of theory as optimised by Chemshell/Turbomole/
DL_Poly at the B3LYP/def2-SVP level of theory.

| | Reactant | Hydride | Reactant | Hydride |
|---|---|---|---|---|
| | Complex | Transfer TS | Complex | Transfer TS |
| | QM/MM_{QM} | QM/MM_{QM} | QM/MM | QM/M |
| | Energy | Energy | Energy | Energy |
| Frame | (a.u.) | (a.u.) | (a.u.) | (a.u.) |
| 11178 | −2776.624501 | −2776.61182 | −2781.4575 | −2781.4422 |
| 11597 | −2776.514423 | −2776.505042 | −2780.7295 | −2780.7233 |
| 12101 | −2776.518086 | −2776.504994 | −2781.0316 | −2781.0174 |
| 12109 | −2776.535704 | −2776.530682 | −2780.9039 | −2780.9125 |
| 12111 | −2776.520333 | −2776.512664 | −2781.0527 | −2781.0418 |
| 12140 | −2776.53096 | −2776.520045 | −2781.0661 | −2781.051 |
| 12141 | −2776.570838 | −2776.543221 | −2781.402 | −2781.3774 |
| 25807 | −2776.61554 | −2776.577539 | −2781.2225 | −2781.1786 |
| 25842 | −2776.573737 | −2776.531913 | −2781.6194 | −2781.5729 |

### Electrostatic Transition State Barrier Energy Calculation (Q20)

The process for electrostatic calculations (referred to here as Q20) is distinct to other processes reported elsewhere [28, 37] at least in that a larger region (optionally including several fragments other than the substrate) are included in the core region which encompasses all the main atoms involved in a change in partial atomic charges during the activation step. Additionally, the process detailed in this example also differs from previous work at least in part by including the addition of restraints, scoring of zero-charged mutants (by effect of MD simulation), scoring of fully equilibrated mutants during molecular dynamics including mutants outside of the active site and the log-normal correction to the measured effects, as well as in the application to evaluate a plurality of mutants. For the calculations, the system was split into the core region (see FIG. 13), and the external region (containing the rest of the system). The previously optimised reactant complex (RC) and transition state (TS) structures (originally sampled from the 1117.8 ns timepoint of the unrestrained MD) were used in this parameterisation (where as the skilled person understands, the RC is a local minimum in the “potential energy surface” (energy vs. spatial coordinates), and the TS is a maximum for only the reaction coordinate of the reaction path, but a minimum for all other coordinates) and were optimised by a QM/MM methodology. Note that any electronic structure method optimisation such as a DFT cluster methodology could be used instead. It is also possible to use a procedure involving a plurality of frames to obtain plurality of optimised reactant complex and optimised transition state structures (as demonstrated in Example 6). The isolated, optimised QM regions were extracted and DFT models for partial charge estimations were calculated at the uM06/6-31G* level of theory with Gaussian 09 [50, 75, 76]. An implicit solvent model (SMD) with ε=35.68 (acetonitrile) was used to simulate the true enzyme environment [77]. Note that a different approach based on gas phase models or a different implicit solvent may also be employed for equivalent results (see Examples 4 to 7). Partial atomic charges were obtained from a population analysis using the CM5 model [78] for the core region. It is noted that other methods of calculating the partial atomic charges (such as a Hirshfeld population analysis or a Mulliken population analysis) would give similar results. The difference of partial atomic charges (AQᵢ), calculated from the CM5 population analysis, was determined for each atom i using the difference between transition state (TS) and reactant complex (RC) charges, Equation (4) and Table 6 and Table 7.

$$\\ \begin{matrix}Δ {Q}_{i} = {Q}_{i}^{{}_{} TS} - {Q}_{i}^{{}_{} RC} & ( 4 )\end{matrix}$$

TABLE 6
Cartesian coordinates and CM5 Hirshfield partial charges for
the reactant complex. Structure corresponds to QM/MM optimised
QM region from frame 11178. CM5 population analysis performed
by Gaussian 09 at the uM06/6-31G* level of theory.

| Atom | X | Y | Z | CM5 Charge |
|---|---|---|---|---|
| C | 4.9529 | −4.9786 | 5.1884 | −0.0068 |
| H | 4.5272 | −5.7438 | 5.8224 | 0.1368 |
| C | 4.5553 | −3.6215 | 5.0122 | −0.0158 |
| N | 5.9755 | −5.1659 | 4.3451 | −0.4855 |
| N | 5.3615 | −3.0506 | 4.0006 | −0.2963 |
| C | 6.2359 | −4.0301 | 3.5960 | 0.1205 |
| H | 7.1233 | −3.8649 | 3.0000 | 0.1487 |
| C | 1.4161 | −2.3326 | −3.6256 | 0.3125 |
| C | −0.6977 | −1.2488 | −2.9622 | 0.3927 |
| C | 1.0550 | −1.1741 | −1.3903 | 0.3060 |
| C | 1.9478 | −1.9177 | −2.2832 | 0.1759 |
| C | 3.6343 | −2.0077 | −0.7368 | 0.1204 |
| C | 4.9656 | −2.3802 | −0.4784 | −0.0720 |
| C | 5.5415 | −2.2718 | 0.7794 | −0.0092 |
| C | 7.0129 | −2.5822 | 0.9093 | −0.2331 |
| C | 4.7002 | −1.8234 | 1.8366 | 0.0077 |
| C | 5.2464 | −1.7275 | 3.2536 | −0.0162 |
| C | 3.4039 | −1.3403 | 1.5717 | −0.0911 |
| C | 2.8463 | −1.3607 | 0.2636 | 0.1414 |
| N | 0.1150 | −1.9751 | −3.8426 | −0.4331 |
| N | −0.1729 | −0.8888 | −1.7417 | −0.4459 |
| N | 3.1481 | −2.2854 | −1.9827 | −0.3258 |
| N | 1.5926 | −0.8325 | −0.1374 | −0.3912 |
| O | 2.0777 | −2.9758 | −4.4221 | −0.3611 |
| O | −1.8463 | −1.0015 | −3.2857 | −0.3885 |
| H | −0.3625 | −2.3373 | −4.6880 | 0.3721 |
| H | 5.5517 | −2.7155 | −1.3392 | 0.1185 |
| H | 7.4722 | −2.1230 | 1.7979 | 0.1068 |
| H | 7.5409 | −2.1683 | 0.0349 | 0.1061 |
| H | 7.2152 | −3.6676 | 0.9255 | 0.1102 |
| H | 4.6200 | −1.0698 | 3.8655 | 0.1378 |
| H | 6.2567 | −1.2909 | 3.2448 | 0.1367 |
| H | 2.8386 | −0.9053 | 2.4041 | 0.1320 |
| C | 6.1224 | −1.1797 | −3.9160 | −0.0173 |
| C | 5.8717 | −2.5610 | −4.0088 | −0.1111 |
| C | 6.9254 | −3.4700 | −4.1960 | −0.1128 |
| C | 8.2450 | −3.0086 | −4.2867 | −0.1174 |
| C | 8.5036 | −1.6307 | −4.2074 | −0.1136 |
| C | 7.4508 | −0.7242 | −4.0244 | −0.1117 |
| C | 5.5965 | 1.9802 | −2.7677 | −0.1721 |
| C | 4.7595 | 2.1359 | −4.0412 | −0.0669 |
| N | 4.8058 | 0.8146 | −4.7052 | −0.5861 |
| H | 5.6005 | 0.7871 | −5.3434 | 0.3072 |
| H | 3.7135 | 2.3784 | −3.7718 | 0.0955 |
| H | 5.1051 | 2.9197 | −4.7324 | 0.1102 |
| H | 5.4012 | 2.7632 | −2.0183 | 0.0948 |
| H | 6.6762 | 2.0018 | −2.9950 | 0.0937 |
| C | 5.1705 | 0.5775 | −2.3066 | −0.1662 |
| H | 5.9012 | 0.0985 | −1.6345 | 0.0883 |
| H | 4.2033 | 0.6382 | −1.7743 | 0.0873 |
| C | 4.9818 | −0.2162 | −3.6451 | 0.0131 |
| H | 4.0625 | −0.8178 | −3.5945 | 0.0983 |
| H | 4.8342 | −2.9029 | −3.9253 | 0.0999 |
| H | 6.7291 | −4.5460 | −4.2748 | 0.1138 |
| H | 9.0613 | −3.7270 | −4.4044 | 0.1139 |
| H | 9.5326 | −1.2619 | −4.2634 | 0.1138 |
| H | 7.6712 | 0.3442 | −3.9299 | 0.1118 |
| H | 3.7144 | −3.1027 | 5.4765 | 0.1308 |
| H | 1.1108 | −0.1778 | 0.4477 | 0.3920 |

TABLE 7
Cartesian coordinates and CM5 Hirshfield partial charges for
the transition state. Structure corresponds to QM/MM optimised
QM region for frame 11178. CM5 Population analysis performed
by Gaussian 09 at the uM06/6-31G* level of theory.

| Atom | X | Y | Z | Q − CM5 |
|---|---|---|---|---|
| C | 4.9529 | −4.9786 | 5.1884 | −0.00827 |
| H | 4.5272 | −5.7438 | 5.8224 | 0.13609 |
| C | 4.5553 | −3.6215 | 5.0122 | −0.01702 |
| N | 5.9755 | −5.1659 | 4.3451 | −0.48699 |
| N | 5.3615 | −3.0506 | 4.0006 | −0.29736 |
| C | 6.2359 | −4.0301 | 3.5960 | 0.11914 |
| H | 7.1233 | −3.8649 | 3.0000 | 0.14748 |
| C | 1.5579 | −2.2252 | −3.6769 | 0.28404 |
| C | −0.5946 | −1.1885 | −3.0421 | 0.37502 |
| C | 1.1931 | −1.0370 | −1.5166 | 0.27959 |
| C | 2.0941 | −1.6914 | −2.4189 | 0.09584 |
| C | 3.7725 | −1.8610 | −0.8016 | 0.10292 |
| C | 5.0732 | −2.2805 | −0.4965 | −0.08753 |
| C | 5.5984 | −2.2418 | 0.7890 | −0.01436 |
| C | 7.0606 | −2.5780 | 0.9517 | −0.23434 |
| C | 4.7274 | −1.8265 | 1.8276 | −0.00822 |
| C | 5.2398 | −1.7274 | 3.2509 | −0.01887 |
| C | 3.4314 | −1.3560 | 1.5256 | −0.09754 |
| C | 2.9302 | −1.2984 | 0.1961 | 0.12386 |
| N | 0.2263 | −1.9381 | −3.8917 | −0.44491 |
| N | −0.0659 | −0.7896 | −1.8428 | −0.46627 |
| N | 3.3818 | −1.9790 | −2.1299 | −0.38719 |
| N | 1.6865 | −0.7386 | −0.2203 | −0.42003 |
| O | 2.2166 | −2.9025 | −4.4680 | −0.38942 |
| O | −1.7539 | −0.9613 | −3.3769 | −0.41834 |
| H | −0.2470 | −2.3473 | −4.7129 | 0.36407 |
| H | 5.7025 | −2.5854 | −1.3377 | 0.11590 |
| H | 7.5039 | −2.1373 | 1.8580 | 0.10564 |
| H | 7.6187 | −2.1637 | 0.0969 | 0.10411 |
| H | 7.2478 | −3.6663 | 0.9583 | 0.10894 |
| H | 4.5906 | −1.0836 | 3.8539 | 0.13584 |
| H | 6.2450 | −1.2795 | 3.2675 | 0.13445 |
| H | 2.8287 | −0.9618 | 2.3527 | 0.12655 |
| C | 6.1101 | −1.3485 | −3.7610 | −0.01911 |
| C | 5.7655 | −2.6634 | −4.1127 | −0.10830 |
| C | 6.7636 | −3.5637 | −4.5202 | −0.10969 |
| C | 8.1014 | −3.1572 | −4.5912 | −0.11062 |
| C | 8.4442 | −1.8358 | −4.2659 | −0.11006 |
| C | 7.4568 | −0.9380 | −3.8424 | −0.10680 |
| C | 5.5492 | 1.9602 | −2.7303 | −0.15433 |
| C | 4.3416 | 1.8729 | −3.6706 | −0.04330 |
| N | 4.4141 | 0.4779 | −4.1125 | −0.49621 |
| H | 3.8432 | 0.1536 | −4.8978 | 0.35138 |
| H | 3.3913 | 2.0800 | −3.1376 | 0.10531 |
| H | 4.3874 | 2.5450 | −4.5452 | 0.11882 |
| H | 5.4817 | 2.7837 | −2.0048 | 0.10545 |
| H | 6.4807 | 2.0896 | −3.3074 | 0.10188 |
| C | 5.5393 | 0.5649 | −2.0807 | −0.15427 |
| H | 6.5046 | 0.2696 | −1.6457 | 0.10320 |
| H | 4.7791 | 0.5282 | −1.2771 | 0.09780 |
| C | 5.0907 | −0.3491 | −3.2351 | 0.07774 |
| H | 4.2259 | −1.1947 | −2.7687 | 0.21111 |
| H | 4.7164 | −2.9676 | −4.0467 | 0.10256 |
| H | 6.5070 | −4.5978 | −4.7741 | 0.11566 |
| H | 8.8706 | −3.8778 | −4.8822 | 0.11656 |
| H | 9.4864 | −1.5048 | −4.3149 | 0.11623 |
| H | 7.7409 | 0.0796 | −3.5556 | 0.11603 |
| H | 3.7144 | −3.1027 | 5.4765 | 0.13017 |
| H | 1.1834 | −0.1181 | 0.3838 | 0.38031 |

For the rest of the solvated enzyme system (the external region), partial atomic charges (qⱼ) were assigned based on the same charges previously assigned to the MM region in the QM/MM calculations as found in the publicly available CGenFF and 036-protein parameter files [71-73]. Other equivalent partial atomic charges may also be used such as the ff14SB parameters used for amber force field simulations. Partial atomic charges for linker atoms were redistributed to the closest bonded atoms (i.e. bonds between atoms in the QM region and atoms not in the QM regions are split and capped by adding H atoms), although this is optional and may not be performed. For any set of system coordinates including the solvent environment and counterions, the Q20 calculation consists of a summation of the electrostatic Coulombic interactions between each atom j of the external region towards the previously calculated charge difference for each atom i of the core region (ΔQᵢ) for each set of coordinates of the MD simulation, Equation (5). Specifically, the Q20 energy summation is over all external residues and their constituent atomic point charges (qⱼ) of the external region and distances to the core atoms (rgi), and Coulomb's constant (c) which is 332/e² kcal×Å×mol⁻¹ [37] to provide energy in kcal×mol⁻¹ if charges are elementary (i.e., multiples of electronic charge e), and distance is in A. Note that this is a linear sum and can be easily decomposed into sub-sums due to individual residues etc.

$$\\ \begin{matrix}Δ Δ {G}_{Q 20}^{{}_{} ‡} = c {\sum }_{j ∈ external} {\sum }_{i ∈ core} \frac{{q}_{j} Δ {Q}_{i}}{{r}_{ji}} & ( 5 )\end{matrix}$$

### Temperature-Factors

For calculation of temperature factors, the first step was to perform a minimum root-mean-square deviation (RMSD) alignment of all conformations of each respective MD simulation onto the reference crystal structure (protein data bank code 2BVF). The second step was to again perform a minimum RMSD alignment of the C_{α} atoms from all conformations onto the average Cₐ coordinates from the previous alignment. The temperature factors (Bᵢ) were calculated using the Equation (6) for each of the C_{α} vectors (xᵢ) according to previous x-ray crystallography methodology (calculated in Å²) [79].

$$\\ \begin{matrix}{B}_{i} = \frac{8}{3} {π}^{{}_{} 2} 〈 {( {x}_{i} - \overline{{x}_{ι}} )}^{2} 〉 & ( 6 )\end{matrix}$$

### Generalised Correlation Coefficient Matrix

The generalized correlation measure stems from the independence of random variables. Two random variables are independent, if and only if, their joint distribution is the product of their marginal distributions. If the variables are correlated, then the mutual information provides a well-defined and complete measure of correlation, which yields values in the range [0, ∞). The mutual information was calculated between all C_{α} atoms throughout the restrained MD simulation of D2. This was mapped back to a value in the range [0,1] using a previously derived transformation to a Pearson-like correlation value, to produce a generalized correlation coefficient [80]. This results in a 2D-matrix of values (FIG. 11b) for the whole protein with values of 1 (total correlation) along the diagonal (self-interaction) and the off-diagonal terms showing the extent of correlation between each individual amino acid from 0 (zero correlation) to 1 (full correlation).

### Conclusions

In this example, it is shown that in 6-hydroxy-D-nicotine oxidase, electrostatics are a main contributor to the fluctuations in the activation energies of hydride transfer due to enzyme dynamics, as has been validated through comprehensive QM/MM calculations. The resulting combined MD and electrostatics methodology correctly identifies D352 as highly energetically favourable for catalysis (previously recognized by site directed mutagenesis [6]), and provides insight into other unexplored sites, such as D316. It is demonstrated that the electrostatic fluctuations are normally distributed, and therefore the turnover number is not only dependent on the average of the instantaneous activation barrier energy but is also determined by the spread of the barrier energies, which is in turn dictated by global enzyme dynamics. A proof-of-concept analysis shows how distal mutations can result in perturbations to enzyme global dynamics and it is postulated that these changes are propagated via cooperative interactions throughout the enzyme. Combining these approaches therefore, in principle, allows the relative effect on turnover of mutations anywhere in the enzyme to be estimated. DE is a combinatorial search problem, for which the search space is reduced massively if one knows which residues to prioritise for mutation. The ability to do this as set down here could allow DE to be accelerated by facilitating theoretical prioritization of amino acids throughout the whole enzyme and thereby also working towards ameliorating the dead-end problem of inescapable local optima due to fixation on the active site. It also provides insight into the fundamental relationship between enzyme dynamics and catalysis, and improves the understanding of distal amino acids. The detailed process advances a much-needed toolbox of theoretical methods for accelerated protein engineering, potentially benefitting key applications such as chiral synthesis of APIs.

### Example 2: Rapid Improvement of Both Enzyme Activity and Thermal Stability by Rational Directed Evolution Using a Multiply Degenerate Full-Length Gene Library and Supercharging: Application to an Oxidoreductase (EC-1)

### Introduction

Example 1 reports a theoretical methodology for the prediction of enzyme activity based on the dynamic and electrostatic effects of mutations. In this example, this is used as an objective function to rank a series of conservative mutations of 6-HDNO to reduce and enrich the essentially infinite mutational space of the enzyme. Amine oxidases are a valuable family of enzymes that have gained special interest in DE due to their broad scope of activity towards several API-relevant substrates [5]. An early success of protein engineering has been reported on monoamine oxidase (MAO-N), which has yielded derivative enzymes capable of (S)-selective oxidation of primary, secondary, and tertiary amines [93-95]. Another flavin-dependent amine oxidase, 6-hydroxy-d-nicotine oxidase (6-HDNO), has been recently targeted for DE for its substrate selectivity, but unlike MAO-N, 6-HDNO is an (R)-selective amine oxidase [96,44], which opens new opportunities in API synthesis. A 6-HDNO derivative with a double mutation (E350L/E352D) was discovered with a broader substrate scope and better activity towards a panel of structurally diverse and synthetically relevant cyclic amines [6], but the activity of this mutant relative to similar MAO-N mutants remained low, making 6-HDNO an excellent target for further optimisation.

A functionally enhanced library was constructed employing small degenerate codons to target several sites simultaneously using computational methods and PCR-based full-length gene synthesis. Following a single screening, a variant with a significant increase in activity was found containing three amino acid substitutions outside the active site. The work in this example further shows that the method is complimentary to site directed mutagenesis and enzyme stabilization methods, and in doing so, produces a fast and stable 6-HDNO derivative with a total of eight mutations from the wild type. This method holds promise for rapid engineering of enzymes to meet the continual and urgent need for new biological catalysts in industrial biosynthetic pathways including API manufacture, and to deliver environmentally sustainable chemistry.

### Results

### Initial Optimisation of the 6-HDNO Sequence to the Substrate of Interest

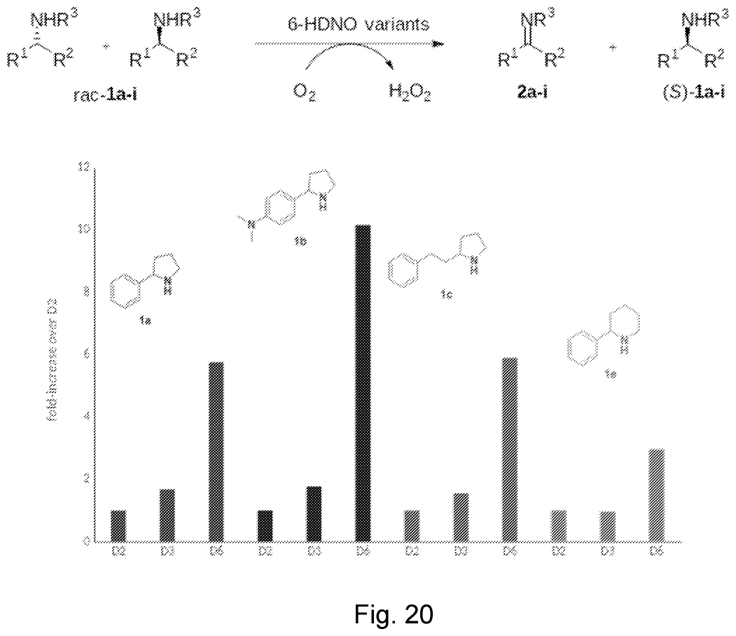

(R)-phenyl pyrrolidine (FIG. 20, labelled PPY) was chosen as the substrate of interest for optimisation being an example from a class of relatively unexplored but important heterocyclic compounds with potential use as a precursor in synthetic routes for APIs but still having undesirably low activity on 6-HDNO D2. Phenyl pyrrolidine derivatives have significant therapeutic potential, for example as antiviral, antibacterial and anticancer drugs and as new tools for mechanism of action studies [98-101]. While chiral amines are generally relevant in API biosynthetic pathways [4], an enzyme targeting these residues could in principle also be used for other diverse catalytic processes including cyclic secondary amines.

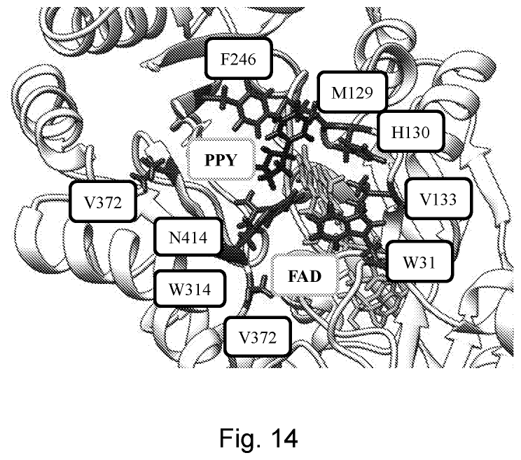

The activity towards PPY conversion in the starting enzyme (referred to as HDNO D2) [6] was low as it had not been specifically optimised for this substrate, making colony-based screening more difficult and time consuming, while it was also recognised that some easier to find targets could still be uncovered near the active site by standard (e.g., random) approaches. Therefore, an initial search was performed using site directed mutagenesis with no rational guidance, targeting nine active site residues not previously explored to identify a baseline mutant with increased activity towards PPY that could be used in further colony-based screening optimisation rounds. The list of targeted positions (FIG. 14) and the respective degenerate codons used for site directed mutagenesis are shown in Table 8.

TABLE 8
Targeted positions, codon degeneracy and amino acids for
site directed mutagenesis experiments on the first round
of DE. A total of 9 sites were targeted using site directed
mutagenesis experiments close to the active site.

| Target | Codon | |
|---|---|---|
| residue | degeneracy | Coded amino acids |
| M129 | DBG | A, G, M, L, S, R, T, W, V |
| V133 | VBC | A, G, I, L, P, S, R, T, V |
| N414 | VAW | E, D, H, K, N, Q |
| S416 | DBG | A, G, N, L, S, R, T, W, V |
| V372 | VBC | A, G, I, L, P, S, R, T, V |
| W314 | NDT | C, D, G, F, I, H, L, N, S, |
| | | R, V, Y |
| H130 | HDT | C, F, I, H, L, N, S, R, Y |
| F246 | NWT | D, F, I, H, L, N, V, Y |
| W31 | NWT | D, F, I, H, L, N, V, Y |

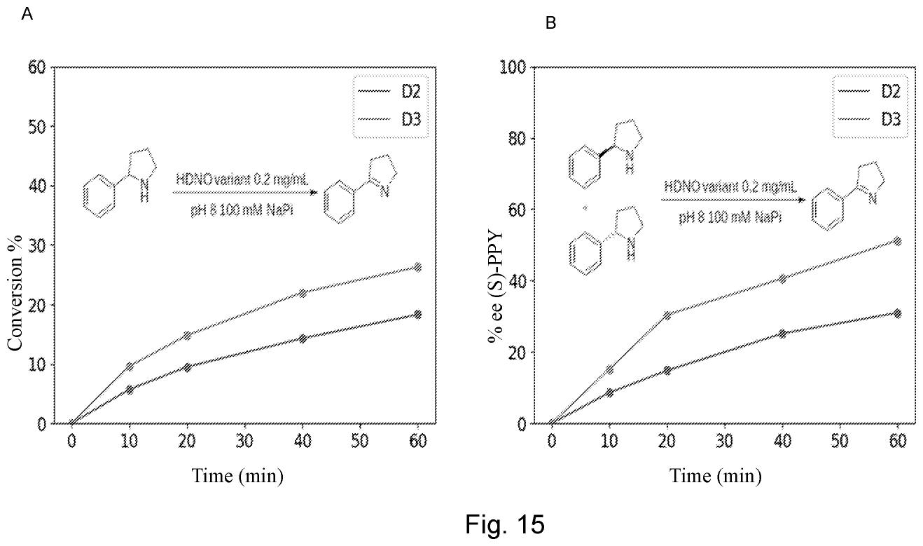

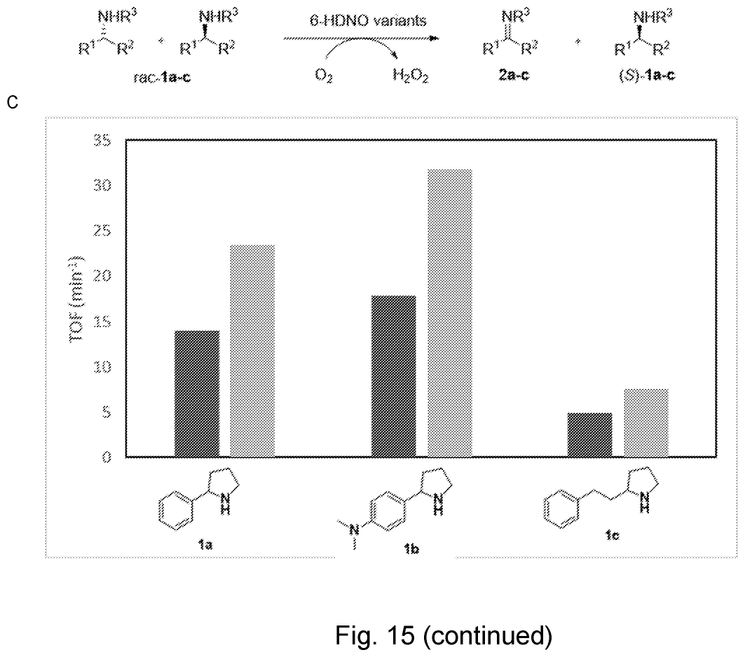

As can be observed in FIG. 14, no rational methodology was used to identify these sites beyond their proximity to the putative active site and the fact that they had not been explored previously. A mutant identified as N414H (here termed HDNO D3) was expressed (see Table 9 for the protein and DNA sequences of HDNO D2 and D3), purified and its activity was compared to that of the HDNO D2 variant (FIG. 15A). An almost twofold increase in activity towards the oxidation of PPY was observed on biotransformation measurements.

TABLE 9
HDNO D2 and D3 DNA and protein sequences.

| | |
|---|---|
| HDNO D2 | ATGGTTAGCAGCAAACTGGCAACACCGCTGAGCATTCAGGGTGAAGTTATTTATCCG |
| DNA | GATGATAGCGGTTTTGATGCCATTGCCAATATTTGGGATGGTCGTCATCTGCAGCGT |
| sequence | CCGAGCCTGATTGCACGTTGTCTGAGTGCCGGTGATGTTGCAAAAAGCGTTCGTTAT |
| (SEQ ID | GCATGTGATAATGGTCTGGAAATTTCAGTTCGTAGCGGTGGTCATAATCCGAATGGTT |
| NO: 1) | ATGCAACCAATGATGGTGGTATTGTTCTGGATCTGCGTCTGATGAATAGCATTCATAT |
| | TGATACCGCAGGTAGCCGTGCACGTATTGGTGGTGGTGTTATTAGCGGTGATCTGGT |
| | TAAAGAAGCAGCAAAATTTGGTCTGGCAGCAGTTACCGGTATGCATCCGAAAGTTGG |
| | TTTTTGTGGTCTGGCCCTGAATGGTGGTGTGGGTTTTCTGACCCCGAAATATGGCCT |
| | GGCAAGCGATAACATTCTGGGTGCAACCCTGGTTACCGCAACAGGTGATGTGATTTA |
| | TTGTAGTGATGATGAACGTCCGGAACTGTTTTGGGCAGTTCGTGGTGCAGGTCCGAA |
| | TTTTGGTGTTGTTACCGAAGTTGAAGTTCAGCTGTATGAACTGCCTCGTAAAATGCTG |
| | GCAGGTTTTATTACCTGGGCACCGAGCGTTAGCGAACTGGCAGGTCTGCTGACCAG |
| | CCTGCTGGATGCACTGAATGAAATGGCAGATCATATCTATCCGAGCGTTTTTGTTGGT |
| | GTGGATGAAAATCGTGCACCGAGTGTTACCGTTTGTGTTGGTCATCTGGGTGGTCTG |
| | GATATTGCAGAACGTGATATTGCACGTCTGCGTGGCCTGGGTCGTACCGTTAGCGAT |
| | AGCATTGCCGTTCGTAGCTATGATGAAGTTGTTGCGCTGAATGCAGAAGTTGGTAGC |
| | TTTGAAGATGGTATGAGCAATCTGTGGATTGATCGTGAAATTGCAATGCCGAATGCAC |
| | GTTTTGCAGAAGCAATTGCAGGTAACCTGGATAAATTTGTGAGCGAACCGGCAAGCG |
| | GTGGTAGCGTTAAACTGTTGATTGATGGTATGCCGTTTGGTAATCCGAAACGTACAC |
| | CGGCACGTCATCGTGATGCAATGGGTGTTCTGGCACTGGCAGAATGGTCAGGTGCA |
| | GCACCGGGTAGCGAGAAATATCCTGAACTGGCACGTGAACTGGATGCAGCACTGCT |
| | GCGTGCGGGTGTTACCACCAGTGGTTTTGGCCTGCTGAATAATAACAGCGAAGTTAC |
| | CGCAGAAATGGTTGCCGAAGTGTATAAACCGGAAGTTTATAGTCGCCTGGCAGCCGT |
| | TAAACGTGAATATGATCCGGAAAATCGTTTTCGCCACAACTATAACATCGATCCGGAA |
| | GGTAGCTAA |
| HDNO D2 | MVSSKLATPLSIQGEVIYPDDSGFDAIANIWDGRHLQRPSLIA |
| protein | RCLSAGDVAKSVRYACDNGLEISVRSGGHNPNGYATNDGGIV |
| sequence | LDLRLMNSIHIDTAGSRARIGGGVISGDLVKEAAKFGLAAVTG |
| (SEQ ID | MHPKVGFCGLALNGGVGFLTPKYGLASDNILGATLVTATGDVI |
| NO: 2) | YCSDDERPELFWAVRGAGPNFGVVTEVEVQLYELPRKMLAG |
| | FITWAPSVSELAGLLTSLLDALNEMADHIYPSVFVGVDENRAP |
| | SVTVCVGHLGGLDIAERDIARLRGLGRTVSDSIAVRSYDEVVA |
| | LNAEVGSFEDGMSNLWIDREIAMPNARFAEAIAGNLDKFVSE |
| | PASGGSVKLLIDGMPFGNPKRTPARHRDAMGVLALAEWSGA |
| | APGSEKYPELARELDAALLRAGVTTSGFGLLNNNSEVTAEMV |
| | AEVYKPEVYSRLAAVKREYDPENRFRHNYNIDPEGSStop |
| HDNO D3 | ATGGTTAGCAGCAAACTGGCAACACCGCTGAGCATTCAGGGTGAAGTTATTTATCCG |
| DNA | GATGATAGCGGTTTTGATGCCATTGCCAATATTTGGGATGGTCGTCATCTGCAGCGT |
| sequence | CCGAGCCTGATTGCACGTTGTCTGAGTGCCGGTGATGTTGCAAAAAGCGTTCGTTAT |
| (SEQ ID | GCATGTGATAATGGTCTGGAAATTTCAGTTCGTAGCGGTGGTCATAATCCGAATGGTT |
| NO: 3) | ATGCAACCAATGATGGTGGTATTGTTCTGGATCTGCGTCTGATGAATAGCATTCATAT |
| | TGATACCGCAGGTAGCCGTGCACGTATTGGTGGTGGTGTTATTAGCGGTGATCTGGT |
| | TAAAGAAGCAGCAAAATTTGGTCTGGCAGCAGTTACCGGTATGCATCCGAAAGTTGG |
| | TTTTTGTGGTCTGGCCCTGAATGGTGGTGTGGGTTTTCTGACCCCGAAATATGGCCT |
| | GGCAAGCGATAACATTCTGGGTGCAACCCTGGTTACCGCAACAGGTGATGTGATTTA |
| | TTGTAGTGATGATGAACGTCCGGAACTGTTTTGGGCAGTTCGTGGTGCAGGTCCGAA |
| | TTTTGGTGTTGTTACCGAAGTTGAAGTTCAGCTGTATGAACTGCCTCGTAAAATGCTG |
| | GCAGGTTTTATTACCTGGGCACCGAGCGTTAGCGAACTGGCAGGTCTGCTGACCAG |
| | CCTGCTGGATGCACTGAATGAAATGGCAGATCATATCTATCCGAGCGTTTTTGTTGGT |
| | GTGGATGAAAATCGTGCACCGAGTGTTACCGTTTGTGTTGGTCATCTGGGTGGTCTG |
| | GATATTGCAGAACGTGATATTGCACGTCTGCGTGGCCTGGGTCGTACCGTTAGCGAT |
| | AGCATTGCCGTTCGTAGCTATGATGAAGTTGTTGCGCTGAATGCAGAAGTTGGTAGC |
| | TTTGAAGATGGTATGAGCAATCTGTGGATTGATCGTGAAATTGCAATGCCGAATGCAC |
| | GTTTTGCAGAAGCAATTGCAGGTAACCTGGATAAATTTGTGAGCGAACCGGCAAGCG |
| | GTGGTAGCGTTAAACTGTTGATTGATGGTATGCCGTTTGGTAATCCGAAACGTACAC |
| | CGGCACGTCATCGTGATGCAATGGGTGTTCTGGCACTGGCAGAATGGTCAGGTGCA |
| | GCACCGGGTAGCGAGAAATATCCTGAACTGGCACGTGAACTGGATGCAGCACTGCT |
| | GCGTGCGGGTGTTACCACCAGTGGTTTTGGCCTGCTGAATCATAACAGCGAAGTTAC |
| | CGCAGAAATGGTTGCCGAAGTGTATAAACCGGAAGTTTATAGTCGCCTGGCAGCCGT |
| | TAAACGTGAATATGATCCGGAAAATCGTTTTCGCCACAACTATAACATCGATCCGGAA |
| | GGTAGCTAA |
| HDNO D3 | MVSSKLATPLSIQGEVIYPDDSGFDAIANIWDGRHLQRPSLIA |
| protein | RCLSAGDVAKSVRYACDNGLEISVRSGGHNPNGYATNDGGIV |
| sequence | LDLRLMNSIHIDTAGSRARIGGGVISGDLVKEAAKFGLAAVTG |
| (SEQ ID | MHPKVGFCGLALNGGVGFLTPKYGLASDNILGATLVTATGDVI |
| NO: 4) | YCSDDERPELFWAVRGAGPNFGVVTEVEVQLYELPRKMLAG |
| | FITWAPSVSELAGLLTSLLDALNEMADHIYPSVFVGVDENRAP |
| | SVTVCVGHLGGLDIAERDIARLRGLGRTVSDSIAVRSYDEVVA |
| | LNAEVGSFEDGMSNLWIDREIAMPNARFAEAIAGNLDKFVSE |
| | PASGGSVKLLIDGMPFGNPKRTPARHRDAMGVLALAEWSGA |
| | APGSEKYPELARELDAALLRAGVTTSGFGLLNHNSEVTAEMV |
| | AEVYKPEVYSRLAAVKREYDPENRFRHNYNIDPEGSStop |

To verify that this increase was due to a rise in activity and not to a loss in enantioselectivity, the reaction was also monitored by chiral HPLC. The new variant was completely selective towards the oxidation of the (R)-enantiomer (FIG. 15B). The D2 and D3 HDNO variants were tested against other 2-substituted pyrrolidines, and a similar improvement in the catalytic activity across the panel was observed (FIG. 15C). No further increase in activity was identified from the set of site directed mutagenesis (SDM) experiments conducted at these nine designated sites.

### Site Selection for Directed Evolution Using Molecular Dynamics Simulation.

A rational strategy was employed to identify key target residues outside of the active site space, with the potential for enhanced catalytic activity and to be used in the design of an enhanced DE library. Only sites that were outside of the N- and C-terminus regions (residues 1 to 5 and 454 to 459) and contained one of ten designated amino acids (Ala, Cys, lie, Lys, Met, Phe, Ser, Thr, Tyr and Val) were considered for mutation, which restricted the full 459 amino acids in 6-HDNO to 245 possible sites. The choice of mutants and positions that are included is largely arbitrary from a theoretical point of view and is problem dependent. For example, choices to restrict the full set of possibilities could be made to suit the experimental system or to restrict mutations that are likely to maintain or increase overall protein stability. In this example, a limited subset of neutral amino acid mutations were chosen, for exemplification of the principle that catalysis can be driven via global enzyme dynamics rather than direct electrostatic effects, since these amino acids are considered to have negligible direct electrostatic effects on catalysis). This set could have been extended to a wider range of uncharged amino acids, such as Asn, Trp and GIn (see the further examples below that use a wider range of mutants, including charged and neutral amino acids, including Cys and include mutations in the N- and C-terminus regions). To specify each mutant, a random combination of two of the restricted sites were first selected, and then a random point mutation at each of these sites from one of the ten designated amino acids to another one of these ten designated amino acids. This selection process allowed the possibility that no mutation could occur at one of the two selected sites (if the random selection resulted in the selection of the same amino acid as the original) but not at both, hence producing a mixture of single- and double-point mutations.

From this mutational space, a set of 23 single and 213 double mutants distributed throughout the enzyme was produced (see Table 10 for the list of mutants). Consideration of double mutations accelerated the overall speed of searching the sites of the enzyme, but also had another potentially more important effect. While this comes at a price of not being able to as easily deconvolute which of the two sites are responsible for catalytic effect, it does allow the possibility of evaluating potential synergies at disparate sites, which can be exploited by a combinatorial library to generate large increases in k_{cat}. Overall, this allowed 207 sites throughout the enzyme to be considered (some more than once) out of the total of 245 for theoretical analysis of their suitability for inclusion in a DE library.

The list of mutants was ordered in terms of potential increase in k_{cat} by calculating an objective function based on the methodology described in Example 1 using restrained molecular dynamics (MD) simulations. Briefly, a harmonic restraint of 2.2 Å was added between PPY and the flavin cofactor (FAD) of each 6-HDNO mutant to hold them in a near attack conformation and 50 ns of restrained molecular dynamics (MD) performed on each. These MD trajectories were used to produce coordinates (sampled every 0.1 ns) for the estimation of changes in hydride transfer activation barriers during enzyme dynamics using an electrostatic methodology (Q20). The Q20 model was previously parametrized for this reaction step in HDNO by using QM/MM and DFT methods to quantify the changes in partial atomic charges, which allows rapid estimates of instantaneous change in barrier height due to the enzyme (ΔΔG^{‡}_{Q20}) from conformations sampled from MD simulations (without using electrostatics from water). For each simulation, the mean change in barrier height due to the enzyme (μ_{Q20}) could be estimated using mean statistics and used as an objective function to rank the mutants (more negative values are better). The full list of estimated effective change in barrier height for all the mutants considered here is also shown in column 4 of Table 10.

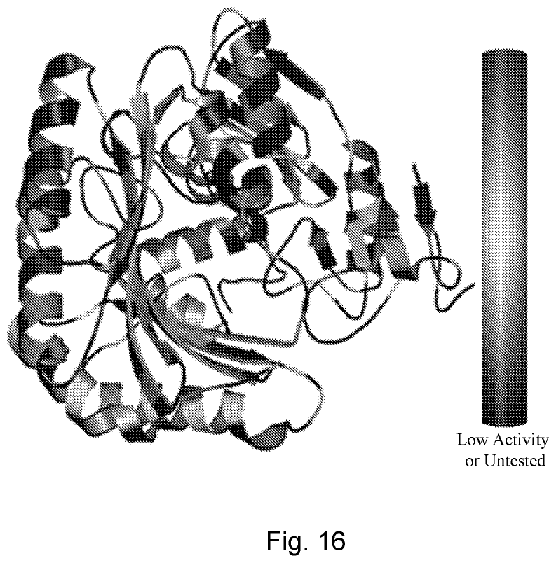

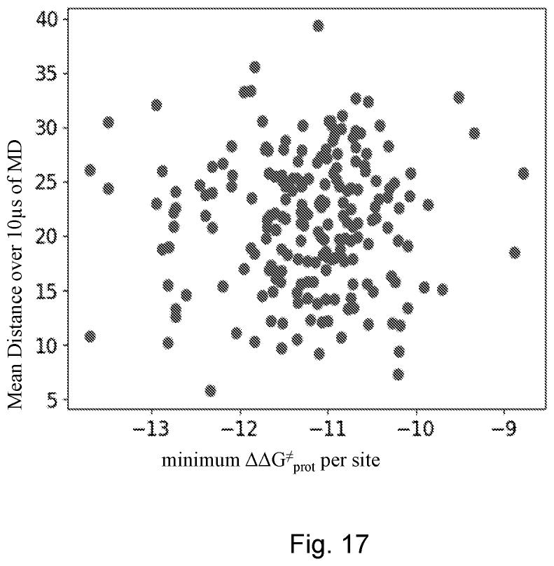

Here μ_{Q20} was used instead of ΔΔG^{‡}_{Q20EFF} due to this presenting lower noise levels in short MD simulations (see Examples 1 and 6). Individual sites were ranked for inclusion in the library based on the best theoretically predicted increase in k_{cat} (based on lower μ_{Q20}). For any enzyme that contained a mutation at that site, either at a single point or as part of a double point mutation (Table 10). For example, sites 43 (best amino acid I, mutant Y242F_A43I) and 242 (best amino acid F, mutant Y242F_A43I) were the most highly ranked sites because the mutant Y242F/A431 had a μ_{Q20} of −13.7 kcal×mol⁻¹. A dynamic range of μ_{Q20} from this value to −8.8 kcal×mol⁻¹ for A254T/T283S were obtained, which would correspond to a relative range of ˜10⁴ mol⁻¹ in terms of k_{cat} between the best and worst mutants. It was confirmed that the sites explored in silico were distributed all over the enzyme, as intended. The best catalytic activity found per site was also displayed on the protein 3D-structure with a false-colour heatmap dependent on the predicted ΔΔG^{‡}_{Q20EFF} (FIG. 16). There was no clustering of the best target sites within the overall 3D-structure and no obvious relation to, for example, secondary structure was found either. Several distant sites (with no obvious connection to the active site) were identified that were predicted to have a positive effect on catalytic rate. In fact, perhaps counterintuitively, no significant correlation was observed between the proximity to the active site and k_{cat} within this theoretical setting, as confirmed from a scatterplot of μ_{Q20} against average distance to the active site calculated from a 10 μs MD simulation of the HDNO D2 parent enzyme (FIG. 17).

TABLE 10
Raw activity scores of generated mutants.

| Mutant | μ_{Q20} | Mutant | μ_{Q20} | Mutant | μ_{Q20} |
|---|---|---|---|---|---|
| A103S_V261L | −10.76 | I158V_T419S | −10.93 | S285I_A331S | −10.53 |
| A103T_L205M | −11.11 | I171M_A331S | −10.68 | S339A_T362V | −10.32 |
| A103T_M237L | −11.69 | I17V_L313I | −11.68 | S385C | −12.31 |
| A103V_F409Y | −10.87 | I241M_A298S | −10.18 | S385C_Y242F | −10.77 |
| A103V_V431I | −11.32 | I241V_T258A | −11.59 | S40V_V347I | −10.85 |
| A119T_Y172F | −10.20 | I269V | −10.85 | S47T_S407A | −10.32 |
| A124M_A382S | −11.15 | I27V_T419S | −12.75 | S54A_L375M | −10.27 |
| A139S_L226M | −11.52 | I288V_I330L | −10.14 | S54A_M310L | −10.95 |
| A139S_V431I | −10.71 | I288V_V426M | −10.08 | S54T_V296I | −9.69 |
| A154T_Y432F | −10.46 | I30F_F121Y | −10.13 | S69A_L375M | −10.09 |
| A161S_S287T | −10.47 | I30V_F121Y | −10.69 | S69T_L234M | −8.88 |
| A166M | −10.49 | I30V_L411I | −10.83 | S93C_A382S | −11.60 |
| A166T_A211T | −10.63 | I315V | −10.56 | S93T_I274V | −10.74 |
| A184T | −9.95 | I319V_A324S | −10.66 | T127I_S285T | −11.10 |
| A184V_V261I | −11.16 | I319V_L412V | −10.74 | T148A_V431I | −12.72 |
| A188V | −10.32 | I330F_V423M | −11.31 | T162A_A238S | −10.60 |
| A211C_T228F | −10.57 | I330L_F337Y | −8.98 | T162S_F182Y | −11.41 |
| A211S_L280M | −11.67 | I42V_L313M | −10.87 | T162S_L391M | −10.33 |
| A217T | −12.08 | I84V_V85L | −10.59 | T165A_F192Y | −11.48 |
| A224T_A382F | −10.35 | L10I_T98A | −10.69 | T165A_L400M | −10.30 |
| A224V_A233V | −11.08 | L10M_A392M | −10.11 | T165V_M422V | −10.10 |
| A233T_S292T | −10.86 | L114M_L373I | −11.35 | T167V_A392I | −11.87 |
| A238S_S339T | −11.14 | L114V_A376S | −9.30 | T196S_A324T | −10.38 |
| A238T | −12.80 | L140M_F356Y | −9.90 | T362S_A382T | −11.46 |
| A254S_S346A | −11.34 | L147F | −10.58 | T79A_L226V | −10.54 |
| A254T_S385T | −10.87 | L147F_Y442F | −9.34 | T98A_T162A | −10.52 |
| A254T_T283S | −8.78 | L153F_A184V | −12.73 | V109M_I454V | −10.71 |
| A26F_M310L | −11.23 | L153M | −11.55 | V115I_V423T | −10.71 |
| A26T | −10.85 | L153M_I330M | −10.97 | V133I_L264M | −10.82 |
| A270T_L280I | −10.59 | L153M_M370L | −11.52 | V133M_A270T | −12.33 |
| A270T_L373I | −10.30 | L153M_Y242F | −10.05 | V133M_F182Y | −10.35 |
| A270T_M370L | −11.65 | L163F_L210M | −10.50 | V133M_V284I | −9.75 |
| A270V_A374S | −10.49 | L163V_S339T | −11.49 | V170I_A436V | −10.41 |
| A270V_L277M | −12.39 | L181I_L435M | −11.86 | V195L_F337Y | −10.66 |
| A28L_A381T | −10.90 | L181M_L230M | −12.31 | V198L_Y293F | −11.02 |
| A28T_I288V | −11.59 | L202M_I351V | −10.26 | V200I_A217S | −10.29 |
| A28T_L223M | −10.74 | L202M_V247I | −11.05 | V247I_I351V | −11.23 |
| A28T_V51I | −10.93 | L205M_A298S | −9.87 | V257I_L399M | −10.91 |
| A298T_A342S | −9.97 | L223M_L230F | −11.35 | V257I_V338I | −11.08 |
| A324T_M354L | −11.02 | L223M_T419L | −10.61 | V259M_T406A | −10.28 |
| A329S_V347I | −12.88 | L226I_L334M | −10.06 | V261I_F448Y | −10.79 |
| A342L_I454V | −11.03 | L227M_Y452F | −11.66 | V284I | −11.13 |
| A364M_A398M | −11.53 | L227V_C260Y | −10.85 | V284I_V438L | −10.08 |
| A382S_A437V | −11.11 | L230M_M354L | −10.10 | V284I_Y452F | −11.21 |
| A382S_V438I | −11.03 | L230V | −11.45 | V290I | −10.33 |
| A392S_A420S | −9.86 | L267M_S407T | −10.67 | V290I_L395M | −10.72 |
| A402T_L411M | −11.19 | L313F | −12.20 | V290I_V426L | −11.83 |
| A43I | −12.71 | L391M_A398T | −9.37 | V296I_S379A | −10.78 |
| A48S_F337Y | −10.43 | L41I_T362S | −10.92 | V297I_S311T | −11.52 |
| A48T_V431I | −10.57 | L46M_L147M | −11.34 | V303I_S339L | −10.93 |
| A52T_A211T | −10.96 | L46M_L181F | −11.72 | V404I_A437T | −13.49 |
| A78L_S379I | −10.24 | L63M_A270T | −11.00 | V423I_L435M | −10.78 |
| A7S_M370L | −11.11 | L88M_M370V | −11.30 | V51I_Y388F | −10.19 |
| A7T_S101T | −10.25 | L88M_T165A | −10.65 | V55I_V247I | −10.93 |
| A99M_A301T | −10.52 | L88M_T196A | −10.42 | V55Y_L88V | −11.70 |
| A99T_V194I | −11.29 | M209L_L313M | −10.72 | V67I_L267M | −11.53 |
| C136S_V164I | −11.12 | M237L_F326Y | −11.52 | V67I_S346C | −9.68 |
| C136Y_Y172F | −10.47 | M321L_A436Y | −11.50 | V85L_I330V | −10.83 |
| C173S_S379A | −8.80 | M91L_A188S | −11.05 | Y151F_A301S | −10.85 |
| C173S_S433C | −10.71 | M91L_A327S | −11.30 | Y151F_S219A | −10.71 |
| C173Y_A381C | −10.90 | M91L_I105F | −10.65 | Y172F_A397V | −12.09 |
| C173Y_I269V | −10.89 | M91L_L202M | −9.97 | Y172F_T196S | −10.20 |
| C260A_M321I | −11.65 | M91L_L277M | −10.72 | Y18F_I30V | −11.62 |
| C45Y | −10.76 | S111F_A211V | −9.98 | Y203F | −10.80 |
| C45Y_A402T | −11.29 | S11A_S305C | −11.83 | Y242F | −10.63 |
| C59F_A154I | −10.54 | S11C_S111C | −10.19 | Y242F_A184T | −11.73 |
| F121Y | −12.45 | S11T | −10.63 | Y242F_A43I | −13.70 |
| F121Y_A224S | −10.58 | S155V_S219C | −11.75 | Y242F_L153M | −10.50 |
| F121Y_S244T | −12.04 | S174C_V200I | −11.01 | Y242F_M321L | −12.19 |
| F146Y_A376M | −12.82 | S221A_S285T | −9.52 | Y242F_Y452F | −12.61 |
| F182Y_V372I | −9.70 | S22A_L90I | −10.74 | Y388F_V438M | −10.46 |
| F246Y_S379I | −11.00 | S22C_S93A | −10.65 | Y452F | −10.12 |
| F306Y_A402S | −10.21 | S22T_A436S | −10.97 | Y57F_F356Y | −11.95 |
| I105V_A224M | −10.50 | S244A_S287T | −11.60 | Y57F_V426I | −9.59 |
| I105V_V164I | −10.96 | S244T_A398S | −10.83 | Y77F_A289S | −11.30 |
| I110V_A376S | −11.06 | S244T_T406I | −10.78 | Y77F_L313M | −9.93 |
| I110V_A382V | −9.99 | S244T_V249L | −10.19 | Y77F_L86M | −10.82 |
| I110V_S287A | −10.76 | S256C_A424M | −10.07 | Y77F_V185A | −10.33 |
| I12M | −10.83 | S256T_L399F | −11.28 | | |

Design of a Directed Evolution Recombinant Library with Multiple Site Degeneracy

A PCR-based gene synthesis methodology was used to build a de novo full-length recombinant gene for HDNO D2. The protein sequence of HDNO D2 was randomly reverse translated into a DNA sequence, and the resulting full-length DNA sequence was split into 26 overlapping oligonucleotides, while optimising the codons for E. coli and homogenising the annealing temperature by adjustment of the overlap GC content. The construct was assembled in vitro, and any incorrectly annealed bases were corrected using the proof-reading activity of a high-fidelity polymerase. Overlap extension PCR was then used to generate the final full-length recombinant gene. The new HDNO D2 gene variant was expressed and verified to have the same protein sequence and activity as the previously reported HDNO D2 enzyme ([6] and see Table 9 for the previously reported sequences used here) using solid phase screening. This full-length gene design was then used to introduce genetic diversity to create a library suitable for DE screening and selection.

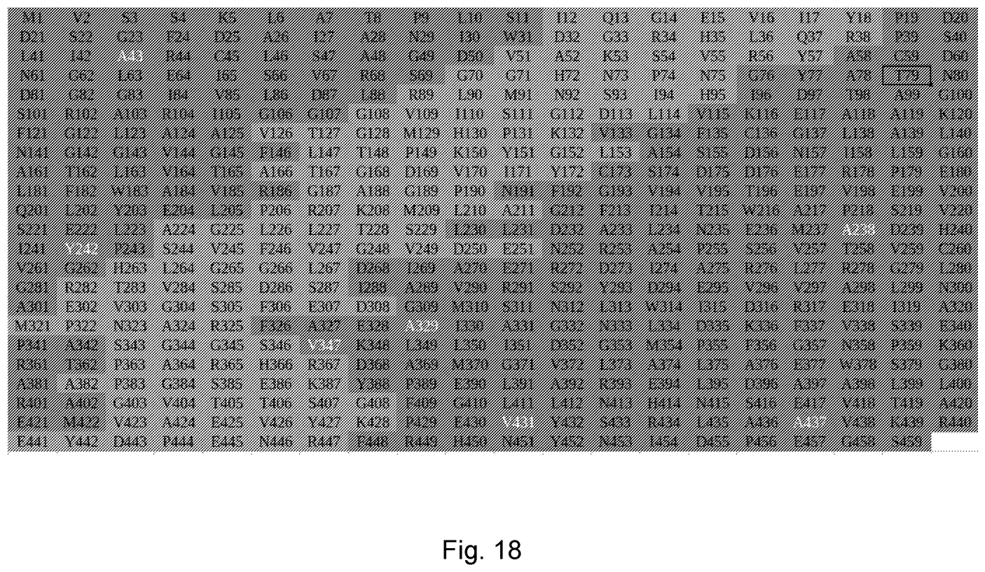

The HDNO 03 mutation was introduced into the library by resynthesizing oligonucleotide number 22 with a degenerate codon (MAT) at the position of N414, which codes for either Asn or His. Due to the nature of this PCR-based gene synthesis methodology, oligonucleotide overlap is essential for assembly and such regions accounting to about a third of the gene are not readily available for the insertion of genetic variability using degenerate codons (FIG. 18). The ranking from the scored MD simulations was used to determine preferential sites based on further practical criteria such as equal distribution of sites throughout the enzyme, avoidance of oligonucleotide overlap regions, the availability of a small degenerate codon that included both the original HDNO D2 amino acid and those predicted by the simulations, and the efficiency by which the degeneracy could be substituted into the sequence by using minimal oligonucleotide resynthesis. It should be noted that the rate estimations from this methodology are aimed at library enrichment, rather than accurate quantification of k_{cat}. While the selection was done manually in this case it could be done automatically. For example, sites could be selected as those with the most negative value that are feasible and practical given a set of experimental capabilities or limitations and/or that satisfy other criteria such as optimising of other properties (e.g. thermal or solvent stability) or avoiding a predetermined set of residues. For larger sets of mutations, and particularly where the simulation length is shorter (less than 50 ns) and hence introduces more noise into the predictions, a more optimal approach may be to use statistical analysis and/or machine learning techniques (vide infra). In other words, the site selection can be made based on highly ranked sites that satisfy practical and economic considerations. Typical restraints towards the selection of high-ranking sites might include one or more of: avoiding certain residues for their known or possible involvement in the reaction mechanism, disulphide bridge formation, cofactor binding; simplification of experiment (e.g., size and diversity of libraries associated with combinations of degenerate codons); and co-optimisation of other parameters such as thermal stability. In this example, the predictions were based on single and double mutations based on the assumption that producing focussed libraries at multiple beneficial sites increases the chances of discovering synergistic epistatic effects (namely effects that are not obtained by the individual mutations but by the combination of two or more mutations due to interactions between amino acids) that can significantly improve enzyme turnover numbers. In later examples it is shown how that concept can be extended by making predictions that are based on simultaneous mutation at three or more sites to increase the chances of producing a mutant with beneficial epistatic effects.

In this case, an efficient combinatorial oligonucleotide library could be constructed that simultaneously contained seven high-ranking target sites (see Table 11 for the top 20 ranked sites) with a diverse set of small degenerate codons (Table 15) within a set of five degenerate oligonucleotides (sites 238 and 242 could be included in the same oligonucleotide and likewise sites 431 and 437. A visual representation of the location of the sites of genetic variability within the library and how these sites fit into the non-overlap regions of the full-length gene is shown in FIG. 18 (see green for non-overlap regions). It can be observed that some of the best ranking sites, such as 404, could not be included in this oligonucleotide design, but if such sites were mandated then it would, in principle, be possible to redesign the oligonucleotides to move the overlap regions to other parts of the protein sequence, which would result in varying the possible selection of sites in the library. In fact, given a ranking list, the library design is very amenable to process standardisation and automation.

TABLE 11
Site-Ranking according to dynamic μ_{Q20} barriers (top 20 ranked
sites only) and selection for full length gene based DE library design

| | μ_{Q20} | Best | | Selected |
|---|---|---|---|---|
| Site | kcal/mol | amino acid | Mutant | for Library |
| 43 | −13.7032 | I | Y242F_A43I | YES |
| 242 | −13.7032 | F | Y242F_A43I | YES |
| 437 | −13.4867 | T | V404I_A437T | YES |
| 404 | −13.4867 | I | V404I_A437T | NO |
| 329 | −12.883 | S | A329S_V347I | YES |
| 347 | −12.883 | I | A329S_V347I | YES |
| 146 | −12.818 | Y | F146Y_A376M | NO |
| 376 | −12.818 | M | F146Y_A376M | NO |
| 238 | −12.7987 | T | A238T | YES |
| 419 | −12.7535 | S | I27V_T419S | NO |
| 27 | −12.7535 | V | I27V_T419S | NO |
| 184 | −12.7315 | V | L153F_A184V | NO |
| 153 | −12.7315 | F | L153F_A184V | NO |
| 431 | −12.7243 | I | T148A_V431I | YES |
| 148 | −12.7243 | A | T148A_V431I | NO |
| 452 | −12.6109 | F | Y242F_Y452F | NO |
| 121 | −12.4496 | Y | F121Y | NO |
| 270 | −12.3909 | V | A270V_L277M | NO |
| 277 | −12.3909 | M | A270V_L277M | NO |
| 133 | −12.3303 | M | V133M_A270T | NO |

### Screening of the Rational Directed Evolution Library and Characterisation of HDNO D6

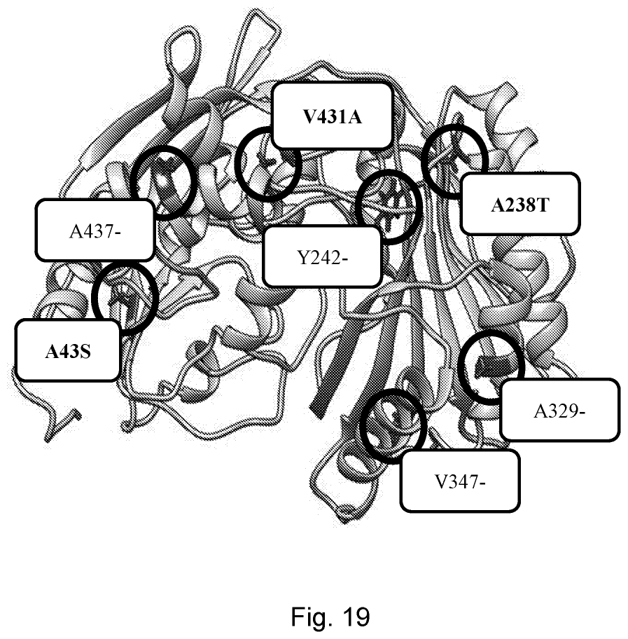

The newly generated recombinant DNA library, which had a maximum possible genetic diversity in the order of 10⁸, was transformed into E. coli cells. From this, circa 16000 individual colonies were grown and screened for their ability to express the enzyme and catalyse the oxidisation of PPY using a horse radish peroxidase based solid phase assay. Thus, the screening assay covered only a small fraction of the possible mutants present in the library. After a 20 min incubation period, several colonies were observed with noticeably increased activity toward oxidation of PPY. The most active colony was selected and sequenced and corresponded to a protein containing six amino acid mutations compared to wild-type 6-HDNO. These mutations were identified as: A43S, A238T, E350L, E352D, N414H, and V431A; the new mutations introduced by the library are distributed throughout the HDNO protein and not localised to the active site (FIG. 19). The new active mutant, henceforth referred to as HDNO D6, was subsequently expressed and purified on a milligram scale for more detailed characterization using analytical scale biotransformation assays. The rapid discovery of this mutant also confirms the hypothesis that beneficial site predictions based on single and double mutations increase the chances of discovering synergistic epistatic effects from activity screening of focussed libraries containing these sites (note that the three new mutations in D6 were never tested together in the MD experiments).

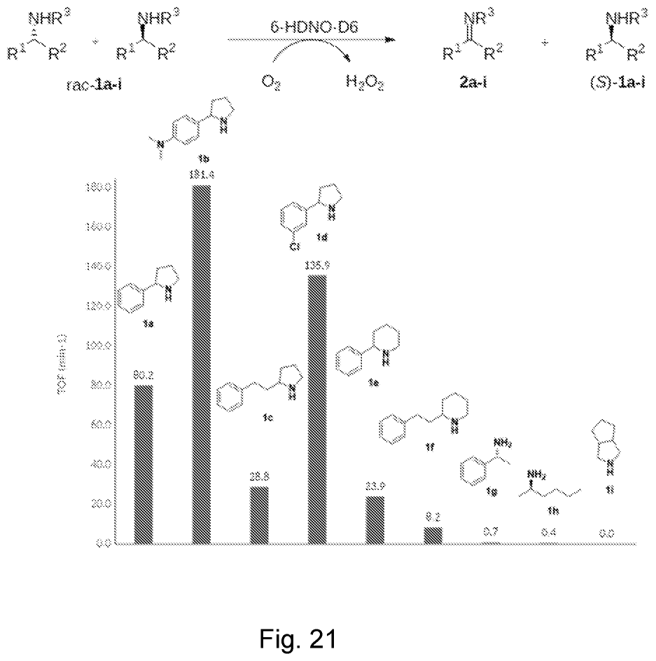

The turnover frequency (TOF) of HDNO D6 was determined for a diverse set of amine substrates (including PPY); using gas chromatography to monitor the oxidised product yield (see FIG. 20 and FIG. 21). Activity towards PPY represented a 5.8-fold increase in TOF with respect to previously reported results for HDNO D2 ([6], Table 12 and FIG. 20 compound 1a) and an improvement of 3.3-fold when compared to HDNO D3. Other selected amine-containing substrates, which were neither directly targeted by the computational library design, nor included in the solid-phase screening, also saw a general improvement in their TOF numbers. This comprised a panel of primary and secondary amines, containing both piperidine and pyrrolidine heterocycles and aliphatic chains. As would be expected for a library rationally designed using PPY, the increases in TOF numbers were the greatest for secondary amines and pyrrolidine derivatives. For the 2-phenylethyl derivative of pyrrolidine (FIG. 20 compound 1c), the observed increase was comparable with PPY, while the greatest increase in TOF number (10.2-fold) compared with HDNO D2 was found for the larger molecular mass compound 2-(4-dimethylaminophenyl)-pyrrolidine (see FIG. 20 compound 1 b). A large increase in TOF was also observed for the halogenated 3-chloro phenyl derivative of PPY (see Table 10 and FIG. 21 compound 1d). For piperidine containing molecules, while the overall TOF values remained low, increases were also observed in HDNO D6 compared with HDNO D2. For example, for phenyl piperidine (FIG. 20 compound 1e) a significant increase of 3.1-fold only was obtained. Measurable activity was also found in primary amines. Specifically, under biotransformation, the substrates methylbenzylamine and 2-aminohexane show quantifiable activity in HDNO D6; methylbenzylamine also showed similar measurable oxidation rates using HDNO D2. In HDNO D3, the activity to methylbenzylamine was about 20% lower from that found in HDNO D6. Thus, HDNO D6 maintains activity towards primary amines found in HDNO D2, while increasing general rates toward the secondary amines tested here. However, even in HDNO D6, no measurable activity was found for a prototypical fused pyrrolidine ring structure (Table 12, FIG. 21 compound 1i).

TABLE 12
Turnover frequencies (TOF, min⁻¹) and activity increase over HDNO D2
of HDNO D6 across a series of secondary and primary amines. Reaction
conditions: 10 mM substrate, 0.2 mg ml⁻¹ enzyme (D2) or 0.05 mg mL⁻¹
(D6), 30° C. in pH 8 100 mM buffer. TOF (min⁻¹) calculated using
conversions after 10 min. n.d.: not determined. n.a.: no activity or
too low to be accurately determined.

| Substrate | | TOF (min⁻¹) | Fold increase vs D2 |
|---|---|---|---|
| 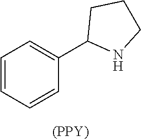 | 1a | 79.8 | 5.8 |
| 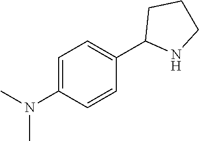 | 1b | 181.2 | 10.2 |
| 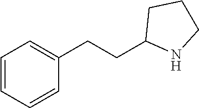 [SMILES: c1ccc(CCC2CCCN2)cc1] | 1c | 28.8 | 5.9 |
| 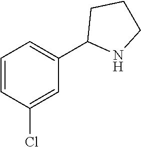 [SMILES: Clc1cccc(C2CCCN2)c1] | 1d | 135.6 | n.d. |
| 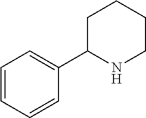 [SMILES: c1ccc(C2CCCCN2)cc1] | 1e | 24.6 | 3.1 |
| 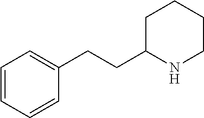 [SMILES: c1ccc(CCC2CCCCN2)cc1] | 1f | 8.4 | n.a |
| 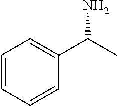 | 1g | 0.66 | n.a. |
| 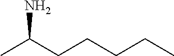 | 1h | 0.42 | n.a. |
| 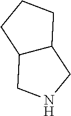 | 1i | Not active | n.a. |

### Stability Improvement of the Optimised HDNO D6 Enzyme to Produce HDNO D8.

During the characterisation of HDNO D6, a decrease in both enzyme expression levels, and stability was observed compared with the less active mutants. This progressive reduction in stability as more amino acids are mutated (to increase activity) has been reported previously [102]. To quantify this, the thermal stabilities of both HDNO 03 and D6 were determined by incubating the purified enzymes at 50° C. for 60 min. Whereas 62% residual activity was observed for HDNO D3 after 60 min, HDNO D6 was completely inactivated after only 15 min. Therefore, a standard stabilisation process was applied to the HDNO D6 mutant. There are several well-established approaches for this task including random mutagenesis, stabilisation of flexible regions, generation of salt bridges or the introduction of disulphide bonds, among others. Another amenable approach is enzyme supercharging, which involves modifying only the surface residues to either increase or decrease the net protein charge, with the effect of increasing folding stability [103]. Experiments were performed to demonstrate that the supercharging method for enzyme stabilisation is both effective and compatible with the rational library design DE approach based on dynamics and electrostatics. A method, based on average number of neighbouring atoms per sidechain atom, was employed to select mutations via their surface accessibility, which could be targeted to increase the surface charge and hence stabilise the protein. The HDNO protein has a high negative charge (of about −20e including covalently bound flavin dinucleotide). Therefore, to increase the overall negative charge, the considered mutations were from a specific set of either neutral or positively charged amino acids (Asn, GIn, Arg and Lys) to negative amino acids (Asp and Glu), with a surface accessibility (AvNASPA) score less than 100 (see Table 13). These 20 predicted surface mutations were inserted into the HDNO D3 enzyme individually, and, where viable, the proteins expressed, purified, and tested for activity and thermostability.

TABLE 13
Sorted AvNAPSA scored mutants for stabilization.

| | | | AvNAPSA | | |
|---|---|---|---|---|---|
| Rank | Site | Residue | score | Mutation | Variant |
| 1 | 252 | ASN | 55.8 | D | N252D |
| 2 | 282 | ARG | 56.7 | E | R282E |
| 3 | 207 | ARG | 59.7 | E | R207E |
| 4 | 13 | GLN | 59.8 | E | Q13E |
| 5 | 120 | LYS | 61.6 | E | K120E |
| 6 | 272 | ARG | 63.3 | E | R272E |
| 7 | 360 | LYS | 63.5 | E | K360E |
| 8 | 401 | ARG | 63.8 | E | R401E |
| 9 | 325 | ARG | 66.9 | E | R325E |
| 10 | 208 | LYS | 69.6 | E | K208E |
| 11 | 428 | LYS | 76.7 | E | K428E |
| 12 | 5 | LYS | 79.0 | E | K5E |
| 13 | 253 | ARG | 79.4 | E | R253E |
| 14 | 440 | ARG | 85.7 | E | R440E |
| 15 | 387 | LYS | 90.8 | E | K387E |
| 16 | 56 | ARG | 93.7 | E | R56E |
| 17 | 361 | ARG | 94.6 | E | R361E |
| 18 | 276 | ARG | 97.4 | E | R276E |
| 19 | 116 | LYS | 97.9 | E | K116E |
| 20 | 92 | ASN | 99.4 | D | N92D |

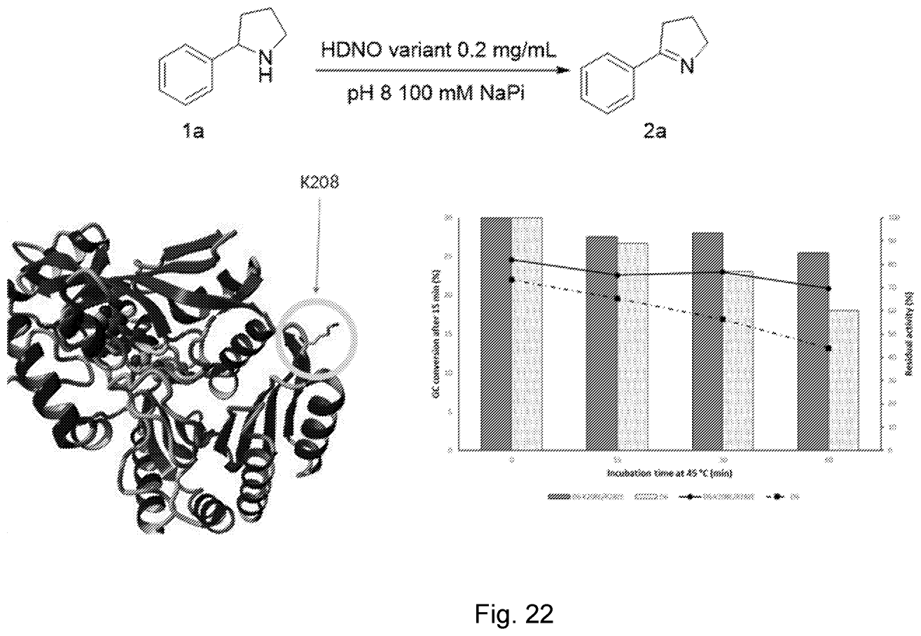

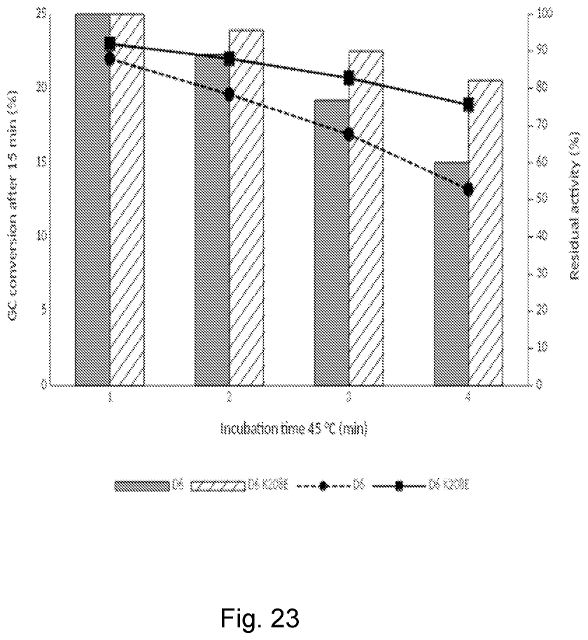

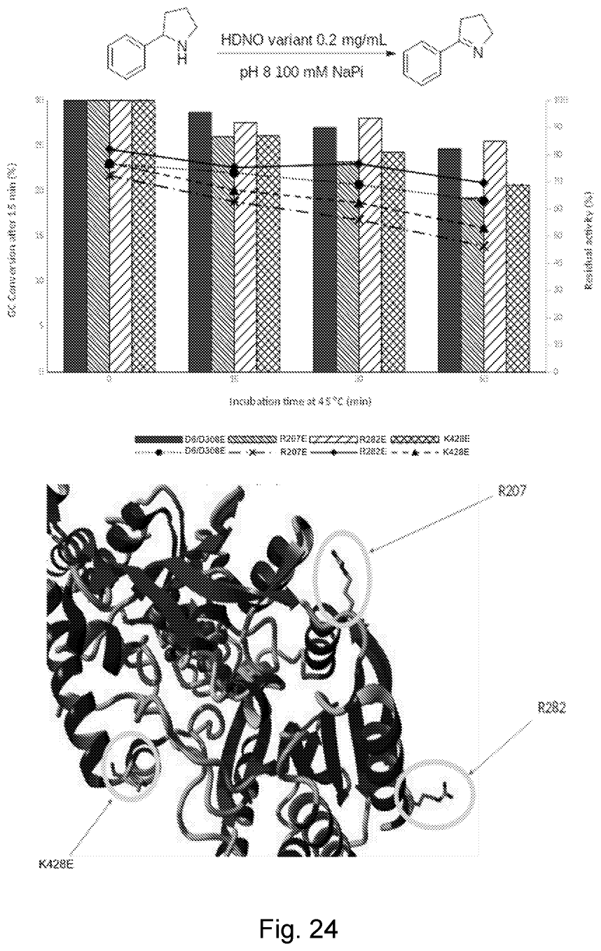

Of the predicted surface mutations, several of them (namely D308E, R207E, R282E, K428E and K208E) were successfully expressed and were confirmed to have some activity and were subsequently tested for their effects towards thermal stabilization by supercharging. Using similar conditions to those used for biotransformations of PPY in the HDNO D3 and D6 enzymes, all supercharged mutations either presented a neutral or positive effect on activity. An initial set of thermal stability measurements were made for K208E on both HDNO 03 and HDNO 06 variants using gas chromatography measurements of activity taken every 15 min from incubations at 50° C. (see FIG. 22). An increase in residual activity of 32% for HDNO D3/K208E compared to HDNO D3 was observed after 60 min. However, while no residual activity was observed after 60 min at 5000 for the HDNO D6/K208E mutant, after 60 min at 4500 it maintained over 80% of its residual activity, which was 36% higher than that for HDNO D6 (see FIG. 23). Further measurements at 4500 revealed that two other mutants of HDNO D6 (R282E and D308E) showed significant increased thermal stability as compared to HDNO D6 (see FIG. 24).

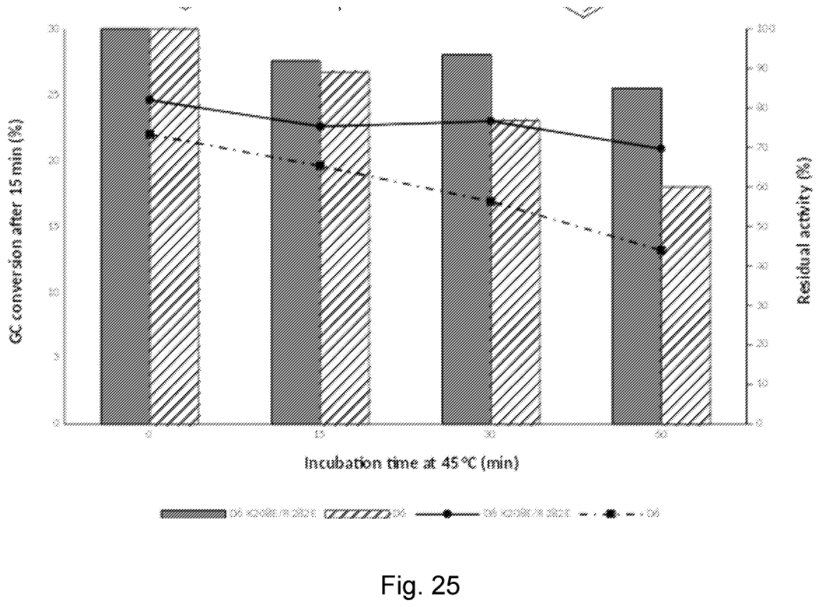

Subsequent investigation of the double mutant HDNO D6/K208E/R282E (now termed HDNO D8) showed that it retained 85% of residual activity after 60 min at 45° C. (indicating a 42% increase in thermal stability over HDNO D6), while serendipitously also displaying a 1.2-fold improvement in activity towards PPY at non-elevated temperatures (see Table 14 and FIG. 25). In producing the final HDNO D8 enzyme, proof of concept has been shown that this enzyme could be rationally improved in both activity and thermal stability by using a customized degenerate full-length gene library followed by supercharging.

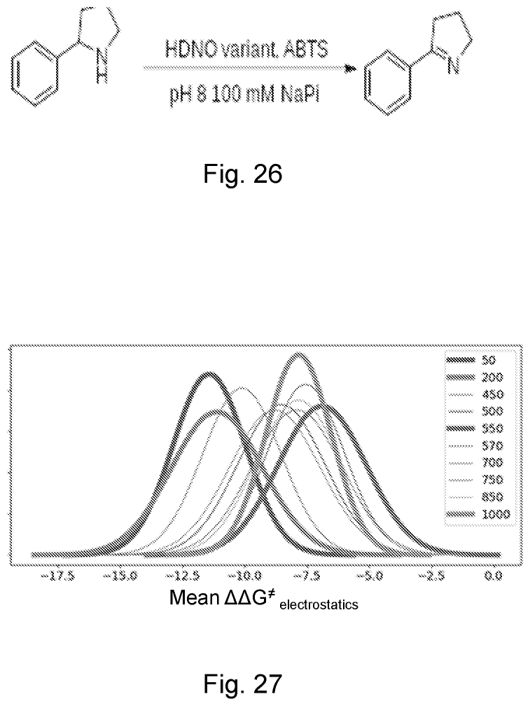

TABLE 14
Kinetic parameters for D2 and improved variants in the oxidation
of 2-phenylpyrrolidine using the HRP-ABTS assay (FIG. 26).
K_{M} and kcat values calculated using Prism7.

| variant | kcat (s⁻¹) | K_{M} (mM) | kcat/K_{M} |
|---|---|---|---|
| D8 D352E | 1.22 ± 0.12 | 2.49 ± 0.67 | 0.49 |
| D8 | 2.83 ± 0.28 | 4.01 ± 0.91 | 0.71 |
| D6 | 1.01 ± 0.11 | 0.88 ± 0.33 | 1.15 |
| D3 | 0.71 ± 0.05 | 0.49 ± 0.14 | 1.47 |
| D2 | 0.50 ± 0.06 | 1.06 ± 0.44 | 0.48 |

Evolved monoamine oxidase (MAO-N) enzymes were previously shown to have good activity and selectivity towards the (S)-enantiomer of PPY, with reported k_{cat} of 2.50 s⁻¹ for variant N336S and 2.13 s⁻¹ for variant N336S/1246M [104]. While biocatalytic conversion of the opposite (R)-enantiomer of PPY (and other related chiral (R)-amines) have been previously shown to be catalysed stereo-specifically by 6-HDNO, this was at a much-reduced rate compared with MAO-N. The new variants HDNO D6 and HDNO D8 described here show activities of 1.01 s⁻¹ and 2.83 s⁻¹ respectively, as measured using biotransformation assays for the PPY (R)-enantiomer, which are equivalent to the activities measured for the aforementioned MAO-N variants on the opposite enantiomer.

While out of the scope of this current work, it is noted that the newly generated HDNO D6 variant shows measurable activity towards the primary amine substrates (R)-methylbenzylamine and (R)-2-aminohexane. High levels of catalytic activity for the (S)-enantiomer of methylbenzylamine have been achieved in the most progressed MAO-N variants after many rounds of DE [104, 105]. Despite HDNO D6 or HDNO D8 not presently being as active towards primary amines, these new mutants have significant potential for future improvement and application in the manufacture of APIs.

### Materials and Methods

### Site Directed Mutagenesis.

Plasmid pET16b-HDNO (E350L/E352D) [6] served as template for the construction of site directed mutagenesis libraries. Introduction of the different mutations in single positions was achieved by inverse PCR using the appropriate primers. Primers were synthesised by Eurofins Genomics and prepared as (100 pmol/μl) by reconstituting the lyophilized primer (as supplied) in the prescribed amount of dH₂O. PCR reactions were carried out in thin-walled 200 μl PCR tubes, using reagent as supplied in the Phusion DNA Polymerase kit (NEB) using the Q5 inverse PCR protocol. Amplification of the target was checked on a 1% agarose DNA gel containing 0.01% SYBR-Safe reagent. Template was digested with, Dpnl for 1 hour at 37° C. The reaction mixture was purified using a PCR clean up kit (Qiagen). The purified linear DNA was subjected to a ligation protocol followed by transformation into DH5a (NEB) competent cells according to the manufacturers protocol. Single colonies were picked, grown in 5 ml LB overnight at 37° C. The plasmid DNA was isolated using a mini-prep kit (Qiagen) following the manufactures protocol. The introduction of the corresponding mutations was confirmed by sequencing (Eurofins Genomics).

### Gene Expression

Plasmids containing genes for the different variants were used to transform E. coli BL21 (DE3) competent cells for gene expression. Overnight cultures were prepared by inoculating 4 ml of LB containing 100 μg ml⁻¹ ampicillin with a single colony and incubated for 16 h at 37° C., while shaking continuously at 250 r.p.m. The overnight culture was then added to a flask containing 400 ml autoinduction media and 100 μg ml⁻¹ ampicillin and incubated for 3 days at 20° C. with shaking at 200 r.p.m. The cells were then harvested by centrifugation at 4,000 r.p.m. for 20 min and the cell pellets stored at −20° C. prior to purification.

### Purification

For purification, the cell pellets were thawed and resuspended in buffer A (100 mM NaPi, 300 mM NaCl, 30 mM imidazole pH 8). Cells were disrupted by ultrasonication using 30 s on and 30 s off cycles (20 repeats) using a Soniprep 150 (MSE UK Limited, London), and the suspension was centrifuged at 16,000 r.p.m. for 30 min to yield a clear lysate. The N-terminal Hiss-tagged proteins were purified using immobilised-metal affinity chromatography by loading onto a 5 ml HisTrap FF column (GE Healthcare UK Limited, Chalfont St. Giles). The column was subsequently washed with 15 ml buffer A and eluted with buffer B (100 mM NaPi, 300 mM NaCl, 300 mM imidazole pH 8). Fractions were collected and protein concentration measured using nanodrop spectrophotometer. Fractions containing protein were combined and concentrated using a Vivaspin 6, 30 kDa cut-off spin column (GE Healthcare UK Limited, Chalfont St. Giles) and the purified protein was desalted on a PD-10 column (Merck Life Science UK Limited, Gillingham) using 100 mM NaPi, 300 mM NaCl buffer pH 8. Purified protein was short-termed stored at 40C or snap-frozen and stored at −80° C. prior to use.

### Generation of an Initial 6-HDNO Model

The setup of a 3D-model for the HDNO D2 enzyme with (R)-phenyl pyrrolidine (PPY) was performed as explained in Example 1. In brief, an initial HDNO D2 model was prepared by insertion of a double mutation (E350L/E352D) into a wild type crystal structure of 6-HDNO (protein data bank entry 2BVF) including the covalently attached flavin dinucleotide [44]. This was solvated in a cubic box of water and 50 ns of NPT molecular dynamics (MD) equilibration (298 K, 1 atm) was performed, followed by a 1 μs of NVT MD simulation. The substrate was then positioned in the active site cavity of the final set of 3D-coordinates. Starting from this configuration, a 10 μs NVT simulation was performed with the substrate free to move. At 1212 ns the substrate was found to be in a near attack conformation for hydride transfer and these coordinates were extracted and the substrate harmonically restrained to the 6-HDNO flavin isoalloxazine ring. This 3D-structure (including solvent and harmonic restraint), which has been described in detail in Example 1, was used as the starting point for all subsequent protein mutant structures.

### Molecular Dynamics (MD) Simulation

All molecular modelling and MD simulations were performed using OpenMM [53], with the AMBER force-field parameters for protein [51], the general AMBER force field (GAFF) for the substrate and flavin adenine dinucleotide molecules [48] and TIP3P for water [52]. Electrostatics were modelled by the particle mesh Ewald (PME) method with a 0.9 nm cut-off, switched at 0.75 nm, and error tolerance 5×10⁻⁴. Hydrogen atoms were fixed with SHAKE and water molecules kept rigid (constraint tolerance 1×10⁻⁵). The hydrogen mass was increased to 4 amu using the hydrogen mass repartitioning method [54], allowing a time-step of 4 fs with the Langevin integrator. Temperature was kept constant at 298 K using a collision rate of 0.1 μs⁻¹. Alternative software for performing MD are known in the art and include e.g. CHARMm, Tinker, Gromacs. Any such alternative could be used to produce equivalent conformational sampling results. Further, MD software parameters could also be modified within reasonable ranges from the parameters used herein without expecting a significant impact on the results. For example, the use of different solvent models or periodic boundary conditions or the use of the NPT ensemble instead of NVT is envisaged.

### In Silico Mutagenesis and Mutant MD Simulations

A total of 23 single and 213 double mutant variants were randomly generated within an allowed subset of conservative mutations, including only mutations of sites corresponding to any of the 10 conservative amino acids: Ala, Cys, lie, Lys, Met, Phe, Ser, Thr, Tyr and Val. In this case, the term “conservative” refers to mutations having a low impact on charge and electrostatics, i.e., substitution of a neutral amino acid for another neutral amino acid. Mutations involving a removal or insertion of a cysteine were assumed to correspond to protonated or uncharged variants, and it was assumed that no disulphide bonds were broken or formed in the process. Additionally, residues 1 to 5 and 454 to 459 were intentionally avoided due to these regions being less restrained to the protein structure as this would likely result in a weaker mutation-response signal. Residue 72 was also avoided because it corresponds to a crucial His residue that is covalently bound to the flavin cofactor in 6-HDNO.

Mutations were inserted into the 6-HNDO 3D-structural model by modifying the appropriate side chain atoms and leaving the main chain untouched. Each mutant model was energy minimized to a tolerance of 10 kcal mol⁻¹, followed by a simulated annealing protocol to remove any unwanted steric clashes involving mutated residues. In this protocol, an NVT MD simulation was performed over a time (t) of 1.1 ns at 298 K. During this period, the non-bonded potentials of the mutated atoms (van der Waals and electrostatics) were reduced to fractional values of the original value and ramped-up overtime according to

$$\\ {( \frac{t}{1.1} )}^{2} .$$

The resultant annealed coordinates and velocities were used to start 50 ns production NVT MD simulations (with fully restored non-bonded potentials). For each mutant simulation, the coordinates were saved at 0.1 ns intervals for further analysis.

### Ranking by Dynamic Scoring and Selection of Degenerate Codons

The mutant scoring methodology was performed as described in Example 1 (and all the previously calculated parameters were reemployed here). In brief, the reactant complex and first transition state representing a hydride transfer activation step were optimised in an electrostatically embedded QM/MM model implemented in ChemShell [66, 67] at the B3LYP/def2-SVP level of theory for the QM region with the Turbomole Software. Alternative methods to QM/MM can be used, such as using a DFT cluster model. Thus, other software packages implementing DFT models could be used such ORCA (https://orcaforum.kofo.mpg.de/app.php/portal), Q-Chem (https://www.q-chem.com/), NWChem (https://www.nwchem-sw.org/), and Gaussian (https://gaussian.com/) could also be used. The classical interactions with the MM region were calculated by DL_Poly [69] with a CHARMM forcefield and CGenFF and C36-protein parameters [71, 72], except for the flavin adenine dinucleotide cofactor and the substrate, where parameters were generated by SwissParam [74]. The substrate and FAD cofactor are not common residues in proteins and therefore their parameters are not already published and needed to be generated. Methods other than SwissParam could also be used for this, including de novo generating parameters with DFT software or using the general force field (GAFF) from molecular modelling software AMBER. During electrostatic scoring, the coordinates were divided into a core region (closely related to the chemical change) and an external region (containing the rest of the system), where MM charges are assigned based on typical force field partial atomic charges for protein residues. In this case the C36 protein parameters were also used for this purpose but other equivalent parameters such as the ff14SB typically used for the AMBER force field could also be employed. A calculation of partial atomic charges of the reactant complex and the transition state geometries was performed for every atom (to calculate a change in partial atomic charges for each atom in the core region). In this case, the partial atomic charges were calculated using the Gaussian software [50] via a CM5 population analysis [78] on the core region atoms as previously optimised by a QM/MM method. Alternative methods to the QM/MM methodology include the use of DFT cluster models, where any of DFT or quantum chemistry software may be used (e.g., ORCA Chemistry, Gaussian, Q-Chem). Similarly, the partial atomic charge calculation can be performed by any DFT or quantum chemistry software by many acceptable methods, e.g., by changing the DFT functional (e.g., BP86, B3LYP, M06) and/or the basis set (e.g., 6-31G*, 3-21G*, def2-SVP). Partial atomic charge calculations were performed by a CM5 population analysis at the B3LYP/6-31G* level of theory with an implicit water model [61, 62, 63, 77]. Any reasonable implicit solvent model or a gas phase model may result in equivalent results. The ΔΔG^{‡} score for a specific frame constitutes a summation over all the Coulombic interactions of the external region with the difference of partial atomic charges in the active region for each coordinate set extracted from a mutant MD simulation (see Example 1), and all mutant MD simulations were scored using this process. The data was post-processed to extract the most promising sites for improved activity, by ranking the sites based on the lowest score found for all mutants that included that site, reflecting the maximum potential effect caused by a mutation at that position.

Due to the nature of PCR-based gene synthesis, oligonucleotide overlap is essential for assembly and these regions (about a third of the gene) are not readily available for the insertion of genetic variability using degenerate codons. The ranking from the molecular dynamics simulations was then used to determine preferential sites based on further practical criteria such as equal distribution of sites throughout the enzyme, avoidance of oligonucleotide overlap regions, the availability of a small degenerate codon that included both the original HDNO D2 amino acid and those predicted by the simulations, and the efficiency by which the degeneracy could be substituted into the sequence by using minimal oligonucleotide resynthesis. After obtaining the list of best ranking sites for mutagenesis, a process for the selection of degenerate codons was followed with the objective of creating an efficient library of manageable size, no amino acid redundancy and lower PCR bias. Therefore, each selected degenerate codon had at most a multiplicity of eight (inclusive of the HDNO D2 mutation) and with no stop codons. Furthermore, it was intended that all the mutant variants that had a higher-ranking score for mutations on the target site were included. No explicit multi-objective optimisation strategy was followed other than choosing a suitable degenerate codon with a low multiplicity that would comply with these requirements.

### Rational Library Construction

The construction of the DE library was made by error-corrected PCR based de novo full gene synthesis [7]. Oligonucleotides sequences were optimised to be suitable for the E. coli host, as well as for adjustment of annealing temperatures on the ligation sites followed by removal of miss-annealing nucleotides [115]. The sequence was split into a construct comprising 26 oligonucleotides based on the sequence of the HDNO-D2 enzyme (see Table 9). A first test transformation was based on the D2 variant alone. A subsequent library was generated by the inclusion of small degenerate codons with no stop codons (maximum multiplicity eight, see above) that were selected based on the previously calculated rankings based on the scoring methodology. Several high-ranking sites were selected as per the library design described in Table 15. A total of 7 degenerate codons were introduced in 5 degenerate oligonucleotides. An additional dual degeneracy (degenerate codon MAT) was included on residue N414 to allow for both HDNO D2 and HDNO D3 variants to be included in the library. The oligonucleotide libraries were synthesised by GeneMill (University of Liverpool, UK).

TABLE 15
Target residues, degenerate codons (IUPAC one letter nucleotide
codes) and coded amino acids (IUPAC one letter amino acid
codes) for the construction of the PCR based synthetic gene
library. A series of small degenerate codons were used at
each selected site and inserted in 5 degenerate oligonucleotides
out of 26 that were required for the full protein.

| Target | Codon | |
|---|---|---|
| residue | degeneracy | Coded amino acids |
| A43 | NYT | A, I, L, F, P, S, T, V |
| A238 | DYT | A, I, F, S, T, V |
| Y242 | NWT | N, D, H, I, L, F, Y, V |
| A329 | RNT | A, N, D, G, I, S, T, V |
| V347 | NYT | A, I, L, F, P, S, T, V |
| N414 | MAT | N, H |
| V431 | NYT | A, I, L, F, P, S, T, V |
| A437 | NYT | A, I, L, F, P, S, T, V |

### Selection of Protein Stabilizing Mutants

The surface residues of HDNO D3 were identified based on their on average number of neighbouring atoms per sidechain atom (AvNAPSA), as described previously [103, 116]. The algorithm calculates the average number of protein atoms within a set distance (10 Å) of the atoms within a particular residue side chain. In this case the averaging was additionally performed over 10000 coordinate sets from a 1 μs simulation of HDNO D3. A value of less than 100 was typically taken to indicate a surface exposed residue. The HDNO protein has a high negative charge (of about −20e including covalently bound flavin dinucleotide), so the aim was to increase the total negative charge of the enzyme to supercharge it. Therefore, to increase the overall negative charge, the considered sidechains were restricted to a specific set of either neutral or positively charged amino acids (Asn, Gln, Arg and Lys). With these restrictions and a cut-off of 100 on the AvNAPSA score, a total of 20 amino acids were selected as targets for supercharging (see Table 13). For Asn, Gin and Arg the indicated conservative net-negative mutation was to Glu, while for Asn it was Asp.

A subset of mutants was selected and inserted into the HDNO D3 and D6 variants by site directed mutagenesis either alone or in pairs to also check for interactions that would further stabilise the enzyme. Namely, D3-K208E, D6-K208E, D6-R207E, D6-K282E, D6-K428E and D6-K208E/K282E mutant proteins were expressed and purified (as described below) and incubated at elevated temperatures (45° C. or 50° C. for 1 h for D6 variants) using a constant temperature water bath incubator. Aliquots were taken every 15 min and activities were measured by biotransformation and gas chromatography (as described below). The % residual activity corresponds to the ratio between the activity observed after incubation at each specified time over the activity of the enzyme without any thermal treatment.

Solid Phase Screening A solid phase screen method was used to screen libraries. E. coli BL21 (DE3) competent cells were transformed with either a library or the wildtype as described above. The transformation reaction was plated on a HyBond membrane (Merck Life Science UK Limited, Gillingham) on LB containing 100 μg ml 1 ampicillin and grown overnight at 30° C. The membrane was then transferred to a second LB plate containing 100 μg ml⁻¹ ampicillin and 1 mM IPTG and the protein expression induced for 6 h at 25° C. after which membranes were kept frozen at −20° C. until use. Membranes were freeze-thawed three times (using liquid N₂) before being placed on filter paper containing 0.1 mg ml⁻¹ horse-radish peroxidase (HRP) (Merck Life Science UK Limited, Gillingham) in pH 8.0, 0.1 M potassium phosphate buffer. The membranes were left at room temperature for 1 h to ensure removal of any cellular H₂O₂. The membrane was then transferred to another filter paper containing a solution of 0.1 mg ml⁻¹ HRP, 3,3′-diaminobenzidine, made from 1 tablet per 15 ml of SigmaFast (Merck Life Science UK Limited, Gillingham), and 10 mM substrate. Colonies that turned dark red or brown indicated that the expressed protein was active on the substrate. Active colonies were picked and added to a 5 ml mixture of LB and 100 μg ml⁻¹ ampicillin and grown overnight at 37° C. with 250 r.p.m. shaking. Plasmid DNA was extracted from fully grown colonies using a mini-prep kit (QIAPrep, Qiagen). The plasmids were sequenced (Eurofins genomics) to determine the mutations associated with increased activity.

### Chromatography

Chiral normal phase HPLC was performed on an Agilent HPLC system (G1379A degasser, G1312A binary pump, a G1367A well plate autosampler unit, a G1316A temperature-controlled column compartment and a G1315C diode array detector) (Agilent Technologies Inc., Santa Clara, CA) equipped with a CHIRALCEL OD-H (250 mm length, 4.6 mm diameter, 5 μm particle size) analytical column (Daicel Corp., Osaka, Japan). The typical injection volume was 10 μL and chromatograms were monitored at 265 nm. Gas chromatographic (GC) analysis was performed on an Agilent 6850 GC (Agilent Technologies Inc., Santa Clara, CA) with a flame ionization detector and autosampler equipped with a HP-1 column of length 30 m, 0.32 mm inner diameter and 0.25 μm film thickness (Agilent Technologies Inc., Santa Clara, CA).

### Biotransformations

A typical 500 μL reaction mixture in a 2 mL tube contained 10 mM amine, 0.05 to 1 mg mL⁻¹ of purified HDNO variant in pH 8 100 mM NaPi buffer. Reactions were incubated at 30° C. with 250 r.p.m shaking for different reaction times, after which they were quenched by the addition of 50 μL of 10 M NaOH and extracted twice with 500 μL tert-butyl methyl ether (HPLC grade, Merck Life Science UK Limited, Gillingham). The organic fractions were combined and dried over anhydrous MgSO₄ and analysed by GC-FID (conversions) or HPLC (enantiomeric ratios) on a chiral stationary phase (see Chromatography section for details). Turnover frequencies (TOF, min⁻¹) were calculated as the moles of product formed/moles of enzyme min⁻¹ based on conversions obtained by GC-FID analysis (see Chromatography method). The same response factor was considered for both substrate and product.

### Kinetics

Kinetic parameters of different HDNO variants were determined using the ABTS-HRP assay. Samples of 20 μL of substrate at different concentrations in DMSO (0.05 mM-10 mM, 10% DMSO final concentration) was diluted in 130 μL of reacting solution which contained 0.2 mg ml⁻¹ HRP (IV, Sigma) and 0.4 mg ml⁻¹ ABTS in 100 mM pH 8 NaPi buffer. The assay was started by adding 50 μL purified enzyme (typically 0.1-2 mg ml⁻¹ concentration). Production of the reduced ABTS was measured using a Tecan infinite M200 microplate reader (Tecan Group Limited, Mannedorf, Switzerland) at 420 nm (E=36 mM⁻¹ cm⁻¹) and 30° C. for 10 min. Measurements were made in triplicate and a 1:1 ratio for the oxidation of substrate to the production of hydrogen peroxide was assumed. Rate was plotted against substrate concentration and Vₘₐₓ and K_{M} values extracted using non-linear regression analysis with a fit to the Michaelis-Menten equation using Prism software (GraphPad Software, San Diego, CA). Protein concentrations for kinetic parameters were determined using the Bradford assay following supplier's instructions using bovine serum albumin (BSA) standards for calibration (Merck Life Science UK Limited, Gillingham).

### Conclusions

The typical increase in enzyme activity expected from an iterative DE experiment is around 5-fold [107], although different values are reported, which in turn depends on several factors, such as the initial activity of the substrate or the evolvability of the enzyme for the substrate [108]. Therefore, a direct comparison of changes in activity obtained by different DE processes is not straightforward as larger increases are expected from slower enzymes [109] and further improvements are normally poorer due to diminished returns [110]. Other factors that make comparisons between DE approaches challenging include significant variations in measurement conditions, and the total resources employed, which can be difficult to estimate. In this case, a rapid proof-of-concept approach involved a two-step DE process and yielded a 5.8-fold increase in activity between HDNO D2 and HDNO D6 towards PPY conversion. This compares favourably with other DE experiments (from all but the most extensive undertakings), particularly considering that the reported yield does not measure improvement from the wild-type enzyme baseline, where activity towards PPY was extremely low. It also provides some initial evidence that the rational approach described here offers benefits in terms of speed of optimisation, while also enabling mutations to be inserted outside of the active site. The methodology described here is also differentiated from other approaches in endeavouring to perform truly rational protein reengineering, while being compatible with standard (active) site directed mutagenesis and temperature stabilisation approaches, which should enable more rapid optimisation for both research and industrial purposes.

Escaping local maxima of activity and moving to other areas of mutant space where activity is even greater remains an important challenge for DE. Therefore, a strategy favouring diversity with a tight control over the generation of experimental variants might result in the most effective strategy in DE when combined with a rational approach. It is anticipated that further iterations targeting the same substrate and enzyme can result in additional gains.

In the current work, only low multiplicity degenerate codons were employed during both SDM and PCR-based de novo gene synthesis. Alternatively, a fully designed synthetic gene library might be a cost-effective alternative to produce any in silico predicted mutant from multi-site combinatorial methodology or even the generation of de novo enzymes [85, 112, 114]. Additionally, increased computational resources would allow more accurate enzyme models, and larger data sets are undoubtedly beneficial to obtain better predictions. Fortunately, computational hardware continues to get faster, and although the vast space of enzyme variants will remain practically infinite, the number of solutions that result in dramatically improved enzyme activity is also expected to be vast and diverse.

Thus, a methodology for the design of intelligent libraries for directed evolution by a process of dynamic testing of catalytic effects guided by a series of in silico mutations has been presented. By employing this methodology, we have successfully accelerated the process of protein reengineering of a stable and more active HDNO D8 mutant for the oxidation of (R)-phenyl pyrrolidine and other relevant amines through a series of directed evolution rounds. Moreover, we have demonstrated that rationally targeting distal sites with non-charged residues is possible, and by delivering a fully stable engineered HDNO D8 product, we propose this technology can be used as a stand alone or in combination with other semi-rational and empirical approaches to accelerate directed evolution. Larger improvements per DE round may be possible by increasing the number of mutants (see Examples 4-7, even if using shorter simulations) and/or the length of each mutant simulation. Both should increase the accuracy of the resultant library predictions and are only limited by the computational power available. Furthermore, emerging new computational hardware for performing MD and quantum mechanical calculations, together with the benefits of technologies such as machine learning (see Examples 4-7) as well as fully synthetic genes can further leverage the effectiveness of the methodology.

### Example 3: Rationally Accelerated Directed Evolution with Machine Learning, and Application to an Oxidoreductase (EC-1)

### Introduction

Recently, machine learning (ML) and other computational methods have had increased success in rationalising the relationship between protein sequence, 3D structure, and function. ML methods have also found their way into novel methodologies for the acceleration of DE processes [122, 123, 124, 125]. Existing applications of ML in DE have been based on protein sequence activity relationship models (ProSAR) where a score (or set of scores) based on properties of interest (such as activity and stability) are obtained for each mutant sequence as labels or dependent variables and the sequence is encoded into a numerical matrix for ML modelling [126, 149]. Different approaches to encode the protein sequence have been previously proposed, including one hot encoding (where a group of bits are used to encode each amino acid and the allowed combinations of values are those with a single high bit and all the others low), encoding the residues into a sequence of real numbers (by representing one or more amino acid properties from a database, such as the AAindex [127]), and variations including embedded encoding [113] or the addition of a fast Fourier transform (FFT) step to the sequence-encoded data [128]. After data has been encoded, diverse ML models can be employed to fit and predict the properties of interest, such as linear regression, Bayesian models, random forests, support vector machines or artificial neural networks. ProSAR models employed in DE normally present different degrees of fitness, often due to intractable reasons such as mutant landscape smoothness [113]. Fitted or trained ML models can be used to deconvolute the effects of individual mutations (or even the effects complicated mutant-mutant interactions if sufficient and adequate data is available) to make predictions of relevant enzymatic properties, help to identify improved protein mutants and design experimental libraries during DE iterations. These experimentally driven methodologies are useful when standard (i.e., random) DE techniques fail to make significant gains after many rounds of evolution [85, 129].

Experimentally based methodologies present major challenges in the exploration of mutant diversity even with the largest libraries, as only a few sites can be tested simultaneously [13]. Given the vast amount of possible mutants, DE by standard techniques (e.g., epPCR or random selection of sites for site directed mutagenesis) is slow and inefficient and in need of further guidance to improve performance, such as by using computational predictions. ML models have been used to provide predictions based on previous experimental results to further improve standard DE processes. However, ML methods require large amounts of data and can only confidently predict the effect of mutants in the space that has been sampled. Considering that only a fraction of mutants can be experimentally measured in practice (especially mutant-mutant interactions or mutations containing more than one mutant), such an approach is severely limited by the resources (time and money) available. Moreover, ML models have not been able to show their full potential and have arguably had only a relatively limited impact on DE processes because they are generally only introduced at later stages (when sufficient experimental data is available). The general sparsity of experimental data with respect to protein mutation sites also means that only a small fraction of the protein landscape can be targeted by these ML models (particularly outside of the active site), restricting the increases in catalytic turnover improvement that can be achieved [122, 111].

The inventors have recognised that an efficient alternative to experimentally led ML data generation could depend instead on computational methodologies to estimate enzymatic properties such as catalytic activity based on protein sequences and structural data. Such a computational strategy should be fast enough to circumvent conformational sampling problems found for computationally based rate estimations and allow the generation of a large and diverse dataset of mutants to be of practical use to fit ML models to guide experimental DE strategies, and accelerate the finding of new and otherwise undetected enzyme variants with significantly improved catalytic turnover [130, 131, 132, 133].

Genetic diversification and screening using standard experimentally based DE methodologies, while suboptimal, has been found to be extremely useful in the development of new and improved protein mutants and the resulting enzymes have found applications in active pharmaceutical ingredient (API) synthesis, including complex chiral compound synthesis [13, 118]. One class of enzymes that have seen an early benefit from DE are monoamine oxidases (MAO), such as MAO-N and 6-hydroxy-D-nicotine oxidase (6-HDNO). MAO-N catalyses a range of (S)-selective primary, secondary and tertiary amines relevant to API synthesis and has been significantly improved through many rounds of DE [106, 6, 95, 4], while the related enzyme, 6-HDNO has a similar chemical reactivity but has been shown to target the opposite enantiomers instead, including synthetically relevant substrates such as the primary amine (R)-methylbenzylamine (AMBA) or the secondary amine (R)-2-phenylpyrrolidine (PPY). Significant improvements have been obtained for 6-HDNO as described in Examples 1 and 2, resulting in a more active and stable D8 variant after a series of conventional [6] rounds of DE followed by a computationally guided proof-of-concept round of evolution (see Examples 1 and 2).

While ML based on experimental data alone is impractical (as described above, due to the immense resources required to significantly explore the mutational landscape of proteins) the computational methods for the estimation of enzymatic activity reported in Examples 1 and 2 make it feasible to generate the datasets and diversity necessary to effectively train any ML model on enzyme turnover and provide predictions of turnover for protein mutant at any site in the enzyme (and particularly outside the active site). ML is an ideal approach to exploit the benefits provided by the previously described computational predictions based on MD. Hence, elaborating on the previous inventions reported in Examples 1 and 2, this example introduces ML to the process for the DE of 6-HDNO from Arthrobacter nicotinovorans. In this example ML is used to rationally drive DE experiments based on large and diverse datasets of over 360000 mutants and MD simulations generated from a series of distinct starting conformations (with a diversity larger than could reasonably be produced experimentally using the same resources). These datasets were used to fit ML models and used to produce global predictions (i.e., predictions for every site in the protein), with the aim of designing efficient DE libraries capable of discovering better and otherwise inaccessible enzyme variants. The efficacy of the process was experimentally validated by generating a diverse set of highly active variants (some including multiple mutations) based on two independent rationally guided DE libraries.

### Results

### Molecular Dynamics and in Silico Mutant Scoring

A comprehensive mutational and conformational dataset of over 360000 enzyme variants was generated with the aim of developing a series of computational prediction processes using ML models that can be used to design DE libraries. Each protein sequence contained three additional mutant residues beyond the original 6-HDNO D3 (E350L/E352D/N414H) sequence. The starting conformation for each new mutant was randomly selected from five frames from a 1 μs MD simulation of 6-HDNO D3. For each new generated mutant, a further 1 ns of MD was performed. Other time frames as possible, for example depending on the computing resources available. For example, Example 7 below uses fewer mutants (1000) and longer time frames (50 ns). Thus, timeframes as long as practical given the resources available may be used. All new generated mutants were scored according to the methodology described in Example 1, which entails the estimation of catalytic activity based on the electrostatic component of the free energy barrier (namely ΔΔG^{‡}_{Q20}) for each set of coordinates (also referred to herein as frames) saved during the 1 ns MD simulations. For this work ΔΔG^{‡}_{Q20} values were calculated based on the hydride transfer activation step for the substrate PPY and the scores (μ_{Q20}) were based on the mean protein effect calculated from 10 frames (1 every 0.1 ns). The bulk of data resulting from all the generated mutants for each conformation is presented in a series of histograms (FIG. 27). FIG. 27 displays the different distributions of enzyme activities produced for each conformation. The μ_{Q20} scores from each conformation give very distinct distributions (i.e., distinct values for the mean and variance of the populations of μ_{Q20} scores), even though an equally balanced, diverse, and large random mutation set was used to produce the data within each conformational subset.

The current methodology is efficient enough to enable the fast exploration of both the mutational and conformational landscape, by rapidly generating a large dataset of MD simulations. The methodology is also fast enough to address and solve problems associated with poor conformational sampling, which is one of the main problems found in previous computational enzyme rate predictions that use protein structural data [130, 131, 132, 133]. The current scoring methodology benefits from an increased number of mutants in the dataset and there is no theoretical upper limit to the amount of data that can be included (only a practical limit based on, e.g., computational resources).

The current Example used data based on over 360000 1 ns simulations, equivalent to 300 μs of contiguous MD simulation, and totaling circa 2 million ΔΔG^{≠} estimations for individual structures across several conformations. This is up to 20 times the amount of MD simulation data compared to the work of Example 2, and accounts for over 700 times the number of scored mutants found in that Example. Moreover, triple mutants were created to increase the sampling density and thus produce circa 1 million single mutations. Introducing more than one individual mutation at a time makes it additionally possible to sense epistatic (i.e., mutant-mutant interactional) effects. Although the noise in the calculated ΔΔG^{≠} datasets based on huge numbers of 1 ns MD simulations can be quite large and difficult to interpret manually, this noise is significantly reduced by the deconvolution performed by training ML models.

### Machine Learning Models

It is often necessary to test a variety of different ML methods to find an optimal or acceptable method for the specific data being modelled, which may depend also on the nature of the enzyme and the amount of data. Therefore, a diverse set of ML models were compared to model the encoded data, including multi-linear regression (LR), lasso regularised linear regression (Lasso), support vector regression (SVR) and artificial neural networks (see Table 16). However, it is possible to use many more different ML methods including Logistic regression, random forest regression, k-nearest neighbour regression, and many more in addition to a virtually unlimited diversity in neural network architectures, including the use of deep learning neural networks vibrational auto-encoders. Different ML methods are readily available and can be easily tested in a modular manner. Moreover, a series of different encoding methodologies were also tested (see methodology), namely: random FFT and random NonFFT, AAindex FFT and one hot encoded. The inclusion of a fast Fourier transform (FFT) has been suggested to improve performance of ProSAR models [164], and shows improved resilience to overfitting (as demonstrated herein). The performance of each ML model was measured by correlation coefficients towards unseen test and validation data.

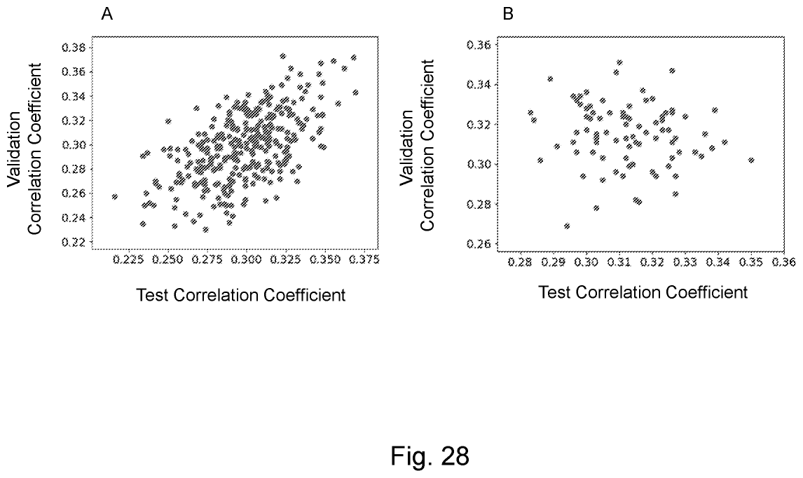

Results on LR models confirmed that the diversity introduced by randomly varying the encoding vectors can have a significant impact on improving the predictive capacity of the ensemble of models. It can also have an impact on the performance of individual models (some iterations will be encoded to present better model performance). This is readily observed in a meta-correlation between the individual test and validation correlation coefficients obtained from a set of distinct LR models (each trained on distinctly random encoded FFT data, FIG. 28A). This shows that the apparent variability in individual measured model performances (based on correlation coefficient values) is not only caused by random sampling fluctuations in the test set (by random train and test data splitting), but by true changes in model performance due to the random variations in the encoding. In fact, freezing the random-encoding process to a single encoded dictionary (while still maintaining the random data splitting of train, test and validation sets) resulted in no significant meta-correlation between the test and validation correlation coefficients (FIG. 28B), while the test and validation scores still had random and independent fluctuations (due to random data splitting into test and validation sets).

ProSAR models involving standard linear regression and other ML methodologies (that involve encoding the protein sequence based on amino acid properties from the AAindex) have been found to yield promising results [134, 135]. The current results also suggest that ProSAR models can be encoded by random encoding sets successfully. For an AAndex encoded ProSAR model, feature reduction processes are normally employed (leaving only the most productive features to improve the predictive performance of the models over unseen data [136]). Similarly, in the herein defined random encoding, some encoding dictionaries can potentially be further selected based on better performance. In other words, any number of encoding sets can be generated using the random encoding strategy and encoding vectors that perform best can be selected manually or using a ML approach. Furthermore, individual models encoded by a fully random strategy can be encoded to an unlimited complexity (by contrast with strategies based on the AAindex which are limited by the number of properties available in the AAindex database) while compensating for overfitting with a regularisation parameter adjustment, which can out-perform models based on the AAindex properties alone (see FIG. 48 corresponding to Example 5, where an random encoding method based on an encoding complexity of N=750 is compared to an AAindex index based encoding strategy which involved encoding 553 distinct properties).

### Ensemble Modelling

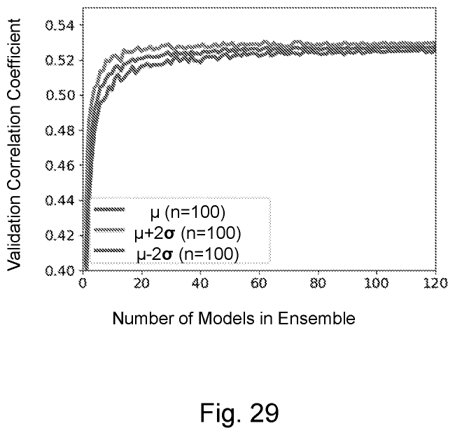

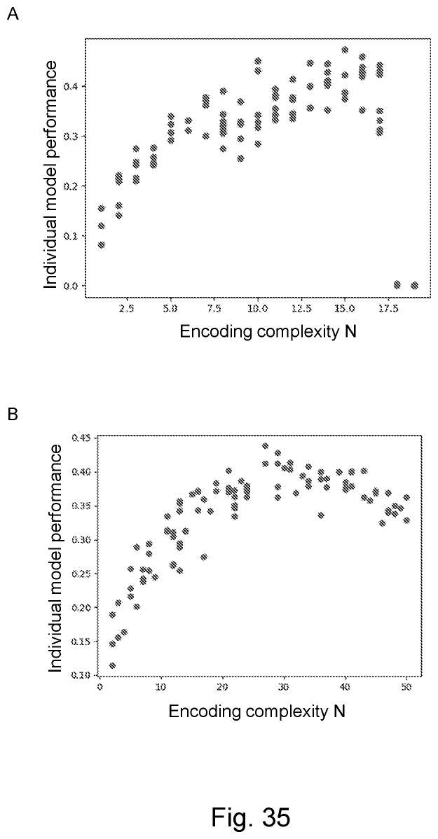

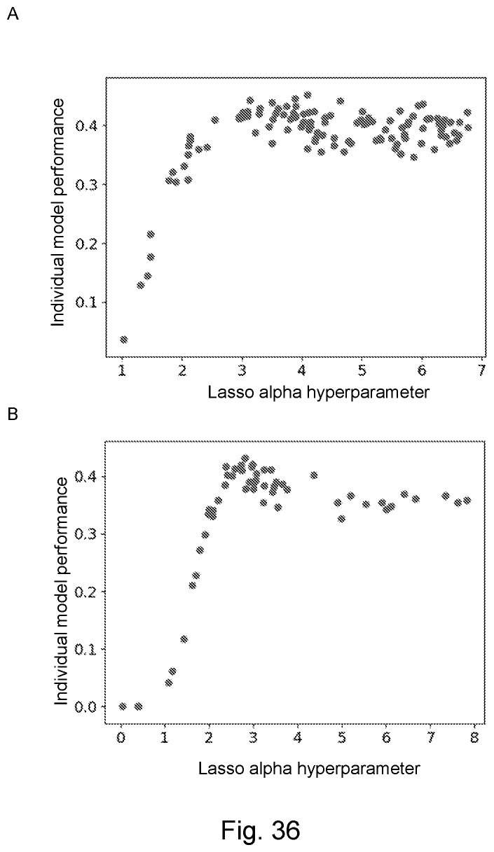

A subsequent analysis was made to test the performance of ensemble model approaches over individual models. Grid search analyses were also performed to assess the effects of encoding complexity on individual model performance and to optimise hyperparameters (see FIG. 35 and FIG. 36). A summary of the mean performance of individual models and ensemble processes for different ML variations is presented in Table 16, which includes a comparison between different ML methods, including one hot encoded, AAindex encoded and randomly encoded ProSAR models towards predicting enzyme activity based on conformation 570 ns (selected arbitrarily). For each ensemble, the predictions were calculated as mean (μ_{Q20}) predictions per mutant for all models in the ensemble. On an individual model basis, Lasso models had the best performance; AAindex models had a similar (best mean) predictive performance of 0.443 when encoded with 28 properties. Upon ensemble modelling, all ML variations show improved performance, and LR and Lasso models significantly gained in performance when aggregating 25 random LR FFT models and 25 random LR Non-FFT models (with ensemble performances of 0.519 and 0.511 and representative gains of up to 0.11 and 0.132, respectively, over individual model performances). The best overall performance in the ensemble was found in the neural network models (fully random FFT encoded), which achieved a 0.528 correlation coefficient to the validation set (which represents a 0.102 improvement from individual models on average). By sampling ensemble models from a pool of 750 LR FFT random models, it was observed that the average ensemble performance consistently improves with the addition of more models to the ensemble but is affected by diminishing returns at increasing computational demand and thus it was decided to test for either 10 or 25 models per ensemble only (see FIG. 29).

Further improvements on these individual and ensemble models may be possible by biasing the random encoding process (as discussed above), and/or by the introduction of alternative artificial neural network architectures (such as e.g. recursive neural networks, long short term memory networks, variational auto encoder neural networks, etc.), and/or by increasing the encoding complexity (see Example 5). The neural network methodology used in this Example is also not intended to be the most optimal example, but an exemplar that works proficiently. Other ML methodologies can be used instead and specific parameters such as the number of nodes, dimensions, regularisation, normalisation, dropout parameters as well as the general architecture can be modified to suit specific problems (which by trial and error may yield better performance and may also depend on the specific data that they are being trained for).

TABLE 16
Performance of different individual and ensemble ML models (LR, SVR, Lasso
and artificial neural network models), with different random and one-hot
encoding strategies based on a dataset generated from the seed conformation
at 570.0 ns. All Lasso models were trained with a regularisation a parameters
specifically optimised for each variation on the current data.

| | | Mean | | |
|---|---|---|---|---|
| | | Performance | Performance | Performance |
| | Encoding | of individual | of Ensemble | of Ensemble |
| Model | Complexity | models | of 10 Models | of 25 Models |
| LR random FFT | 28 | 0.407 | 0.511 | 0.519 |
| LR, random NonFFT | 12 | 0.379 | 0.499 | 0.511 |
| SVR FFT random | 12 | 0.359 | 0.388 | n.a. |
| c = 1.0 | | | | |
| Lasso random FFT | 28 | 0.413 | 0.509 | 0.521 |
| α = 10^{-4.0} | | | | |
| Random, artificial | 28 | 0.426 | 0.518 | 0.528 |
| neural network FFT | | | | |
| One hot encoded Lasso | 20 | 0.395 | 0.406 | 0.407 |
| α = 10^{-2.75} | (binary) | | | |
| LR randomly selected | 28 | 0.418 | 0.512 | 0.524 |
| AAindex FFT | | | | |
| Lasso randomly selected | 28 | 0.443 | 0.518 | 0.525 |
| AAindex FFT α = 10^{-3.0} | | | | |

n.a. = not available.

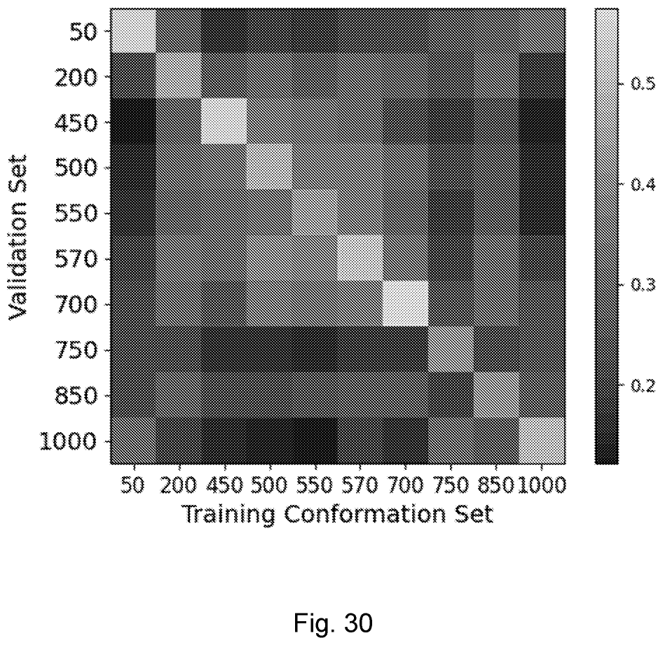

A series of models were trained to investigate whether ML models trained on data from one seed conformation could confidently predict data generated from another seed conformation (and to establish whether a multi-conformational approach is beneficial in improving predictions compared to only using a single seed conformation). A set of 25 FFT Lasso models (based on encoding the protein sequence with encoding complexity N=28 random encoding vectors per amino acid) were trained on subsets of training data for each seed conformation (for ensemble ML modelling). These were used to generate predictions and quantify performances (measured by correlation coefficients) on a series of models trained and tested on scored data, generated from a diversity of seed conformations (see FIG. 30). The diagonal of the matrix represents the self-correlation (the self-correlation is a measure of the correlation between models trained on and tested against data from the same seed conformation). The non-diagonal values (cross correlations) represent the relative measures of cross compatibility of the mutant data between different seed conformation sets (by measuring the performance of models trained on one seed conformation but tested for on data from a different seed conformation). The results obtained show no universality in data from a single conformation. Self-validation correlation coefficients are overall better (up to of 0.575 for conformation 700 ns) than cross correlations. There is a visible diagonal across the matrix plot that supports the use of multiple conformational data for more representative modelling. While increasing the number of sampled seed conformations may prove increasingly beneficial, the choice of how many seed conformations to sample depends on the availability of computational resources. Even a single conformation will provide predictive capacity in practical use, but it is expected that as hardware improves (or more resources are available) more conformations could be sampled. There is no upper limit other than for practical considerations, and any number of conformations may be used.

### Model Aggregation and Global Predictions

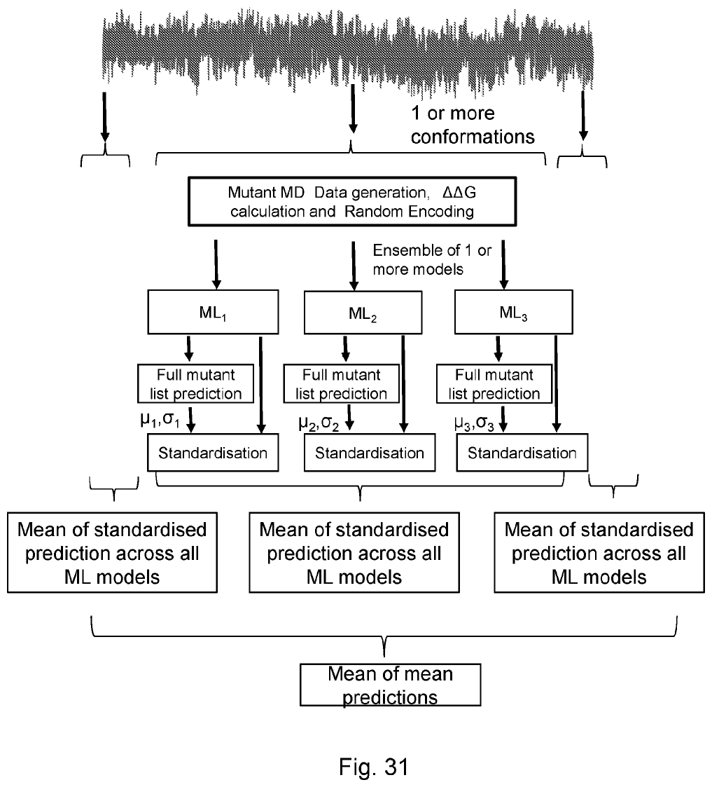

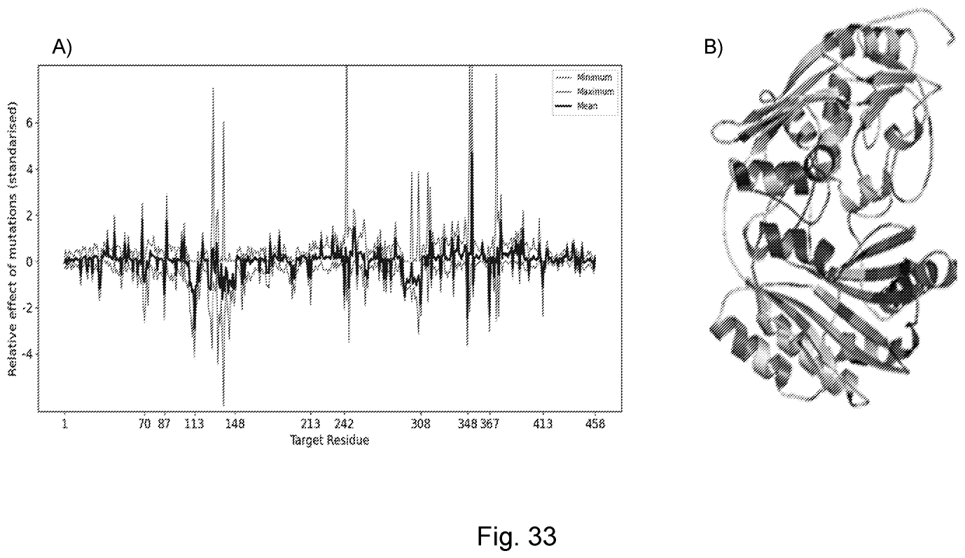

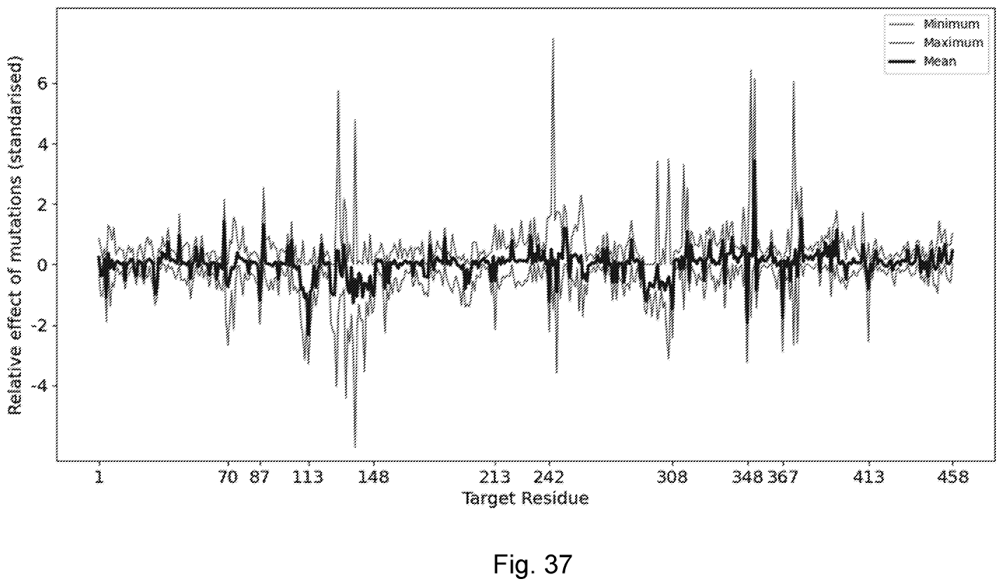

The results from the self and cross-validation of the ML ensemble models suggest that a method based on a diversity of data generated from different seed conformations may produce more representative and accurate predictions. Therefore, a series of ML models were trained on data from distinct seed conformations (see FIG. 31 to visualise the complete process). Due to the linear shifts and changes in variability between data form different conformations, a standardisation step was also included for each ML model such that an overall mean of 0.00 and a standard deviation of 1.00 was obtained for a given diverse and unbiased set of mutants (spanning out to the full space of mutant possibilities; in this case that was a set of single mutants, including each possible amino acid substitution for each site on the enzyme). Therefore, following the two-level model aggregation process (multi conformation, and multiple models per conformation), a full in silico site directed mutagenesis potential map was obtained based on predictions for all possible single mutants in the EC1 6-HDNO enzyme (see Methods). FIG. 33 shows the full prediction result based on 250 neural network models (25 per conformation). Equivalent results were obtained for other ML variations (see FIG. 37 for the regularised Lasso FFT model in silico site directed mutagenesis potential map). The relative μ_{Q20} fluctuations due to mutations were predicted to be several standard deviations better or worse than the mean. Hence, the in-silico site directed mutagenesis potential map can be used as guidance in ranking potential effects of mutations towards enzymatic activity. From this analysis, sites 113 and 348 have a high-ranking position, with the potential in a DE library to improve catalytic activity. In contrast, residue 352 corresponds to the overall worst target site predicted for the D3 HDNO variant. This is consistent with the results on Example 1 and with the fact that site 352 corresponds to a previously inserted beneficial mutation found early on in active site directed mutagenesis [6]. The computational predictions reveal a diverse set of possibilities, where the best-ranking target sites are found to be distributed all over the entire enzyme. Note that in this example, the effect of mutations that alter the charge of the residue (replacing charged/non-charged residues with non-charged/charged residues) is also assessed, as mutants are not limited to conservative mutations. As mentioned above, the methods described herein are not limited in practice to any types of mutations. In particular, the methods can assess the impact of a mutation on the dynamics (through its impact on electrostatics) as well as any direct effect of a change in charge on the reaction.

### Experimental Validation

A final step was to validate the computational process by designing one or more DE libraries containing a good diversity of high-ranking mutants and testing them experimentally. This may be performed using many approaches depending on the required level of control over genetic diversification and the available technology [13]. Fully synthetic genes or full de novo protein designs [141] are be an acceptable (and readily available) solution and a good balance of diversity and control over genetic diversification can be achieved by PCR based gene synthesis or equivalent mutant library construction processes [142]. In PCR based gene synthesis a series of degenerate codons are selected to introduce amino acid degeneracy at a series of specified sites. Commonly NNK or NDT degenerate codons with high multiplicities are employed experimentally to introduce the largest possible number of mutant variants at a target site. However, based on a computational strategy, smaller codon multiplicities can be more efficient by allowing more sites to be targeted per screening iteration within a library of limited size. This also helps to avoid common problems encountered within large multiplicity codons (such as amplification bias [83] and the undesirable presence of stop codons). Moreover, a good compromise can be obtained by only considering a subset of degenerate codons (e.g., with multiplicity of 12 or lower) that contain no stop codons and code only once for each amino acid (i.e., a one-to-one relationship between constituent codons and amino acids). These restrictions result in a set of 267 distinct possible degenerate codons. A further restriction can be incorporated to force the selection of only codons that additionally contain the wild-type amino acid at each site (such that a library with, e.g., seven degenerate mutation sites can produce libraries containing enzymes with less than seven mutations). For 6-HDNO, accounting for all the allowed codon-combinations and removing site H72 (covalently bound to the cofactor), the total number of ways that a distinct triple codon library experiment could be conducted (with the imposed constraints) is over 10¹³. This number is very restrictive, even for ML based scoring, considering that each of these libraries comprises a group of up to 1728 different mutants (and the process would escalate steeply if the number of desired sites is increased).

An in silico generated full site saturation map such as that of FIG. 33A can already work as a linear deconvolution of individual mutations to quickly assess any DE library and can also work as a first pre-selection process. Based on this rationale, two rationally designed experiments using a OE-PCR (overlap extension-PCR) based de novo gene synthesis methodology were conducted as a validation and qualitative assessment of the performance of the computational DE process The experiments were separated into two independent libraries to facilitate the experimental work. In all cases, a series of libraries were prepared based on the HDNO-D3 (E350L/E352D/N414H) sequence. HDNO-D3 is a sufficiently active and stable mutant uncovered in Example 2 making it a practical candidate for further DE validations. For the OE-PCR based gene synthesis, a series of oligonucleotide sequences representing the D3 variant were synthesised. Furthermore, for each experiment a series of specific target sites were chosen to incorporate degenerate codons (as per the intended library designs). All the selected degenerate codons were selected to encode for the WT amino acid at each position, and to contain no stop codons and to only encode once for every amino acid (as rationalised above). Thus, the libraries comprised a selection of small codons and specific high scoring sites for experimental validation based on the computational process. Specifically, the following sites and codons were used: 242 (degenerate codon KWC, encoding for any of Y, F, D, V; the former one letter codes are based on the IUPAC nucleotide code and the latter based on the IUPAC amino acid codes), 348 (degenerate codon VAS encoding for any of E, D, H, K, N, Q) and 353 (degenerate codon RDC encoding for any of D, G, I, N, S, V) were selected for the first validation experiment with a maximum diversity of 144. Sites 109 (degenerate codon RWG encoding for any of E, K, M, V), 112 (degenerate codon RBC encoding for any of A, G, I, S, T, V) and 113 (degenerate codon RDC encoding for any of D, G, I, N, S, V) were selected for the second experiment and had a maximum diversity of 144. The sites selected and built into these libraries have relative predicted ranks (based on the neural network ML process) of 7, 2, 28, 25, 12 and 1 selected from the 458 possible sites, respectively. Incubation and screening parameters were identical for all DE experiments.

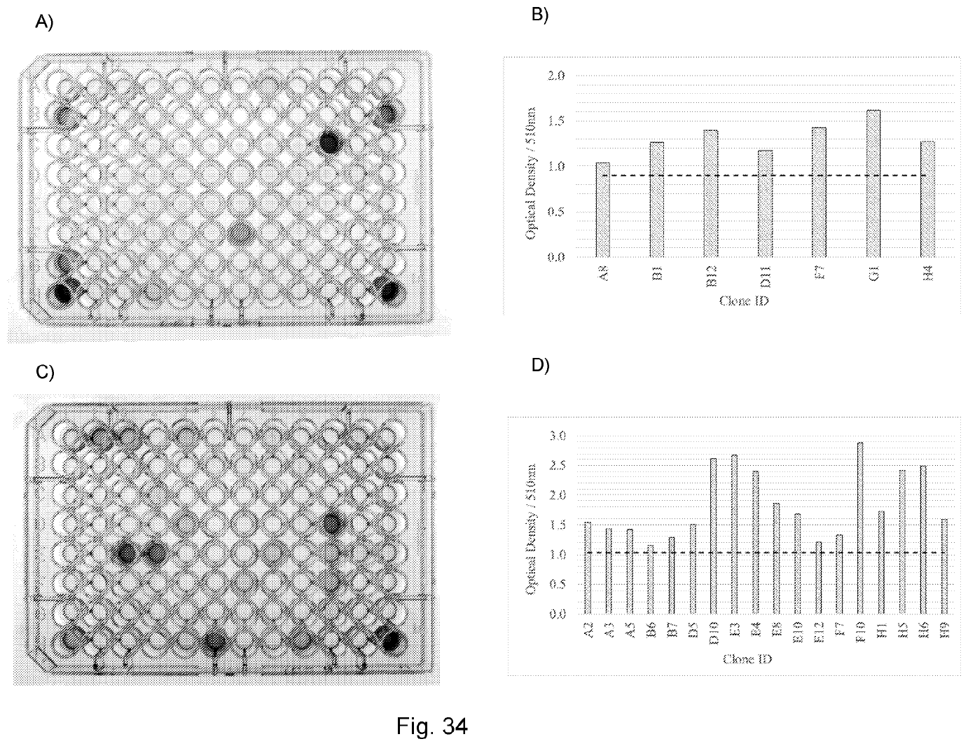

Enzyme variants were expressed on microtitre plates and were examined by an enzyme assay screen, to directly compare their efficacy in producing active variants. Upon inspection several clones displaying enzymatic activity were observed on both computationally guided experiments (see FIG. 34). These clones were sent for DNA sequencing and shown to contain multiple mutations. This confirms that mutations with catalytic activity were found independently within each computationally-guided library, thus providing evidence that the herein described computational method is effective for the acceleration of DE.

### Materials and Methods

### Mutant Generation and Molecular Dynamics

Mutant variants were randomly generated to contain three additional mutations beyond the D3 parent sequence. This was set arbitrarily for a good balance between introduction of noise and allowing a fast exploration of mutant space (a different number of mutations could have been used instead, see Example 6). All amino acids were allowed except for Cys to avoid disulphide bond deletions and formations, but this will have virtually no practical effect because the removal of one amino acid still leaves a huge potential for the identification of mutants. Further, it is also possible to introduce and target cysteine residues if they are properly modelled, see Example 6 where Cys residues were also targeted and Cys was also introduced. A random parent conformation from the D3 simulation was assigned to each mutant variant. Sites 1-10 and 449 to 459 were not targeted due to their location being close to the N and C terminus (again this will have virtually no practical effect, but they can be easily targeted if required, see Example 6). Likewise, mutant residues that had already been introduced into the D3 variant (350, 352 and 414) were avoided (as they are the result of previous protein engineering efforts) and site 72 (which is a covalent link to the FAD cofactor) was also not targeted. Over 360000 mutant variants were generated and assigned to 10 different conformations. As discussed above, even a single conformation delivers practically useful results. Conversely, with more computational resources this number of 10 could easily be further extended to more conformations with no upper limit. Molecular dynamics (MD) simulations were conducted using the OpenMM software [53] with the AMBER force field and protein parameters for the enzyme [51], the General AMBER Force Field (GAFF) [48] for the substrate, flavin adenine dinucleotide (FAD) moiety and counter-ions, and the TIP3P water parameters for the solvent [52]. Although OpenMM was used in this exemplification any MD software (such as CHARMm, AMBER, Tinker, Gromacs etc.) or energy-based ensemble generating algorithm (such as Monte Carlo and enhanced sampling techniques) could be used if suitable parameters and protein and water models can be constructed. The 10 μs MD simulation for the 6-HDNO D3 variant (that was produced in Example 1 and used in Example 2) was employed in the present work as the base for the generation of starting structures for all mutants generated. Structures from simulation time 9050 ns, 9200 ns, 9450 ns, 9500 ns, 9550 ns, 9570 ns, 9700 ns, 9750 ns, 9850 ns and 10000 ns were used as starting structures for mutation insertion. These time points used are arbitrary; although it is advantageous to use a diversity of conformations. It is also advantageous that the MD simulation has time to approach thermodynamic equilibrium. Therefore, conformations were selected towards the end of the 10 μs MD simulation. The length of MD simulation may be particulary important in cases where the starting structure is not directly available as a crystal or NMR structure, such as in this case, where a homology model based on three amino acid changes from the crystal structure was used. Mutations were inserted by modifying the side-chain chemical structure computationally and repacking the protein to contain the new residues (in this example PyRosetta [145] was used for this purpose but any algorithm that can repack a protein after mutation to achieve something approximating the free energy minimum could be used) after which a comprehensive energy minimization was performed with explicit water present (in this case the AmberTools sander module was used [146] but any algorithm that could perform said minimisation could be used), followed by a 1 ns MD simulation. The amount of 1 ns MD is only limited by the computational resources and longer than 1 ns might be performed if practical, see Example 7 for an approach using 50 ns MD simulations. The MD coordinates of each mutant were saved every 0.1 ns for subsequent scoring. This frequency of saving is largely arbitrary, provided that it is short enough to allow multiple conformations to be saved per simulation. The saving frequency may be limited at the upper end by the length of the integration step in the MD simulation used (in this case, 4 fs), as well as any data storage limitations. The saving frequency may be limited at the lower end by the length of the MD simulation as multiple conformations should be saved per simulation. Higher frequencies may be better than shorter ones, although there may be diminishing returns in this regard. For example, high frequencies (>100 ns⁻¹) put large demands on data storage without significant improvement. Under the current methodology a new frame was generated every 4 fs in the volatile computer memory but only saved to non-volatile storage every 0.1 ns for further processing (to use the available resources effectively).

Mutant ΔΔG^{‡}_{Q20} Scoring

Estimation of the changes in hydride transfer barriers ΔΔG^{‡}_{Q20} (based on MD data for each mutant) followed the method described in Example 1 and used in Example 2. The barriers (that affect catalytic rate) were estimated for each 0.1 ns of MD simulation (for a total of 10 frames) to obtain a final mean score μ_{Q20} for each mutant. The solvent and counter-ions contributions were not included in this computation to reduce noise levels but could easily be introduced, see Example 6. Reduced noise levels can be obtained by increasing the length of each MD simulation and the amount of mutant data generated. Partial atomic charges in the external system were derived from C36 protein parameters [71, 72]. Alternative parameters, such as from the ff14SB set, or de novo generated by DFT methods could be substituted for these. For the internal system (referred to above as “core” or “reactive centre”), the partial atomic charges were derived from single point DFT calculations on the reactant complex and transition state structures by CM5 population analysis [78] (alternatives such as the Hirshfeld population analysis or the Mulliken population analysis could be substituted as shown in Examples 4 to 7). The reactant complex and transition state structures were obtained from QM/MM optimisations using ChemShell software [66, 67], QM calculations of the QM/MM embedded models were performed using Turbomole software [68] and MM calculations were handled by DL_Poly software [69] (a DFT-cluster calculation could also have been employed here, instead of the QM/MM model, as demonstrated in Examples 4 to 7).

### Sequence Encoding for Machine Learning

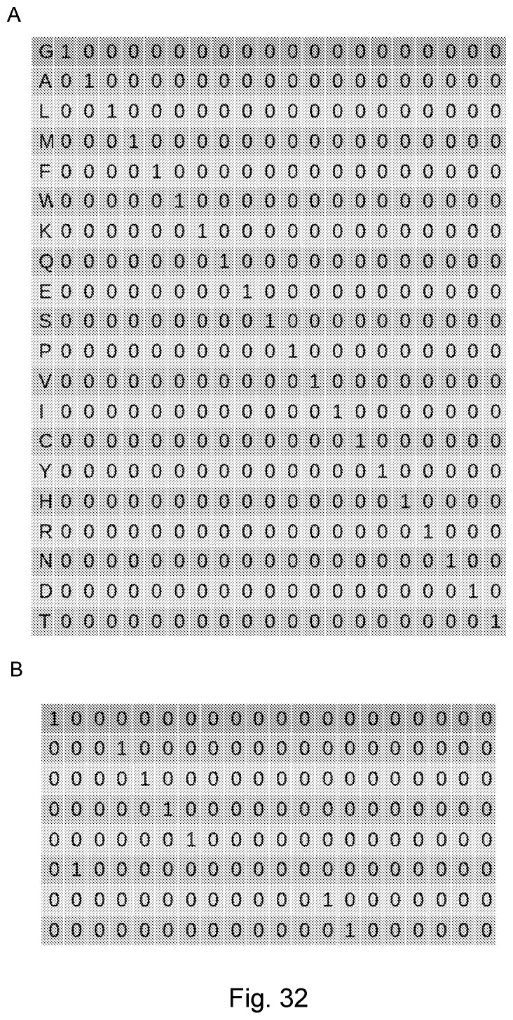

Four encoding methods were used, namely random FFT, random NonFFT, randomly selected AAindex FFT and one hot encoded. The one hot encoded methodology encoded each distinct amino acid as a distinct binary vector containing only zeros except for a unique position associated with the amino acid being encoded. The lookup table that is shown in FIG. 32A was used to encode the protein sequences in the current Example, comprising a set of 20 distinct encoding vectors representing the 20 natural amino acids. Note that a different number of encoding vectors may be used to include additional cases, such as protonation states or non-natural amino acids. Based on this lookup table, the sequence of amino acids “GMFWKAIC” (SEQ ID NO:5) would encode into the matrix shown in FIG. 32B, for example.

The random encoding methodology used a different type of lookup table, of variable size N×M, where M is the number of types of amino acids (e.g. 20 for only all the natural amino acids), and N is the encoding complexity, which can be 1 (or any larger integer number). Hence M encoding vectors of size N are generated. Each resulting matrix was then filled up with numerical values such that for each amino acid and for each random encoding vector, a series of real numbers (each between 0 and 1) were generated randomly to construct a look up table. Table 17 shows an example look-up table for N=2 (containing 2 random vectors, each with a set of randomly generated numbers). Thus, based on Table 17, the sequence of amino acids “GMFWKAIC” (SEQ ID NO:5) would be encoded as the sequence shown in Table 18. For every ML model, a new random encoding table was generated, which was used to encode every mutant sequence for that model (either for training or prediction of new mutants). N values between 1 (see Example 7) and 750 have been used, where larger values were also explored as part of grid searches (up to N=2000) (see Example 5).

TABLE 17
Exemplification of a random encoding dictionary or lookup
table based on an encoding complexity of N = 2.

| Amino | Random | Random |
|---|---|---|
| acid | encoding vector 1 | encoding vector 2 |
| G: | 0.06935 | 0.12290 |
| A: | 0.76822 | 0.42561 |
| L: | 0.98967 | 0.02218 |
| M: | 0.25765 | 0.93948 |
| F: | 0.85802 | 0.84639 |
| W: | 0.87556 | 0.96635 |
| K: | 0.17351 | 0.03165 |
| Q: | 0.76259 | 0.58961 |
| E: | 0.17569 | 0.04167 |
| S: | 0.95240 | 0.32876 |
| P: | 0.03483 | 0.27070 |
| V: | 0.14624 | 0.06489 |
| I: | 0.25395 | 0.51371 |
| C: | 0.55990 | 0.32258 |
| Y: | 0.41115 | 0.34190 |
| H: | 0.67236 | 0.77826 |
| R: | 0.27053 | 0.87571 |
| N: | 0.52673 | 0.20701 |
| D: | 0.99215 | 0.32514 |
| T: | 0.64703 | 0.99435 |

TABLE 18
Encoded sequence for amino acids “GMFWKAIC” (SEQ ID NO: 5) based
on the random encoding dictionary exemplified in Table 17.

| | | | | | | | | |
|---|---|---|---|---|---|---|---|---|
| Encoded | 0.06935 | 0.25765 | 0.85802 | 0.87556 | 0.17351 | 0.76822 | 0.25395 | 0.5599 |
| vector 1 | | | | | | | | |
| Encoded | 0.1229 | 0.93948 | 0.84639 | 0.96635 | 0.03165 | 0.42561 | 0.51371 | 0.32258 |
| vector 2 | | | | | | | | |

When an FFT was additionally performed, the FFT transform was performed on each encoded vector independently. The first datapoint of each transform was ignored and only a subset of datapoints of each transform was included up to a specific number of datapoints, based on the following rules: IF an even number of residues are encoded THEN the number of datapoints included is the total number of residues divided by 2 (and ignoring the first datapoint of the FFT), OR IF an odd number of residues are encoded THEN the number of included datapoints is the total encoded minus 1 and then divided by 2 (and ignoring the first datapoint of the FFT). For the “GMFWKAIC” (SEQ ID NO:5) amino acid sequence processing of the resultant FFT datapoints in this method would result in two vectors of size three (based on an encoding complexity of N=2): (0.83142752, 0.96078934, 0.88367072) and (1.15125525, 1.20795329, 0.51925024). The FFT calculations were performed using the scipy FFT implementation in python3 [147](but any equivalent procedure can be substituted).

The AAindex encoding methodology first defined an encoding complexity of N for each model (any integer from N=1 up to the maximum number of properties available). Next, instead of generating a fully random set of real numbers (as used in the random encoding method), a series of properties were randomly selected from the AAIndex database (although other databases can be substituted). A lookup table was then generated based on these vectors, resulting in a similar table to that used in the random encoding approach. The remaining steps are identical to the fully random encoding. In particular, an optional FFT step may also be performed as described above.

### Encoding Complexity

The encoding complexity N works as a as a hyperparameter for the ML model. Larger N values, i.e., higher encoding complexity, result in a higher learning capacity, but low N values result in model under-fitting. Overfitting due to large N values can be avoided by the inclusion of an FFT (see FIG. 35 and FIG. 36) and/or further compensated by adjusting regularisation (while also increasing computational demand) by using, for example, regularised Lasso models, see Example 5. Indeed, it has been shown that increasing the encoding complexity can provide individual Lasso models with better performance for a random encoding approach than is possible using the AAindex database.

### Machine Learning and Aggregation

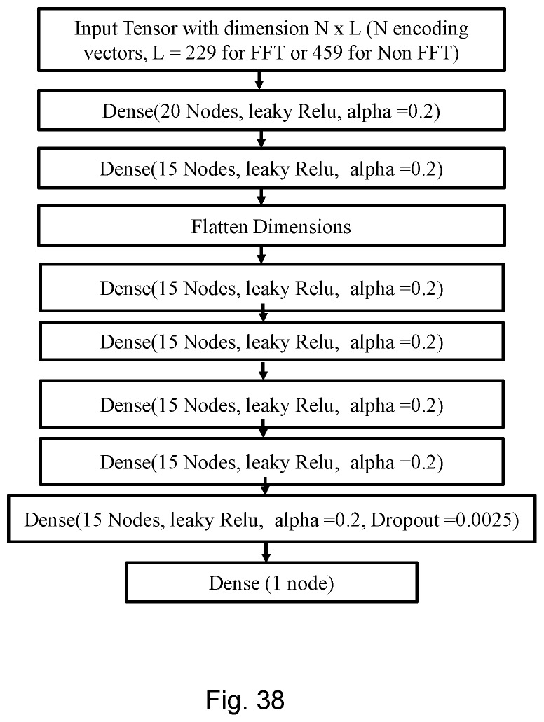

Several ML models (including multilinear regression, regularised Lasso, SVR and artificial neural networks) were employed. Neural networks were implemented in python3 with the Keras module and a TensorFlow backend and consisted of a series of dense artificial neural networks models (see FIG. 38 for the neural network architecture). The rest of the ML models were implemented in python3 by the sklearn library. Alternative and more efficient architectures may be obtained by further varying the design parameters, such as number of nodes, regularisation, dropout, normalisation or by implementing different types of architectures, such as recursive neural networks. Furthermore, model performance may depend on other aspects such as the nature of the enzyme and the amount of mutant data used for training.

For ensemble modelling, each individual ML model had a unique set of encoding vectors and was trained using data from a specific conformation. Training iterations were individually split into a 70% training set, 15% test set and 15% validation set by the sklearn splitting function. Furthermore, artificial neural networks were trained for 30000 cycles of five epochs each with batches of size of 1000. During each cycle only half of the training data was randomly used (by a random split). For each artificial neural network, the performance of each training iteration was assessed using correlation coefficients on the test set. The best model state (defined by its weights) was saved to memory and further used for model predictions. The performance of the best model state (measured by correlation coefficients on the test sets) was in each case confirmed by making a similar performance measurement on the validation sets.

### Site Directed Mutagenesis Potential Map

For each ensemble of ML models, a site directed mutagenesis map was obtained. Every possible single mutant in the enzyme sequence was predicted for each fully trained ML model. These predictions were then standardised (for each model output) to a mean of 0 and a standard deviation of 1. The calculated mean and variance for each model was stored for further model standardisation. For each single mutant prediction, a mean across all models in the ensemble was then calculated. Furthermore, a site-specific mean, maximum and minimum (based on all possible amino acid substitutions per site, e.g., 20) was calculated to obtain a metric that could be calculated at each site, see FIG. 33.

### Computationally Guided Library Construction

The construction of the rational DE libraries was made by OE-PCR based de novo full gene synthesis. A D3 HDNO DNA sequence was optimised for the E. coli host (the annealing temperatures were adjusted on the ligation sites and miss-annealing nucleotides were removed [115]). The gene was synthesized by Integrated DNA Technologies Inc. (Coralville, USA) and cloned into pBbE2k plasmid. Overlapping degenerate mutagenic primers were designed as defined in Example 2 to enable extension PCR and permitting multiple mutations at several sites within the D3 HDNO sequence. The libraries were generated by the inclusion of small degenerate codons with no stop codons based on the previously calculated rankings based on the scoring methodology: library 1 (V109RWG G112RBC D113RDC) and library 2 (Y242 KWC, K348 VAS, G353 RDC).

### Enzyme Activity Assay

E. coli BL21 (DE3) competent cells were transformed with the computationally guided libraries 1 and 2 described above and cultured onto LB agar plates containing 100 μg ml1 kanamycin. In short, 380 individual colonies from each library were picked and inoculated into individual microtitre plate wells to express HDNO protein, as defined in section 2. Each of the 380×1 mL LB cultures were induced with 10 nM tetracycline and incubated at 20° C. overnight, shaking at 180 rpm. Cultures were then centrifuged at 2250×g to pellet cells and resuspended in 100 μL of BugBuster protein extraction reagent (Merck) and incubated for 15 minutes at room temperature to lyse cells. 5 μL of lysed cell material was used in the enzyme activity assay.

Screening of enzyme activity was performed using an absorbance-based assay, which detects the hydrogen peroxide produced by the oxidase during catalysis. This hydrogen peroxide is used by horseradish peroxidase to oxidise substrates 2, 4, 6-tribromo-3-hydroxybenzoic acid and 4-aminoantipyrine, which generates a pink colour that can be quantified by measuring absorbance at 510 nm. The assay consisted of 95 μL assay buffer containing the above horseradish peroxidase and substrates and 10 mM amine substrate, and the assay was initiated by addition of 5 μL lysed cell material. Following incubation at room temperature for 15 minutes the absorbance at the plate was measured at 510 nm using a microplate plate reader. Quantification was performed by comparing absorbance to a standard curve, derived using pure hydrogen peroxide at known concentrations.

### Conclusions

A comprehensive methodology for the rational design of DE libraries based on short (1 ns) MD simulations and conformational sampling was demonstrated to enable improved protein engineering. An unprecedented set of over 360,000 conformationally diverse MD simulations were performed for a set of distinct mutant variants of 6-HDNO. The enzyme turnover activity was estimated for each mutant based on MD and electrostatics. An exemplar ML based process was then employed to generate predictions in a complete computational enzyme design process (other ML methodologies can be substituted and are anticipated to work in an identical fashion in the process described herein, including but not limited to Bayesian models, random forests, k-nearest neighbours, deep learning, and alternative encoding, autoencoder and variational autoencoder methodologies). The approach was successfully validated in a series of experiments, which clearly demonstrate efficacy by generating a diverse set of active mutants in two very small but efficient DE libraries. The inventors believe that this low-risk and high return process represents a quantum leap in protein engineering that could be compounded through many rounds of evolution, to rapidly develop active mutant variants with mutations outside the active site that cannot be found by other state of the art methodologies. Finally, while further high-ranking libraries could readily be tested for improved enzyme activity (in addition to the ones already tested, leading to an accumulation of mutations) the process described in this example could be repeated in full once a better variant has been identified (including generating a new parent MD, new mutant MD data, and new ML models to predict the best ranking sites).

### Example 4: Accelerating Directed Evolution with Machine Learning Based on Dynamics-Driven Predictions of Enzyme Catalytic Turnover Number Applied to a Hydrolase (EC-3)

### Introduction

Examples 1 to 3 describe new methods for accelerating DE (as compared with traditional DE methodologies) and have been validated on the protein 6-HDNO (an EC-1 oxidoreductase enzyme for the catalysis of phenylpyrrolidine).

The inventors have built on the successes described in Examples 1 to 3 to illustrate the application of the methodology to other enzymes, in this case an example is shown of how computationally guided DE of α-amylase would be performed. The inventors have used the previously described methodology to design more efficient evolutionary libraries and that could be used in DE iterations to discover better variants with a higher catalytic rate of maltose hydrolysis. α-amylase is an EC-3 hydrolase enzyme found commonly in nature across plants, animals, and microorganisms where they are involved in catalysing the hydrolytic breakdown of starch molecules into glucose sub-units. Moreover, these enzymes play a key role in many industrial sectors where they are found in a diverse range of processes such as in the production of food, textiles, paper, and detergents. Amylases have the potential to be used in many other applications such as in the synthesis of active pharmaceutical ingredients (API), where a hydrolysis step is required, potentially increasing the efficiency of chemical processes with innovative bioprocess routes [150, 151]. Amylases are produced on industrial scales and already significant research has been directed towards improving their stability and catalytic performance through DE (e.g., [152-154]).

The inventors recognised that further protein engineering of α-amylases by DE may prove crucial for the incorporation of these enzymes into a wider range of industrial applications as well as improving efficiency and sustainability of current processes. DE is an iterative method that consists of alternating the generation of populations with different degrees of genetic diversity by various mutagenesis techniques and selecting the best variants according to a desired property. This can be achieved by the computationally guided technology described in Examples 1 to 3. Furthermore, the use of this technology for library and codon optimisation is also described in this Example.

### Results

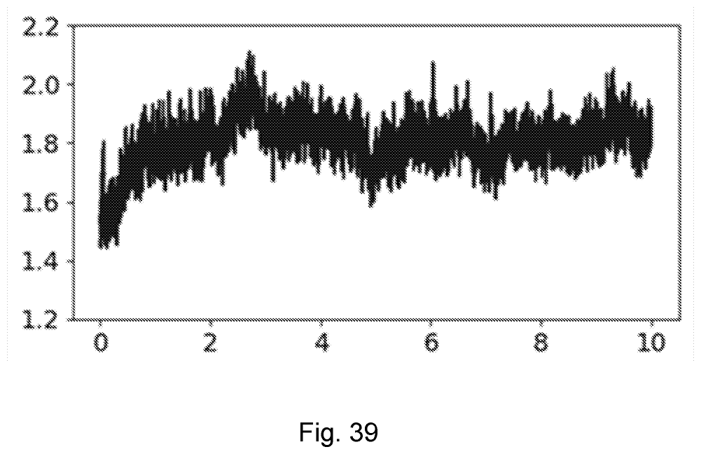

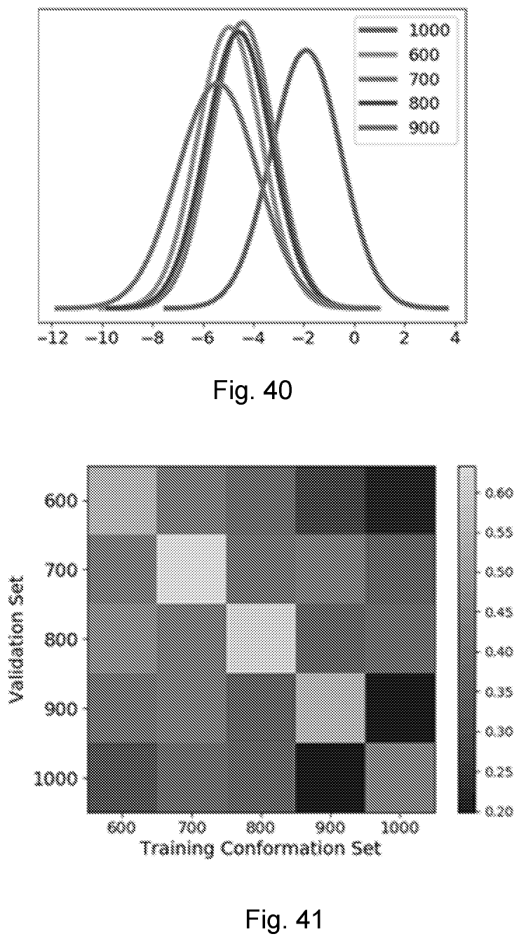

A fast exploration of potential targets for the DE of the human pancreatic α-amylase enzyme was performed following the process described in Example 3, with the aim of generating enhanced libraries (enhanced meaning including variants with increased catalytic turnover number), for use in DE iterative improvement. A system comprising the amylase protein and a substrate (maltose) was prepared. A total of 1 μs of molecular dynamics (MD) was performed to equilibrate the wild type (WT) system and generate a set of diverse structures (a low RMSD was observed, which demonstrates a stable and equilibrated system, see FIG. 39). Five structures representing different starting conformations were selected for mutant generation. A set of over 45000 random triple mutants were generated and a specific starting conformation was randomly assigned to each variant. Following the computational preparation procedure of the mutant simulations (as described in Example 2), a total of 1 ns of MD was performed on each mutant variant. The Q20 scoring methodology was then used to score each sampled conformation from the mutant MD simulations and a single pQ₂₀ score was obtained for each mutant (as described in Example 1). As observed in Example 3, the PQ20 scores from each conformation produced distinct distributions (i.e., distinct values for the mean and variance of the populations of pQ20 scores), even though an equally balanced, diverse, and large random mutation set was used in generating the data within each conformational subset (see FIG. 40). Therefore, a representative sample of seed conformations was additionally used to generate the mutant data. In this case, five seed conformations were used (compared to 10 in Example 3). As discussed above, even one seed conformation can be used to produce acceptable results; an increased number of seed conformations and mutant data, including increased lengths of each individual MD run, may produce better results, with no upper limit, and the main restriction is the availability of computational resources.

The difference between seed conformations became clearer by comparing the compatibility of the predictions on each conformational dataset using a series of trained ML models (as described in Example 3). A total of 30 neural network models were used on each conformational dataset (150 models in total). These models used random-FFT ProSAR (random encoding method) protein data encoding and an encoding complexity of 17 (N=17 random encoding vectors; N is the number of random numbers that are generated to encode for each distinct type of amino acid in the sequence, see methods in Example 3) employing the same neural network architecture used in Example 3, and a self-correlation and cross-correlation matrix (FIG. 41) was obtained. The diagonal of the matrix represents the self-correlation, which is a measure of the correlation between models trained on and tested against data from the same seed conformation. The non-diagonal values (cross correlations) represent the relative measures of cross compatibility of the mutant data between different seed conformation sets, by measuring the performance of models trained on one seed conformation but tested for on data from a different seed conformation. The low cross-correlations between different seed conformations indicated that using more than one seed conformation was beneficial to predict the effects of mutants based on the current methods.

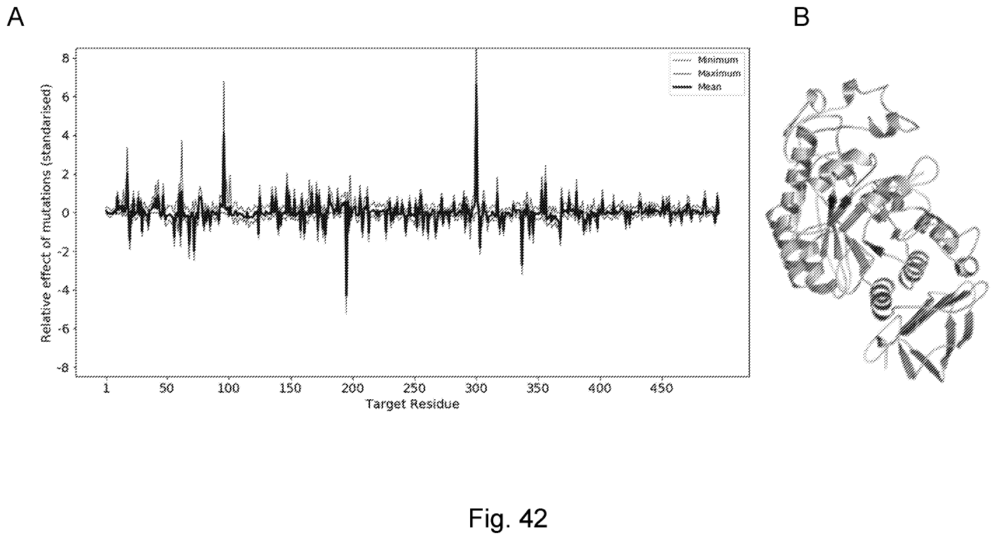

A full in silico site directed mutagenesis prediction map was generated, which locates the best residues (sites) for mutagenesis in the design of high-performance DE libraries (based on the same method described in Example 3 and FIG. 31). In this process, a series of ProSAR artificial neural network models were employed based on a random encoding methodology, followed by an FFT of the protein sequence data. A total of 30 models per conformation were used for each ensemble process with an encoding complexity of N=17 (see encoding methods in Example 3 for full details). A heatmap was produced from the same data to visualise the distribution and intensity of different site candidates (see FIG. 42). Residues of potential positive and negative impact on the rate of reaction (catalytic turnover number) can be observed to be distributed across the entire enzyme space, with some residues playing a more important role than others. The lack of a simple pattern or rule supports the need for a computations approach to guide the type and diversity of mutations during DE experiments. Based on these results, residue Glu300 was predicted to be the worst target for mutagenesis. Glu300 has previously been identified as a catalytically relevant residue [155], which confirms this result and supports the fact that it should not be targeted experimentally. Conversely, residue Arg195 was predicted to be the best option for mutagenesis in a library of mutations during a DE experiment.

It is noted that the methods described in Examples 1 to 3 are aimed at enriching DE libraries by measuring the impact of mutations on the electrostatic component of the rate of reaction. However, other enzymatic properties such as enzyme stability, pH tolerance, substrate diffusion to the active site, non-electrostatic components of rate of reaction and/or any other unforeseen properties, may also be important factors that determine enzyme activity and can all be potentially impacted by any mutation. Therefore, a diverse library designed for increased turnover numbers may still yield a large group of inactive variants. For this reason, the current process works best by testing several high-ranking targets, which can benefit from a combinatorial approach (by simultaneously testing many targets) when designing a library for use in DE experiments.

Several molecular biology approaches can be employed interchangeably to produce the genetic diversity (and hence diversity of proteins in a library) used in DE experiments. All such methods may produce similar results. Previous experimental validations of the technology (see Example 2 and 3) have been performed effectively based on error-corrected PCR based or OE-PCR de novo full gene synthesis. This gene synthesis technology works by constructing a set of oligonucleotides that can be annealed together and amplified into a DNA library of mutants that can then be translated into the protein variants [14]. The experimental approach is limited by the total amount of screening possible (due to the available resources), and there is also a potential negative impact from amplification bias [142]. Therefore, smaller libraries may be the most practical solution and may also produce the best results. For the computational process, a good compromise is found in designing libraries that have mutagenesis at a small number of sites (e.g., three sites) to reduce the combinatorial space. However, that said, there are still hundreds of degenerate codons that can be chosen at each site and need to be predicted by the computational method. A further reduction can be made by selecting from a reduced set of degenerate codons (e.g., codons that code for a maximum 12 amino acids without repetition, contain no stop codons and are forced to include the wild-type amino acid).

A selection of the best target sites was made based on the in-silico site directed mutagenesis map that described the potential for improvement of catalytic turnover number. Based on an average of all predicted single mutants per site, targets Pro57, Arg195 and Arg337 were identified as the best target sites. As the skilled person understands, more than three sites could be targeted if desired. This subset of sites can be targeted with a total of approximately 2123525 distinct library combinations (or specific combinatorial experiments), with the codon reductions in place that were described above. A thorough analysis using the full set of ML models to predict every mutant activity resulted in the identification of a specific example library with the following positions (and degenerate codons): 57 (SMV), 195 (SVM), and 337 (SDG) as an enriched library of a maximum diversity of 576 different variants. However, any similarly high-ranking library may be an equally good candidate for DE experiments on this enzyme.

### Materials and Methods

### Initial System Preparation and MD

The initial system was set up based on the crystal structure 1B2Y from the Brookhaven protein data bank (PDB) [156]. Molecular dynamics (MD) simulations were performed using the OpenMM software [157] employing an AMBER force field. AMBER protein parameters were employed (ff14SB) for the enzyme [51] and the General AMBER Force Field (GAFF) [48] for the substrate and counter-ions and parameters form the TIP3P model was used for the water solvent [52]. During all MD simulations a set of asymmetric harmonic restraints were imposed on the substrate and protein to limit the conformational space into structures resembling a near attack conformation (NAC). The restraints were imposed by manual inspection based on the proposed mechanism of reaction with the intention of holding the key residues and the substrate in a NAC during the MD simulations. The series of restraints consisted of Glu233-OE2 to the glycosidic oxygen of maltose (2.9 Å), Asp197-OD1 to C1 carbon of maltose (3.1 Å) and Asp-OD2 to 06 oxygen of maltose (2.8 Å), all with a force constant of 1000 kJ×Å⁻². The wild type (VVT) enzyme was subject to a 1 μs MD simulation and structures corresponding to timeframes 600 ns, 700 ns, 800 ns, 900 ns and 1000 ns were extracted for the later construction of mutant MD simulations.

### In Silico Mutagenesis and Mutant Scoring

A set of over 45000 triple mutants were generated randomly by targeting any site other than the first 10 residues on the N terminus as well as the last 10 residues of the C terminus of the enzyme, due to the higher dynamic variability typically observed in these regions (additionally residues Asp197 and Glu233, which were identified as relevant residues in the mechanism of reaction a priori, were excluded and any Cys residues, since they could be involved in disulphide bridge formation; similarly, no residues were mutated into Cys for the same reason). Mutants were generated by modifying the side chain structures computationally from the 3D-structure of the randomly assigned seed conformation. All mutants were then prepared for MD simulation (as described in Example 2), before running a 1 ns MD simulation. All the sampled coordinates (comprising 10 sampled conformations, separated linearly by 0.1 ns of simulation time) were scored by the Q20 methodology described in Example 1.

### Transition State Search and Q20 Parameterisation

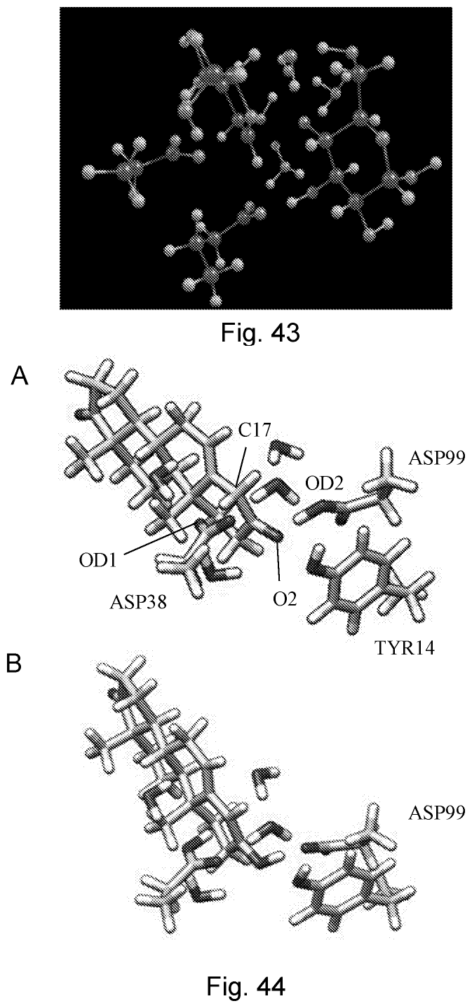

The conformation at timeframe 11.0 ns from the wild-type (WT) MD simulation was used as a base structure to search and obtain the rate-limiting transition state (TS) 3D-structure and the reactant complex (RC) 3D-structure via DFT cluster model optimisations at the BP86/3-21G level of theory [19-21] (see FIG. 43). The cluster model consisted of key residues Glu233, Asp197, the maltose substrate and several water residues (as water molecules play a part in the reaction mechanism in this case by forming a water chai to transfer a proton). An analytical vibrational frequency calculated by the DFT method on the transition state structure confirmed the right activation step [155, 160, 161]. The change in partial atomic charges was calculated using a Hirshfeld population analysis [162] at the B3LYP/6-31G* D3BJ level of theory [159, 62, 61]. All DFT calculations were performed using the ORCA Chemistry software package [60]. These partial atomic charges were used to parameterise the Q20 scoring methodology (as described in Example 1). Alternative methods to obtain the RC and TS 3D-structures could also be used, for example, a QM/MM methodology, as demonstrated in Examples 1 to 3. Alternative DFT functionals (e.g., BP86, BLYP, M06) or ab initio methods (e.g., MP2, MP3) could also be used with a variety of basis sets (e.g., 6-31G*, def2-SVP, 3-21G*), optionally with the empirical dispersion correction (D3BJ) to which other equivalent alternatives have also been reported (e.g., D3, D2).

### Machine Learning Ensemble

A series of neural network models were trained for ensemble predictions as described in Example 3. In short, all data was encoded following a random encoding ProSAR methodology, where no amino acid properties are required, including a FFT step on the encoded data. The predictions of a series of neural network ML models were grouped into subsets and each ML model was trained on data from specific seed conformations. Unseen validation and test subsets were created to monitor the performance (by calculating correlation coefficients) of the models. A set of 30 artificial neural network models were generated for each conformation set (150 models in total).

### In Silico Site Directed Mutagenesis Potential Map

For each ML model, a prediction of catalytic turnover improvement was obtained for every possible single-mutant. These predictions were then standardised for each model output to a mean of 0 and a standard deviation of 1, while the calculated mean and variance for each model was stored for further model standardisation. For each single mutant prediction, a mean across all models in the ensemble was then calculated. Furthermore, a site-specific mean, maximum and minimum (based on all possible amino acid substitutions per site, e.g., 20) was calculated to obtain a metric that could be calculated at each site (shown in FIG. 42), with best sites identified as 57, 195 and 337.

### In Silico PCR Based DE Library

Once a set of high-ranking sites had been selected, further optimisation was performed to choose the specific codons for a DE experiment with PCR based full gene synthesis. While the site directed mutagenesis potential is useful as a quick guide toward site ranking, further scoring by ML is used to assess individual mutants in a specific library. Therefore, for the optimisation of codon libraries, the full ensemble of ML is used to score each possible mutation in each codon combination. The ML models predictions are standardised based on the parameters obtained during the previous step (site directed mutagenesis potential map generation). Based on these predictions, the median score of each possible library was compared to select the highest-ranking library. A total 2123525 distinct library combinations were possible for these sites considering the codon restrictions described previously (including the wild-type amino acid in the degenerate codon, no stop codons, only encoding once for each amino acid, and encoding for no more than 12 amino acids). A thorough analysis was performed to identify the sites and degenerate codons.

### Conclusions

A comprehensive in silico study was performed using the methods described in Examples 1 to 3 to predict and map the effect of directed evolution combinatorial libraries guiding DE experiments in improving the catalytic activity of the human pancreatic alpha amylase enzyme in the hydrolysis of maltose. The methodology described herein enables the exploration and mapping of the full mutational landscape, including locations outside of the active site (which are normally not a target in state-of-the-art protein engineering). The methodology allows DE iterative experiments to access unique enzyme variants with faster rates of reaction due to improved catalytic turnover number. Further improvements in the process are anticipated as computational hardware becomes more efficient, machine learning models become more proficient, and it can be scaled up to larger datasets. It was possible to perform the process of site selection of unrelated enzymes in this Example and Example 3 in an essentially identical manner, and the inventors therefore propose that this as a suitable general methodology for protein engineering of enzymes where the 3D-structure is either known form experiment or can be calculated from previously known 3D-structures (such as in homology modelling), and for which a reaction mechanism has been proposed, which is optionally based on e.g., other similar substrates or enzyme or optionally based on purely theoretical considerations (e.g., by DFT calculations).

### Example 5: Accelerating Directed Evolution with Machine Learning Based on Dynamics-Driven Predictions of Enzyme Catalytic Turnover Number Applied to an Isomerase (EC-5)

The inventors have built on the successes described in Examples 1 to 4 to illustrate the application of the methodology to other enzymes. This example shows how computationally guided DE of ketosteroid isomerase would be performed. In this example, the inventors have used the previously described methodology to design more efficient evolutionary libraries, which could be used in DE iterations to discover better variants with a higher catalytic rate of cholesterol isomerisation. In showing this the inventors additionally propose a general method for protein engineering that can be applied to enzymes that have known or calculable 3D structures.

Isomerases catalyse interconversions in the spatial arrangement of atoms and are involved in the central metabolism of most living organisms. Isomerases have also been recognised as having important applications in organic synthesis, biotechnology, and drug discovery [165]. Ketosteroid isomerase plays a crucial role in the conversion of cholesterol into testosterone in many living organisms and microbial systems and the inventors recognise that the enzyme may thus be repurposed for the synthesis of active pharmaceutical ingredients by directed evolution and in light of this interest present a series of results from proof-of-concept application of the current technology for this enzyme.

### Results

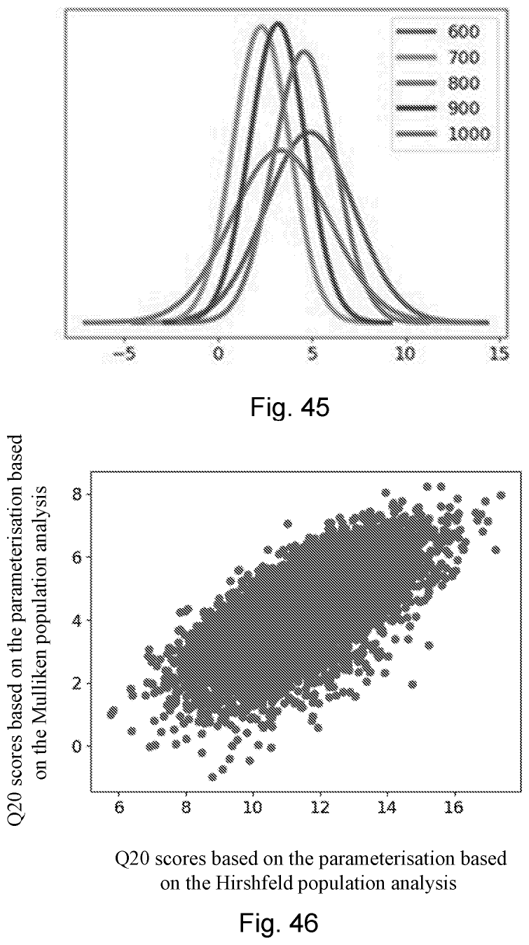

The aim of this Example was to make a computer prediction for a library that contained variants of ketosteroid isomerase enzyme (KSI, an EC-5 isomerase enzyme) with improved enzyme turnover number for use in DE experiments. The exploration of potential mutation sites followed the same procedures as Examples 1 to 4. A system comprising the KSI protein and a substrate (5-androstene-3,7-dione) was prepared and a total of 1 μs of molecular dynamics (MD) was performed to equilibrate the wild type (WT) system and generate a set of diverse seed conformations. Five frames from the simulation, representing different starting seed conformations, were selected for subsequent mutant generation. A set of 50000 random triple mutant variants was generated and one of the five starting conformations was randomly assigned to each variant. Each mutant was prepared from its starting conformation (using methods described in Examples 2 and 4) and a 1 ns MD simulation was performed on each (10 conformations were saved at 0.1 ns intervals). The Q20 scoring methodology was employed (as previously described in Examples 1 to 3) to score each frame of the mutant MD simulations and a single μ_{Q20} score was obtained for each mutant (as described in Example 1). The Q20 score was parameterised based on DFT models of the enzyme (see FIG. 43). As observed in Example 3, the P_{Q20} scores from each conformation produced distinct distributions (i.e., distinct values for the mean and variance of the populations of¹μ_{Q20} scores), even though an equally balanced, diverse, and large random mutation set was used in generating the data within each conformational subset (see FIG. 45).

The performance of different methods for parameterising the Q20 model (using both the Hirshfeld population analysis and the Mulliken population analysis for the calculation of partial atomic charges) was evaluated by comparing their resultant calculated ΔΔG^{‡}_{Q20} scores. Every saved frame of the wild type 1 μs MD simulation was scored using both methods (at 0.1 ns intervals that resulted in 10000 ΔΔG^{‡}_{Q20} scores per method) and a clear correlation between them (see FIG. 46) confirmed the interchangeably of these methods in the computational predictions described herein.

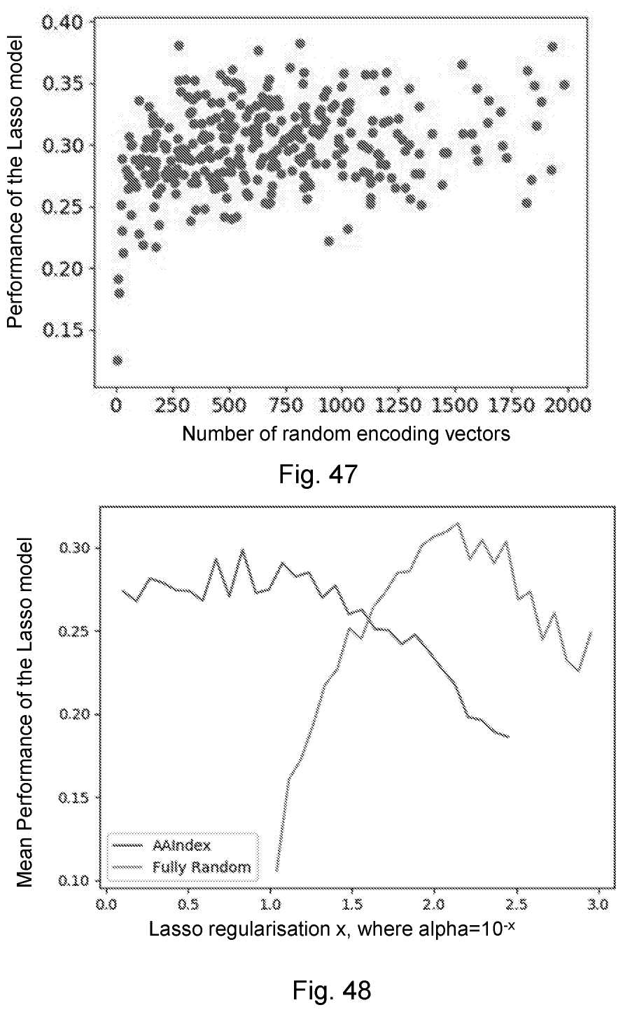

A series of ML models were used (as previously described in Example 3 and 4) to analyse mutant datasets associated with each seed conformation individually. However, a variation was introduced by using a single Lasso model for each seed conformation instead of an ensemble of ML models. The inventors recognise that although some additional noise is expected, the models will perform sufficiently well in practice, therefore demonstrating that the approach using multiple seed conformations is beneficial but not necessary. The Lasso models were grid-search optimised for an encoding complexity of N=750 random vectors, resulting in an encoding complexity that is larger than what is possible with a limited set of amino acid properties, such as the AAindex with under 600 properties (see FIG. 47 for grid search of encoding complexity). A further test was performed to compare the performance of the AAindex utilising a series of 553 amino acid properties against the performance of a fully random model with a complexity of 750 encoding vectors. FIG. 48 shows the mean model performance obtained by the regularised Lasso models over a grid search of the α-hyperparameter (based on seed conformation 900 ns, which corresponded to the best model performance). The random encoded models surpassed the best performance of the AAindex property encoded models.

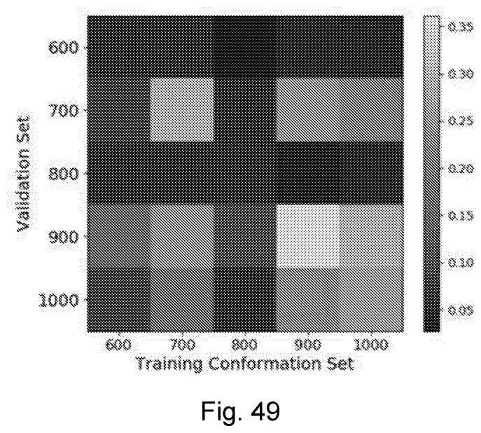

Further analysis was therefore restricted to using only the random encoding models, and a self- and cross-correlation matrix (FIG. 49) was calculated. The diagonal of the matrix represents the self-correlation, which is a measure of the correlation between models trained on and tested against data from the same seed conformation. The non-diagonal values (cross correlations) represent the relative measures of cross compatibility of the mutant data between different seed conformation sets, by measuring the performance of models trained on one seed conformation but tested for on data from a different seed conformation. The low cross-correlations between different seed conformations indicated that using more than one seed conformation was beneficial to predict the effects of mutants based on the current methods.

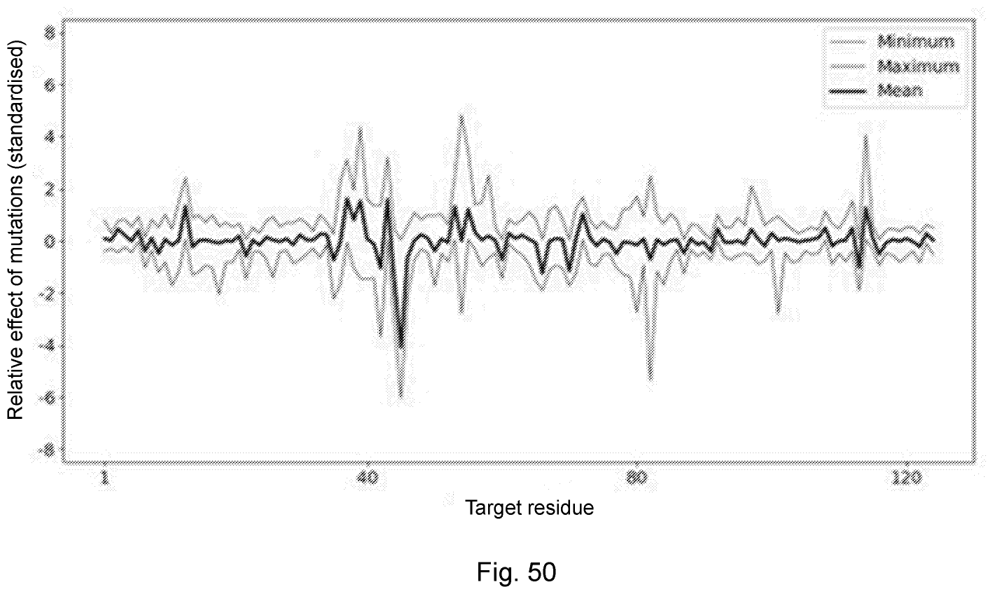

Following a standardised aggregation of the ML models (based on five seed conformations), a full in silico site directed mutagenesis potential map was generated. The site directed mutagenesis potential map of FIG. 50 presents the best residues for mutagenesis for their potential in the design of highly effective DE libraries. Table 19 displays the items with the highest calculated average potential per site, with the top three sites identified as 45, 44 and 66, in descending order of relevance. This subset of sites can be targeted with a total 1244825 distinct library combinations (using the same codon reductions as described in Examples 3 and 4). Further analysis resulted in the identification of the following sites (and degenerate codons): 44 (SMW, encoding for any of A, D, E, H, P, Q), 45 (VDA, encoding for any of D, E, G, H, Q, R) and 66 (DDG, encoding for any of E, G, K, L, M, R, V, W), resulting in a library with a maximum diversity of 288 different variants. However, any similarly high-ranking library may be an equally good candidate for libraries that could be used in DE experiments to improve catalytic turnover number.

TABLE 19
The top ranked sites based on the average potential based on
the standardised mean predictions from a set of regularised
Lasso models trained from data from 5 distinct conformations.

| Rank | Site | Score |
|---|---|---|
| 1 | 45 | −4.09195 |
| 2 | 44 | −1.47206 |
| 3 | 66 | −1.25145 |
| 4 | 70 | −1.16156 |
| 5 | 42 | −1.05477 |
| 6 | 113 | −1.02604 |
| 7 | 35 | −0.74 |
| 8 | 60 | −0.73618 |
| 9 | 82 | −0.71067 |
| 10 | 46 | −0.6457 |
| 11 | 22 | −0.60323 |
| 12 | 116 | −0.51921 |
| 13 | 77 | −0.4988 |
| 14 | 87 | −0.48731 |
| 15 | 9 | −0.48327 |

### Methods

### Initial System Preparation and MD

The initial system was set up based on the 1OHP crystal structure from the protein data bank, and residue numbers referred to in this example use the same numbering (the sequence starting with amino acids: MNTP). Molecular dynamics (MD) simulations were performed following the same procedures as in Examples 3 and 4. During all MD simulations a set of asymmetric harmonic restraints were imposed on the substrate and protein to limit the conformational space into structures resembling a near attack conformation (NAC). The restraints comprised: Asp99 OD2 to substrate 02 (2.5 Å) and Asp38 OD1 to substrate C17 (3.5 Å), and all had a force constant of 1000 kJ×Å⁻² (see FIG. 44 for atom names). The wild type (WT) enzyme was subjected to a 1 μs MD simulation and structures corresponding to timeframes 600 ns, 700 ns, 800 ns, 900 ns and 1000 ns were extracted for the generation of mutants. As previously discussed, the frame selection was arbitrary, but sufficient to allow diversity. Conformations were selected from the latter half of the simulation, where the simulation is closer to thermodynamic equilibrium.

### In Silico Mutagenesis and Mutant Scoring

A set of 50000 triple mutants were generated randomly targeting any site, except for residues Tyr14, Asp38 and Asp99, which were identified a priori as essential residues in the mechanism of reaction. Any sites containing Cys residues were also excluded, and no residues were mutated into Cys (to avoid the formation of disulphide bridges). Note that the impact of this restriction is minimal for the construction of libraries because it has a negligible effect on the size of the possible number of protein mutants. The first 10 residues on the N terminus as well as the last 10 residues of the C terminus of the enzyme were also left unchanged due to the higher dynamic variability typically observed on these regions. However, these could also be included with no added technical challenge (as demonstrated in Example 6). Mutants were generated by modifying the side chain structures computationally from the 3D-structure of the randomly assigned seed conformation. All mutants were then prepared for MD simulation (as described in Example 2), before running a 1 ns MD simulation. All the sampled coordinates (10 per 1 ns) were scored by the Q20 methodology described in Example 1.

### Transition State Search and Q20 Parameterisation

The conformation at timeframe 240.0 ns from the wild-type (VVT) MD simulation was used as a base structure to search and obtain the rate-limiting transition state (TS) 3D-structure and the reactant complex (RC) 3D-structure via DFT cluster model optimisations at the BP86/3-21G level of theory [19-21]. The DFT cluster models included the substrate and residues Tyr14 and Asp38 as well as several water residues (see FIG. 44 for RC and TS structure) and the core region of the Q20 scorer was set to include residues 14, 38, 99 and the substrate. A transition state optimisation was performed based on the mechanistic model proposed previously [166]. Alternative DFT functionals (e.g., BP86, BLYP, M06) or ab initio methods (e.g., MP2, MP3) could also be used with a variety of basis sets (e.g., 6-31G*, def2-SVP, 3-21G*), optionally with the empirical dispersion correction (D3BJ) to which other equivalent alternatives have also been reported (e.g., D3, D2).

### Machine Learning Models

A series of regularised Lasso models were trained and aggregated for predictions as described in Examples 3 and 4. However, instead of using an ensemble of models to train on the data of each seed conformation, only one Lasso model was for each seed conformation, respectively. All data were encoded following a random encoding ProSAR methodology, followed by an FFT process. A single Lasso model was fitted to each mutant set corresponding to a single seed conformation (5 models in total), and a large encoding complexity (N=750) was used for each model. FIG. 47 shows a grid search for the encoding complexity at α=10⁻².

### Site Directed Mutagenesis Potential Map

For each ML model, a prediction of catalytic turnover improvement was obtained for every possible single-mutant. These predictions were then standardised for each model output to a mean of 0 and a standard deviation of 1, while the calculated mean and variance for each model was stored for further model standardisation. For each single mutant prediction, a mean across all models in the ensemble was then calculated. A site-specific mean, maximum and minimum (based on all possible amino acid substitutions per site, e.g., 20) was calculated to obtain a metric that could be calculated at each site (this is shown in FIG. 50). The best sites were identified as 45, 44 and 66.

### ML Hyperparameter Optimisation

A grid search was performed to optimise the Lasso regularisation factor based on a series of ML models, encoded with complexity of N=750 and with the AAindex using 553 properties (see FIG. 48). A total of 300 models were trained with both types of encoding, within a range of regularisation parameter values, with the intention of identifying a maximum. FIG. 48 displays the mean performance of the models binned into 30 bins across the range of the horizontal axis based on the conformation from 900 ns, for which the best model performances were obtained.

### In Silico PCR Based DE Library

Once a set of high-ranking sites had been selected, further optimisation was performed to choose the specific codons for a DE experiment with PCR based full gene synthesis. Although the site directed mutagenesis potential is useful as a guide to rank sites, further scoring is necessary by ML to assess individual mutants in a specific library. Therefore, for the optimisation of codon libraries, the previously trained ML models were used to score each possible mutation in each codon combination. The predictions from the ML models were standardised based on the parameters obtained during the previous step (the site directed mutagenesis potential map generation). Based on these predictions, the median score of each possible library was compared to select the highest-ranking library. A total 1244825 distinct library combinations were found possible for these sites (using the following codon reductions: codons must include the wild-type amino acid, no stop codons, only encoding once for each amino acid, and encoding for no more than 12 amino acids). Further analysis was performed to identify the sites and degenerate codons).

### Conclusions

In the current Example the inventors build on previous demonstrations of the invention by expanding the exemplification to the isomerase enzyme class (belonging to EC-5). These results suggest the applicability of the herein described invention for the computational guidance of DE across enzyme classes. Moreover, some parameters, such how the Q20 model is parameterised, have been modified to exemplify that such variations can be introduced with no practical impact on the processes described herein. In contrast to Examples 1 to 3, a DFT cluster approach was used to obtain the necessary optimised reactant complex and transition state to parameterise the scoring function. The inventors further demonstrated the feasibility of using a single ML model for each seed conformation to generate predictions of sites that can be used in DE experiments to improve the catalytic turnover number.

### Example 6: Accelerating Directed Evolution with Machine Learning Based on Dynamics-Driven Predictions of Enzyme Catalytic Turnover Number Applied to a Transferase (EC-2)

The inventors have built on the successes described in Examples 1 to 5 to illustrate the application of the methodology to other enzymes, in this case an example is shown of how computationally guided DE of xanthosine transferase would be performed. The inventors have used the previously described methodology to design more efficient evolutionary libraries and that could be used in DE iterations to discover better variants with a higher catalytic rate of methyl transfer. In showing this the inventors confirm its use in a wider class of enzymes, including those with non-covalently bound cofactors and show how the process can be generalised to essentially any number of mutations in the protein variants, and different QM and MD methods.

Methyl groups are important in pharmaceuticals in modulating biological activity, selectivity, solubility, metabolism and pharmacokinetic/pharmacodynamic properties of biologically active molecules. For example, the cholesterol lowering pharmaceutical lovastatin contains a chiral methyl group, which is central to its pharmacological function, and could be prepared using APIs (active pharmaceutical intermediates) synthesised using methyl transferases. An example methyl transferase, xanthosine methyltransferase (XMT), is involved in the later stages of caffeine biosynthesis, which is an additive in beverages and pharmaceuticals [167]. Hence, this enzyme may be further engineered either for a more efficient caffeine biosynthesis or for its re-purposing in API biosynthetic production. The aim of this example was to make predictions that could be used to generate enhanced DE libraries for the improvement of the enzyme turnover number of XMT by following the procedures of Examples 1 to 5. The XMT is also an example of an EC-2 transferase enzyme, which has also not been studied in any of the previous examples.

### Results

A system comprising the XMT protein, cofactor (S)-adenosyl-L-methionine (SAM) and the substrate xanthosine was prepared and a total of 1 μs of molecular dynamics (MD) were performed to equilibrate the wild type (WT) enzyme and generate a set of diverse structures. Five structures representing different starting seed conformations were selected for subsequent mutant generation. A set of over 20000 random triple mutant variants were generated and one of the five starting seed conformations was randomly assigned to each variant. Each mutant was prepared from its starting conformation (using methods described in Examples 2, 4 and 5) and a 1 ns MD simulation was performed for each mutant variant, encompassing 10 saved coordinates (one each 0.1 ns). The Q20 scoring methodology was employed (as previously described in Examples 1 to 3) to score each coordinate set of the mutant MD simulations (10 frames per mutant).

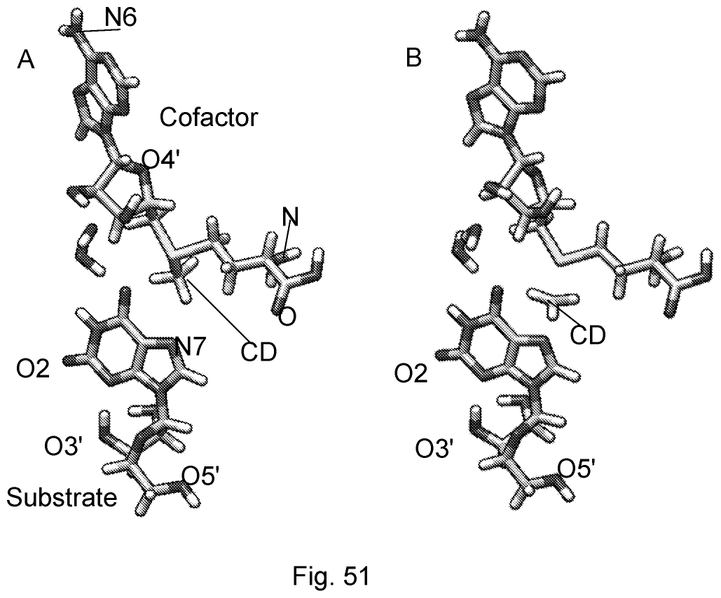

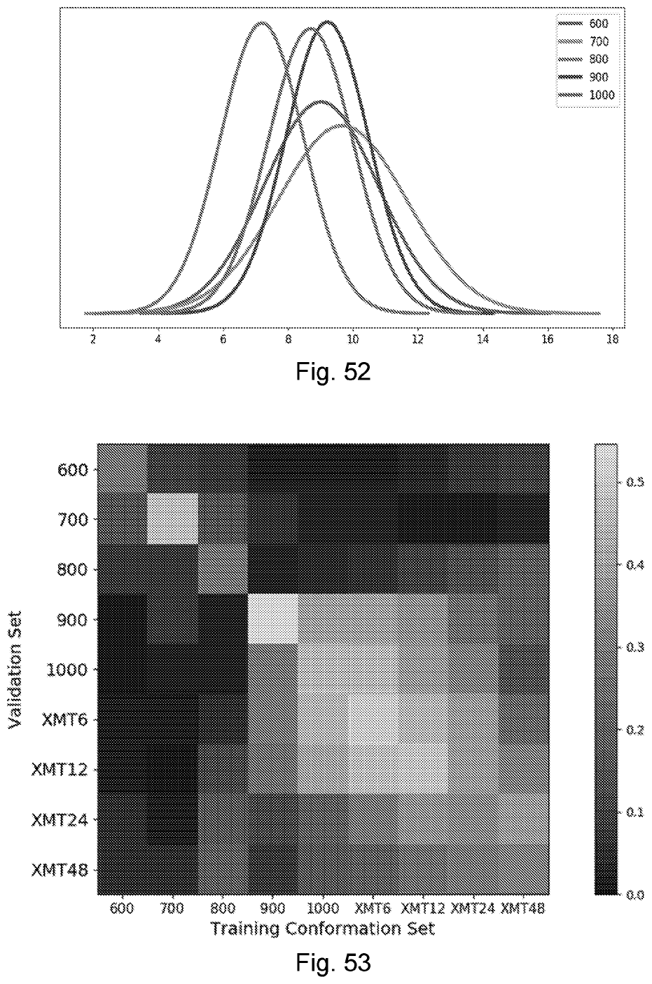

In a deviation from previous methods, a plurality of conformations was used for the parameter generation of the Q20 scoring methodology (namely conformations of timeframes 2257 ns, 3653 ns and 5210 ns) to further improve model reliability and a DFT cluster model was built for each for the optimisation of the transition state and reactant complex structures. The difference of partial atomic charges was calculated for each frame based on a Hirshfeld population analysis and a mean value of the three frames was saved as a parameter for the Q20 scoring methodology (see FIG. 51 for the model corresponding to timeframe 3653 ns). The resulting parameters were used to score the mutant MD simulations and a single μ_{Q20} score was obtained for each mutant. The distribution of the μ_{Q20,Protein} scores (which considers only the protein in the external region of the Q20 electrostatics) obtained for each set of seed conformations (see FIG. 52) demonstrate the conformational diversity associated to the datasets.

A total of 30 regularised Lasso models were trained on each data subset (resulting in 150 models in total) based on random FFT protein data encoding with a complexity of N=40. A series of further datasets were generated from the seed conformation from timeframe 1000 ns by either inserting 6 single mutations per mutant (namely set XMT6), 12 single mutants (namely set XMT12), 24 single mutants (namely set XMT24) or 48 single mutants (namely set XMT48) to establish the effects of inserting a different number of mutants into each variant. A total of 4250 random mutants were generated for each of these datasets respectively and the same ML procedure was used.

A self- and cross-correlation matrix (FIG. 53) was obtained comparing the performance and independence of all the datasets. The low cross-correlations between different seed conformations on the triple mutant data sets indicated that using more than one seed conformation was beneficial to predict the effects of mutants based on the current methods. Furthermore, no significant change in performance was found in the XMT6, XMT12, XMT24 and XMT48 datasets when compared to the triple mutant dataset (from the 1000 ns conformation). A slight drop in model performance was observed for the cross and self-correlation coefficients corresponding to data sets with increasing number of mutations (especially for the XMT24 and XMT48 datasets), but it is recognised that the effect is minimal for practical purposes, and any number of mutants can be used interchangeably for the current invention without any technical challenge. It is noted that introducing more mutations into each sequence may also be beneficial in increasing the amount of epistatic (mutant-mutant interactions) effects, which can be measured by ML methods and can improve DE outcomes. Moreover, falling model performance (from the introduction of more individual mutants per site) can be compensated by longer MD simulations and larger datasets (i.e. increasing the number of mutants and/or conformations), which in turn is only limited by time and computational resources.

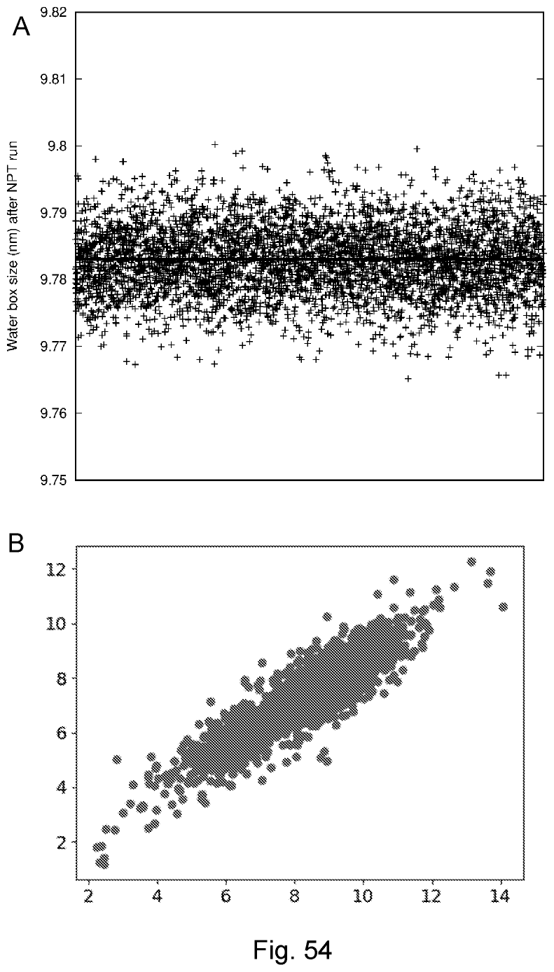

An initial analysis was made where the MD was performed using the NPT approach (instead of the NVT standard method) to confirm whether this ensemble could be substituted in the herein described methodology. In this analysis all the mutants corresponded to the seed conformation at 1000 ns (a total of 4167 mutants). As expected, under NPT conditions there was some fluctuation in the size of the solvent water box during MD (see FIG. 54A). Comparison of μ_{Q20,Protein} calculations using NVT versus NPT for the same mutants (and only using the 1000 ns seed conformation) confirmed that these methodologies are equivalent for practical purposes with a correlation coefficient of 0.782.

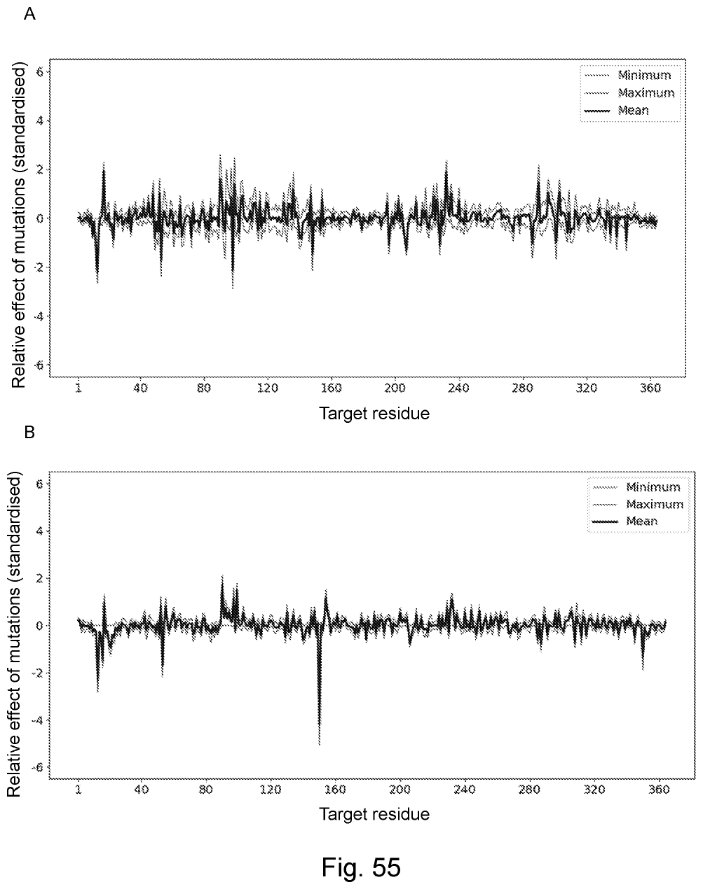

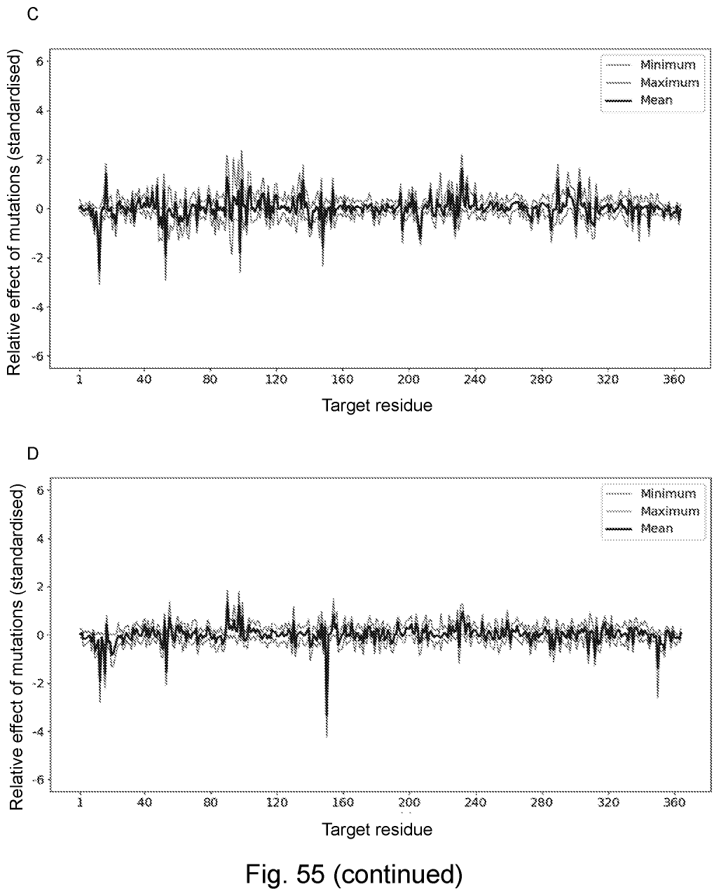

For the mutant scoring based on the Q20 methodology, the impact of solvent effects and of the variance (lognormal correction) in the ranking was assessed (see Equation (2) of Example 1). Therefore, four sets of scores were obtained. The first set (μ_{Q20,Protein} set) considered only the protein in the external region of the Q20 electrostatics. The second set also considered the solvent correction (μ_{Q20,solvent} set). The third set was based on the first set but included the variance correction (ΔΔG^{‡}_{Q20EFF, Protein} set) and the fourth set was based on the second set but included the variance correction (ΔΔG^{‡}_{Q20EFF,Solvent} set). A full in silico site directed mutagenesis potential map was generated for each set of scores based on a series of Lasso models (based on ensembles of 30 random encoding FFT models per seed conformation, see FIG. 55). Table 20 shows the average performance obtained for the ML models for each data set. It was observed that the noise levels increased in the solvent corrected sets (2nd and 4th) resulting in poorer predictive ML model performance. However, a minimal effect on performance was observed from the addition of the lognormal correction to any set. However, the increased noise levels from the incorporation of solvent into the scoring function result in site directed potential maps where fewer residues are observable over the noise baseline (see B and D in FIG. 55). In all cases good targets for DE can be identified, while in practice the increased noise can be compensated by longer MD simulations or larger datasets (and this is only limited by computational resources and time).

TABLE 20
Mean model performance per seed conformation for each set of scores data (average of 30
Lasso models for each conformation and score set. Models encoding complexity N = 40).

| | Mean r² for | Mean r² for | Mean r² for | Mean r² for |
|---|---|---|---|---|
| | models trained | models trained | models trained | models trained |
| Seed | on μ_{Q20, Protein} | on μ_{Q20, Solvent} | on ΔΔG_{Q20EFF, Protein}^{‡} | on ΔΔG_{Q20EFF, Solvent}^{‡} |
| conformation | data | data | data | data |
| 600 ns | 0.2903 | 0.0610 | 0.229 | 0.0470 |
| 700 ns | 0.3641 | 0.1178 | 0.292 | 0.0479 |
| 800 ns | 0.2101 | 0.0628 | 0.161 | 0.0428 |
| 900 ns | 0.3662 | 0.0184 | 0.352 | 0.0179 |
| 1000 ns | 0.3519 | 0.0461 | 0.306 | 0.0168 |

Table 21 displays the first five sites of the sorted average potential per site based on the μ_{Q20,Protein} scores set, with the top 3 sites identified as 13, 98 and 53 in descending relevance order. This subset of sites can be targeted with a total of 742900 distinct library combinations (using the same codon restrictions as in Examples 3 and 4). Further analysis resulted in the identification of sites (and degenerate codons): 13 (WWNS, encoding for any of F, I, K, L, M, N, Y), 53 (NRG, encoding for any of E, G, K, Q, R, W) and 98 (NDT, encoding for any of C, D, F, G, H, I, L, N, R, S, V, Y), resulting in a library with a maximum diversity of 648 distinct variants, based on the best predicted median performance per codon. Furthermore, Tables 22, 23 and 24 display the first five sites of the sorted average potential per site based on the μ_{Q20,solvent} set scores, the ΔΔG^{‡}_{Q20EFF,Protein} set scores and the ΔΔG^{‡}_{Q20EFF,Solvent} set scores, respectively.

Clearly, although the scores obtained from the solvent-corrected sets have a lower statistical significance (due to the amount and length of MD data available), they still identify sites 13 and 53 in the highest ranking (previously also identified based on the ΔΔG^{‡}_{Q20EFF, Protein} and μ_{Q20,Protein} sets), demonstrating that any of these scoring methods could be used interchangeably in the DE process. As described in previous Examples it would be beneficial to use longer MD simulations and larger datasets to improve the statistical significance, which is only limited by time and computational resources. The inclusion of the lognormal correction has a small impact on the selection of the highest-ranking sites and is expected to become more relevant only for more accurate datasets (using longer MD and more exhaustive mutant generation). In practice, the inventors recognise that any dataset could be used. The solvent-corrected data may be advantageous when sufficient data is available to increase model confidence. This will become feasible by increasing the amount of mutant data available and the length of each MD simulation, meaning that in some cases the solvent-corrected sets may be the best option (limited only by the computational resources to obtain sufficient data).

TABLE 21
Top ranked sites based on average potential calculated by
the aggregated Lasso models trained from the μ_{Q20, Protein}
mutant data generated from 5 distinct seed conformations.

| Rank | Site | Score |
|---|---|---|
| 1 | 13 | −2.23088 |
| 2 | 98 | −2.11588 |
| 3 | 53 | −1.71039 |
| 4 | 148 | −1.46952 |
| 5 | 207 | −1.29264 |

TABLE 22
Top ranked sites based on average potential calculated by
the aggregated Lasso models trained from the μ_{Q20, Solvent}
mutant data generated from 5 distinct seed conformations.

| Rank | Site | Score |
|---|---|---|
| 1 | 150 | −4.18496 |
| 2 | 13 | −2.35012 |
| 3 | 53 | −1.67703 |
| 4 | 16 | −1.47926 |
| 5 | 350 | −1.33663 |

TABLE 23
Top ranked sites based on average potential calculated by the
aggregated Lasso models trained from the ΔΔG_{Q20EFF, Protein}^{‡}
mutant data generated from 5 distinct seed conformations.

| Rank | Site | Score |
|---|---|---|
| 1 | 13 | −2.52503 |
| 2 | 53 | −1.91337 |
| 3 | 98 | −1.78486 |
| 4 | 148 | −1.57913 |
| 5 | 207 | −1.20562 |

TABLE 24
Top ranked sites based on average potential calculated by the
aggregated Lasso models trained from the ΔΔG_{Q20EFF, Solvent}^{‡}
mutant data generated from 5 distinct seed conformations.

| Rank | Site | Score |
|---|---|---|
| 1 | 150 | −3.33724 |
| 2 | 13 | −1.94564 |
| 3 | 16 | −1.61128 |
| 4 | 53 | −1.54238 |
| 5 | 350 | −1.38362 |

### Methods

### Initial System Preparation and MD

The initial system was set up based on crystal structure 2EG5 from the protein data bank (PDB). The first 8 residues (amino acid sequence: MELQQVLR (SEQ ID NO:6)) from the PDB sequence (representing a large flexible tail) were removed in this model. Thus, residue numbering as referred to in this example is offset such that residue 1 corresponds to residue 9 of the PDB structure, and the protein sequence starts MNGG (SEQ ID NO:7). Other than this change the amino acid sequence is identical to that in the PDB structure. Molecular dynamics (MD) simulations were performed following the same procedures of Examples 3 and 4. During all MD simulations a set of harmonic restraints were imposed on the substrate and protein to limit the conformational space into structures resembling a near attack conformation (NAC). The series of restraints comprised: the sulphur atom of Cys151 to O4′ of SAM (3.1 Å), xanthosine N7 to SAM CD (2.5 Å) and xanthosine 02 to the hydroxyl oxygen of Tyr348 (2.5 Å), and all used a force constant of 1000 kJ×Å⁻². The wild type (WT) enzyme was subjected to a 1 μs MD simulation and 3D-structures corresponding to timeframes 600 ns, 700 ns, 800 ns, 900 ns and 1000 ns were extracted for mutant generation. See FIG. 50 for a diagram of the reactant complex (RC) structure and the transition state (TS) structures (including atom name specification of key atoms for the substrate and cofactor).

### In Silico Mutagenesis and Mutant Scoring

A set of 20619 triple mutants were generated randomly, targeting any site except for residues Cys151 and Tyr348, due to their role in restraining the cofactor and substrate in the MD simulations. All other sites were targeted including cysteine-containing sites and the N-terminus and C-terminus residues. Mutants were generated by modifying the side chain structures starting from any seed conformation. All amino acid insertions were allowed, including cysteine since this small protein has no disulphide bonds. All mutants were then minimised before running a 1.0 ns MD simulation. All saved coordinate sets (10 per 1 ns) were scored by the previously described Q20 methodology. Furthermore, a series of 4250 mutants each were also generated containing 6, 12 and 24 mutants respectively (namely sets XMT6, XMT12 and XMT24).

### Transition State Search and Q20 Parameterisation

The coordinates corresponding to frames 2257, 3653 and 5210 from the WT MD simulation (these corresponded to simulation times of 225.7 ns, 365.3 ns and 521.0 ns, and were selected arbitrarily) were used as base structures to obtain a series of optimised transition state structures and an optimised reactant complex structure for each of the frames, via a DFT cluster model method following the same procedures used in Example 4. Each DFT model was defined to include the substrate, the cofactor, and several water residues. Constraints were imposed to preserve the protein conformations of the complex, specifically residues N, N6 and O for the SAM cofactor, and O2 O3′ and O5′ forthe substrate xanthosine (the atom names were as detailed in the PDB structure and see FIG. 50). The change in partial atomic charges was calculated following the same procedures of Example 4 for the parameterisation of the Q20 scorer (only the substrate and xanthosine were included in the core region of the Q20 scorer). For each atom in the core region, the average of the change in partial atomic charges across the three frames was calculated as a representative partial charge change (using the three transition state and three reactant complex structures). The proposed mechanism described in [167] was used as a reference to optimise the TS structure.

### Machine Learning Ensemble

A series of regularised Lasso models were trained and aggregated for ensemble predictions as described in Examples 3 and 4. In short, all the training data was encoded using a random encoding ProSAR methodology, where no amino acid property database was required, followed by performing an FFT on the encoded data. The ML models were grouped into subsets and trained on data from specific conformations only. Unseen validation and test subsets were created to monitor performance and training cycles of the ML models. A set of 30 regularised Lasso models were generated for each conformation set (150 models in total).

### Site Directed Mutagenesis Potential Map

For each ML model, a prediction of catalytic turnover improvement was obtained for every possible single-mutant. These predictions were then standardised for each model output to a mean of 0 and a standard deviation of 1, while the calculated mean and variance for each model was stored for further model standardisation. For each single mutant prediction, a mean across all models in the ensemble was calculated. Furthermore, a site-specific mean, maximum and minimum (based on all possible amino acid substitutions per site, e.g., 20) was calculated to obtain a metric that could be calculated at each site, which is shown in FIG. 53 for each scoring variation.

### In Silico PCR Based DE Library

Once a set of sites had been selected, further optimisation was performed to choose the specific best codons. Each possible library included mutants with more than one individual mutation, and therefore the mutants were individually predicted based on each ML model. The predictions form each ML model were corrected for standardisation based on the specific means and variances previously calculated during the full-enzyme saturation potential map prediction (see FIG. 31 for the standardised process of individual mutant scoring). Each library (defined by a selection of sites and degenerate codons) was then compared by scoring each mutant in the library and calculating the median value of the scores. A total 742900 distinct library combinations are possible for these sites using the following codon restrictions: inclusive of the wild-type amino acid, no stop codons, only encoding once for each amino acid, and encoding for no more than 12 amino acids. The combinatorial libraries were scored and ranked accordingly, resulting in a top selection of sites and degenerate codons.

### Conclusions

In this Example, the inventors built on previous demonstrations of the invention by expanding the exemplification to a transferase (belonging to EC-2) containing an unbound S-adenosyl methionine cofactor. These results further show that the applicability of the herein described invention (for the computational guidance of DE) to any enzyme class potentially and to enzymes containing any of a variety of cofactors. In this example, it was shown that the use of either NVT or NPT conditions for MD, the inclusion of solvent and counter ion effects on the μ_{Q20} electrostatic estimation of mutants will have no practical impact to the predictions. Furthermore, it was shown that introducing more individual mutants into the MD models can result in datasets of similar practical use under the current invention by introducing either 3, 6, 12 or 24 mutations in distinct data sets. Although the noise levels increase, the inventors recognise that further addition of mutants may also help ML models recognise epistatic effects (mutant-mutant interaction or cooperative effects).

### Example 7: Accelerating Directed Evolution with Machine Learning Based on Dynamics-Driven Predictions of Enzyme Catalytic Turnover Number Applied to a Lyase (EC-4)

The inventors have built on the successes described in Examples 1 to 6 to illustrate the application of the methodology to other enzymes, in this case an example is shown of how computationally guided DE of hydroxynitrile lyase would be performed. The inventors have used the previously described methodology to design more efficient evolutionary libraries and that could be used in DE iterations to discover better variants with a higher catalytic rate of cyanohydrin cleavage. In showing this, the inventors confirm its use in a wider class of enzymes and show how the process can be generalised to longer MD simulations. Furthermore, it was recognised that a single seed conformation can be used in practice for ML training for this process.

Hydroxynitrile lyases are valuable enzymes that belong to the EC-4 lyase enzyme class. These enzymes are involved in the asymmetric synthesis of cyanohydrins, which are a series of nitrile-containing compounds actively used in the production of many commercial applications in pharmaceuticals and agrochemicals. For this reason, hydroxynitrile lyases have been a frequent target for protein engineering [169]. The inventors recognise the benefits of engineering new and better enzyme variants of this class and recognise that (R)-hydroxynitrile lyase from the Arabidopsis thaliana (AtHNL), an enantiomerically (R)-selective enzyme of this class, could be engineered and further repurposed for use in many applications (such as in API biosynthesis) and show how the methodology described herein can be used for the computations generation of libraries for hydroxynitrile lyase that can be used in DE of this enzyme to improve its catalytic turnover number.

### Results

A system comprising the AtHNL enzyme and the substrate (R)-mandelonitrile (MAN) was prepared by manually docking the substrate into the active site to adopt a conformation equivalent to that observed previously [168]. A total of 1 μs of molecular dynamics (MD) was performed to equilibrate the wild type (WT) system. Subsequently, the 3D-structure from the last time frame (1000 ns) was selected to generate mutant data for ML training. A set (containing 1000 random triple mutants) was generated and (following the computational preparation procedure of the mutant simulations, as described in Example 2), a total of 50 ns of MD was performed on each mutant variant (comprising 500 sampled conformations, separated linearly by 0.1 ns of simulation time). These MD simulations were 50 times longer than those performed on mutants of Examples 3 to 6.

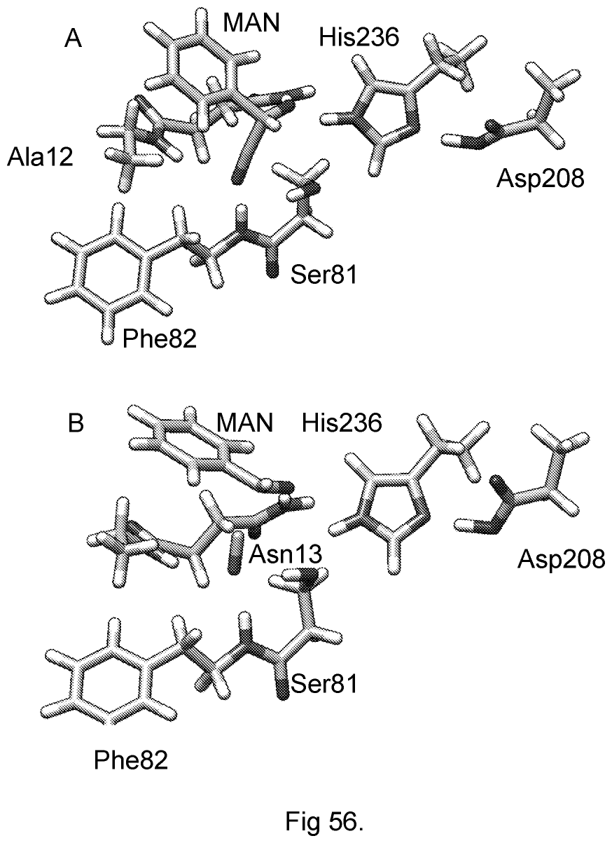

The Q20 methodology was used to score each sampled conformation from the mutant MD simulations and a single μ_{Q20} score was obtained for each mutant (as described in Example 1). The parameters for the Q20 scores were based on DFT cluster optimised models of the enzyme following a similar approach to Examples 4 to 6 (see FIG. 56 for the visualisation of the optimised RC and TS structures). The inventors recognise that although the number of explored mutants is significantly smaller (at only 1000) than in previous Examples 4 to 6, there is also a reduction in noise levels for each scored mutant due to the longer MD simulations used. Increasing the number of mutants and/or the amount of MD to be produced for each mutant is generally beneficial and is only be constrained by the availability of computational resources.

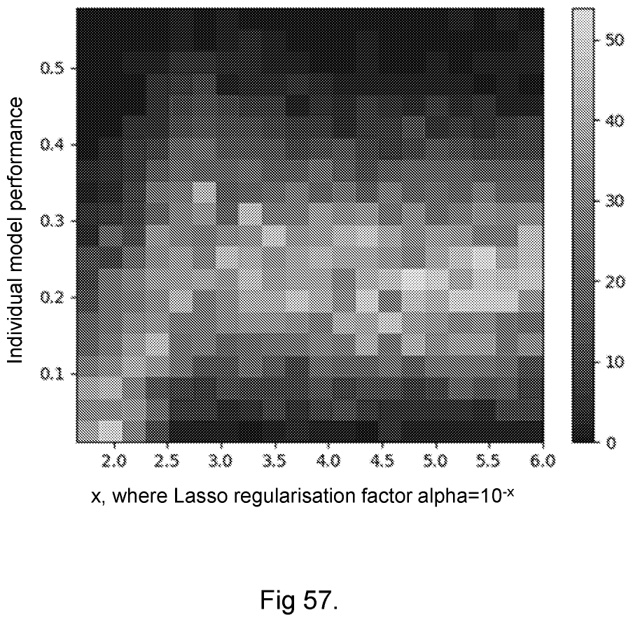

Two different encoding methods were employed. The first method was a random encoding methodology including an FFT as described in Example 3, with an encoding complexity of N=1. To assess these results, a series of 30 Lasso models were employed (α=10-4) and a mean test score of 0.068 was obtained, suggesting that over-fitting was taking place due to the low number of mutants available even at an encoding complexity of N=1. Therefore, a second method of encoding was introduced that was based only on the selection of target site (irrespective of amino acid substitution), with the aim of greatly reducing encoding complexity below that possible with the random FFT encoding methodology. The second method of encoding used a one hot encoding per-site approach. A series of regularised Lasso models was used with this second encoding and a grid search was used to fine tune the α regularisation factor (see FIG. 57). A total of 2000 models were trained in an ensemble, each training on a random subset of 92% of the data (and testing on the remainder). An average performance of 0.273 was obtained by this approach, with a better performance than by the random FFT encoded models. Therefore, it was concluded that ML methods and the associated encoding method can be adapted to suit the amount and type of data.

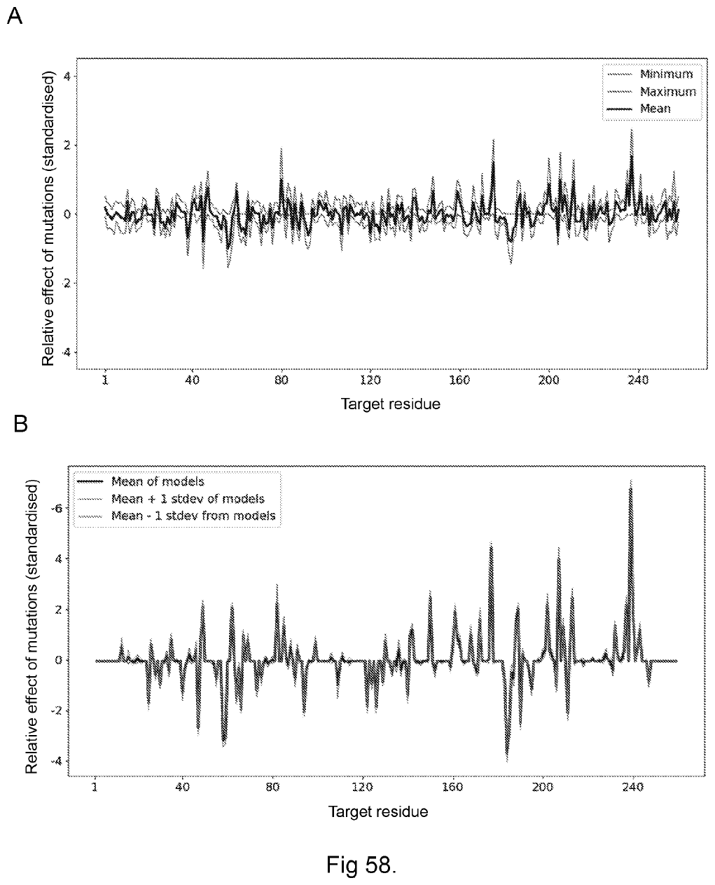

The results from each of the encoding and modelling methods were standardised before aggregation for a mean of 0 and standard deviation of 1.0 and two full in silico site directed mutagenesis potential maps (one for each used encoding method) were generated to visualise the representation of the best residues for mutagenesis for their potential in the design of highly effective DE libraries (see FIG. 58). A better model performance was observed with the one hot per-site encoding, which resulted in a higher confidence in the identification of high-ranking target sites.

Table 25 displays the sorted average potential per site of the one hot per-site encoded results, with the top three sites identified as Asp183, Glu57, and Tyr58 in descending relevance order. Due to the nature of the encoding process, no specific amino acid substitutions could be predicted. However, it may be reasonable to choose a large degenerate codon such as NDT (12 amino acids per site, encoding for any of R, N, D, C, G, H, I, L, F, S, Y, V) or even NNK, resulting in libraries of maximum diversity of 1728 or 8000, respectively, when no more information is available. Thus, the inventors recognise that any size of codon including exhaustive NNK codons might be used when the number of training mutants is low and specific amino acid predictions cannot be made at each site. However, several high-ranking targets are still identified outside the active site. Therefore, this represents an improvement on most DE processes, which generally focus on the active site or regions close to it.

TABLE 25
Highest ranking sites based on the scoring of the top ensemble
of Lasso models. More negative scores are better.

| Rank | Site | Score standard deviations |
|---|---|---|
| 1 | 183 | −3.73311 |
| 2 | 57 | −3.20719 |
| 3 | 58 | −3.11547 |
| 4 | 184 | −2.91458 |
| 5 | 46 | −2.70603 |

### Methods

### Initial System Preparation and MD

The initial system was set up based on the 3DQZ crystal structure from the protein data bank. Molecular dynamics (MD) simulations were performed following the same procedures of Examples 3 and 4. During all MD simulations a set of harmonic restraints were imposed on the substrate and protein to limit the conformational space into structures resembling a near attack conformation (NAC). The series of restraints comprised: His236 to the substrate O1 (3.0 Å), Ala13 N to substrate N1 (3.0 Å), and all used a force constant of 1000 kJ×Å⁻². The wild type (WT) enzyme was subjected to a 1 μs MD simulation and the 3D-structure corresponding to timeframe 1000 ns was extracted for the mutant generation. In silico mutagenesis and Mutant scoring A set comprising 1000 triple mutants was generated randomly, targeting any site except for residues 12, 13, 81, 82, 208 and 236, which had been recognised as potentially relevant residues in the mechanism of reaction a priori. Similarly, any sites containing cysteine residues were avoided and no residues were mutated into cysteine (for reasons described previously). The first 10 residues on the N terminus as well as the last 10 residues of the C terminus of the enzyme were also left unchanged due to the higher dynamic variability typically observed on these regions. Mutants were generated by modifying the side chain structures computationally from the 3D-structure of the parent seed conformation. All mutants were then prepared for MD simulation (as described in Example 2), before running a 50 ns MD simulation on each mutant. All sampled frames (500 per mutant at 0.1 ns intervals) were scored by the previously described Q20 methodology.

### Transition State Search and Q20 Parameterisation

A transition state optimisation was performed based on a mechanistic model proposed previously [168]. An arbitrary frame from the WT MD simulation corresponding to 210.0 ns was used as a base structure to search and obtain a rate-limiting transition state (TS) structure and the optimised reactant complex (RC) structure via a DFT cluster model, following the same procedures used in Example 4. The cluster model included the substrate and residues Asn12, Ala13, Ser81, Phe82 and Asp208 (see FIG. 56 for visualisation of the optimised reactant complex and optimised transition state coordinates, respectively). The change in partial atomic charges was calculated following the procedures of Example 4 to parameterise the Q20 scorer. The core region o for the Q20 scoring was defined to include residues 81 and 236 and the substrate.

### Machine Learning Ensemble

The mutant data was encoded using two distinct methods. The first method employed was random encoding FFT (as described in Example 3). A second method was introduced, namely one hot encoded per-site encoding, to reflect the sites mutated on each variant as a sequence of zeros for all residues except for the mutated residues for which a one was assigned. Therefore, a sequence of length 258 represented each variant. A series of 2000 regularised Lasso models were employed to model the data, training each on a randomly split set of 92% training data and tested against the remaining 8%. The regularisation parameter a was also optimised to increase the mean model performance (see FIG. 57). An identity matrix of size 258×258 was used as the input to predict a set of mutants (representing a list of mutants, each mutant containing a single mutation and together covering every possible site). Each output was standardised to a mean of 0 and standard deviation of 1 for each model, before a mean of all the models was obtained as a final calculation. No specific codon optimisations were performed due to the one hot encoding used in this example (which is not residue specific), while large codons may be used in a combinatorial library to target these sites and explore a significant subset (via e.g., NDT) or a comprehensive set (via e.g., NNK) of the mutants experimentally.

### Conclusions

In this Example, the inventors built on to previous exemplification by expanding to a lyase class enzyme (belonging to EC4), which further confirms that the applicability of the herein described invention (for the computational guidance of DE) extends to any enzyme class. Moreover, some parameters have been modified to exemplify that such variations can be introduced, including the use of longer MD simulations (of 50 ns instead of 1 ns as demonstrated in Examples 3 to 6) to obtain the μ_{Q20} electrostatic estimation of mutants for use in ML as well as with shorter lists of mutants from a single seed conformation (1000 mutants only). Although the ML performance decreases from the smaller dataset, it is recognised that the encoding methodology can improve performance by encoding only for the site of mutation (irrespective of the amino acid substitution) when there is a smaller amount of mutant data. The inventors further recognise that the best ML method may vary depending on the nature and amount of data. It is also recognised that when only the site of the mutation can be reliably predicted then larger codons such as fully degenerate NNK or NDT codons can be used at these sites. Further addition of mutants (which will result in improving models and experimental success) and the extension of the MD simulations beyond 50 ns are beneficial, but these do not pose technical difficulties per se that need to be addressed by variations in the methodology, they are external limitations, imposed by the computational resources and time available.

### REFERENCES

1. Honig, M.; Sondermann, P.; Turner, N. J.; Carreira, E. M., Enantioselective Chemo- and Biocatalysis: Partners in Retrosynthesis. Angew. Chem. Int. Ed. 2017, 56, 8942-8973.
2. Sheldon, R. A. A.; Brady, D.; Bode, M. L. L., The Hitchhiker's Guide to Biocatalysis: Recent Advances in the use of Enzymes in Organic Synthesis. Chem. Sci. 2020, 11, 2587-2605.
3. Mangas-Sanchez, J.; Sharma, M.; Cosgrove, S. C.; Ramsden, J. I.; Marshall, J. R.; Thorpe, T. W.; Palmer, R. B.; Grogan, G.; Turner, N. J., Asymmetric Synthesis of Primary Amines Catalyzed by Thermotolerant Fungal Reductive Aminases. Chem. Sci. 2020, 11, 5052-5057.
4. Herter, S.; Medina, F.; Wagschal, S.; Benhaim, C.; Leipold, F.; Turner, N. J., Mapping the Substrate Scope of Monoamine Oxidase (MAO-N) as a Synthetic Tool for the Enantioselective Synthesis of Chiral Amines. Bioorgan. Med. Chem. 2018, 26, 1338-1346.
5. Ghislieri, D.; Green, A. P.; Pontini, M.; Willies, S. C.; Rowles, I.; Frank, A.; Grogan, G.; Turner, N. J., Engineering an Enantioselective Amine Oxidase for the Synthesis of Pharmaceutical Building Blocks and Alkaloid Natural Products. J. Am. Chem. Soc. 2013, 135, 10863-10869.
6. Heath, R. S.; Pontini, M.; Bechi, B.; Turner, N. J., Development of an R-Selective Amine Oxidase with Broad Substrate Specificity and High Enantioselectivity. ChemCatChem 2014, 6, 996-1002.
7. Currin, A.; Swainston, N.; Day, P. J.; Kell, D. B., Synthetic Biology for the Directed Evolution of Protein Biocatalysts: Navigating Sequence Space Intelligently. Chem. Soc. Rev. 2015, 44, 1172-1239.
8. Arnold, F. H., Directed Evolution: Bringing New Chemistry to Life. Angew. Chem. nt. Ed. 2018, 57, 4143-4148.
9. Suel, G. M.; Lockless, S. W.; Wall, M. A.; Ranganathan, R., Evolutionarily Conserved Networks of Residues Mediate Allosteric Communication in Proteins. Nat. Struct. Biol. 2003, 10, 59-69.
10. Wong, K. F.; Selzer, T.; Benkovic, S. J.; Hammes-Schiffer, S., Impact of Distal Mutations on the Network of Coupled Motions Correlated to Hydride Transfer in Dihydrofolate Reductase. Proc. Natl. Acad. Sci. U.S.A. 2005, 102, 6807-6812.
11. Reetz, M. T.; Wang, L. W.; Bocola, M., Directed Evolution of Enantioselective Enzymes: Iterative Cycles of CASTing for Probing Protein-Sequence Space. Angew. Chem. nt. Ed. 2006, 45, 1236-1241.
12. Reetz, M. T.; Carballeira, J. D., Iterative Saturation Mutagenesis (ISM) for Rapid Directed Evolution of Functional Enzymes. Nat. Protoc. 2007, 2, 891-903.
13. Qu, G.; Li, A.; Acevedo-Rocha, C. G.; Sun, Z.; Reetz, M. T., The Crucial Role of Methodology Development in Directed Evolution of Selective Enzymes. Angew. Chem. Int. Ed. Engl. 2020, 59, 13204-13231.
14. Currin, A.; Swainston, N.; Day, P. J.; Kell, D. B., SpeedyGenes: an Improved Gene Synthesis Method for the Efficient Production of Error-Corrected, Synthetic Protein Libraries for Directed Evolution. Protein Eng. Des. Sel. 2014, 27, 273-80.
15. Romero-Rivera, A.; Garcia-Borras, M.; Osuna, S., Computational Tools for the Evaluation of Laboratory-Engineered Biocatalysts. ChemComm 2017, 53, 284-297.
16. Eyring, H., The Activated Complex in Chemical Reactions. J. Chem. Phys. 1935, 3, 107-115.
17. Ahmadi, S.; Herrera, L. B.; Chehelamirani, M.; Hostas, J.; Jalife, S.; Salahub, D. R., Multiscale Modeling of Enzymes: QM-cluster, QM/MM, and QM/MM/MD: A Tutorial Review. Int. J. Quantum Chem. 2018, 118, e25558.
18. Lonsdale, R.; Harvey, J. N.; Mulholland, A. J., A Practical Guide to Modelling Enzyme-Catalysed Reactions. Chem. Soc. Rev. 2012, 41, 3025-38.
19. Himo, F., Recent Trends in Quantum Chemical Modeling of Enzymatic Reactions. J. Am. Chem. Soc. 2017, 139, 6780-6786.
20. van der Kamp, M. W.; Mulholland, A. J., Combined Quantum Mechanics/Molecular Mechanics (QM/MM) Methods in Computational Enzymology. Biochemistry 2013, 52, 2708-28.
21. Senn, H. M.; Thiel, W., QM/MM Methods for Biomolecular Systems. Angew. Chem. Int. Ed. Engl. 2009, 48, 1198-229.
22. Quesne, M. G.; Borowski, T.; de Visser, S. P., Quantum Mechanics/Molecular Mechanics Modeling of Enzymatic Processes: Caveats and Breakthroughs. Chem. Eur. J. 2016, 22, 2562-2581.
23. Martins-Costa, M. T. C.; Ruiz-Lopez, M. F., Reaching Multi-Nanosecond Timescales in Combined QM/MM Molecular Dynamics Simulations through Parallel Horsetail Sampling. J. Comp. Chem. 2017, 38, 659-668.
24. Boehr, D. D.; Dyson, H. J.; Wright, P. E., An NMR Perspective on Enzyme Dynamics. Chem. Rev. 2006, 106, 3055-3079.
25. Shaw, D. E.; Maragakis, P.; Lindorff-Larsen, K.; Piana, S.; Dror, R. O.; Eastwood, M. P.; Bank, J. A.; Jumper, J. M.; Salmon, J. K.; Shan, Y. B.; Wriggers, W., Atomic-Level Characterization of the Structural Dynamics of Proteins. Science 2010, 330, 341-346.
26. Jindal, G.; Slanska, K.; Kolev, V.; Damborsky, J.; Prokop, Z.; Warshel, A., Exploring the Challenges of Computational Enzyme Design by Rebuilding the Active Site of a Dehalogenase. Proc. Natl. Acad. Sci. U.S.A. 2019, 116, 389-394.
27. McGeagh, J. D.; Ranaghan, K. E.; Mulholland, A. J., Protein dynamics and enzyme catalysis: Insights from simulations. Biochim. Biophys. Acta Proteins Proteom. 2011, 1814, 1077-1092.
28. Warshel, A., Dynamics of Enzymatic-Reactions. Proc. Natl. Acad. Sci. U.S.A. 1984, 81, 444-448.
29. Villa, J.; Warshel, A., Energetics and Dynamics of Enzymatic Reactions. J. Phys. Chem. B 2001, 105, 7887-7907.
30. Romero-Rivera, A.; Garcia-Borras, M.; Osuna, S., Role of Conformational Dynamics in the Evolution of Retro-Aldolase Activity. ACS Catal. 2017, 7, 8524-8532.
31. Jimenez-Oses, G.; Osuna, S.; Gao, X.; Sawaya, M. R.; Gilson, L.; Collier, S. J.; Huisman, G. W.; Yeates, T. O.; Tang, Y.; Houk, K. N., The Role of Distant Mutations and Allosteric Regulation on LovD Active Site Dynamics. Nat. Chem. Biol. 2014, 10, 431-436.
32. Hammes-Schiffer, S.; Watney, J. B., Hydride Transfer Catalysed by Escherichia coli and Bacillus subtilis Dihydrofolate Reductase: Coupled Motions and Distal mutations. Proc. Royal Soc. B 2006, 361, 1365-1373.
33. Edwards, S. J.; Soudackov, A. V.; Hammes-Schiffer, S., Impact of Distal Mutation on Hydrogen Transfer Interface and Substrate Conformation in Soybean Lipoxygenase. J. Phys. Chem. B 2010, 114, 6653-6660.
34. Wang, L.; Goodey, N. M.; Benkovic, S. J.; Kohen, A., Coordinated Effects of Distal Mutations on Environmentally Coupled Tunneling in Dihydrofolate Reductase. Proc. Natl. Acad. Sci. U.S.A. 2006, 103, 15753-15758.
35. Leidner, F.; Yilmaz, N. K.; Schiffer, C. A., Deciphering Complex Mechanisms of Resistance and Loss of Potency through Coupled Molecular Dynamics and Machine Learning. bioRxiv 2020, 2020.06.08.139105.
36. Kamerlin, S. C. L.; Warshel, A., The Empirical Valence Bond Model: Theory and Applications. Wiley Interdiscip. Rev. Comput. Mol. Sci. 2011, 1, 30-45.
37. Frushicheva, M. P.; Cao, J.; Chu, Z. T.; Warshel, A., Exploring Challenges in Rational Enzyme Design by Simulating the Catalysis in Artificial Kemp Eliminase. Proc. Natl. Acad. Sci. U.S.A. 2010, 107, 16869-16874.
38. Bradshaw, R. T.; Dziedzic, J.; Skylaris, C. K.; Essex, J. W., The Role of Electrostatics in Enzymes: Do Biomolecular Force Fields Reflect Protein Electric Fields? J. Chem. Inf. Model 2020, 60, 3131-3144.
39. Warshel, A.; Sharma, P. K.; Kato, M.; Xiang, Y.; Liu, H. B.; Olsson, M. H. M., Electrostatic Basis for Enzyme Catalysis. Chem. Rev. 2006, 106, 3210-3235.
40. Prah, A.; Franciskovic, E.; Mavri, J.; Stare, J., Electrostatics as the Driving Force Behind the Catalytic Function of the Monoamine Oxidase A Enzyme Confirmed by Quantum Computations. ACS Catal. 2019, 9, 1231-1240.
41. Warshel, A., Multiscale Modeling of Biological Functions: From Enzymes to Molecular Machines (Nobel Lecture). Angew. Chem. Int. Ed. 2014, 53, 10020-10031.
42. Ryde, U., A Fundamental View of Enthalpy-Entropy Compensation. Medchemcomm 2014, 5, 1324-1336.
43. Lumry, R., Uses of Enthalpy-Entropy Compensation in Protein Research. Biophys. Chem. 2003, 105, 545-557.
44. Koetter, J. W. A.; Schulz, G. E., Crystal Structure of 6-Hydroxy-D-Nicotine Oxidase from Arthrobacter nicotinovorans. J. Mol. Biol. 2005, 352, 418-428.
45. Fiser, A.; Do, R. K. G.; Sali, A., Modeling of Loops in Protein Structures. Protein Sci. 2000, 9, 1753-1773.
46. Olsson, M. H. M.; Sondergaard, C. R.; Rostkowski, M.; Jensen, J. H., PROPKA3: Consistent Treatment of Internal and Surface Residues in Empirical pKa Predictions. J. Chem, Theor. Comp. 2011, 7, 525-537.
47. Brandsch, R.; Hinkkanen, A. E.; Mauch, L.; Nagursky, H.; Decker, K., 6-Hydroxy-D-Nicotine Oxidase of Arthrobacter oxidans—Gene Structure of the Flavoenzyme and its Relationship to 6-Hydroxy-L-Nicotine Oxidase. Eur. J. Biochem. 1987, 167, 315-320.
48. Wang, J. M.; Wang, W.; Kollman, P. A.; Case, D. A., Automatic Atom Type and Bond Type Perception in Molecular Mechanical Calculations. J. Mol. Graph. Model. 2006, 25, 247-260.
49. Bayly, C. I.; Cieplak, P.; Cornell, W. D.; Kollman, P. A., A Well-Behaved Electrostatic Potential Based Method Using Charge Restraints for Deriving Atomic Charges—the RESP Model. J. Phys. Chem. 1993, 97, 10269-10280.
50. Frisch, M. J.; Trucks, G. W.; Schlegel, H. B.; Scuseria, G. E.; Robb, M. A.; Cheeseman, J. R.; Scalmani, G.; Barone, V.; Petersson, G. A.; Nakatsuji, H.; Li, X.; Caricato, M.; Marenich, A. V.; Bloino, J.; Janesko, B. G.; Gomperts, R.; Mennucci, B.; Hratchian, H. P.; Ortiz, J. V.; Izmaylov, A. F.; Sonnenberg, J. L.; Williams; Ding, F.; Lipparini, F.; Egidi, F.; Goings, J.; Peng, B.; Petrone, A.; Henderson, T.; Ranasinghe, D.; Zakrzewski, V. G.; Gao, J.; Rega, N.; Zheng, G.; Liang, W.; Hada, M.; Ehara, M.; Toyota, K.; Fukuda, R.; Hasegawa, J.; Ishida, M.; Nakajima, T.; Honda, Y.; Kitao, O.; Nakai, H.; Vreven, T.; Throssell, K.; Montgomery Jr., J. A.; Peralta, J. E.; Ogliaro, F.; Bearpark, M. J.; Heyd, J. J.; Brothers, E. N.; Kudin, K. N.; Staroverov, V. N.; Keith, T. A.; Kobayashi, R.; Normand, J.; Raghavachari, K.; Rendell, A. P.; Burant, J. C.; lyengar, S. S.; Tomasi, J.; Cossi, M.; Millam, J. M.; Klene, M.; Adamo, C.; Cammi, R.; Ochterski, J. W.; Martin, R. L.; Morokuma, K.; Farkas, O.; Foresman, J. B.; Fox, D. J. Gaussian 09 Revision D.01, Gaussian Inc., Wallingford, C T, 2016.
51. Maier, J. A.; Martinez, C.; Kasavajhala, K.; Wickstrom, L.; Hauser, K. E.; Simmerling, C., FF14SB: Improving the Accuracy of Protein Side Chain and Backbone Parameters from FF99SB. J. Chem, Theor. Comp. 2015, 11, 3696-3713.
52. Jorgensen, W. L.; Chandrasekhar, J.; Madura, J. D.; Impey, R. W.; Klein, M. L., Comparison of Simple Potential Functions for Simulating Liquid Water. J. Chem. Phys. 1983, 79, 926-935.
53. Eastman, P.; Swails, J.; Chodera, J. D.; McGibbon, R. T.; Zhao, Y. T.; Beauchamp, K. A.; Wang, L. P.; Simmonett, A. C.; Harrigan, M. P.; Stern, C. D.; Wiewiora, R. P.; Brooks, B. R.; Pande, V. S., OpenMM 7: Rapid Development of High Performance Algorithms for Molecular Dynamics. PLoS Comput. Biol. 2017, 13, e1005659.
54. Hopkins, C. W.; Le Grand, S.; Walker, R. C.; Roitberg, A. E., Long-Time-Step Molecular Dynamics through Hydrogen Mass Repartitioning. J. Chem, Theor. Comp. 2015, 11, 1864-1874.
55. Fitzpatrick, P. F., Oxidation of Amines by Flavoproteins. Arch. Biochem. Biophys. 2010, 493, 13-25.
56. Abe, Y.; Shoji, M.; Nishiya, Y.; Aiba, H.; Kishimoto, T.; Kitaura, K., The Reaction Mechanism of Sarcosine Oxidase Elucidated Using FMO and QM/MM Methods. Phys. Chem. Chem. Phys. 2017, 19, 9811-9822.
57. Cakir, K.; Erdem, S. S.; Atalay, V. E., ONIOM Calculations on Serotonin Degradation by Monoamine Oxidase B: Insight into the Oxidation Mechanism and Covalent Reversible Inhibition. Org. Biomol. Chem. 2016, 14, 9239-9252.
58. Karasulu, B.; Thiel, W., Amine Oxidation Mediated by N-Methyltryptophan Oxidase: Computational Insights into the Mechanism, Role of Active-Site Residues, and Covalent Flavin Binding. ACS Catal. 2015, 5, 1227-1239.
59. Repic, M.; Vianello, R.; Purg, M.; Duarte, F.; Bauer, P.; Kamerlin, S. C. L.; Mavri, J., Empirical Valence Bond Simulations of the Hydride Transfer Step in the Monoamine Oxidase B Catalyzed Metabolism of Dopamine. Proteins 2014, 82, 3347-3355.
60. Neese, F., Software Update: the ORCA Program System, Version 4.0. Wiley Interdiscip. Rev. Comput. Mol. Sci. 2018, 8, e1327.
61. Lee, C. T.; Yang, W. T.; Parr, R. G., Development of the Colle-Salvetti Correlation-Energy Formula into a Functional of the Electron-Density. Phys. Rev. B 1988, 37, 785-789.
62. Becke, A. D., Density-Functional Thermochemistry 0.3. The Role of Exact Exchange. J. Chem. Phys. 1993, 98, 5648-5652.
63. Stephens, P. J.; Devlin, F. J.; Chabalowski, C. F.; Frisch, M. J., Ab Initio Calculation of Vibrational Absorption and Circular-Dichroism Spectra Using Density-Functional Force-Fields. J. Phys. Chem. 1994, 98, 11623-11627.
64. Weigend, F.; Ahlrichs, R., Balanced Basis Sets of Split Valence, Triple Zeta Valence and Quadruple Zeta Valence Quality for H to Rn: Design and Assessment of Accuracy. Phys. Chem. Chem. Phys. 2005, 7, 3297-3305.
65. Grimme, S.; Ehrlich, S.; Goerigk, L., Effect of the Damping Function in Dispersion Corrected Density Functional Theory. J. Comp. Chem. 2011, 32, 1456-1465.
66. Sherwood, P.; de Vries, A. H.; Guest, M. F.; Schreckenbach, G.; Catlow, C. R. A.; French, S. A.; Sokol, A. A.; Bromley, S. T.; Thiel, W.; Turner, A. J.; Billeter, S.; Terstegen, F.; Thiel, S.; Kendrick, J.; Rogers, S. C.; Casci, J.; Watson, M.; King, F.; Karlsen, E.; Sjovoll, M.; Fahmi, A.; Schafer, A.; Lennartz, C., QUASI: A General Purpose Implementation of the QM/MM Approach and its Application to Problems in Catalysis. J. Mol. Sctruc. Theochem 2003, 632, 1-28.
67. Kastner, J.; Carr, J. M.; Keal, T. W.; Thiel, W.; Wander, A.; Sherwood, P., DL-FIND: An Open-Source Geometry Optimizer for Atomistic Simulations. J. Phys. Chem. A 2009, 113, 11856-11865.
68. Ahlrichs, R.; Bar, M.; Haser, M.; Horn, H.; Kolmel, C., Electronic-Structure Calculations on Workstation Computers—the Program System Turbomole. Chem. Phys. Lett. 1989, 162, 165-169.
69. Smith, W.; Forester, T. R., DL_POLY_2.0: A General-Purpose Parallel Molecular Dynamics Simulation Package. J. Mol. Graphics 1996, 14, 136-141.
70. Todorov, I. T.; Smith, W.; Trachenko, K.; Dove, M. T., DL_POLY_3: New Dimensions in Molecular Dynamics Simulations via Massive Parallelism. J. Mater. Sci. 2006, 16, 1911-1918.
71. Huang, J.; Rauscher, S.; Nawrocki, G.; Ran, T.; Feig, M.; de Groot, B. L.; Grubmuller, H.; MacKerell, A. D., CHARMM36m: an Improved Force Field for Folded and Intrinsically Disordered Proteins. Nat. Methods 2017, 14, 71-73.
72. Gutierrez, I. S.; Lin, F. Y.; Vanommeslaeghe, K.; Lemkul, J. A.; Armacost, K. A.; Brooks, C. L.; MacKerell, A. D., Parametrization of Halogen Bonds in the CHARMM General Gorce Field: Improved Treatment of Ligand-Protein Interactions. Bioorgan. Med. Chem. 2016, 24, 4812-4825.
73. Vanommeslaeghe, K.; Hatcher, E.; Acharya, C.; Kundu, S.; Zhong, S.; Shim, J.; Darian, E.; Guvench, O.; Lopes, P.; Vorobyov, I.; MacKerell, A. D., CHARMM General Force Field: A Force Field for Drug-Like Molecules Compatible with the CHARMM All-Atom Additive Biological Force Fields. J. Comp. Chem. 2010, 31, 671-690.
74. Zoete, V.; Cuendet, M. A.; Grosdidier, A.; Michielin, O., SwissParam: A Fast Force Field Generation Tool for Small Organic Molecules. J. Comp. Chem. 2011, 32, 2359-2368.
75. Zhao, Y.; Truhlar, D. G., The M06 Suite of Density Functionals for Main Group Thermochemistry, Thermochemical Kinetics, Noncovalent Interactions, Excited States, and Transition Elements: Two New Functionals and Systematic Testing of Four M06-Class Functionals and 12 Other Functionals. Theor. Chem. Acc. 2008, 120, 215-241.
76. Petersson, G. A.; Allaham, M. A., A Complete Basis Set Model Chemistry. 2. Open-Shell Systems and the Total Energies of the 1st-Row Atoms. J. Chem. Phys. 1991, 94, 6081-6090.
77. Marenich, A. V.; Cramer, C. J.; Truhlar, D. G., Universal Solvation Model Based on Solute Electron Density and on a Continuum Model of the Solvent Defined by the Bulk Dielectric Constant and Atomic Surface Tensions. J. Phys. Chem. B 2009, 113, 6378-6396.
78. Marenich, A. V.; Jerome, S. V.; Cramer, C. J.; Truhlar, D. G., Charge Model 5: An Extension of Hirshfeld Population Analysis for the Accurate Description of Molecular Interactions in Gaseous and Condensed Phases. J. Chem, Theor. Comp. 2012, 8, 527-541.
79. Frauenfelder, H.; Petsko, G. A.; Tsernoglou, D., Temperature-Dependent X-Ray-Diffraction as a Probe of Protein Structural Dynamics. Nature 1979, 280, 558-563.
80. Lange, O. F.; Grubmuller, H., Generalized Correlation for Biomolecular Dynamics. Proteins 2006, 62, 1053-1061.
81. Reetz, M. T.; Kahakeaw, D.; Lohmer, R., Addressing the Numbers Problem in Directed Evolution. ChemBioChem 2008, 9, 1797-1804.
82. Li, A. T.; Acevedo-Rocha, C. G.; Sun, Z. T.; Cox, T.; Xu, J. L.; Reetz, M. T., Beating Bias in the Directed Evolution of Proteins: Combining High-Fidelity on-Chip Solid-Phase Gene Synthesis with Efficient Gene Assembly for Combinatorial Library Construction. ChemBioChem 2018, 19, 221-228.
83. Kille, S.; Acevedo-Rocha, C. G.; Parra, L. P.; Zhang, Z. G.; Opperman, D. J.; Reetz, M. T.; Acevedo, J. P., Reducing Codon Redundancy and Screening Effort of Combinatorial Protein Libraries Created by Saturation Mutagenesis. ACS Synth. Biol. 2013, 2, 83-92.
84. Jochens, H.; Bornscheuer, U. T., Natural Diversity to Guide Focused Directed Evolution. ChemBioChem 2010, 11, 1861-1866.
85. Moore, J. C.; Rodriguez-Granillo, A.; Crespo, A.; Govindarajan, S.; Welch, M.; Hiraga, K.; Lexa, K.; Marshall, N.; Truppo, M. D., “Site and Mutation”-Specific Predictions Enable Minimal Directed Evolution Libraries. ACS Synth. Biol. 2018, 7, 1730-1741.
86. Lutz, S., Beyond Directed Evolution—Semi-Rational Protein Engineering and Design. Curr. Opin. Biotechnol. 2010, 21, 734-743.
87. Schwarte, A.; Genz, M.; Skalden, L.; Nobili, A.; Vickers, C.; Melse, O.; Kuipers, R.; Joosten, H. J.;
Stourac, J.; Bendl, J.; Black, J.; Haase, P.; Baakman, C.; Damborsky, J.; Bornscheuer, U.; Vriend, G.; Venselaar, H., NewProt—a Protein Engineering Portal. Protein Eng. Des. Sel. 2017, 30, 441-447.
88. Bendl, J.; Stourac, J.; Sebestova, E.; Vavra, O.; Musil, M.; Brezovsky, J.; Damborsky, J., HotSpot Wizard 2.0: Automated Design of Site-Specific Mutations and Smart Libraries in Protein Engineering. Nucleic Acids Res. 2016, 44, W479-W487.
89. Pavlova, M.; Klvana, M.; Prokop, Z.; Chaloupkova, R.; Banas, P.; Otyepka, M.; Wade, R. C.; Tsuda, M.; Nagata, Y.; Damborsky, J., Redesigning Dehalogenase Access Tunnels as a Strategy for Degrading an Anthropogenic Substrate. Nat. Chem. Biol. 2009, 5, 727-733.
90. Chica, R. A.; Doucet, N.; Pelletier, J. N., Semi-Rational Approaches to Engineering Enzyme Activity: Combining the Benefits of Directed Evolution and Rational Design. Curr. Opin. Biotechnol. 2005, 16, 378-384.
91. Heath, R. S.; Birmingham, W. R.; Thompson, M. P.; Taglieber, A.; Daviet, L.; Turner, N. J., An Engineered Alcohol Oxidase for the Oxidation of Primary Alcohols. ChemBioChem 2019, 20, 276-281.
92. Alley, E. C.; Khimulya, G.; Biswas, S.; AlQuraishi, M.; Church, G. M., Unified Rational Protein Engineering with Sequence-Based Deep Representation Learning. Nat. Methods 2019, 16, 1315-+. 22-5. Ghislieri, D.; Green, A. P.; Pontini, M.; Willies, S. C.; Rowles, I.; Frank, A.; Grogan, G.; Turner, N. J., Engineering an Enantioselective Amine Oxidase for the Synthesis of Pharmaceutical Building Blocks and Alkaloid Natural Products. J. Am. Chem. Soc. 2013, 135, 10863-10869.
93. Alexeeva, M.; Enright, A.; Dawson, M. J.; Mahmoudian, M.; Turner, N. J., Deracemization of α-Methylbenzylamine using an Enzyme Obtained by in vitro Evolution. Angew. Chem. Int. Ed. 2002, 41, 3177-3180.
94. Carr, R.; Alexeeva, M.; Enright, A.; Eve, T. S.; Dawson, M. J.; Turner, N. J., Directed Evolution of an Amine Oxidase Possessing both Broad Substrate Specificity and High Enantioselectivity. Angew. Chem. Int. Ed. Engl. 2003, 42, 4807-10.
95. Dunsmore, C. J.; Carr, R.; Fleming, T.; Turner, N. J., A Chemo-Enzymatic Route to Enantiomerically Pure Cyclic Tertiary Amines. J. Am. Chem. Soc. 2006, 128, 2224-2225.
96. Brühmüller, M.; Decker, K.; MOhler, H., Covalently Bound Flavin in D-6-Hydroxynicotine Oxidase from Arthrobacter oxidans Purification and Properties of d-6-Hydroxynicotine Oxidase. Eur. J. Biochem. 1972, 29, 143-151.
97. Cantú Reinhard, F. G.; Kell, D. B.; Almond, A., Using Dynamics to Predict Enzyme Catalytic Turnover Number for use in Prioritization of Directed Evolution Distal Amino Acid Mutations: Application to 6-Hydroxy-D-Nicotine Oxidase (6-HDNO) from Arthrobacter nicotinovorans. In preparation 2021.
98. Gao, M. X.; Nie, C. B.; Li, J. Y.; Song, B. B.; Cheng, X. R.; Sun, E. Y.; Yan, L.; Qian, H., Design, Synthesis and Biological Evaluation of N-1-(Isoquinolin-5-yl)-N-2-Phenylpyrrolidine-1,2-Dicarboxamide Derivatives as Potent TRPV1 Antagonists. Bioorg. Chem. 2019, 82, 100-108.
99. Haak, A. J.; Girtman, M. A.; Ali, M. F.; Carmona, E. M.; Limper, A. H.; Tschumperlin, D. J., Phenylpyrrolidine Structural Mimics of Pirfenidone Lacking Antifibrotic Activity: A New Tool for Mechanism of Action Studies. Eur. J. Pharmacol. 2017, 811, 87-92.
100. Kandeel, M.; Yamamoto, M.; AI-Taher, A.; Watanabe, A.; Oh-Hashi, K.; Park, B. K.; Kwon, H. J.; Inoue, J. I.; AI-Nazawi, M., Small Molecule Inhibitors of Middle East Respiratory Syndrome Coronavirus Fusion by Targeting Cavities on Heptad Repeat Trimers. Biomol. Ther. 2020, 28, 311-319.
101. Choi, H. S.; Rucker, P. V.; Wang, Z. C.; Fan, Y.; Albaugh, P.; Chopiuk, G.; Gessier, F.; Sun, F. X.; Adrian, F.; Liu, G. X.; Hood, T.; Li, N. X.; Jia, Y.; Che, J. W.; McCormack, S.; Li, A.; Li, J.; Steffy, A.; Culazzo, A.; Tompkins, C.; Phung, V.; Kreusch, A.; Lu, M.; Hu, B.; Chaudhary, A.; Prashad, M.; Tuntland, T.; Liu, B.; Harris, J.; Seidel, H. M.; Loren, J.; Molteni, V., (R)-2-Phenylpyrrolidine Substituted Imidazopyridazines: A New Class of Potent and Selective Pan-IRK Inhibitors. ACS Med. Chem. Lett. 2015, 6, 562-567.
102. Tokuriki, N.; Stricher, F.; Serrano, L.; Tawfik, D. S., How Protein Stability and New Functions Trade Off. PLoS Comput. Biol. 2008, 4, e1000002.
103. Lawrence, M. S.; Phillips, K. J.; Liu, D. R., Supercharging Proteins can Impart Unusual Resilience. J. Am. Chem. Soc. 2007, 129, 10110-10112.
37-104. Carr, R.; Alexeeva, M.; Dawson, M. J.; Gotor-Fernandez, V.; Humphrey, C. E.; Turner, N. J., Directed Evolution of an Amine Oxidase for the Preparative Deracemisation of Cyclic Secondary Amines. ChemBioChem 2005, 6, 637-639.
105. Batista, V. F.; Galman, J. L.; Pinto, D.; Silva, A. M. S.; Turner, N. J., Monoamine Oxidase: Tunable Activity for Amine Resolution and Functionalization. ACS Catal. 2018, 8, 11889-11907.
106. Duan, J. Q.; Li, B. B.; Qin, Y. C.; Dong, Y. J.; Ren, J.; Li, G. Y., Recent Progress in Directed Evolution of Stereoselective Monoamine Oxidases. Bioresour. Bioprocess. 2019, 6.
107. Nannemann, D. P.; Birmingham, W. R.; Scism, R. A.; Bachmann, B. O., Assessing Directed Evolution Methods for the Generation of Biosynthetic Enzymes with Potential in Drug Biosynthesis. Future Med. Chem. 2011, 3, 803-819.
108. Romero, P. A.; Arnold, F. H., Exploring Protein Fitness Landscapes by Directed Evolution. Nat. Rev. Mol. Cell Biol. 2009, 10, 866-876.
109. Goldsmith, M.; Tawfik, D. S., Directed Enzyme Evolution: Beyond the Low-Hanging Fruit. Curr. Opin. Struct. Biol. 2012, 22, 406-412.
110. Tokuriki, N.; Jackson, C. J.; Afriat-Jurnou, L.; Wyganowski, K. T.; Tang, R. M.; Tawfik, D. S., Diminishing Returns and Tradeoffs Constrain the Laboratory Optimization of an Enzyme. Nat. Commun. 2012, 3, 1257.
111. Schober, M.; MacDermaid, C.; Ollis, A. A.; Chang, S.; Khan, D.; Hosford, J.; Latham, J.; Ihnken, L. A. F.; Brown, M. J. B.; Fuerst, D.; Sanganee, M. J.; Roiban, G. D., Chiral Synthesis of LSD1 Inhibitor GSK2879552 Enabled by Directed Evolution of an Imine Reductase. Nat. Catal. 2019, 2, 909-915.
112. Siegel, J. B.; Zanghellini, A.; Lovick, H. M.; Kiss, G.; Lambert, A. R.; Clair, J. L. S.; Gallaher, J. L.; Hilvert, D.; Gelb, M. H.; Stoddard, B. L.; Houk, K. N.; Michael, F. E.; Baker, D., Computational Design of an Enzyme Catalyst for a Stereoselective Bimolecular Diels-Alder Reaction. Science 2010, 329, 309-313.
113. Yang, K. K.; Wu, Z.; Arnold, F. H., Machine-Learning-Guided Directed Evolution for Protein Engineering. Nat. Methods 2019, 16, 687-694.
114. Yeung, N.; Lin, Y. W.; Gao, Y. G.; Zhao, X.; Russell, B. S.; Lei, L. Y.; Miner, K. D.; Robinson, H.; Lu, Y., Rational Design of a Structural and Functional Nitric Oxide Reductase. Nature 2009, 462, 1079-1082.
115. Swainston, N.; Currin, A.; Day, P. J.; Kell, D. B., GeneGenie: Optimized Oligomer Design for Directed Evolution. Nucleic Acids Res. 2014, 42, W395-W400.
116. Der, B. S.; Kluwe, C.; Miklos, A. E.; Jacak, R.; Lyskov, S.; Gray, J. J.; Georgiou, G.; Ellington, A. D.; Kuhlman, B., Alternative Computational Protocols for Supercharging Protein Surfaces for Reversible Unfolding and Retention of Stability. Plos One 2013, 8, e64363.
117. Schomburg, I.; Jeske, L.; Ulbrich, M.; Placzek, S.; Chang, A.; Schomburg, D., The BRENDA Enzyme Information System—From a Database to an Expert System. J. Biotechnol. 2017, 261, 194-206.
118. Sheludko, Y. V.; Fessner, W. D., Winning the Numbers Game in Enzyme Evolution—Fast Screening Methods for Improved Biotechnology Proteins. Curr. Opin. Struct. Biol. 2020, 63, 123-133.
119. Cantú Reinhard, F. G.; Mangas-Sanchez, J.; Heath, R. S.; Kell, D. B.; Turner, N. J.; Almond, A., Rapid Improvement of both Enzyme Activity and Thermal Stability by Rational Directed Evolution using a Multiply Degenerate Full-Length Gene Library and Supercharging: Application to 6-Hydroxy-D-Nicotine Oxidase (6-HDNO) from Arthrobacter nicotinovorans. In preparation 2021.
120. Packer, M. S.; Liu, D. R., Methods for the Directed Evolution of Proteins. Nat. Rev. Genet. 2015, 16, 379-394.
121. Turner, N. J., Directed Evolution Drives the Next Generation of Biocatalysts. Nat. Chem. Biol. 2009, 5, 568-574.
122. Wu, Z.; Kan, S. B. J.; Lewis, R. D.; Wittmann, B. J.; Arnold, F. H., Machine Learning-Assisted Directed Protein Evolution with Combinatorial Libraries. Proc. Natl. Acad. Sci. U.S.A. 2019, 116, 8852-8858.
123. Senior, A. W.; Evans, R.; Jumper, J.; Kirkpatrick, J.; Sifre, L.; Green, T.; Qin, C. L.; Zidek, A.; Nelson, A. W. R.; Bridgland, A.; Penedones, H.; Petersen, S.; Simonyan, K.; Crossan, S.; Kohli, P.; Jones, D. T.; Silver, D.; Kavukcuoglu, K.; Hassabis, D., Improved Protein Structure Prediction using Potentials from Deep Learning. Nature 2020, 577, 706-710.
124. Yang, J. Y.; Anishchenko, I.; Park, H.; Peng, Z. L.; Ovchinnikov, S.; Baker, D., Improved Protein Structure Prediction using Predicted Interresidue Orientations. Proc. Natl. Acad. Sci. U.S.A. 2020, 117, 1496-1503.
125. Cheng, F.; Zhu, L. L.; Schwaneberg, U., Directed Evolution 2.0: Improving and Deciphering Enzyme Properties. ChemComm 2015, 51, 9760-9772.
126. Fox, R., Directed Molecular Evolution by Machine Learning and the Influence of Nonlinear Interactions. J. Theor. Biol. 2005, 234, 187-199.
127. Kawashima, S.; Pokarowski, P.; Pokarowska, M.; Kolinski, A.; Katayama, T.; Kanehisa, M., AAindex: Amino Acid Index Database, Progress Report 2008. Nucleic Acids Res. 2008, 36, D202-D205.
128. Cadet, F.; Fontaine, N.; Li, G. Y.; Sanchis, J.; Chong, M. N. F.; Pandjaitan, R.; Vetrivel, I.; Offmann, B.; Reetz, M. T., A Machine Learning Approach for Reliable Prediction of Amino Acid Interactions and its Application in the Directed Evolution of Enantioselective Enzymes. Sci. Rep. 2018, 8, 16757.
129. Sandberg, W. S.; Terwilliger, T. C., Engineering Multiple Properties of a Protein by Combinational Mutagenesis. Proc. Natl. Acad. Sci. U.S.A. 1993, 90, 8367-8371.
130. Hu, L. H.; Soderhjelm, P.; Ryde, U., On the Convergence of QM/MM Energies. J. Chem. Theor. Comp. 2011, 7, 761-777.
131. Heimdal, J.; Ryde, U., Convergence of QM/MM Free-Energy Perturbations Based on Molecular-Mechanics or Semiempirical Simulations. Phys. Chem. Chem. Phys. 2012, 14, 12592-12604.
132. Duarte, F.; Amrein, B. A.; Blaha-Nelson, D.; Kamerlin, S. C. L., Recent Advances in QM/MM Free Energy Calculations using Reference Potentials. Biochim. Biophys. Acta Gen. Subj. 2015, 1850, 954-965.
133. Lodola, A.; Mor, M.; Zurek, J.; Tarzia, G.; Piomelli, D.; Harvey, J. N.; Mulholland, A. J., Conformational Effects in Enzyme Catalysis: Reaction via a High Energy Conformation in Fatty Acid Amide Hydrolase. Biophysical Journal 2007, 92, L20-L22.
134. Hasan, M. M.; Kurata, H., GPSuc: Global Prediction of Generic and Species-specific Succinylation Sites by aggregating multiple sequence features. PLoS One 2018, 13, e0200283.
135. Fontaine, N. T.; Cadet, X. F.; Vetrivel, I., Novel Descriptors and Digital Signal Processing-Based Method for Protein Sequence Activity Relationship Study. Int. J. Mol. Sci. 2019, 20, 5640.
136. Chen, Z.; Zhao, P.; Li, F. Y.; Leier, A.; Marquez-Lago, T. T.; Wang, Y. N.; Webb, G. I.; Smith, A. I.; Daly, R. J.; Chou, K. C.; Song, J. N., iFeature: a Python Package and Web Server for Features Extraction and Selection from Protein and Peptide Sequences. Bioinformatics 2018, 34, 2499-2502.
137. Hibbert, E. G.; Senussi, T.; Costelloe, S. J.; Lei, W. L.; Smith, M. E. B.; Ward, J. M.; Hailes, H. C.; Dalby, P. A., Directed Evolution of Transketolase Activity on Non-Phosphorylated Substrates. J. Biotechnol. 2007, 131, 425-432.
138. Morley, K. L.; Kazlauskas, R. J., Improving Enzyme Properties: When are Closer Mutations Better? Trends Biotechnol. 2005, 23, 231-237.
139. Park, S.; Morley, K. L.; Horsman, G. P.; Holmquist, M.; Hult, K.; Kazlauskas, R. J., Focusing Mutations into the P. fluorescens Esterase Binding Site Increases Enantioselectivity more Effectively than Distant Mutations. Chem. Biol. 2005, 12, 45-54.
140. Dalby, P. A., Strategy and Success for the Directed Evolution of Enzymes. Curr. Opin. Struct. Biol. 2011, 21, 473-480.
141. Marcos, E.; Silva, D. A., Essentials of de novo Protein Design: Methods and Applications. Wiley Interdiscip. Rev. Comput. Mol. Sci. 2018, 8, e1374.
142. Li, A. T.; Sun, Z. T.; Reetz, M. T., Solid-Phase Gene Synthesis for Mutant Library Construction: The Future of Directed Evolution? ChemBioChem 2018, 19, 2023-2032.
143. Cui, H. Y.; Cao, H.; Cai, H. Y.; Jaeger, K. E.; Davari, M. D.; Schwaneberg, U., Computer-Assisted Recombination (CompassR) Teaches us How to Recombine Beneficial Substitutions from Directed Evolution Campaigns. Chem. Eur. J. 2020, 26, 643-649.
144. Kamerlin, S. C. L.; Warshel, A., The Empirical Valence Bond Model: Theory and Applications. Wiley Interdiscip. Rev. Comput. Mol. Sci. 2011, 1, 30-45.
145. Chaudhury, S.; Lyskov, S.; Gray, J. J., PyRosetta: a Script-Based Interface for Implementing Molecular Modeling Algorithms using Rosetta. Bioinformatics 2010, 26, 689-691.
146. Case, D. A.; Cheatham, T. E.; Darden, T.; Gohlke, H.; Luo, R.; Merz, K. M.; Onufriev, A.; Simmerling, C.; Wang, B.; Woods, R. J., The AMBER Biomolecular Simulation Programs. J. Comp. Chem. 2005, 26, 1668-1688.
147. Cooley, J. W.; Tukey, J. W., An Algorithm for Machine Calculation of Complex Fourier Series. Math. Comput. 1965, 19, 297-301.
148. Wilson, D. S.; Keefe, A. D., Random Mutagenesis by PCR. Curr. Protoc. Mol. Biol. 2000, 51, 8.3.1-8.3.9.
149. Fox, R. J.; Davis, S. C.; Mundorff, E. C.; Newman, L. M.; Gavrilovic, V.; Ma, S. K.; Chung, L. M.; Ching, C.; Tam, S.; Muley, S.; Grate, J.; Gruber, J.; Whitman, J. C.; Sheldon, R. A.; Huisman, G. W., Improving Catalytic Function by ProSAR-Driven Enzyme Evolution. Nat. Biotechnol. 2007, 25, 338-344.
150. de Souza, P. M. and P. de Oliveira Magalhaes, Application of microbial alpha-amylase in industry—A review. Braz J Microbiol, 2010. 41(4): p. 850-61.
151. Farooq, M. A., et al., Biosynthesis and industrial applications of α-amylase: a review. Archives of Microbiology, 2021.
152. Bessler, C., et al., Directed evolution of a bacterial α-amylase: Toward enhanced pH-performance and higher specific activity. Protein Science, 2009. 12(10): p. 2141-2149.
153. Huang, L., et al., Directed evolution of α-amylase from Bacillus licheniformis to enhance its acid—stable performance. Biologia, 2019. 74(10): p. 1363-1372.
154. Wang, C.-H., et al., Simultaneously Improved Thermostability and Hydrolytic Pattern of Alpha—Amylase by Engineering Central Beta Strands of TIM Barrel. Applied Biochemistry and Biotechnology, 2020. 192(1): p. 57-70.
155. Pinto, G. P., et al., Establishing the Catalytic Mechanism of Human Pancreatic α-Amylase with QM/MM Methods. Journal of Chemical Theory and Computation, 2015. 11(6): p. 2508-2516.
156. Nahoum, V., et al., Crystal structures of human pancreatic α-amylase in complex with carbohydrate and proteinaceous inhibitors. Biochemical Journal, 2000. 346(1): p. 201-208.
157. Gentleman, R., et al., OpenMM 7: Rapid development of high performance algorithms for molecular dynamics. PLOS Computational Biology, 2017. 13(7).
158. Ditchfield, R., W. J. Hehre, and J. A. Pople, Self-Consistent Molecular-Orbital Methods. IX. An Extended Gaussian-Type Basis for Molecular-Orbital Studies of Organic Molecules. The Journal of Chemical Physics, 1971. 54(2): p. 724-728.
159. Hehre, W. J., R. Ditchfield, and J. A. Pople, Self-Consistent Molecular Orbital Methods. XII. Further Extensions of Gaussian-Type Basis Sets for Use in Molecular Orbital Studies of Organic Molecules. The Journal of Chemical Physics, 1972. 56(5): p. 2257-2261.
160. Kosugi, T. and S. Hayashi, Crucial Role of Protein Flexibility in Formation of a Stable Reaction Transition State in an a-Amylase Catalysis. Journal of the American Chemical Society, 2012. 134(16): p. 7045-7055.
161. Nielsen, J. E., et al., Electrostatics in the active site of an alpha-amylase. European Journal of Biochemistry, 1999. 264(3): p. 816-824.
162. Hirshfeld, F. L., Bonded-atom fragments for describing molecular charge densities. Theoretica Chimica Acta, 1977. 44(2): p. 129-138.
163. Anandakrishnan, R. et al. H++3.0: automating pK prediction and the preparation of biomolecular structures for atomistic molecular modeling and simulation. Nucleic Acids Res., 40(W1):W537-541. (2012).
164. Cadet, F., Fontaine, N., Vetrivel, I. et al. Application of fourier transform and proteochemometrics principles to protein engineering. BMC Bioinformatics 19, 382 (2018).
165. Sergio Martinez Cuesta, Syed Asad Rahman, Janet M. Thornton. Chemistry and evolution of the isomerases. Proceedings of the National Academy of Sciences February 2016, 113 (7) 1796-1801.
166. van der Kamp M W, Chaudret R, Mulholland A J. QM/MMmodelling of ketosteroid isomerase reactivity indicates that active site closure is integral to catalysis. FEBS J. 2013 July; 280(13):3120-31. doi: 10.1111/febs.12158. Epub 2013 Feb. 27. PMID: 23356661.
167. Qian P, Guo H B, Yue Y, Wang L, Yang X, Guo H. Understanding the Catalytic Mechanism of Xanthosine Methyltransferase in Caffeine Biosynthesis from QM/MM Molecular Dynamics and Free Energy Simulations. J Chem Inf Model. 2016 Sep. 26; 56(9):1755-61. doi: 10.1021/acs.jcim.6b00153. Epub 2016 Aug. 15. Erratum in: J Chem Inf Model. 2016 Nov. 28; 56(11):2280. PMID: 27482605.
168. Zhu W, Liu Y, Zhang R. A QM/MM study of the reaction mechanism of (R)-hydroxynitrile lyases from Arabidopsis thaliana (AtHNL). Proteins. 2015 January; 83(1):66-77. doi: 10.1002/prot.24648. Epub 2014 Nov. 20. PMID: 25052541.
169. Mohammad Dadashipour and Yasuhisa Asano. Hydroxynitrile Lyases: Insights into Biochemistry, Discovery, and Engineering. ACS Catal. 2011, 1, 9, 1121-1149

All references cited herein are incorporated herein by reference in their entirety and for all purposes to the same extent as if each individual publication or patent or patent application was specifically and individually indicated to be incorporated by reference in its entirety.

1. A computer-implemented method of predicting catalytic activity for a candidate mutant enzyme, wherein the candidate mutant enzyme differs from a reference enzyme by one or more amino acids, the method comprising:
providing a set of parameters from a molecular simulation of the reference enzyme, wherein a region of the enzyme (QM region) comprising at least part of the active site and a substrate of the enzyme is optimised with a quantum mechanics method;
performing a molecular dynamics simulation with the candidate mutant enzyme and a substrate of the enzyme to obtain a plurality of conformations each associated with a set of atomic coordinates;
estimating the electrostatic component of the activation barrier (ΔΔG^{‡}_{Q20}) for each of the plurality of conformations of the candidate mutant enzyme, using the parameters from the molecular simulation of the reference enzyme and the set of atomic coordinates associated with the respective conformation, thereby obtaining a plurality of estimates of the electrostatic component of the activation barrier (ΔΔG^{‡}_{Q20});
and determining a score (ΔΔG^{‡}_{Q20EFF}, μ_{Q20}) based on the plurality of estimates of the electrostatic component of the activation barrier, wherein the score is indicative of the effective activation barrier (ΔΔG^{‡}) of the candidate mutant enzyme.

2. The method of claim 1, wherein the method further comprises defining a core region that includes one or more of the atoms of the QM region, and an external region that includes the remaining atoms of the enzyme, and wherein the set of parameters from the molecular simulation of the reference enzyme comprises: the changes to the partial charges of the atoms in the core region (ΔQᵢ) that occur during the formation of the transition state for a particular conformation of the reference enzyme from the reaction complex, and partial atomic charges for atoms in the external region;
optionally wherein a change in partial atomic charges for each atom in the core region is obtained for each of a plurality of conformations, and a representative change of partial atomic charges for each atom in the core region is obtained as the mean value across each of the plurality of conformations, and/or wherein the change in charges is calculated via a population analysis method including Mulliken population analysis, Hirshfeld population analysis, CM5 population analysis.

3. The method of claim 2, wherein the parameters from the molecular simulation of the reference enzyme comprise the partial charge difference between the transition state and the reaction complex for each atom of the core region (ΔQᵢ) and estimating the electrostatic component of the activation barrier for a conformation of the candidate mutant enzyme comprises calculating electrostatic Coulombic interactions between:
each atom of the external region; and
the partial charge difference between the transition state and the reaction complex for each atom of the core region, optionally wherein estimating the electrostatic component of the activation barrier for a conformation of the candidate mutant enzyme summing the electrostatic Coulombic interactions over all pairs of external and core atoms, preferably using Equation (5):
$$\\ \begin{matrix}Δ Δ {G}_{Q 20}^{{}_{} ‡} = c {\sum }_{j ∈ external} {\sum }_{i ∈ core} \frac{{q}_{j} Δ {Q}_{i}}{{r}_{ji}} & ( 5 )\end{matrix}$$
where ΔΔG^{‡}_{Q20} is the estimate of the electrostatic component of the activation barrier, qⱼ is the partial charge for atom j of the external region, ΔQᵢ is the partial charge difference between the transition state and the reaction complex for atom i of the core region, external region and distances to the core atoms rⱼᵢ is the distance between atoms i and j in the set of atomic coordinates associated with the conformation, and c is a constant.

4. The method of any preceding claim, wherein the score is indicative of the turnover number of the candidate mutant enzyme, optionally wherein the turnover number is exponentially dependent on the score for the candidate mutant enzyme and/or wherein the method further comprises obtaining a score based on the score indicative of the turnover number and one or more other properties.

5. The method of any preceding claim, wherein determining a score (ΔΔG^{‡}_{Q20EFF}, μ_{Q20}) based on the plurality of estimates of the electrostatic component of the activation barrier comprises calculating one or more statistical parameters of the distribution of estimates of the electrostatic component of the activation barrier (ΔΔG^{‡}_{Q20}) for the plurality of conformations of the candidate mutant enzyme, optionally wherein the statistical parameters comprise the average (μ_{Q20}) and the standard deviation (σ_{Q20}) of the distribution of estimates and/or wherein determining the score (ΔΔG^{‡}_{Q20EFF}) comprises using Equation (2):
$$\\ \begin{matrix}Δ Δ {G}_{Q 20 EFF}^{{}_{} ‡} = {μ}_{Q 20} - \frac{{σ}_{Q 20}^{{}_{} 2}}{2 RT} & ( 2 )\end{matrix}$$
wherein μ_{Q20} is the average and σ_{Q20} is the standard deviation of the distribution of estimates, and RT is the product of the gas constant and temperature, and/or wherein the statistical parameters comprise the average (μ_{Q20}) and wherein the score is the average (μ_{Q20}) or is based on the average as the only statistical parameter of the distribution of estimates of the electrostatic component of the activation barrier.

6. The method of any preceding claim, wherein performing a molecular dynamics simulation with the candidate mutant enzyme and substrate comprises:
performing a molecular dynamics simulation with the candidate mutant enzyme, the substrate and one or more cofactors, and/or
performing a molecular dynamics simulation with the candidate mutant enzyme, substrate and any cofactor in a near attack conformation, optionally wherein performing a molecular dynamics simulation with the candidate mutant enzyme and substrate comprises performing a molecular dynamics simulation using one or more harmonic constraints that maintain the enzyme, the substrate and any cofactors in a near attack conformation.

7. The method of any preceding claim, wherein the candidate mutant enzyme differs from the reference enzyme by one or more amino acids and/or wherein the candidate mutant enzyme differs from the reference enzyme by one or more amino acids outside of the active site, and/or wherein the candidate mutant enzyme differs from the reference enzyme by 1, 2 or 3 amino acids, by up to 6 amino acids, by up to 12 amino acids, by up to 24 amino acids, by up to 48 amino acids, or by 1, 2, 3, 6 or 12 amino acids.

8. The method of any preceding claim, wherein performing a molecular dynamics simulation with the candidate mutant enzyme and substrate comprises performing a molecular dynamics simulation for a period of at least 0.1 ns, at least 1 ns, at least 5 ns, at least 10 ns, at least 20 ns, at least 30 ns, at least 40 ns, about 1 ns or about 50 ns, and/or wherein the plurality of conformations corresponds to a plurality of times of the molecular dynamics simulation.

9. The method of any preceding claim, wherein performing a molecular dynamics simulation with the candidate mutant enzyme and substrate comprises obtaining a conformation from a molecular dynamics simulation of the reference enzyme, substituting the one or more mutant amino acids in the conformation, and optionally performing a molecular dynamics for a period of time to allow the conformation to equilibrate prior to obtaining the plurality of conformations and/or performing simulated annealing to remove steric clashes involving mutated residues and/or performing a rotamer conformation search and minimisation to remove steric clashes.

10. A computer-implemented method of predicting catalytic activity for a candidate mutant enzyme, wherein the candidate mutant enzyme differs from a reference enzyme by one or more amino acids, the method comprising:
providing a candidate mutant enzyme as an input to a machine learning model that has been trained to take as input a candidate enzyme sequence and produce as output a score indicative of the effective activation barrier of the candidate mutant enzyme, wherein the machine learning model has been trained using training data comprising a plurality of candidate mutant enzyme sequences and corresponding scores indicative of the effective activation barrier (ΔΔG^{‡}) of the candidate mutant enzyme obtained using the method of any of claims 1 to 9.

11. The method of claim 10, wherein the machine learning model comprises a plurality of individual machine learning models wherein each individual machine learning model has been trained to take as input a candidate enzyme sequence and produce as input a score indicative of the effective activation barrier of the candidate mutant enzyme,
optionally wherein the machine learning model comprises one or more ensembles of individual machine learning models.

12. The method of claim 10 or claim 11, wherein each individual machine learning model has been trained using training data comprising a plurality of candidate mutant enzyme sequences and corresponding scores indicative of the effective activation barrier of the candidate mutant enzyme obtained using the method of any of claims 1 to 9, wherein the scores have been obtained by performing a molecular dynamics simulation with the candidate mutant enzyme and substrate using the same starting conformation from a molecular dynamics simulation of the reference enzyme, optionally wherein the machine learning model comprises individual machine learning models that have been trained using training data comprising scores that have been obtained performing a molecular dynamics simulation with the candidate mutant enzyme and substrate using a respective starting conformation from a molecular dynamics simulation of the reference enzyme, wherein the respective starting conformations used for at least two of the individual machine learning models are different from each other.

13. The method of claim 12, wherein the machine learning model comprises a plurality of ensembles of individual machine learning models, wherein each individual machine learning model has been trained using training data comprising a plurality of candidate mutant enzyme sequences and corresponding scores obtained by performing a molecular dynamics simulation with the candidate mutant enzyme and substrate using the same starting conformation from a molecular dynamics simulation of the reference enzyme,
optionally wherein each respective one of the plurality of ensembles of individual machine learning models comprises individual machine learning models that have been trained using training data comprising scores obtained by performing a molecular dynamics simulation with the candidate mutant enzyme and substrate using a respective starting conformation from a molecular dynamics simulation of the reference enzyme.

14. The method of any of claims 11 to 13, wherein the scores produced by each individual machine learning model or the combined scores produced by each ensemble are standardised, optionally wherein the scores are standardised using parameters defined based on scores obtained for a common set of mutant enzyme sequences, optionally wherein the common set of mutant enzyme sequences comprises candidate mutant enzymes with mutations that together cover any position associated with a mutation in a candidate enzyme for which a prediction is to be obtained.

15. The method of any of claims 11 to 14, wherein the optionally standardised combined scores produced for the same sequence by each ensemble are combined into a single score for each candidate enzyme sequence, for example a mean or median score.

16. The method of any of claims 10 to 15, wherein the machine learning model has been trained using training data comprising a plurality of candidate mutant enzyme sequences that each differ from the same reference enzyme by more than one amino acid, or by at least 1, at least 2, at least 3, between 3 and 6, between 3 and 24, between 3 and 48, between 3 and 12, 1, 2, 3, 4, 5, 6, 12, 24 or 48 amino acids; and/or
wherein the machine learning model has been trained using training data comprising at least 1000, at least 10,000, at least 50,000, at least 100,000, at least 200,000 or at least 300,000 candidate mutant enzyme sequences; and/or
wherein the machine learning model has been trained using training data comprising a plurality of candidate mutant enzyme sequences that differ from the reference enzyme by at least one amino acid, wherein the plurality of candidate mutant enzyme sequences together comprise mutations at each position of the reference enzyme apart from excluded positions, optionally wherein excluded positions comprise one or more of key catalytic residues, cysteine residues, N terminus residues and C terminus residues; and/or wherein each candidate mutant enzyme comprises one or more randomly selected mutations at a randomly selected position; and/or
wherein the machine learning model or each of the individual machine learning models is selected from: a regression model, optionally a linear regression model or derivative thereof such as a multiple linear regression model or a Lasso regularised linear regression model, a support vector regression model, and a neural network model such as a dense neural network model.

17. The method of any of claims 10 to 16 wherein the machine learning model or each individual machine learning model takes as input a candidate enzyme sequence that is encoded using an encoding dictionary where each amino acid is represented by a vector of size N,
optionally wherein each element of the vector is:
an amino acid property from a randomly selected set of amino acid properties, optionally from the AAindex amino acid properties database, or
a random number, optionally wherein the real random number is selected between 0 and 1;
a 0 or a 1, wherein the vector has size N equal to the number of different amino acids considered, and each vector contains a single 1 or a single 0 at a position specific for the amino acid being encoded;
a 0 or a 1, wherein the vector has size N=1, and the element is equal to 0 if the residue is not mutated and 1 otherwise, or vice-versa; and/or
optionally wherein the resulting encoded sequence of numbers is subject to a fast Fourier transform procedure for each encoded vector and the real part of the FFT result is used to encode the protein sequence data.

18. A computer-implemented method of providing a site directed mutagenesis potential map for a reference enzyme, the method comprising:
providing a plurality of candidate mutated enzymes, wherein the candidate mutant enzyme differs from the reference enzyme by at least one amino acid at a plurality of positions that together form a mapped region;
predicting the catalytic activity of each of the plurality of candidate mutated enzymes using the method of any preceding claim thereby obtaining for each candidate mutated enzyme a score indicative of the in the effective activation barrier of the candidate mutant enzyme; and
combining the scores for the plurality of candidate mutated enzymes into one or more position-specific metrics indicative of the potential for mutant-associated catalytic improvement at the position.

19. The method of claim 18, wherein combining the scores for the plurality of candidate mutated enzymes into one or more position-specific metrics comprises obtaining one or more position-specific metrics for each position in the mapped region based on the scores obtained for candidate mutated enzymes of the plurality of candidate mutated enzymes that comprise a mutation at the respective position, optionally wherein the one or more position-specific metrics comprise a mean or median score, a maximum score and/or a minimum score for the candidate mutated enzymes of the plurality of candidate mutated enzymes that comprise a mutation at the respective position.

20. A method of providing a candidate enzyme with improved catalytic activity compared to a reference enzyme, the method comprising:
providing a plurality of candidate mutated enzymes, wherein the candidate mutant enzyme differs from a reference enzyme by one or more amino acids;
predicting the catalytic activity of each of the plurality of candidate mutated enzymes using the method of any of claims 1 to 17 thereby obtaining for each candidate mutated enzyme a score indicative of the effective activation barrier of the candidate mutant enzyme; and
ranking the plurality of candidate mutated enzymes on the basis of the scores obtained, thereby identifying candidate mutant enzymes that are likely to have improved catalytic activity.

21. The method of claim 20, wherein the plurality of candidate mutated enzymes differ from the reference enzyme at a plurality of candidate positions that together span any region of the enzyme, optionally excluding one or more residues a priori identified to be directly involved in the mechanism of reaction and/or any cysteine residues and/or any residues in the N terminal and/or C terminal region and/or any residues known to covalently bond a cofactor and/or any residues which have been selected to impose restraints in the molecular dynamics simulation, and/or wherein the plurality of candidate mutated enzymes have been selected using a site directed mutagenesis potential map generated using the method of claim 18 or claim 19.

22. A method of providing a candidate mutant enzyme with improved catalytic activity compared to a reference enzyme, the method comprising:
providing a site directed mutagenesis potential map for a reference enzyme using the method of claim 18 or claim 19, and
identifying one or more candidate position(s) that is/are associated with one or more candidate mutant enzymes likely to have improved catalytic activity based on the one or more position-specific metrics, optionally wherein the method further comprises providing one or more candidate mutant enzymes comprising mutations at the one or more candidate position(s) and predicting their catalytic activity using the method of any of claims 1 to 17.

23. The method of any of claims 20 to 22, further comprising:
identifying key catalytic residues by any recombinant technique such as site directed mutagenesis, wherein the reference enzyme comprises the key catalytic residues and/or selecting one or more candidate positions in the enzyme for experimental validation based on a combination of criteria including:
the ranked scores associated with the candidate mutant enzymes or the one or more position-specific metrics; and
one or more of: the location of the positions in the enzyme, and one or more criteria associated with a specific gene synthesis methodology.

24. The method of claim 23, further comprising designing and/or providing a library for PCR-based gene synthesis and/or solid phase gene synthesis and/or full de novo gene synthesis and/or site directed mutagenesis that comprises degenerate codons for the selected candidate positions, optionally wherein the one or more criteria associated with a specific gene synthesis methodology comprise one or more of: avoidance of oligonucleotide overlap regions, availability of a degenerate codon that includes both the reference amino acid and the mutated amino acid, and efficiency by which the degeneracy can be substituted into the sequence by using minimal new oligonucleotide synthesis.

25. The method of any of claims 20 to 24, further comprising:
obtaining one or more of the identified candidate mutant enzymes, optionally by expressing a gene library designed based on the one or more identified candidate mutants, and/or testing one or more of the identified candidate mutant enzymes for one or more properties including catalytic activity and/or testing one or more of the identified candidate mutant enzymes for one or more properties for a property other than catalytic activity; and/or
subjecting an identified candidate enzyme to further optimisation and/or a stabilisation process, optionally wherein the stabilisation process is selected from random mutagenesis, stabilisation of flexible regions, generation of salt bridges, introduction of disulphide bonds, and enzyme supercharging, preferably wherein the stabilisation process is enzyme supercharging; and/or
selecting an identified candidate mutant enzyme or a further optimised version thereof and repeating the method of any of claims 20 to 24 using the selected enzyme as a reference enzyme.
